(19) United States
(12) Patent Application Publication
(10) Pub. No.: US 2007/0059709 A1
(43) Pub. Date: Mar. 15, 2007

(54) Staphylococcus aureus antibacterial target genes

(76) Inventors: Bret Benton, Burlingame, CA (US); Ving J. Lee, Los Altos, CA (US); Francois Malouin, Los Gatos, CA (US); Patrick K. Martin, Sunnyvale, CA (US); Molly B. Schmid, Menlo Park, CA (US); Dongxu Sun, Cupertino, CA (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 10/624,725

(22) Filed: Jul. 21, 2003

Related U.S. Application Data
(63) Continuation of application No. 09/527,745, filed on Mar. 17, 2000, now Pat. No. 6,638,718.
(62) Division of application No. 09/265,315, filed on Mar. 9, 1999, now Pat. No. 6,187,541.
(62) Division of application No. 08/714,918, filed on Sep. 13, 1996, now Pat. No. 6,037,123.
(60) Provisional application No. 60/009,102, filed on Dec. 22, 1995.

(51) Int. Cl. C12Q 1/68; G01N 33/554; A61K 31/4196; A61K 31/4439

(52) U.S. Cl. 435/6; 435/7.32; 514/341; 514/383

(57) ABSTRACT

This disclosure describes isolated or purified deoxyribonucleotide (DNA) sequences, useful for the development of antibacterial agents, which contain the coding sequences of bacterial pathogenesis genes or essential genes, which are expressed in vivo. It further describes isolated or purified DNA sequences which are portions of such bacterial genes, which are useful as probes to identify the presence of the corresponding gene or the presence of a bacteria containing that gene. Also described are hypersensitive mutant cells containing a mutant gene corresponding to any of the identified sequences and methods of screening for antibacterial agents using such hypersensitive cells. In addition it describes methods of treating bacterial infections by administering an antibacterial agent active against one of the identified targets, as well as pharmaceutical compositions effective in such treatments.

### RELATED APPLICATIONS

This application claims priority to Martin et al., STAPHYLOCOCCUS AUREUS ANTIBACTERIAL TARGET GENES, U.S. Provisional Application No. 60/003,798, filed Sep. 15, 1995, and to Benton et al., STAPHYLOCOCCUS AUREUS ANTIBACTERIAL TARGET GENES, U.S. Provisional Application No. 60/009,102, filed Dec. 22, 1995, which are incorporated herein by reference including drawings.

### BACKGROUND

This invention relates to the field of antibacterial treatments and to targets for antibacterial agents. In particular, it relates to genes essential for survival of a bacterial strain in vitro or in vivo.

The following background information is not admitted to be prior art to the pending claims, but is provided only to aid the understanding of the reader.

Despite the development of numerous antibacterial agents, bacterial infections continue as a major, and currently increasing, medical problem. Prior to the 1980s, bacterial infections in developed countries could be readily treated with available antibiotics. However, during the 1980s and 1990s, antibiotic resistant bacterial strains emerged and have become a major therapeutic problem. There are, in fact, strains resistant to essentially all of the commonly used antibacterial agents, which have been observed in the clinical setting, notably including strains of Staphylococcus aureus. The consequences of the increase in resistant strains include higher morbidity and mortality, longer patient hospitalization, and an increase in treatment costs. (B. Murray, 1994, New Engl. J. Med. 330:1229-1230.) Therefore, there is a pressing need for the development of new antibacterial agents which are not significantly affected by the existing bacterial resistance mechanisms.

Such development of new antibacterial agents can proceed by a variety of methods, but generally fall into at least two categories. The first is the traditional approach of screening for antibacterial agents without concern for the specific target.

The second approach involves the identification of new targets, and the subsequent screening of compounds to find antibacterial agents affecting those targets. Such screening can involve any of a variety of methods, including screening for inhibitors of the expression of a gene, or of the product of a gene, or of a pathway requiring that product. However, generally the actual target is a protein, the inhibition of which prevents the growth or pathogenesis of the bacterium. Such protein targets can be identified by identifying genes encoding proteins essential for bacterial growth.

### SUMMARY

Each pathogenic bacterial species expresses a number of different genes which are essential for growth of the bacteria in vitro or in vivo in an infection, and which are useful targets for antibacterial agents. This invention provides an approach to the identification of those genes, and the use of those genes, and bacterial strains expressing mutant forms of those genes, in the identification, characterization, and evaluation of targets of antibacterial agents. It further provides the use of those genes and mutant strains in screening for antibacterial agents active against the genes, including against the corresponding products and pathways. Such active compounds can be developed into antibacterial agents. Thus, this invention also provides methods of treating bacterial infections in mammals by administering an antibacterial agent active against such a gene, and the pharmaceutical compositions effective for such treatment.

For the Staphylococcus aureus essential genes identified in this invention, the essential nature of the genes was determined by the isolation of growth conditional mutants of Staphylococcus aureus, in this case temperature sensitive mutants (ts mutants). Each gene was then identified by isolating recombinant bacteria derived from the growth conditional mutant strains, which would grow under non-permissive conditions but which were not revertants. These recombinant bacteria contained DNA inserts derived from the normal (i.e., wild-type) S. aureus chromosome which encoded non-mutant products which replaced the function of the products of the mutated genes. The fact that a clone having such a recombinant insert can complement the mutant gene product under non-permissive conditions implies that the insert contains essentially a complete gene, since it produces functional product.

The Staphylococcal genes described herein have either been completely sequenced or have been partially sequenced in a manner which essentially provides the complete gene by uniquely identifying the coding sequence in question, and providing sufficient guidance to obtain the complete sequence and equivalent clones. For example, in some cases, sequences have been provided which can be used to construct PCR primers for amplification of the gene from a genomic sequence or from a cloning vector, e.g., a plasmid. The primers can be transcribed from DNA templates, or preferably synthesized by standard techniques. The PCR process using such primers provides specific amplification of the corresponding gene. Therefore, the complete gene sequence is obtainable by using the sequences provided.

In a first aspect, this invention provides a method of treating a bacterial infection in a mammal by administering a compound which is active against a bacterial gene selected from the group of genes corresponding to SEQ ID NO. 1-105. Each of these genes has been identified as an essential gene by the isolation of growth conditional mutant strains, and the complementation in recombinant strains of each of the mutated genes under non-permissive conditions, by expression from artificially-inserted DNA sequences carrying genes identified by the specified sequences of SEQ ID NO. 1-105. In particular embodiments of this method, the infection involves a bacterial strain expressing a gene corresponding to one of the specified sequences, or a homologous gene. Such homologous genes provide equivalent biological function in other bacterial species. Also in a preferred embodiment, the compound has a structure described by the general structure below:

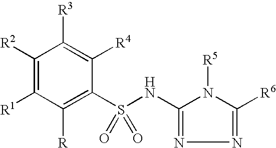

in which

R, R¹, R², and R³ are independently H, alkyl (C₁-C₅), or halogen;

R⁴ is H, alkyl (C₁-C₅), halogen, SH, or S-alkyl (C₁-C₃);

R⁵ is H, alkyl (C¹-C⁵), or aryl (C₆-C₁₀);

R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);

or

R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;

in which

R⁷, R⁸, R⁹, and R¹⁰ are independently H, alkyl (C₁-C₅), halogen, fluoroalkyl (C₁-C₅);

or

R⁷ and R⁸ together are —CH═CH—CH═CH—.

The term “alkyl” refers to a branched or unbranched aliphatic hydrocarbon group, e.g., methyl, ethyl, n-propyl, iso-propyl, and tert-butyl. Preferably the group includes from 1 to 5 carbon atoms and is unsubstituted, but alternatively may optionally be substituted with functional groups which are commonly attached to such chains, e.g., hydroxyl, fluoro, chloro, aryl, nitro, amino, amido, and the like.

The term “halogen” refers to a substituent which is fluorine, chlorine, bromine, or iodine. Preferably the substituent is fluorine.

The term “pyridyl” refers to a group from pyridine, generally having the formula C₅H₄N, forming a heterocyclic ring, which may optionally be substituted with groups commonly attached to such rings.

The term furyl refers to a heterocyclic group, having the formula C₄H₃O, which may be either the alpha or beta isomer. The ring may optionally be substituted with groups commonly attached to such rings.

The term “thienyl refers to a group from thiophen, generally having a formula C₄H₃S

The term “aryl” refers to an aromatic hydrocarbon group which includes a ring structure in which the electrons are delocalized. Commonly, aryl groups contain a derivative of the benzene ring. The ring may optionally be substituted with groups commonly attached to aromatic rings, e.g., OH, CH₃, and the like.

The term “fluoroalkyl” refers to an alkyl group, as described above, which one or more hydrogens are substituted with fluorine.

“Treating”, in this context, refers to administering a pharmaceutical composition for prophylactic and/or therapeutic purposes. The term “prophylactic treatment” refers to treating a patient who is not yet infected, but who is susceptible to, or otherwise at risk, of a particular infection. The term “therapeutic treatment” refers to administering treatment to a patient already suffering from an infection

The term “bacterial infection” refers to the invasion of the host mammal by pathogenic bacteria. This includes the excessive growth of bacteria which are normally present in or on the body of a mammal. More generally, a bacterial infection can be any situation in which the presence of a bacterial population(s) is damaging to a host mammal. Thus, a mammal is “suffering” from a bacterial infection when excessive numbers of a bacterial population are present in or on a mammal's body, or when the effects of the presence of a bacterial population(s) is damaging the cells or other tissue of a mammal.

In the context of this disclosure, “bacterial gene” should be understood to refer to a unit of bacterial heredity as found in the chromosome of each bacterium. Each gene is composed of a linear chain of deoxyribonucleotides which can be referred to by the sequence of nucleotides forming the chain. Thus, “sequence” is used to indicate both the ordered listing of the nucleotides which form the chain, and the chain, itself, which has that sequence of nucleotides. (“Sequence” is used in the same way in referring to RNA chains, linear chains made of ribonucleotides.) The gene includes regulatory and control sequences, sequences which can be transcribed into an RNA molecule, and may contain sequences with unknown function. The majority of the RNA transcription products are messenger RNAs (mRNAs), which include sequences which are translated into polypeptides and may include sequences which are not translated. It should be recognized that small differences in nucleotide sequence for the same gene can exist between different bacterial strains, or even within a particular bacterial strain, without altering the identity of the gene.

Thus, “expressed bacterial gene” means that, in a bacterial cell of interest, the gene is transcribed to form RNA molecules. For those genes which are transcribed into mRNAs, the mRNA is translated to form polypeptides. More generally, in this context, “expressed” means that a gene product is formed at the biological level which would normally have the relevant biological activity (i.e., RNA or polypeptide level).

As used herein in referring to the relationship between a specified nucleotide sequence and a gene, the term “corresponds” or “corresponding” indicates that the specified sequence identifies the gene. Therefore, a sequence which will uniquely hybridize with a gene from the relevant bacterium corresponds to that gene (and the converse). In general, for this invention, the specified sequences have the same sequence (a low level of sequencing error or individual variation does not matter) as portions of the gene or flanking sequences. Similarly, correspondence is shown by a transcriptional, or reverse transcriptional relationship. Many genes can be transcribed to form mRNA molecules. Therefore, there is a correspondence between the entire DNA sequence of the gene and the mRNA which is, or might be, transcribed from that gene; the correspondence is also present for the reverse relationship, the messenger RNA corresponds with the DNA of the gene. This correspondence is not limited to the relationship between the full sequence of the gene and the full sequence of the mRNA, rather it also exists between a portion or portions of the DNA sequence of the gene and a portion or portions of the RNA sequence of the mRNA. Specifically it should be noted that this correspondence is present between a portion or portions of an mRNA which is not normally translated into polypeptide and all or a portion of the DNA sequence of the gene.

Similarly, the DNA sequence of a gene or the RNA sequence of an mRNA “corresponds” to the polypeptide encoded by that gene and mRNA. This correspondence between the mRNA and the polypeptide is established through the translational relationship; the nucleotide sequence of the mRNA is translated into the amino acid sequence of the polypeptide. Then, due to the transcription relationship between the DNA of the gene and the mRNA, there is a “correspondence” between the DNA and the polypeptide.

The term “administration” or “administering” refers to a method of giving a dosage of an antibacterial pharmaceutical composition to a mammal, where the method is, e.g., topical, oral, intravenous, transdermal, intraperitoneal, or intramuscular. The preferred method of administration can vary depending on various factors, e.g., the components of the pharmaceutical composition, the site of the potential or actual bacterial infection, the bacterium involved, and the severity of an actual bacterial infection.

The term “active against” in the context of compounds, agents, or compositions having antibacterial activity indicates that the compound exerts an effect on a particular bacterial target or targets which is deleterious to the in vitro and/or in vivo growth of a bacterium having that target or targets. In particular, a compound active against a bacterial gene exerts an action on a target which affects an expression product of that gene. This does not necessarily mean that the compound acts directly on the expression product of the gene, but instead indicates that the compound affects the expression product in a deleterious manner. Thus, the direct target of the compound may be, for example, at an upstream component which reduces transcription from the gene, resulting in a lower level of expression. Likewise, the compound may affect the level of translation of a polypeptide expression product, or may act on a downstream component of a biochemical pathway in which the expression product of the gene has a major biological role. Consequently, such a compound can be said to be active against the bacterial gene, against the bacterial gene product, or against the related component either upstream or downstream of that gene or expression product. While the term “active against” encompasses a broad range of potential activities, it also implies some degree of specificity of target. Therefore, for example, a general protease is not “active against” a particular bacterial gene which produces a polypeptide product. In contrast, a compound which inhibits a particular enzyme is active against that enzyme and against the bacterial gene which codes for that enzyme.

The term “mammal” refers to any organism of the Class Mammalia of higher vertebrates that nourish their young with milk secreted by mammary glands, e.g., mouse, rat, and, in particular, human, dog, and cat.

By “comprising” it is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A DNA containing a specific bacterial gene is obtainable using a shorter, unique probe(s) with readily available molecular biology techniques. If the method for obtaining such gene is properly performed, it is virtually certain that a longer DNA sequence comprising the desired sequence (such as the full coding sequence or the full length gene sequence) will be obtained. Thus, “obtainable by” means that an isolation process will, with high probability (preferably at least 90%), produce a DNA sequence which includes the desired sequence. Thus, for example, a full coding sequence is obtainable by hybridizing the DNA of two PCR primers appropriately derived from the sequences of SEQ ID NO. 1-105 corresponding to a particular complementing clone to a Staphylococcus aureus chromosome, amplifying the sequence between the primers, and purifying the PCR products. The PCR products can then be used for sequencing the entire gene or for other manipulations. Those skilled in the art will understand the included steps, techniques, and conditions for such processes. However, the full coding sequence or full gene is clearly not limited to a specific process by which the sequence is obtainable. Such a process is only one method of producing the final product.

A “coding sequence” or “coding region” refers to an open reading frame (ORF) which has a base sequence which is normally transcribed in a cell (e.g., a bacterial cell) to form RNA, which in most cases is translated to form a polypeptide. For the genes for which the product is normally a polypeptide, the coding region is that portion which encodes the polypeptide, excluding the portions which encode control and regulatory sequences, such as stop codons and promoter sequences.

In a related aspect, the invention provides a method for treating a bacterial infection in a mammal by administering an amount of an antibacterial agent effective to reduce the infection. The antibacterial agent specifically inhibits a biochemical pathway requiring the expression product of a gene corresponding to one of the genes identified in the first aspect above. Inhibition of that pathway inhibits the growth of the bacteria in vivo. In particular embodiments, the antibacterial agent inhibits the expression product of one of the identified genes.

In the context of the coding sequences and genes of this invention, “homologous” refers to genes whose expression results in expression products which have a combination of amino acid sequence similarity (or base sequence similarity for transcript products) and functional equivalence, and are therefore homologous genes. In general such genes also have a high level of DNA sequence similarity (i.e., greater than 80% when such sequences are identified among members of the same genus, but lower when these similarities are noted across bacterial genera), but are not identical. Relationships across bacterial genera between homologous genes are more easily identified at the polypeptide (i.e., the gene product) rather than the DNA level. The combination of functional equivalence and sequence similarity means that if one gene is useful, e.g., as a target for an antibacterial agent, or for screening for such agents, then the homologous gene is likewise useful. In addition, identification of one such gene serves to identify a homologous gene through the same relationships as indicated above. Typically, such homologous genes are found in other bacterial species, especially, but not restricted to, closely related species. Due to the DNA sequence similarity, homologous genes are often identified by hybridizing with probes from the initially identified gene under hybridizing conditions which allow stable binding under appropriately stringent conditions (e.g., conditions which allow stable binding with approximately 85% sequence identity). The equivalent function of the product is then verified using appropriate biological and/or biochemical assays.

In this context, the term “biochemical pathway” refers to a connected series of biochemical reactions normally occurring in a cell, or more broadly a cellular event such as cellular division or DNA replication. Typically, the steps in such a biochemical-pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent, may, but does not necessarily, act directly on the expression product of that particular gene.

The term “in vivo” in the context of a bacterial infection refers to the host infection environment, as distinguished, for example, from growth of the bacteria in an artificial culture medium (e.g., in vitro).

The term “antibacterial agent” refers to both naturally occurring antibiotics produced by microorganisms to suppress the growth of other microorganisms, and agents synthesized or modified in the laboratory which have either bactericidal or bacteriostatic activity, e.g., β-lactam antibacterial agents, glycopeptides, macrolides, quinolones, tetracyclines, and aminoglycosides. In general, if an antibacterial agent is bacteriostatic, it means that the agent essentially stops bacterial cell growth (but does not kill the bacteria); if the agent is bacteriocidal, it means that the agent kills the bacterial cells (and may stop growth before killing the bacteria).

The term, “bacterial gene product” or “expression product” is used to refer to a polypeptide or RNA molecule which is encoded in a DNA sequence according to the usual transcription and translation rules, which is normally expressed by a bacterium. Thus, the term does not refer to the translation of a DNA sequence which is not normally translated in a bacterial cell. However, it should be understood that the term does include the translation product of a portion of a complete coding sequence and the translation product of a sequence which combines a sequence which is normally translated in bacterial cells translationally linked with another DNA sequence. The gene product can be derived from chromosomal or extrachromosomal DNA, or even produced in an in vitro reaction. Thus, as used herein, an “expression product” is a product with a relevant biological activity resulting from the transcription, and usually also translation, of a bacterial gene.

In another related aspect, the invention provides a method of inhibiting the growth of a pathogenic bacterium by contacting the bacterium with an antibacterial agent which specifically inhibits a biochemical pathway requiring the expression product of a gene selected from the group of genes corresponding to SEQ ID NO. 1-105 or a homologous gene. Inhibition of that pathway inhibits growth of the bacterium. In particular embodiments, the antibacterial agent inhibits the expression product of one of the identified genes. Also in preferred embodiment, the antibacterial agent is a compound having a structure as described in the first aspect above.

The term “inhibiting the growth” indicates that the rate of increase in the numbers of a population of a particular bacterium is reduced. Thus, the term includes situations in which the bacterial population increases but at a reduced rate, as well as situations where the growth of the population is stopped, as well as situations where the numbers of the bacteria in the population are reduced or the population even eliminated.

A “pathogenic bacterium” includes any bacterium capable of infecting and damaging a mammalian host, and, in particular, includes Staphylococcus aureus. Thus, the term includes both virulent pathogens which, for example, can cause disease in a previously healthy host, and opportunistic pathogens which can only cause disease in a weakened or otherwise compromised host.

Similarly, the invention provides a method of prophylactic treatment of a mammal by administering a compound active against a gene selected from the group of genes corresponding to SEQ ID NO. 1-105 to a mammal at risk of a bacterial infection.

A mammal may be at risk of a bacterial infection, for example, if the mammal is more susceptible to infection or if the mammal is in an environment in which infection by one or more bacteria is more likely than in a normal setting. Therefore, such treatment can, for example, be appropriate for an immuno-compromised patient.

Also provided is a method of screening for an antibacterial agent by determining whether a test compound is active against one of the genes identified in the first aspect. In a particular embodiment the method is performed by providing a bacterial strain having a mutant form of a gene selected from the group of genes corresponding to SEQ. ID. NOS. 1-105 or a mutant gene homologous to one of those genes. The mutant form of the gene confers a growth conditional phenotype, e.g., a temperature-sensitive phenotype, on the bacterial strain having that mutant form. A comparison bacterial strain having a normal form of the gene is also provided and the two strains of bacteria are separately contacted with a test compound under semi-permissive growth conditions. The growth of the two strains in the presence of the test compound is then compared; a reduction in the growth of the bacterial strain having the mutant form compared to the growth of the bacterial strain having the normal form of the gene indicates that the test compound is active against the particular gene.

In this context, a “mutant form” of a gene is a gene which has been altered, either naturally or artificially, changing the base sequence of the gene, which results in a change in the amino acid sequence of an encoded polypeptide. The change in the base sequence may be of several different types, including changes of one or more bases for different bases, small deletions, and small insertions. By contrast, a normal form of a gene is a form commonly found in a natural population of a bacterial strain. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the bacterial strain having that gene, while a mutant form of a gene suitable for use in these methods does provide such a growth conditional phenotype.

As used in this disclosure, the term “growth conditional phenotype” indicates that a bacterial strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a bacterial strain having a heat-sensitive phenotype) exhibits significantly reduced growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

Thus, “semi-permissive conditions” are conditions in which the relevant culture parameter for a particular growth conditional phenotype is intermediate between permissive conditions and non-permissive conditions. Consequently, in semi-permissive conditions the bacteria having a growth conditional phenotype will exhibit growth rates intermediate between those shown in permissive conditions and non-permissive conditions. In general, such intermediate growth rate is due to a mutant cellular component which is partially functional under semi-permissive conditions, essentially fully functional under permissive conditions, and is non-functional or has very low function under non-permissive conditions, where the level of function of that component is related to the growth rate of the bacteria.

The term “method of screening” means that the method is suitable, and is typically used, for testing for a particular property or effect in a large number of compounds. Therefore, the method requires only a small amount of time for each compound tested, typically more than one compound is tested simultaneously (as in a 96-well microtiter plate), and preferably significant portions of the procedure can be automated. “Method of screening” also refers to determining a set of different properties or effects of one compound simultaneously.

Since the essential genes identified herein can be readily isolated and the gene products expressed by routine methods, the invention also provides the polypeptides encoded by those genes. Thus, the invention provides a method of screening for an antibacterial agent by determining the effects of a test compound on the amount or level of activity of a polypeptide gene product of one of the identified essential genes. The method involves contacting cells expressing such a polypeptide with a test compound, and determining whether the test compound alters the amount or level of activity of the expression product. The exact determination method will be expected to vary depending on the characteristics of the expression product. Such methods can include, for example, antibody binding methods, enzymatic activity determinations, and substrate analog binding assays.

It is quite common in identifying antibacterial agents, to assay for binding of a compound to a particular polypeptide where binding is an indication of a compound which is active to modulate the activity of the polypeptide. Thus, by identifying certain essential genes, this invention provides a method of screening for an antibacterial agent by contacting a polypeptide encoded by one of the identified essential genes, or a biologically active fragment of such a polypeptide, with a test compound, and determining whether the test compound binds to the polypeptide or polypeptide fragment.

In addition, to simple binding determinations, the invention provides a method for identifying or evaluating an agent active on one of the identified essential genes. The method involves contacting a sample containing an expression product of one of the identified genes with the known or potential agent, and determining the amount or level of activity of the expression product in the sample.

In a further aspect, this invention provides a method of diagnosing the presence of a bacterial strain having one of the genes identified above, by probing with an oligonucleotide at least 15 nucleotides in length, which specifically hybridizes to a nucleotide sequence which is the same as or complementary to the sequence of one of the bacterial genes identified above. In some cases, it is practical to detect the presence of a particular bacterial strain by direct hybridization of a labeled oligonucleotide to the particular gene. In other cases, it is preferable to first amplify the gene or a portion of the gene before hybridizing labeled oligonucleotides to those amplified copies.

In a related aspect, this invention provides a method of diagnosing the presence of a bacterial strain by specifically detecting the presence of the transcriptional or translational product of the gene. Typically, a transcriptional (RNA) product is detected by hybridizing a labeled RNA or DNA probe to the transcript. Detection of a specific translational (protein) product can be performed by a variety of different tests depending on the specific protein product. Examples would be binding of the product by specific labeled antibodies and, in some cases, detection of a specific reaction involving the protein product.

As used above and throughout this application, “hybridize” has its usual meaning from molecular biology. It refers to the formation of a base-paired interaction between nucleotide polymers. The presence of base pairing implies that at least an appreciable fraction of the nucleotides in each of two nucleotide sequences are complementary to the other according to the usual base pairing rules. The exact fraction of the nucleotides which must be complementary in order to obtain stable hybridization will vary with a number of factors, including nucleotide sequence, salt concentration of the solution, temperature, and pH.

The term, “DNA molecule”, should be understood to refer to a linear polymer of deoxyribonucleotides, as well as to the linear polymer, base-paired with its complementary strand; forming double-strand DNA (dsDNA). The term is used as equivalent to “DNA chain” or “a DNA” or “DNA polymer” or “DNA sequence”:, so this description of the term meaning applies to those terms also. The term does not necessarily imply that the specified “DNA molecule” is a discrete entity with no bonding with other entities. The specified DNA molecule may have H-bonding interactions with other DNA molecules, as well as a variety of interactions with other molecules, including RNA molecules. In addition, the specified DNA molecule may be covalently linked in a longer DNA chain at one, or both ends. Any such DNA molecule can be identified in a variety of ways, including, by its particular nucleotide sequence, by its ability to base pair under stringent conditions with another DNA or RNA molecule having a specified sequence, or by a method of isolation which includes hybridization under stringent conditions with another DNA or RNA molecule having a specified sequence.

References to a “portion” of a DNA or RNA chain mean a linear chain which has a nucleotide sequence which is the same as a sequential subset of the sequence of the chain to which the portion refers. Such a subset may contain all of the sequence of the primary chain or may contain only a shorter sequence. The subset will contain at least 15 bases in a single strand.

However, by “same” is meant “substantially the same”; deletions, additions, or substitutions of specific nucleotides of the sequence, or a combination of these changes, which affect a small percentage of the full sequence will still leave the sequences substantially the same. Preferably this percentage of change will be less than 20′, more preferably less than 10%, and even more preferably less than 3%. “Same” is therefore distinguished from “identical”; for identical sequences there cannot be any difference in nucleotide sequences.

As used in reference to nucleotide sequences, “complementary” has its usual meaning from molecular biology. Two nucleotide sequences or strands are complementary if they have sequences which would allow base pairing between the strands according to the usual pairing rules. This does not require that the strands would necessarily base pair at every nucleotide; two sequences can still be complementary with a low level of base mismatch such as that created by deletion, addition, or substitution of one or a few (up to 5 in a linear chain of 25 bases) nucleotides, or a combination of such changes.

Further, in another aspect, this invention provides a pharmaceutical composition appropriate for use in the methods of treating bacterial infections described above, containing a compound active on a bacterial gene selected from the group of genes described above and a pharmaceutically acceptable carrier. In a preferred embodiment, the compound has a structure as described in the first aspect above. Also, in a related aspect the invention provides a novel compound having antibacterial activity against one of the bacterial genes described above.

In a further related aspect a method of making an antibacterial agent is provided. The method involves screening for an agent active on one of the identified essential genes by providing a bacterial strain having a mutant form of one of the genes corresponding to SEQ ID NO. 1-105, or a homologous gene. As described above, the mutant form of the gene confers a growth conditional phenotype. A comparison bacterial strain is provided which has a normal form of said gene. The bacterial strains are contacted with a test compound in semi-permissive growth conditions, and the growth of the strains are compared to identify an antibacterial agent. The identified agent is synthesized in an amount sufficient to provide the agent in a therapeutically effective amount to a patient.

A “carrier” or “excipient” is a compound or material used to facilitate administration of the compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as peanut and sesame oils. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Consistent with the usage of “anti-bacterial agent” herein, the term “anti-bacterial activity” indicates that the presence of a particular compound in the growth environment of a bacterial population reduces the growth rate of that population, without being a broad cellular toxin for other categories of cells.

As is described below in the Detailed Description of the Preferred Embodiments, bacterial strains expressing a mutated form of one of the above identified genes, which confers a growth conditional phenotype, are useful for evaluating and characterizing the gene as an antibacterial target and for screening for antibacterial agents. Therefore, this invention also provides a purified bacterial strain expressing a mutated gene which is a mutated form of one of the bacterial genes identified above, where the mutated gene confers a growth conditional phenotype.

Similarly, this invention provides a recombinant bacterial cell containing an artificially inserted DNA construct which contains a DNA sequence which is the same as or complementary to one of the above-identified bacterial genes or a portion of one of those genes. Such cells are useful, for example, as sources of probe sequences or for providing a complementation standard for use in screening methods.

The term “recombinant bacterial cell” has its usual molecular biological meaning. The term refers to a microbe into which has been inserted, through the actions of a person, a DNA sequence or construct which was not previously found in that cell, or which has been inserted at a different location within the cell, or at a different location in the chromosome of that cell. Such a term does not include natural genetic exchange, such as conjugation between naturally occurring organisms. Thus, for example, a recombinant bacterium could have a DNA sequence inserted which was obtained from a different bacterial species, or may contain an inserted DNA sequence which is an altered form of a sequence normally found in that bacteria.

As described above, the presence of a specific bacterial strain can be identified using oligonucleotide probes. Therefore this invention also provides such oligonucleotide probes at least 15 nucleotides in length, which specifically hybridize to a nucleotide sequence which is the same as or complementary to a portion of one of the bacterial chains identified above.

In a related aspect this invention provides an isolated or purified DNA sequence at least 15 nucleotides in length, which has a nucleotide base sequence which is the same as or complementary to a portion of one of the above-identified bacterial genes. In particular embodiments, the DNA sequence is the same as or complementary to the base sequence of the entire coding region of one of the above-identified bacterial genes. Such an embodiment may in addition contain the control and regulatory sequence associated with the coding sequence.

Use of the term “isolated” indicates that a naturally occurring material or organism (e.g., a DNA sequence) has been removed from its normal environment. Thus, an isolated DNA sequence has been removed from its usual cellular environment, and may, for example, be in a cell-free solution or placed in a different cellular environment. For a molecule, such as a DNA sequence, the term does not imply that the molecule (sequence) is the only molecule of that type present.

It is also advantageous for some purposes that an organism or molecule (e.g., a nucleotide sequence) be in purified form. The term “purified” does not require absolute purity; instead, it indicates that the sequence, organism, or molecule is relatively purer than in the natural environment. Thus, the claimed DNA could not be obtained directly from total human DNA or from total human RNA. The claimed DNA sequences are not naturally occurring, but rather are obtained via manipulation of a partially purified naturally occurring substance (genomic DNA clones). The construction of a genomic library from chromosomal DNA involves the creation of vectors with genomic DNA inserts and pure individual clones carrying such vectors can be isolated from the library by clonal selection of the cells carrying the library.

In a further aspect, this invention provides an isolated or purified DNA sequence which is the same as or complementary to a bacterial gene homologous to one of the above-identified bacterial genes where the function of the expression product of the homologous gene is the same as the function of the product of one of the above-identified genes. In general, such a homologous gene will have a high level of nucleotide sequence similarity and, in addition, a protein product of homologous gene will have a significant level of amino acid sequence similarity. However, in addition, the product of the homologous gene has the same biological function as the product of the corresponding gene identified above.

Similarly, the invention provides an isolated or purified DNA sequence which has a base sequence which is the same as the base sequence of a mutated bacterial gene selected from one of the genes identified in the first aspect where the expression of this DNA sequence or the mutated bacterial gene confers a growth conditional phenotype in the absence of expression of a gene which complements that mutation. Such an isolated or purified DNA sequence can have the base sequence which varies slightly from the base sequence of the original mutated gene but must contain a base sequence change or changes which are functionally equivalent to the base sequence change or changes in the mutated gene. In most cases, this will mean that the DNA sequence has the identical bases at the site of the mutation as the mutated gene.

As indicated above, by providing the identified essential genes, the encoded expression products are also provided. Thus, another aspect concerns a purified, enriched, or isolated polypeptide, which is encoded by one of the identified essential genes. Such a polypeptide may include the entire gene product or only a portion or fragment of the encoded product. Such fragments are preferably biologically active fragments which retain one or more of the relevant biological activities of the full size gene product.

Other features and advantages of the invention will be apparent from the following description of the preferred embodiments, and from the claims.

### BRIEF DESCRIPTION OF THE FIGURES

FIG. 1 shows the fold increase in sensitivity toward 12 antibacterial agents and a generally toxic agent for 3 temperature sensitive mutants of Salmonella typhimurium. These are mutants of DNA gyrase subunit A (gyrA212, gyrA215, and gyrA216, grown at a semi-permissive temperature (35_C). Hypersensitivity is observed to antibacterial agents acting on DNA gyrase, but not to other classes of drugs or toxic agents. The data demonstrate that growth conditional mutations in a known target cause hypersensitivity to target inhibitors.

FIG. 2 presents the hypersensitivity profiles of a set of temperature sensitive mutants of Salmonella, for a variety of antibacterial agents with characterized modes of action, compared to the sensitivity profile of wild type.

FIG. 3 illustrates a variety of types of interactions which exist between different essential genes, and which can create differential responses in screens using growth conditional mutants.

FIG. 4 illustrates a possible arrangement of a multichannel screen plate using conditional growth mutants with mutations affecting 5 different cellular processes plus controls.

FIG. 5 illustrates 2 alternative multichannel screen designs in which either multiple compounds are screened using a single growth conditional mutant on each plate, or in which multiple growth conditional mutants are used on each plate to create an inhibition profile of a single compound.

FIG. 6 is a bar graph showing the different heat sensitivity profiles for 6 S. aureus heat sensitive mutant strains. The growth of each strain is shown at 6 different temperatures ranging from 30° C. to 43° C.

FIG. 7 is a bar graph showing the different heat sensitivity profiles for 4 different S. aureus polC heat sensitive mutants and a wild type strain. The growth of each strain is shown at 6 different temperatures ranging from 30° C. to 43° C.

FIG. 8 is a graph showing the differences in hypersensitivity of one S. aureus heat sensitive strain (NT99) toward 30 inhibitory compounds at 3 different temperatures.

FIG. 9 is a diagram for two S. aureus mutants, illustrating that a greater number of growth inhibitory hits are identified at higher temperatures using heat sensitive mutants. Compounds were identified as hits if the growth of the mutant was inhibited by at least 50% and the inhibition of growth of the mutant was at least 30% higher than the inhibition of growth of a wild type strain.

FIG. 10 is a bar diagram illustrating the effect of test compound concentration on the number of hits identified, showing that, in general, more compounds are identified as hits at higher concentrations.

FIG. 11 presents the structures of two compounds which exhibited the same inhibition profiles for a set of temperature sensitive Staphylococcus aureus mutants, showing the structural similarity of the compounds.

FIG. 12 presents the fold increase in sensitivity of a set of Staphylococcus aureus temperature sensitive mutants for a variety of compounds which inhibit growth of Staphylococcus aureus wild type, but which have uncharacterized targets of action.

FIG. 13 illustrates the types of anticipated inhibition profiles of different growth conditional mutants for a variety of test compounds, indicating that the number of mutants affected by a particular compound is expected to vary.

FIG. 14 shows the proportion of compounds (from a total of 65) which significantly inhibited the growth of varying numbers of temperature sensitive mutants in a screen of uncharacterized growth inhibitors of Staphylococcus aureus.

FIG. 15 shows the potency (MIC values) of a number of growth inhibitors which affected 0, 1 or more than 3 temperature sensitive mutants of Staphylococcus aureus in a screen of uncharacterized growth inhibitors.

FIG. 16 shows the number of hits for each of the temperature sensitive mutants of Staphylococcus aureus in a screen of 65 uncharacterized growth inhibitors.

FIG. 17 shows some advantages of a multichannel genetic potentiation screen using growth conditional mutants over traditional biochemical screens with either a known target or an unknown cloned gene.

FIG. 18 illustrates a strategy for selecting dominant lethal mutants for use in screens for antibacterial agents, not requiring hypersensitivity.

FIG. 19A-D are structures of four compounds which were identified as hits on mutant NT94.

FIG. 20 is a partial restriction map of the S. aureus clone insert (complementing mutant NT64), showing the position of the initial left and right sequences obtained.

FIGS. 21-90 are partial restriction maps of each of the S. aureus clone inserts for which sequences are described herein, showing the relative fraction of the insert for which nucleotide sequence is described, as well as the approximate positions of identified open reading frames (ORFs).

### DESCRIPTION OF THE PREFERRED EMBODIMENTS

I. General Approach for Identification of Target Genes

As was briefly described in the Summary above, this invention concerns essential genes in Staphylococcus aureus. This organism is a serious pathogen which frequently carries resistance to a variety of existing antibiotic agents. Such resistant strains of S. aureus are a particular problem in settings where antibacterial agents are intensively used, such as in hospitals. To overcome the therapeutic difficulties posed by the existing resistant strains, it is highly desirable that new classes of antibiotic drugs be found, particularly ones which are active against new bacterial targets While such bacterial targets are usually (though not always) proteins, the targets can be identified by first identifying the bacterial genes which encode proteins (or RNA transcripts) that are essential for growth of the bacteria.

Identification of these genes which are essential for growth of the bacteria was accomplished by isolating conditional lethal mutant strains. Such mutant strains will grow under permissive conditions, but will not grow, or grow very poorly under non-permissive conditions. For the bacterial genes described herein, temperature sensitive mutants provided the growth conditional phenotype. The particular gene in each strain which was mutated to confer a growth conditional phenotype was then identified by isolating recombinant derivatives of the mutant strains. These recombinant strains each contained a DNA insert which, when expressed, would complement the defective gene and thus would allow growth under non-permissive conditions. These DNA inserts were provided by a genomic library of a normal S. aureus chromosome. The ability of the DNA insert in the recombinant strain to complement the defective product of the mutated gene showed that the DNA insert contained essentially a complete gene corresponding to a particular mutated gene. The vectors carrying each of these DNA inserts were constructed such that the S. aureus chromosomal insert could be amplified by PCR using flanking primer sequences. Each of the amplified S. aureus inserts was then partially sequenced, in general from both the 5′ and 3′ ends. This sequencing was, in general, single pass sequencing and, thus, the specified sequences may contain a low level of sequence errors compared to the actual gene sequence. Since the partial sequences at the 5′ and 3′ ends bracket the complete gene, such partial sequences uniquely identify and provide that complete gene without interference from a low level of sequencing error. The complete gene and gene sequence can be reliably obtained by any of several different methods. For example, probes can be constructed based on the partial sequences provided, which can be used to probe genomic or cDNA libraries of S. aureus. Clones containing the corresponding 5′ and 3′ sequences can then be further characterized and sequenced to provide the complete gene. In another approach, the partial 5′ and 3′ sequences can be used to construct PCR primer sequences which can be used to amplify the sequence between those primers and likewise provide the complete gene. In yet another approach, equivalent growth conditional mutant strains can be obtained by following the same or a similar process of mutagenizing the base S. aureus strain, and then likewise obtaining the complete gene by isolating complementing clones which correspond to the sequences provided, from a genomic or cDNA library. It should again be noted that, for any of these approaches, a low level of sequencing error in the sequence presented herein does not matter, since the stringency of the hybridizing conditions can be readily adjusted to provide the appropriately specific binding. While the genes identified in this invention are highly useful as targets for novel antibacterial therapy, the genes and parts of those genes are also useful to provide probes which can be used to identify the presence of a particular bacteria carrying a particular gene. In addition, the growth conditional mutant strains described above are also useful as tools in methods for screening for antibacterial agents which target that gene (targeting the corresponding normal gene). The methods involved in the identification of the mutant strains complementing recombinant clones and the particular genes are described in more detail below.

A. Bacterial Strain Selection

The growth conditional mutant strains and recombinant strains herein are based on S. aureus strain 8325-4. This strain has been the subject of substantial genetic characterization and is appropriate for use in the approach described herein. It is believed to be free of transposons, phage or extrachromosomal elements. Numerous other strains of S. aureus can likewise be used. However, it is advantageous to select a strain which has few, or preferably no, transposons or extrachromosomal elements, as such elements can complicate the genetic analysis.

B. Isolation of Conditional Lethal Mutants (General).

Heat-sensitive mutants were obtained after diethyl sulfate (DES; SIGMA Chemical) mutagenesis of strain 8325-4. Briefly, single colonies were inoculated into LB broth in individual wells of a 96-well microtiter plate and grown overnight (35° C. 18 h). Culture supernatants (10 μl) were diluted into λ-dilution buffer (λdil; 500 μl) and then treated with DES (5 μl). After a short incubation period (20 min at 37° C.), the treated cultures were serially diluted with λdil into microtiter plates. After an additional incubation period (8-12 h. at 37° C.), appropriate dilutions (50 μl each of 10 E-2 and 10 E-3) were plated onto TS agar plates and incubated overnight (30° C. 18 h). The plates were replica-printed onto two Tryptic-soy (TS) plates and incubated either at 30° C. or 43° C. (permissive and non-permissive conditions, respectively). Colonies growing at 30° C. but not at 43° C. were isolated and their ts phenotype was subsequently confirmed in a second round of plating. Only one ts mutant was picked from an original singe-colony culture to assure that the mutants isolated were independent from each other. Independently-derived colonies with the appropriate phenotype are identified by direct screening on rich solid media at a permissive temperature (30° C.), as it obviates, selection of mutants deficient in metabolic pathways, such as aromatic amino acid biosynthesis. No penicillin enrichment is employed, as it would counterselect mutant strains that are strongly bactericidal at the non-permissive temperature. A preliminary collection of 100 independent condition-lethal mutants and 71 non-independent mutants was made. This collection has been supplemented with additional condition-lethal mutants.

C. Creation of the S. aureus Shuttle Library

The S. aureus strain used for the preparation of genomic DNA for library construction as well as for the generation of conditional-lethal (temperature sensitive) mutants described in this document is a derivative of NCTC 8325, designated as 8325-4 (Novick, R. P., 1990). The 8325 parent strain is one of the better-characterized strains of S. aureus, with genetic and physical map data available in the current literature (Pattee, P. A., 1990). The 8325-4 derivative strain has all the chromosomal elements of the parent, with the exception of integrated (i.e., prophage and transposon DNA) and extrachromosomal (i.e., plasmid DNA) elements endogenous to the parent.

Cloning and subcloning experiments utilized the commercially-available E. coli strains JM109 (Promega) and DH5alpha (GIBCO-BRL). All enzymes cited (i.e., restriction endonucleases, ligases and phosphatases) were obtained commercially (NEB, Promega). All DNA cloning and manipulations are described in the current literature (Sambrook, et al., 1989). Parent plasmids pE194 and pUC19 have been described previously (Horinouchi, S. et al., 1982; Yanisch-Perron, C. et al., 1985) Recombinant constructs for use in a S. aureus host were first electroporated (Gene Pulser, BioRad) into S. aureus strain RN4220 (a restriction-deficient but methylase-proficient strain; Novick, R. P., 1990) before transduction into the target strain for complementation and cross-complementation analyses.

D. Library Construction

The shuttle plasmid vector used was pMP16, constructed by cloning the entire length of the natural S. aureus plasmid pE194 (linearized with Cla I) into the Nar I site of pUC19 (Yanisch-Perron et al., 1985). This new construct replicates and offers antibiotic resistance selections in both E. coli and S. aureus. It also provides blue-white screening to facilitate scoring of insert-containing clones. Carefully purified genomic DNA from S. aureus strain 8325-4 was partially digested (Sau3A I) and fragments of 2-8 kb were isolated by sucrose gradient centrifugation. DNA fragments isolated in this manner were then used for constructing two different libraries. In library A, the DNA fragments were directly cloned into pMP16, which had been linearized (Bam HI) and dephosphorylated (CIP). The DNA mixture was ligated (T4 DNA ligase) and transformed into E. coli DH5alpha. Library A thus constructed contains about 60,000 independent clones, 60% of which have inserts. In constructing library B, the ends of the Sau3A I fragments were partially filled with dGTP and dATP, ligated with linearized (Sal I) pMP16 that was partially filled with dCTP and dTTP, and transformed into E. coli. The advantage of partially filling the ends is that DNAs with the same ends can no longer ligate to each other; the majority of the ligation occurs between the vector and inserts, significantly increasing the percentage of insert-containing clones. In addition, the chance that two unrelated insert fragment are fortuitously ligated in the same clone is greatly reduced by using this strategy. Library B consists of 50,000 independent clones with >98% containing inserts. Both library A and library B contain at least a 50-fold representation of the S. aureus genome.

Clones from the two libraries were pooled and plasmid DNA extracted. The DNAs were used to transform S. aureus strain RN4220. About 100,000 erythromycin resistant transformants were pooled and infected with bacteriophage φ11 at a multiplicity of infection (MOI) of 0.01 to generate phage lysates containing the shuttle library plasmids. The lysates were then used to introduce the shuttle plasmids into ts mutants by transduction to isolate complementing clones.

E. Isolation of Complementing Clones (General)

The lysate from library B was first chosen for transduction of the ts mutants because of its higher insert frequency. The ts mutants were grown either in TS broth or on TS agar plates overnight (18 h). The cells were resuspended in TS broth containing CaCl₂ (5 mM) to an OD₆₀₀ between 2-3. The lysate from library B (10-50 μl) was added to the resuspended cells (2 ml) and incubated at 30° C. with slow shaking (20 m). Ice-cold sodium citrate (20 mM; 1 ml) was added and the culture was centrifuged to pellet the cells. After removing the supernatant, the pellet was resuspended in ice-cold sodium citrate (20 mM; 500 μl). A small aliquot (about 1/5000 of the total volume) was plated on a TSA-ery-citrate plate (TS agar containing 5 μg/ml erythromycin and 500 μg/ml sodium citrate) and incubated at 30° C. overnight (18 h). The total number of erythromycin-resistant transductants screened were estimated from this plate; at least 200,000 transductants were screened for each ts mutant to assure that the library population was well represented. The rest of the cells were plated onto the same selection media (3-5 plates), incubated at 30° C. for 5 h and then at 43° C. overnight (18 h). Individual colonies that appeared on the 43° C. plates were isolated and infected with φ11 to generate lysates.

The lysates prepared from these individual colonies were then used to transduce the same ts mutants as described above, using much smaller volumes of cells (0.1 ml) and lysates (1-3 μl) to facilitate testing of large number of lysates. Equal amounts of the transduced cultures were plated onto two sets of TSA-ery-citrate plates and incubated at either 30 or 43° C. Individual lysates that generated similar numbers of transductants at 30 and 43° C. were scored as complementing clones. Among the first 96 ts mutants studied, complementing clones were isolated for 60 (63%) of the mutants; 57 were from library B and 3 were from library A.

To test whether different ts mutants carry mutations in the same or closely linked genes, cross complementation was performed to evaluate the ability of positive clones of one ts mutant to complement another mutant. The results showed that, while some positive clones failed to complement any ts mutants other than their primary mutant, other clones were able to complement additional mutants. Taken together, the cross complementation studies identified 38 loci on the S. aureus chromosome, each consisting of at least one essential gene.

All the positive clones for the 60 ts mutants were twice streaked on TSA-ery-citrate plates and grown at 43° C. to eliminate φ11 prophage from the host cells. Plasmid DNA was extracted from these complementing clones and transformed into E. coli. The plasmids were prepared from the E. coli clones and used for restriction mapping and subcloning of the inserts.

F. Strategy for DNA Sequencing of Complementing Clones (General)

Complementing clones were subcloned into a sequencing vector (pGEM3Zf(+); Promega) containing regions of DNA flanking the multiple cloning site (T7 and SP6 primer annealing sites) to facilitate plasmid-based automated sequencing. Clones larger than 1.54 kB were cut with restriction endonucleases (BamHI, HindIII, EcoRI; NEB) and then subcloned into the same sequencing vector. DNA sequence ladders were generated by thermocycle sequencing procedures based upon the use of fluorescent-labeled primers (one of T7, SP6, M13 forward and M13 reverse; ABI), a thermostable DNA polymerase (AmpliTaq; Perkin Elmer/ABI) and dideoxy terminator chemistry (Sanger, et al, 1977, Proc. Natl. Acad. Sci. USA 74:54463). Data were acquired on an ABI 373A automated DNA sequencer (ABI) and processed using the PRISM sequence analysis software (ABI). The nucleotide sequences disclosed herein represent the range of highest quality data acquired in one pass for each clone. All DNA sequence data are reported with the same directionality, 5′ to 3′, regardless of which strand (i.e., coding or anti-coding) is sequenced. Some DNA sequence is reported using standard IUB codes in cases where sequence ambiguities could not be absolutely resolved in first-pass sequence.

For the sequences identified herein as SEQ ID NO. 1-105, the sequences corresponding to each complementing clone identify and provide the coding sequence (gene) responsible for providing that complementation. Therefore, the sequences corresponding to each complementing clone correspond to a particular essential gene.

G. DNA Sequence Analysis of Complementing Clones Similarity Searching (General)

Sequence data were analyzed for similarity to existing publicly-available database entries both at the nucleic acid level and the (putative) polypeptide level; the current releases and daily cumulative updates of these databases are maintained at the NCBI and are freely accessible. The programs BLASTN (Altschul, et al., 1990, J. Mol. Biol. 215:403-410) and FASTA (Pearson, et al., 1988, Proc. natl. Acad. Sci. USA 85:2444-2448) were used to search the nucleic acid databases GenBank (Release 89.0) and EMBL (Rel. 43.0), while the programs BLASTX and TFASTA were used to search the protein databases SwissProt (Rel. 30.0), PIR (Rel. 45.0) and GenPept (Rel 89.0). For reporting the results of the similarity searching below, the following abbreviations of bacterial species names are used:

Bsu=Bacillus subtilis

Eco=Escherichia coli

Zmo=Zymomonas mobilis

Bme=Bacillus megaterium

Lme=Leuconostoc mesenteriodes

Sxy=Staph. xylosys

Sca=Staph. carnosus

Sau=Staph. aureus

Hin=Haemophilus influenzae

Seq=Strep. equisimilis

Bca=Bacillus caldolyticus

Kpn=Klebsiella pneumoniae

Mle=Mycobacterium leprae

H. DNA Sequence of Complementing Clones

Mutant NT 6-Clone pMP33: an Example of Complementing ORFs with Literature Precedent in Staph. aureus.

The ORF complementing the heat-sensitive phenotype of S. aureus mutant NT6 described here was identified by sequencing subclones of pMP33, an E. coli/S. aureus shuttle vector containing a 2.3 kilobase-pair (kb) insert of parental (i.e. wild-type) genomic DNA. The subclones, pMP1006 (0.5 kb), pMP1007 (0.9 kb) and pMP 1008 (0.9 kb), were generated by EcoRI and HindIII digestion of the parent clone and ligation into pGEM3Zf(+), a commercially available vector (Promega, Inc.) suitable for double-stranded DNA sequencing applications.

PCR-based methods (PRISM Dye Primer DNA Sequencing Kit; ABI, Inc.) were employed to generate DNA sequence data from the SP6 promoter of each of the subclones. Electrophoresis and detection of fluorescently-labelled DNA sequence ladder on an ABI 373A automated DNA sequencer (ABI, Inc.) yielded the following sequence data:

```
SEQ ID NO. 4
subclone 1006, a 500 kb Hind III fragment
1006.seq Length: 400 nt
    1 AAATAATCTA AAAATTGGTA GTNCTCCTTC AGATAAAAAT CTTACTTTAA
  51 CACCATTCTT TTNAACTNNT TCCGTGTTTC TTTTTCTAAG TCCATCCATA
101 TTTTNAATGA TGTCATCTGC TGTTTTATCT TTTAAATCTA ACACTGAGTG
151 ATAACGGATT TGTAGCACAG GATCAAATCC TTTATGGAAT CCAGTATGTT
201 CAAATCCTAA GTTACTCATT TTATCAAAGA ACCAATCATT ACCAGCATTA
251 CCTGTAATCT CGCCATCATG ATTCAAGTAT TGATATGGTA AATATGGATC
301 GNTATGTAGG TATAGHCAAC GATGTTTTTT AACATATTTT GGATAATTCA
351 TTAAAGNAAA AGTGTACGAG TNCTTGATTT TCATANTCAA TCACTGGACC
SEQ ID NO. 5
subclone 1007, a 900 bp Hind III fragment
1007.seq Length: 398 nt
    1 TGCGTGAAAT NACTGTATGG CNTGCNATCT GTAAAGGCAC CAAACTCTTT
  51 AGCTGTTAAA TTTGTAAACT TCATTATCAT TACTCCTATT TGTCTCTCGT
101 TAATTAATTT CATTTCCGTA TTTGCAGTTT TCCTATTTCC CCTCTGCAAA
151 TGTCAAAAAT AATAAATCTA ATCTAAATAA GTATACAATA GTTAATGTTA
201 AAACTAAAAC ATAAACGCTT TAATTGCGTA TACTTTTATA GTAATATTTA
251 GATTTTNGAN TACAATTTCA AAAAAAGTAA TATGANCGTT TGGGTTTGCN
301 CATATTACTT TTTTNGAAAT TGTATTCAAT NTTATAATTC ACCGTTTTTC
351 ACTTTTTNCA AACAGTATTC GCCTANTTTT TTTAAATCAA GTAAACTT
SEQ ID NO. 6
subclone 1008, a 920 bp EcoR I/Hind III fragment
1008.seq Length: 410 nt
    1 GTAATGACAA ATNTAACTAC AATCGCTTAA AATATTACAA AGACCGTGTG
  51 TNAGTACCTT TAGCGTATAT CAACTTTAAT GAATATATTA AAGAACTAAA
101 CGAAGAGCGT GATATTTTAA ATAAAGATTT AAATAAAGCG TTAAAGGATA
151 TTGAAAAACG TCCTGAAAAT AAAAAAGCAC ATAACAAGCG AGATAACTTA
201 CAACAACAAC TTGATGCAAA TGAGCAAAAG ATTGAAGAAG GTAAACGTCT
251 ACAAGANGAA CATGGTAATG AATTACCTAT CTCTNCTGGT TTCTNCTTTA
301 TCAATCCATT TGANGTTGTT TATTATGCTG GTGGTACATC AAATGCATTC
351 CGTCATTTTN CCGGAAGTTA TGCAGTGCAA TGGGAAATGA TTAATTATGC
401 ATTAAATCAT
```

A partial restriction map of clone pMP33 appears in FIG. 23, with open boxes to represent the percentage of the clone for which DNA sequence has been obtained in one pass.

Analysis of these data reveals identity (>90%, including sequence ambiguities in first-pass sequence) at both the nucleotide and (predicted) amino acid-level to the femA gene of S. aureus (Genbank ID M23918; published in Berger-Baechi, B. et al., Mol. Gen. Genet. 219 (1989) 263-269). The nucleotide sequence identities to the Genbank entry indicate that complementing clone pMP33 contains the complete ORF encoding the FemA protein along with the necessary upstream elements for its expression in S. aureus. The figure demonstrates the relative positions of the subclones along with the location of the ORF encoding the FemA protein.

Mutant NT64/Clone pMP98: an Example of Complementing ORFs without Direct Literature Precedent, but Identifiable by Similarity to Genes from Other Bacteria

The ORF(s) complementing the heat-sensitive phenotype of S. aureus mutant NT64 described here were identified by sequencing a subclone of pMP98, an E. coli/S. aureus shuttle vector containing a 2.9 kb insert of parental (i.e. wild-type) genomic DNA. The subclone, pMP1038, was generated by EcoRI and HindIII digestion of pMP98 and ligation into pGEM3Zf(+), a commercially available vector (Promega, Inc.) suitable for use in automated fluorescent sequencing applications. Using fluorescently-labelled dye primers (T7 and SP6; ABI, Inc.), a total of 914 bp of sequence from the two edges of the subclone was generated.

```
SEQ ID NO. 106
subclone 1038, a 2800 bp genomic fragment
1038.sp6 Length: 417 nt
    1 GTGATGGATT AAGTCCTAAA TTTNNATTCG CTTTCTTGTC TTTTTAATCT
  51 TTTTCAGACA TTTTATCGAT TTCACGTTTT GTATACTTAG GATTTAAATA
101 GGCATTAATT GTTTTCTTGT CCAAAAATTG ACCATCTTGA TACAAATATT
151 TATCTGTTGG AAATACTTCT TTACTTAAGT NCAATAAACC ATCTTCAAAG
201 TCGCCGCCAT TATAACTATT TGCCATGTTA TCTTGTAAAA GTCCTCTTGC
251 CTGGNTTTCT TTAAATGGTA ACAATGTACG NTAGTTATCA CCTTGTACAT
301 TTTTATCCGT TGCAATTTCT TNTACTTGAT TTGAACTATT GTTATGTTTT
351 NAATTATCTT TTCCCAGCCT GGGTCATCCT TATGGTTANC ACAAGCAGCG
401 AGTATAAAGG TAGCTGT
SEQ ID NO. 107
1038.t7 Length: 497 nt
    1 TAATGTAGCA ATTACAAGGC CTGAAGAGGT GTTATATATC ACTCATGCGA
  51 CATCAAGAAT GTNATTTGGN CGCCCTCAGT CAAATATGCC ATCCAGNTTT
101 TNAAAGGAAA TTCCAGAATC ACTATTAGAA AATCATTCAA GTGGCAAACG
151 ACAAACGGTA CAACCTNNGG CAAAACCTTT TNCTAAACGC GGNTTTTGTC
201 AACGGNCAAC GTCAACGGNN AANCAAGTAT TNTNATCTGN TTGGAATNTT
251 GGTGGCAANG TGGTGCNTAA NGNCNCCGGG GGGAGGCATT GTNNGTAATT
301 TTAACGNGGA NAATGGCTCN NTCGGNCTNG GTNTTATNTT TTATTCACAC
351 AGGGNCGCGN CANGTTTTTT TTGTNGGATT TTTTTCCCCC NTTTTTNAAA
401 AGGNGGGGTN TTNNGGGTGG CTGNTTTANT NGTCTCNGNG TGGNCGTGNN
451 TCATTNNTTT TTTTNTTNNA TCCAAGCCTT NTATGACTTT NNTTGGG
```

Similarity searches at the nucleotide and (putative) amino acid level reveal sequence identity from the left-most (T7) edge of the clone to the Genbank entry for pcrA, a putative helicase from S. aureus (Genbank ID M63176; published in Iordanescu, S. M. and Bargonetti, J. J. Bacteriol. 171 (1989) 4501-4503). The sequence identity reveals that the pMP98 clone contains a C-terminal portion of the ORF encoding pcrA, but that this ORF is unlikely to be responsible for complementation of the NT64 mutant. The Genbank entry extends 410 bp beyond the 3′ end of the pcrA gene, and does not predict any further ORFs. Similarity searches with data obtained from the right-most (SP6) edge reveal no significant similarities, indicating that the complementing ORF in pMP98 is likely to be unpublished for S. aureus. A partial restriction map of clone pMP98 appears in FIG. 20 (there are no apparent restriction sites for BamH I, EcoR I, or Hind III); the relative position and orientation of the identified (partial) ORF corresponding to the PcrA protein is indicated by an arrow:

From the preliminary sequence data, the following PCR primers were designed:

pMP98(+): 5′-CTG AAG AGG TGT TAT ATA TCA C-3′
pMP98(−): 5′-GTG ATG GAT TAA GTC CTA AAT T-3′

These primers were used to amplify the 2.9 kb genomic DNA fragment in one round of PCR amplification directly from S. aureus genomic DNA (parental strain 8325-4). Similar strategies using PCR primers designed from partial sequences can be used for amplifying the genomic sequence (or a cloned genomic sequence) corresponding to the additional complementing clones described below. Additional primers based upon the obtained sequence were designed to generate further DNA sequence data by primer-walking, using the dye terminator strategy (PRISM DyeDeoxy Terminator Kit; ABI, Inc.).

pMP98.b(+): 5′-CTC AGT CAA ATA TGC CAT CCA G-3′
pMP98.b(−): 5′-CTT TAA ATG GTA ACA ATG TAC G-3′

The following sequence data were obtained, as depicted in the partial restriction map in FIG. 41:

```
clone pMP98
SEQ ID NO. 36
pMP98 Length: 2934 nt
      1 CATGAAATGC AAGAAGAACG TCGTATTTGT TATGTAGCAA TTACAAGGGC
    51 TGAAGAGGTG TTATATATCA CTCATGCGAC ATCAAGAATG TTATTTGGTC
  101 GCCCTCAGTC AAATATGCCA TCCAGATTTT TAAAGGAAAT TCCAGAATCA
  151 CTATTAGAAA ATCATTCAAG TGGCAAACGA CAAACGATAC AACCTAAGGC
  201 AAAACCTTTT GCTAAACGCG GATTTAGTCA ACGAACAACG TCAACGAAAA
  251 AACAAGTATT GTCATCTGAT TGGAATGTAG GTGACAAAGT GATGCATAAA
  301 GCCTGGGGAG AAGGCATGGT GAGTAATGTA AACGAGAAAA ATGGCTCAAT
  351 CGAACTAGAT ATTATCTTTA AATCACAAGG GCCAAAACGT TTGTTAGCGC
  401 AATTTGCACC AATTGAAAAA AAGGAGGATT AAGGGATGGC TGATTTATCG
  451 TCTCGTGTGA ACGRDTTACA TGATTTATTA AATCAATACA GTTATGAATA
  501 CTATGTAGAG GATAATCCAT CTGTACCAGA TAGTGAATAT GACAAATTAC
  551 TTCATGAACT GATTAAAATA GAAGAGGAGC ATCCTGAGTA TAAGACTGTA
  601 GATTCTCCAA CAGTTAGAGT TGGCGGTGAA GCCCAAGCCT CTTTCAATAA
  651 AGTCAACCAT GACACGCCAA TGTTAAGTTT AGGGAATGCA TTTAATGAGG
  701 ATGATTTGAG AAAATTCGAC CAACGCATAC GTGAACAAAT TGGCAACGTT
  751 GAATATATGT GCGAATTAAA AATTGATGGC TTAGCAGTAT CATTGAAATA
  801 TGTTGATGGA TACTTCGTTC AAGGTTTAAC ACGTGGTGAT GGAACAACAG
  851 GTTGAAGATA TTACCGRAAA TTTAAAAACA ATTCATGCGA TACCTTTGAA
  901 AATGAAAGAA CCATTAAATG TAGAAKTYCG TGGTGAAGCA TATATGCCGA
  951 GACGTTCATT TTTACGATTA AATGAAGAAA AAGAAAAAAA TGATGAGCAG
1001 TTATTTGCAA ATCCAAGAAA CGCTGCTGCG GGATCATTAA GACAGTTAGA
1051 TTCTAAATTA ACGGCAAAAC GAAAGCTAAG CGTATTTATA TATAGTGTCA
1101 ATGATTTCAC TGATTTCAAT GCGCGTTCGC AAAGTGAAGC ATTAGATGAG
1151 TTAGATAAAT TAGGTTTTAC AACGAATAAA AATAGAGCGC GTGTAAATAA
1201 TATCGATGGT GTTTTAGAGT ATATTGAAAA ATGGACAAGC CAAAGAAGAG
1251 TTCATTACCT TATGATATTG ATGGGATTGT TATTAAGGTT AATGATTTAG
1301 ATCAACAGGA TGAGATGGGA TTCACACAAA AATCTCCTAG ATGGGCCATT
1351 GCTTATAAAT TTCCAGCTGA GGAAGTAGTA ACTAAATTAT TAGATATTGA
1401 ATTAAGTATT GGACGAACAG GTGTAGTCAC ACCTACTGCT ATTTTAGAAC
1451 CAGTAAAAGT AGCTGGTACA ACTGTATCAA GAGCATCTTT GCACAATGAG
1501 GATTTAATTC ATGACAGAGA TATTCGAATT GGTGATAGTG TTGTAGTGAA
1551 AAAAGCAGGT GACATCATAC CTGAAGTTGT ACGTAGTATT CCAGAACGTA
1601 GACCTGAGGA TGCTGTCACA TATCATATGC CAACCCATTG TCCAAGTTGT
1651 GGACATGAAT TAGTACGTAT TGAAGGCGAA GTTAGCACTT CGTTGCATTA
1701 ATCCAAAATG CCAAGCACAA CTTGTTGAAG GATTGATTCA CTTTGTATCA
1751 AGACAAGCCA TGAATATTGA TGGTTTAGGC ACTAAAATTA TTCAACAGCT
1801 TTATCAAAGC GAATTAATTA AAGATGTTGC TGATATTTTC TATTTAACAG
1851 AAGAAGATTT ATTACCTTTA GACAGAATGG GGCAGAAAAA AGTTGATAAT
1901 TTATTAGCTG CCATTCAACA AGCTAAGGAC AACTCTTTAG AAAATTTATT
1951 ATTTGGTCTA GGTATTAGGC ATTTAGGTGT TAAAGCGAGC CAAGTGTKAG
2001 CAGAAAAATA TGAAACGATA GATCGATTAC TAACGGTAAC TGAAGCGGAA
2051 TTAGTAGAAT TCATGATATA GGTGATAAAG TAGCGCAATC TGTAGTTACT
2101 TATTTAGCAA ATGAAGATAT TCGTGCTTTA ATTCCATAGG ATTAAAAGAT
2151 AAACATGTTA ATATGATTTA TGAAGGTATC CAAAACATCA GATATTGAAG
2201 GACATCCTGA ATTTAGTGGT AAAACGATAG TACTGACTGG TAAGCTACAT
2251 CCAAATGACA CGCAATGAAG CATCTAAATG GCTTGCATCA CCAAGGTGCT
2301 AAAGTTACAA GTAGCGTTAC TAAAAATACA GATGTCGTTA TTGCTGGTGA
2351 AGATGCAGGT TCAAAATTAA CAAAAGCACA AAGTTTAGGT ATTGAAATTT
2401 GGACAGAGCA ACAATTTGTA GATAAGCAAA ATGAATTAAA TAGTTAGAGG
2451 GGTATGTCGA TGAAGCGTAC ATTAGTATTA TTGATTACAG CTATCTTTAT
2501 ACTCGCTGCT TGTGGTAACC ATAAGGATGA CCAGGCTGGA AAAGATAATC
2551 AAAAACATAA CAATAGTTCA AATCAAGTAA AAGAAATTGC AACGGATAAA
2601 AATGTACAAG GTGATAACTA TCGTACATTG TTACCATTTA AAGAAAGCCA
2651 GGCAAGAGGA CTTTTACAAG ATAACATGGC AAATAGTTAT AATGGCGGCG
2701 ACTTTGAAGA TGGTTTATTG AACTTAAGTA AAGAAGTATT TCCAACAGAT
2751 AAATATTTGT ATCAAGATGG TCAATTTTTG GACAAGAAAA CAATTAATGC
2801 CTATTTAAAT CCTAAGTATA CAAAACGTGA AATCGATAAA ATGTCTGAAA
2851 AAGATAAAAA AGACAAGAAA GCGAATGAAA ATTTAGGACT TAATCCATCA
2901 CACGAAGGTG AAACAGATCG ACCTGCAGKC ATGC
```

From this data, a new ORF in the pMP98 clone was identified as having significant similarity to lig, the gene encoding DNA ligase from E. coli: (Genbank ID M30255; published in Ishino, Y., et al., Mol. Gen. Genet. 204 (1986), 1-7). The revised clone map of pMP98, including the predicted size and orientation corresponding to the putative DNA ligase ORF, is shown in FIG. 41:

The DNA ligase protein from E. coli is composed of 671 amino acids; a polypeptide translated from S. aureus DNA sequence acquired above matches the C-terminal 82 amino acids of the E. coli DNA ligase with a 52% sequence identity and a 67% sequence similarity; this level of similarity is considered significant when comparing proteins from Gram-negative and Gram-positive bacteria. Since the predicted coding region of the S. aureus gene for DNA ligase is small enough to be contained within clone pMP98 and the gene for DNA ligase is known to be essential to survival for many bacterial species, NT64 is concluded to contain a ts mutation in the gene for DNA ligase.

Mutant NT42/Clone pMP76: an Example of Complementing ORFs with Unknown Function

The ORF(s) complementing the temperature-sensitive phenotype of S. aureus mutant NT42 described here was identified by sequencing subclones of pMP0076, an E. coli/S. aureus shuttle vector containing a 2.5 kb insert of parental (i.e. wild-type) genomic DNA. The subclones, pMP1026 (1.1 kb) and pMP1027 (1.3 kb), were generated by EcoRI and BamHI digestion of the parent clone and ligation into pGEM3Zf(+), a commercially available vector (Promega, Inc.) suitable for double-stranded DNA sequencing applications.

PCR-based methods (PRISM Dye Primer DNA Sequencing Kit; ABI, Inc.) were employed to generate DNA sequence data from the SP6 and T7 promoters of both of the subclones. Primer walking strategies were used to complete the sequence contig. Electrophoresis and detection of fluorescently-labelled DNA sequence ladder on an ABI 373A automated DNA sequencer (ABI, Inc.) yielded the following sequence data:

```
clone pMP76
SEQ ID NO. 37
pMP76 Length: 2515 nt
      1 CSYCGGWACC CGGGGATCCT CTAGAGTCGA TCGTTCCAGA ACGTATTCGA
    51 ACTTATAATT ATCCACAAAG CCGTGTAACA GACCATCGTA TAGGTCTAAC
  101 GCTTCAAAAA TTAGGGCAAA TTATGGAAGG CCATTTAGAA GAAATTATAG
  151 ATGCACTGAC TTTATCAGAG CAGACAGATA AATTGAAAGA ACTTAATAAT
  201 GGTGAATTAT AAAGAAAAGT TAGATGAAGC AATTCATTTA ACACAACAAA
  251 AAGGGTTTGA ACAAACACGA GCTGAATGGT TAATGTTAGA TGTATTTCAA
  301 TGGACGCGTA CGGACTTTGT AGTCCACATG CATGATGATA TGCCGAAAGC
  351 GATGATTATG AAGTTCGACT TAGCATTACA ACGTATGTTA TTAGGGAGAG
  401 CCTATACAGT ATATAGTTGG CTTTGCCTCA TTTTATGGTA GAACGTTTGA
  451 TGTAAACTCA AATTGTTTGA TACCAAGACC TGAAACTGAA GAAGTAATGT
  501 TGCATTTCTT ACAACAGTTA GAAGATGATG CAACAATCGT AGATATCGGA
  551 ACGGGTAGTG GTGTACTTGC AATTACTTTG AAATGTTGAA AAGCCGGATT
  601 TAAATGTTAT TGCTACTGAT ATTTCACTTG AAGCAATGAA TATGGCTCCG
  651 TAATAATGCT GAGAAGCATC AATCACAAAT ACAATTTTTA ACAGGGGATG
  701 CATTAAAGCC CTTAATTAAT GAAGGTATCA AKTTGAACGG CTTTGATATC
  751 TAATCCMCCA TATATAGATG AAAAAGATAT GGTTACGATG TCTCCMACGG
  801 TTACGARATT CGAACCACAT CAGGCATTGT TTGCAGATAA CCATGGATAT
  851 GCTATTTATG AATCAATCAT GGAAGATTTA CCTCACGTTA TGGAAAAAGG
  901 CAGCCCAGTT GTTTTTGAAA TTGGTTACAA TCAAGGTGAG GCACTTAAAT
  951 CAATAATTTT AAATAAATTT CCTGACAAAA AAATCGACAT TATTAAAGAT
1001 ATAAATGGCC ACGATCGAAT CGTCTCATTT AAATGGTAAT TAGAAGTTAT
1051 GCCTTTGCTA TGATTAGTTA AGTGCATAGC TTTTTGCTTT ATATTATGAT
1101 AAATAAGAAA GGCGTGATTA AGTTGGATAC TAAAATTTGG GATGTTAGAG
1151 AATATAATGA AGATTTACAG CAATATCCTA AAATTAATGA AATAAAAGAC
1201 ATTGTTTTAA ACGGTGGTTT AATAGGTTTA CCAACTGAAA CAGTTTATGG
1251 ACTTGCAGCA AATGCGACAG ATGAAGAAGC TGTAGCTAAA ATATATGAAG
1301 CTAAAGGCCG TCCATCTGAC AATCCGCTTA TTGTTCATAT ACACAGTAAA
1351 GGTCAATTAA AAGATTTTAC ATATACTTTG GATCCACGCG TAGAAAAGTT
1401 AATGCAGGCA TTCTGGCCGG GCCCTATTTC GTTTATATTG CCGTTAAAGC
1451 TAGGCTATCT ATGTCGAAAA GTTTCTGGAG GTTTATCATC AGTTGCTGTT
1501 AGAATGCCAA GCCATTCTGT AGGTAGACAA TTATTACAAA TCATAAATGA
1551 ACCTCTAGCT GCTCCAAGTG CTAATTTAAG TGGTAGACCT TCACCAACAA
1601 CTTTCAATCA TGTATATCAA GATTTGAATG GCCGTATCGA TGGTATTGTT
1651 CAAGCTGAAC AAAGTGAAGA AGGATTAGAA AGTACGGTTT TAGATTGCAC
1701 ATCTTTTCCT TATAAAATTG CAAGACCTGG TTCTATAACA GCAGCAATGA
1751 TTACAGAAAT AMTTCCGAAT AGTATCGCCC ATGCTGATTA TAATGATACT
1801 GAACAGCCAA TTGCACCAGG TATGAAGTAT AAGCATTACT CAACCCAATA
1851 CACCACTTAC AATTATTACA GATATTGAGA GCAAAATTGG AAATGACGGT
1901 AAAGATTRKW MTTCTATAGC TTTTATTGTG CCGAGTAATA AGGTGGCGTT
1951 TATACCAAGT GARSCGCAAT TCATTCAATT ATGTCAGGAT GMCAATGATG
2001 TTAAACAAGC AAGTCATAAT CTTTATGATG TGTTACATTC ACTTGATGAA
2051 AATGAAAATA TTTCAGCGGC GTATATATAC GGCTTTGAGC TGAATGATAA
2101 TACAGAAGCA ATTATGAATC GCATGTTAAA AGCTGCAGGT AATCACATTA
2151 TTAAAGGATG TGAACTATGA AGATTTTATT CGTTTGTACA GGTAACACAT
2201 GTCGTAGCCC ATTAGCGGGA AGTATTGCAA AAGAGGTTAT GCCAAATCAT
2251 CAATTTGAAT CAAGAGGTAT ATTCGCTGTG AACAATCAAG GTGTTTCGAA
2301 TTATGTTGAA GACTTAGTTG AAGAACATCA TTTAGCTGAA ACGACCTTAT
2351 CGCAACAATT TACTGAAGCA GATTTGAAAG CAGATATTAT TTTGACGATG
2401 TCGTATTCGC ACAAAGAATT AATAGAGGCA CACTTTGGTT TGCAAAATCA
2451 TGTTTTCACA TTGCATGAAT ATGTAAAAGA AGCAGGAGAA GTTATAGATC
2501 GACCTGCAGG CATGC
```

Analysis of the DNA sequence data at the nucleotide level reveals no significant similarity to data in the current release of the Genbank or EMBL databases. Analysis of the predicted ORFs contained within clone pMP76 reveals a high degree of similarity to two open reading frames identified in B. subtilis; “ipc29D” and “ipc31D” (EMBL entry Z38002). A partial restriction map of pMP76 is depicted in FIG. 42, along with an open box to indicate the percentage of the clone for which DNA sequence has been obtained. The relative orientation and predicted size of the “ipc29D” ORF is indicated by an arrow:

These two ORFs identified from the EMBL entry Z38002 were predicted from genomic sequence data and are denoted as “putative”; no characterization of expression or function of the predicted gene products has been reported in the literature. A similarity has been noted between the predicted Ipc31D-like polypeptide and the SUA5 gene product from yeast (S. cerevisiae), but functional characterization still remains to be performed. Hence, the ORFs contained within clone pMP76 represent putative polypeptides of uncertain function, but are known to be responsible for restoring a wild-type phenotype to NT42.

In addition to the illustrative sequences described above, the following sequences of clones complementing heat sensitive mutants of S. aureus similarly provide essential genes.

Mutant: NT3

Phenotype: temperature sensitivity

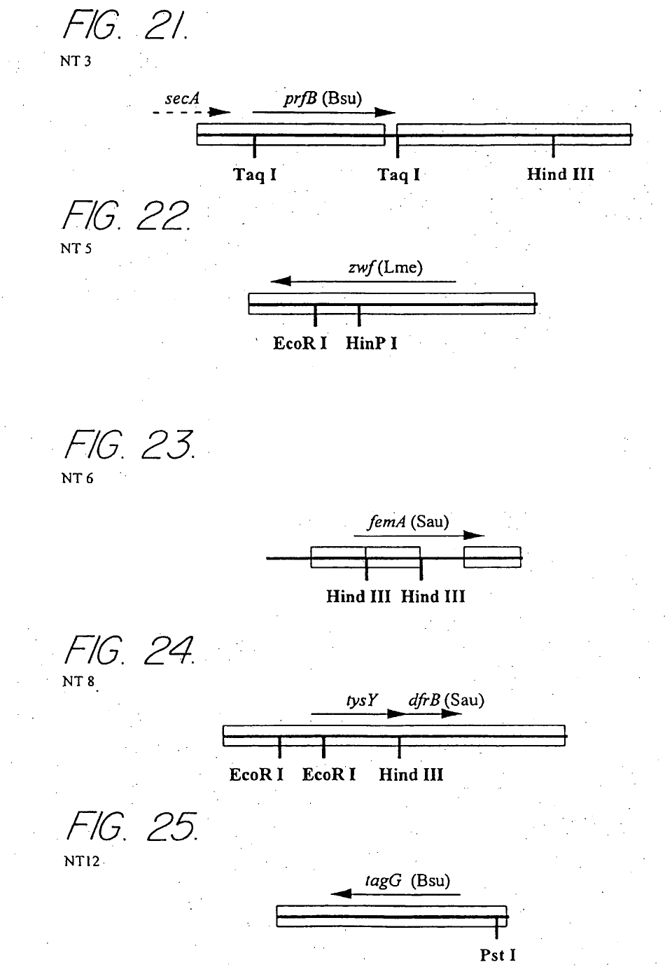

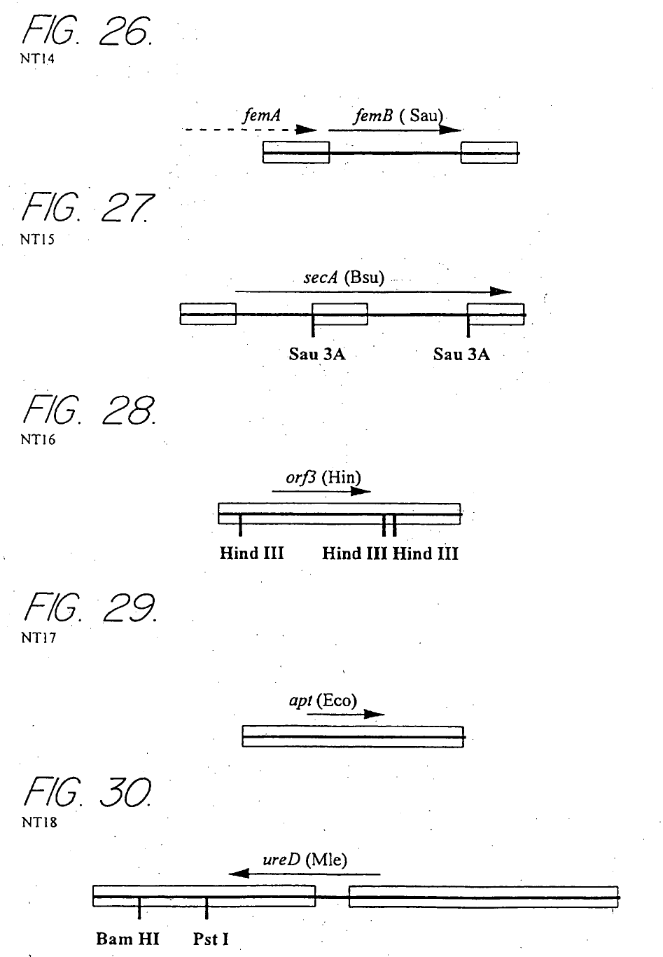

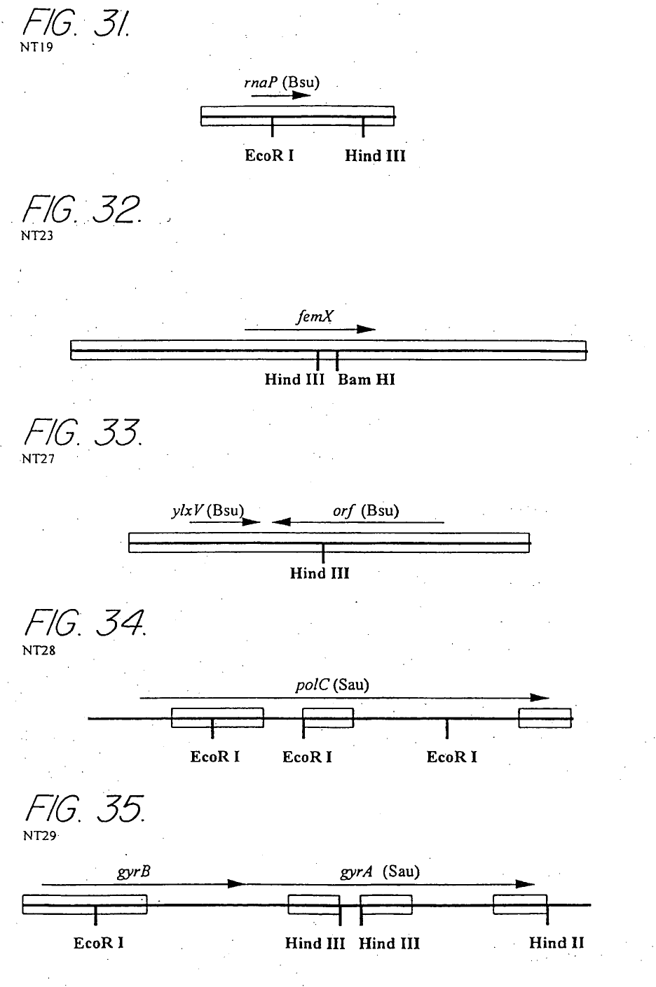

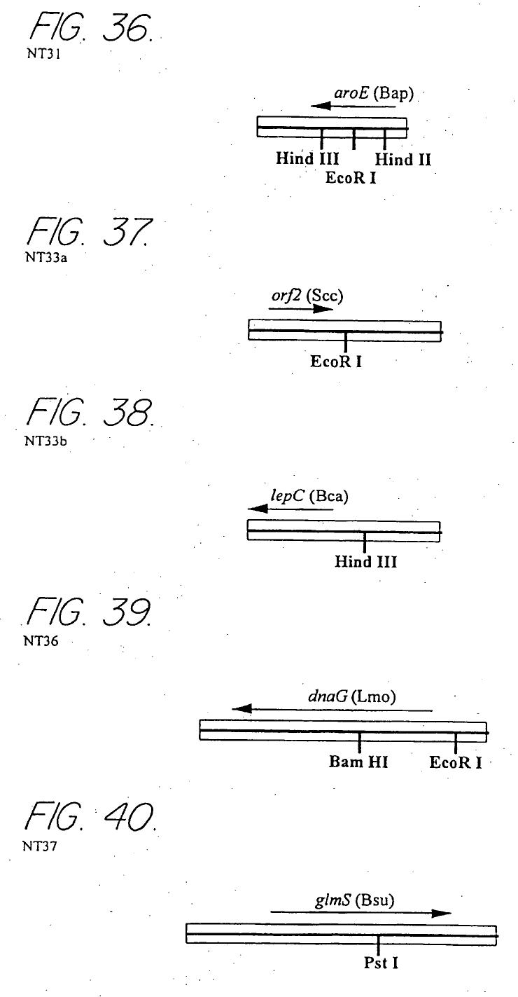

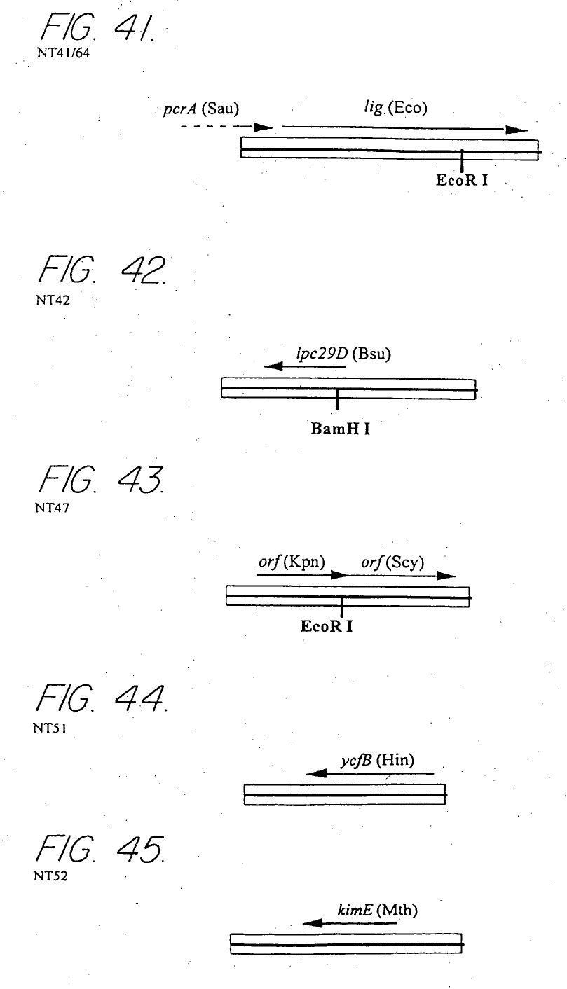

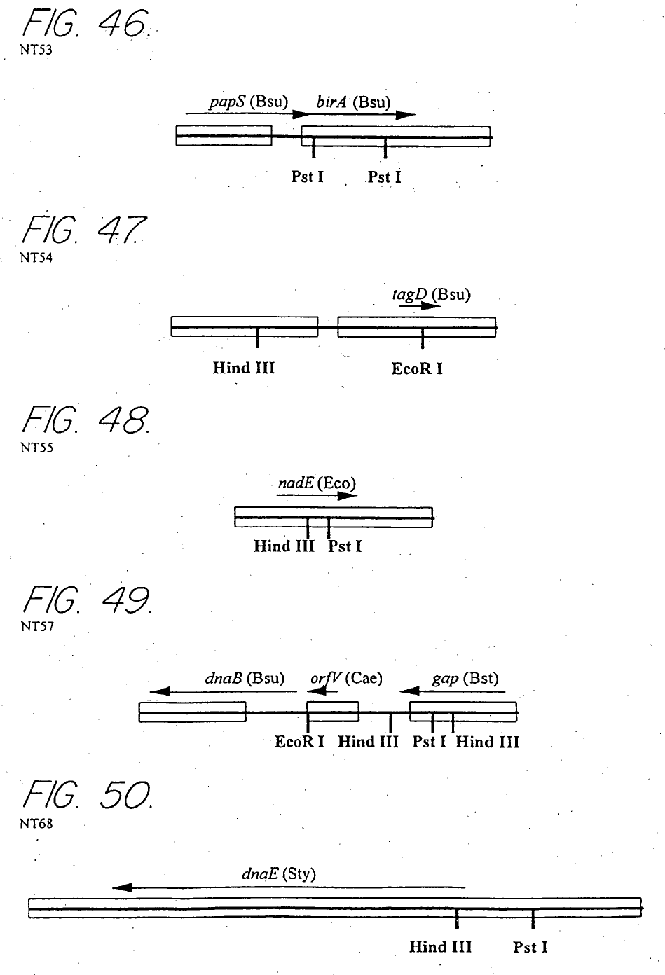

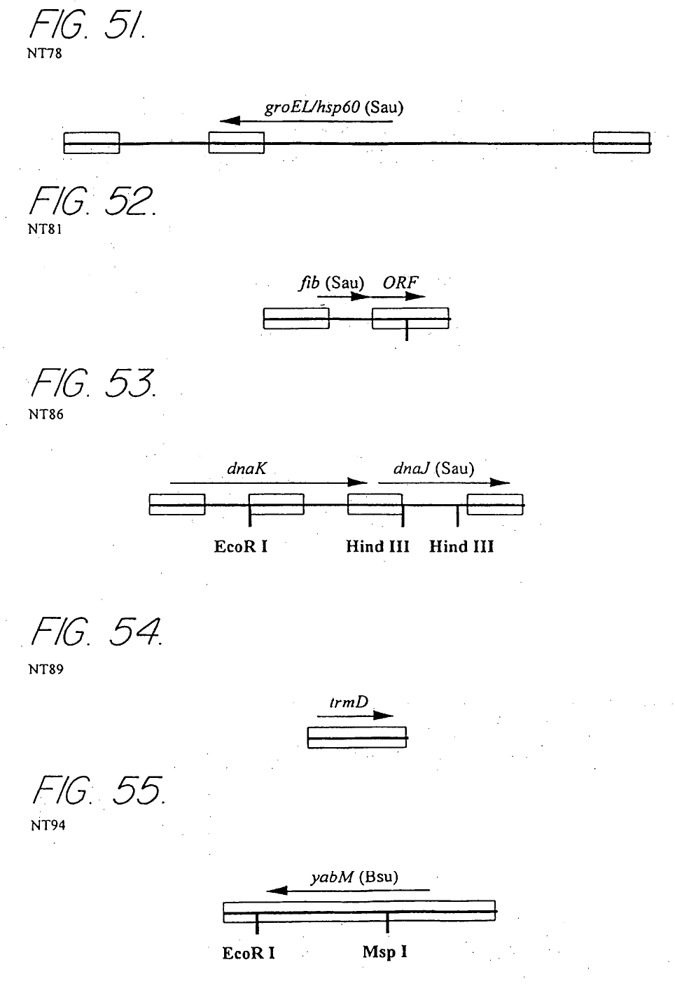

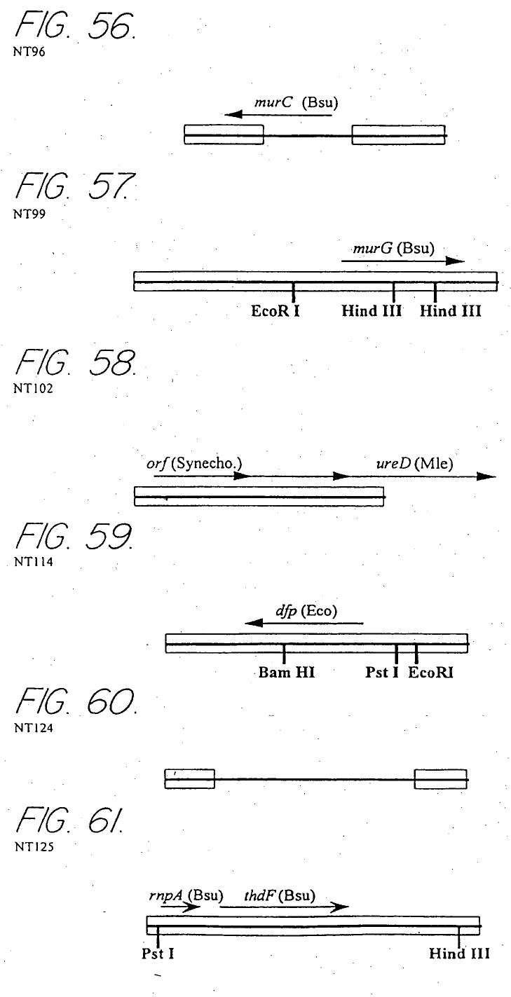

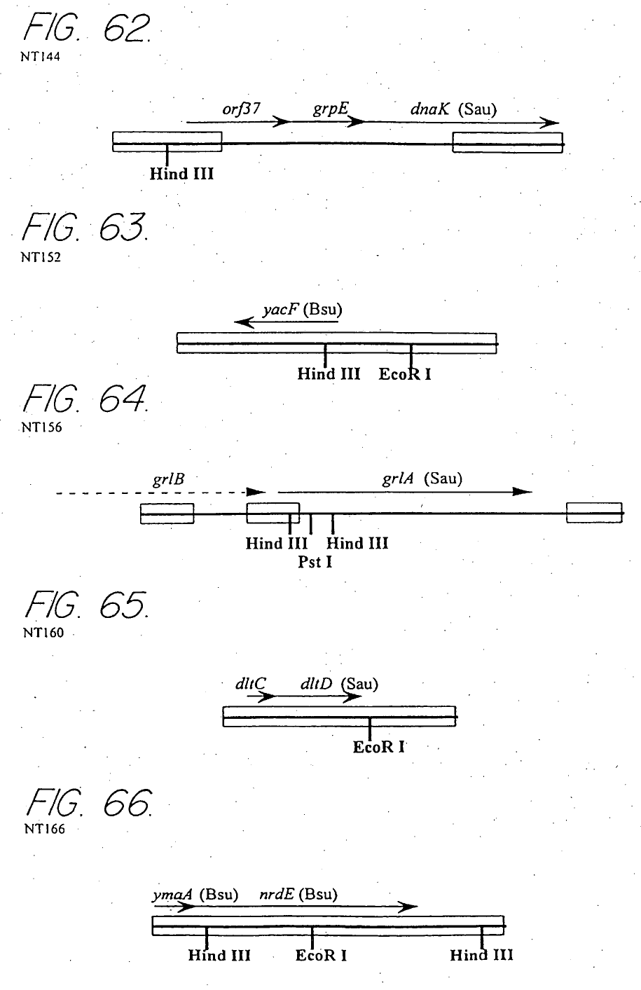

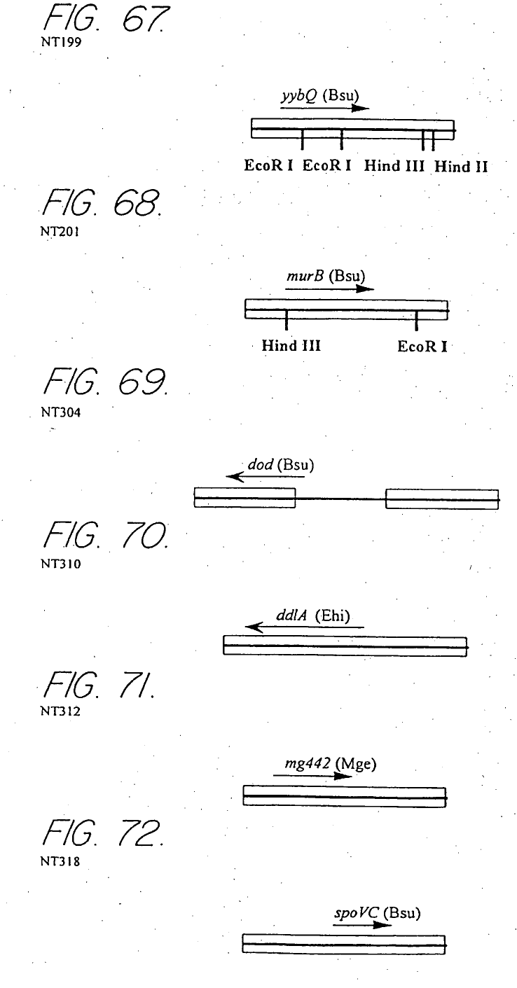

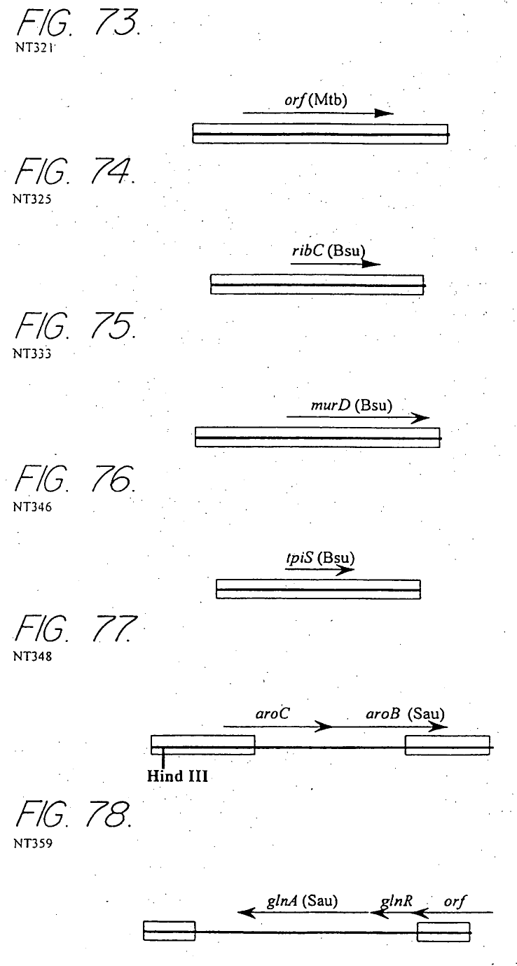

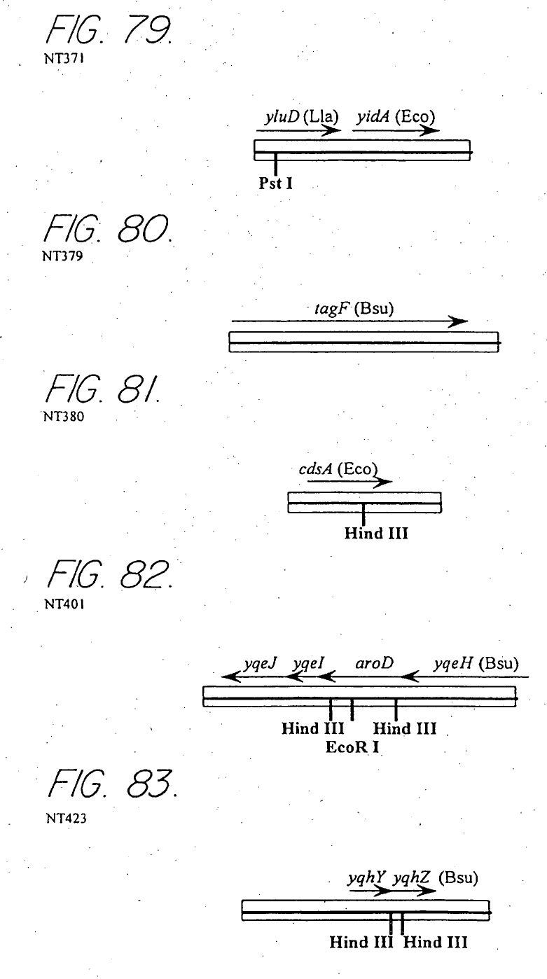

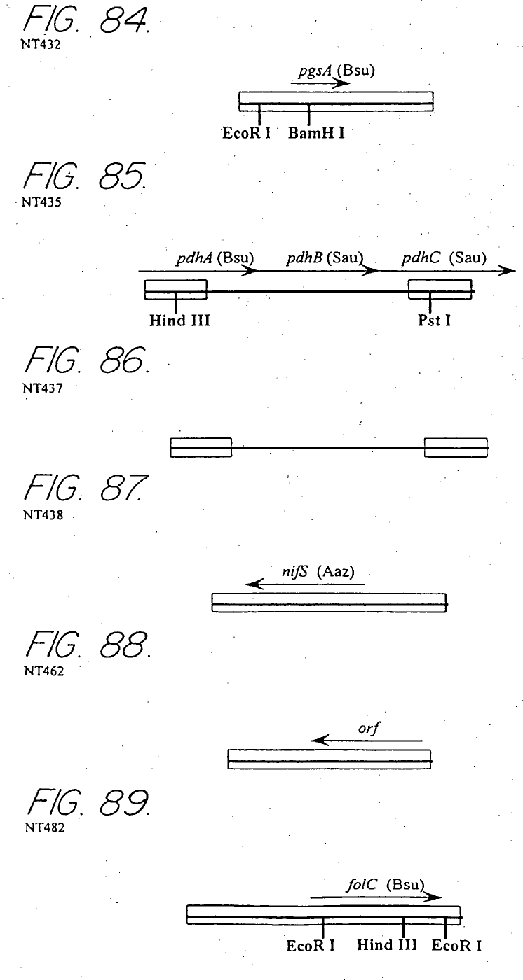

Sequence map: Mutant NT3 is complemented by plasmid pMP27, which contains a 3.9 kb insert of S. aureus genomic DNA. The partial restriction map of the insert is depicted in FIG. 21; open boxes along part of the length of the clone indicate the portions of the clone for which DNA sequence has been obtained (this contig is currently being completed). Database searches at both the nucleic acid and protein levels reveal strong similarity at both the peptide and nucleic acid level to the C-terminal fragment of the SecA protein from S. carnosus (EMBL Accession No. X79725) and from B. subtilis(Genbank Accession No. D10279). Since the complete SecA ORF is not contained within clone pMP27, SecA is unlikely to be the protein responsible for restoring mutant NT3 to a wild-type phenotype. Further strong peptide-level similarities exist between the DNA sequence of a Taq I subclone of pMP27 and the prfB gene, encoding Peptide Release Factor II, of B. subtilis (Genbank D10279; published in Pel et al., 1992, Nucl. Acids Res. 20:4423-4428). Cross complementation analysis (data not shown) suggests that a mutation in the prfB gene is most likely to be responsible for conferring a temperature-sensitive phenotype to mutant NT3 (i.e. it is an essential gene)

DNA sequence data: The following DNA sequence data represents the sequences at the left-most and right-most edges of clone pMP27, using standard M13 forward and M13 reverse sequencing primers, and then extending via primer walking strategies. The sequences below can be used to design PCR primers for the purpose of amplification from genomic DNA with subsequent DNA sequencing.

```
clone pMP27 (forward and reverse contigs)
SEQ ID NO. 1
pMP27.forward Length: 1739 nt
      1 CTCGCAGCCG NYAKYCGWAA ATGGTCCAAT GTACTCCATC CATCACTGCA
    51 TCAACCTTAC CTGTTTCTTC GTTCGTACGA TGATCTTTCA CCATTGAGTA
  101 TGGATGGAAA ACATATGATC TAATTTGGCT TCCCCAGCCG ATTTCTTTTT
  151 GTTCGCCACG AATTTCAGCC ATTTCACGTG CCTGCTCTTC CAATTTTAAT
  201 TGATATAATT TAGACTTTAA CATTTTCATA GCTGCTTCAC GGTTTTTAAT
  251 TTGAGAACGT TCATTTTGGT TATTAACAAC TATACCTGAG GGGTGGTGGG
  301 TAATTCGTAT TGCCGATTCA GTTTTGTTAA TATGCTGACC ACCTGCACCA
  351 GAAGCTCTGA ATGTATCAAC TGTAATATCA TCCGGATTGA TTTCAATCTC
  401 TATTTCATCA TTATTAAAAT CTGGAATAAC GTCGCATGAT GCAAATGATG
  451 TATGACGACG TCCTGATGAA TCAAATGGAG AAATTCGTAC TAGTCGGTGT
  501 ACACCTTTTT CAGCTTTTAA ATAACCATAA GCATTATGCC CTTTGATGAG
  551 CAATGTTACA CTTTTAATCC CCGCTTCATC CCCAGGTAGA TAATCAACAG
  601 TTTCAACTTT AAAGCCTTTC TTCTCAACAA TAACGTTGAT ACATTCTAAA
  651 TAGCATATTA GCCCAATCTT GAGACTCCGT GCCACCTGCA CCAGGATGTA
  701 ACTCTAGAAT TGCGTTATTG GCATCGTGAG GCCCATCTAA TAATAATTGC
  751 AATTCGTATT CATCCACTTT AGCCTTAAAA TTAATGACCT CTTGCTCTAA
  801 GTCTTCTTTC ATTTCCTTCA TCAAATTCTT CTTGTAATAA ATCCCAAGTA
  851 GCATCCATGT CATCTACTTC TGCTTGTAGT GTTTTATAAC CATTAACTAT
  901 TGCTTTTAAC GCATTATTTT TATCTATAAT ATCTTGCGCT TTCGTTTGGT
  951 TATCCCAAAA ATTAGGTTCT GCCATCATTT CTTCATATTC TTGAATATTA
1001 GTTTCTTTGT TCTCTAAGTC AAAGAGACCC CCTAATTTGT GTTAAATCTT
1051 GATTATACTT ATCTATATTT CGTTTGATTT CTGATAATTC CATAGCATTC
1101 GCTCCTATTT ATATTTCAAT TCAAGTCATT GATTTGCATC TTTTATAATG
1151 CTAAATTTTA ACATAATTTT GTTAAATAAC AATGTTAAGA AATATAAGCA
1201 CACTGACAAT TAGTTTATGC ATTTATTGTT TAAAAAWGCA GTACATTTAT
1251 GCATCGACAT ATGCCTAAAC CGATTTTTTA AAACTAAGTA CATAACAACG
1301 TTTAACAACT TCTTCACATT TTTTAAAGTA TTTAACGCTT GTAAAATAAA
1351 AAGACTCCTC CCATAACACA AACTATAGGT GTTTAATTGG AAGGAGTTAT
1401 TTTATATCAT TTATTTTCCA TGGCAATTTT TGAATTTTTT ACCACTACCA
1451 CATGGACAAT CATCGTTACG ACCAACTTGA TCGCCTTTAA CGATTGGTTT
1501 CGGTTTCACT TTTTCTTTAC CATCTTCAGC TGAAACGTGC TTCGCTTCAC
1551 CAAACTCTGT TGTTTTTTCA CGTTCAATAT TATCTTCAAC TTGTACTACA
1601 GATTTTAAAA TGAATTTACA AGTATCTTCT TCAATATTTT GCATCATGAT
1651 ATCAAATAAT TCATGACCTT CATTTTGATA GTCACGTAAT GGATTTTGTT
1701 GTGCATAAGA ACGTAAGTGA ATACCTTGAC GTAATTGAT
pMP27.reverse Length: 2368 nt
SEQ ID NO. 2
      1 CTGCAGGTCG ATCTGCATCT TGATGTTTAT GAAATTCGAG TTGATCTAGT
    51 AATTAAATAA CCAGCTAATA ATGACACTAC ATCAGKAAGA ATAATCCACT
  101 CGTTATGGAA ATACTCTTTA TAGATTGAGG CACCAATTAA AATTAATGTC
  151 AGAATAGTAC CGACCCATTT ACTTCTTGTT ATTACACTAA ATAATACTAC
  201 CAAGACACAT GGAAAGAATG CTGCGCTAAA ATACCATATC ATTCATTTTC
  251 CTCTTTTCTT TTATTTAAAA TGTTCATGGT TGTTTCTCTT AATTCTGTTC
  301 TAGGTATAAA GTTTTCAGTC AACATTTCTG GAATGATATT ATTAATAAAA
  351 TCTTGTACAG ATGCTAAATG GTCAAATTGA ATAATTGTTT CTAGACTCAT
  401 TTCATAAATT TCGAAAAATA ATTCTTCGGG ATTACGKTTT TGTATTTCTC
  451 CAAATGTTTC ATAAAGCAAA TCAATTTTAT CAGCAACTGA AAGTATTTGG
  501 CCTTCTAATG AATCATCTTT ACCTTCTTGC AGTCGTTGCT TATAAACATC
  551 TCTATATTGT AATGGAATTT CTTCTTCAAT AAAGGTCTCT ACCATTTCTT
  601 CTTCAACTTG CGAAAATAAT TTTTTTAATT CACTACTCGC ATATTTAACA
  651 GGTGTTTTTA TATCACCAGT AAACACTTCG GSGAAATCAT GATTTAATGC
  701 TTTTTCATAT AAGCTTTTCC AATTAAYCTT TCTCCATGAT ATTCTTCAAC
  751 TGTTGCTAGA TATTGTGCAA TTTTAGTTAC TTTAAAGGAG TGTGCTGCAA
  801 CATTGTGTTC AAAATATTTA AATTTTCCAG GTAATCTTAT AAGTCTTTCC
  851 ATATCTGATA ATCTTTTAAA ATATTGATGT ACACCCATTT CAATTACCTC
  901 CTCCATTAAT TAATCATAAA TTATACTTTC TTTTTACATA TCAATCAATT
  951 AAATATCATT TAAATATCTT CTTTATATAA CTCTGATTAA ATGATACCAA
1001 AAAATCCTCT CAACCTGTTA CTTAAACAGG CTAAGAGGGT AGTCTTGTCT
1051 TGATATATTA CTTAGTGGAT GTAATTATAT TTTCCTGGAT TTAAAATTGT
1101 TCTTGAAGAT TTAACATTAA ATCCAGCATA GTTCATTTTC AGAAACAGTA
1151 ATTGTTCCMT TTAGGGTTTA CAGATTCAAC AACACCAACA TGTCCATATG
1201 GACCAGCAGC TGTTTGGAAA ATAGCGCCAA CTTCTGGKGT TTTATCTACT
1251 TTTAAATCCT GCAACTTTTG CTGCGTAATT CCAGTTATTT GCATTGCCCC
1301 ATAAACTTCC TATACTTCTA CCTAATTGTG CACGACGATC GAAAGCATAA
1351 TATGTGCAGT TTCCATAAGC ATATAAGTTT CCTCTGTTAG CAACTGATTT
1401 ATTGTAGTTA TGTGCAACAG GTACAGTTGG TACTGATTTT TGTACTTGAG
1451 CAGGTTTGTA TGCTACATTA ACTGTCTTAG TTACTGCTTG CTTAGGTGCT
1501 TGCTTAACTA CTACTTTTTT AGATGCTTGT TGTACAGGTT GTTTTACTAC
1551 CTTTTTAGCT TGGCTTGCTT TTCTTACTGG TGATTTAACC GCTTTAGTTT
1601 GTTTCACTTT ATTTTGAGGC ACAAGTGAAA TCACGTCACC AGGAAAAATT
1651 AAAGGTGTTA CACCAGGATT GTATTGAATA TAATTGATTC AACGTTAAGT
1701 GATGCTCTTA AAGCAATCTT ATATTAATGA ATCGCCAGCA ACTACTGTWT
1751 AAGTTGTCGG TGATTGCGTT TGTGCTTGAA CATTTGATAC ATAATTATGT
1801 TGAACAGGTG TTTTTACTTG TGTGCCATGT TGTTGTGCAT GTGCKGCATT
1851 ATTTAAAGCK AAAAAAGCTA ACACTGACGA AACCGTCACT GWAAGARART
1901 TTTTCATCTK GCTGTCATTC CTTTGCTGTW AGTATTTTAA GTTATGCAAA
1951 TACTATAGCA CAATACATTT TGTCCAAAAG CTAATTGTTA TAACGANGTA
2001 ATCAAATGGT TAACAANATN AANAGAAGAC AACCGTNTAT CATAGNGGNA
2051 AANGTAGNCA TACCATGNAA TTGAGAACGT TNTCAANAAN TAANTCAATA
2101 CCNTGAAAAT CGCCATAGGN AATATTACNA AATGCACACT GCATATGNTG
2151 NTTTAACAAA CACNACTTTT NANAAATATA NTCTAACTCT ATCTACCGAA
2201 TTGNACTTAA ATATTCATAA ANAAATNATA TTCNAAAATC TAATTTACAA
2251 TTTATTTAGC TACCTTTAAA AAANCNNAAA ACCGACGNCC TTTTAGAGCC
2301 TCGGTTTTTA NATATATNTT AATCGTGCGA CATTGTCTGT TTTNAATNTG
2351 ATTCGACTCT AGNCGATC
```

Mutant: NT5
Phenotype: temperature sensitivity
Sequence map: Mutant NT5 is complemented by plasmid pMP628, which contains a 2.5 kb insert of S. aureus genomic DNA. The partial restriction map of the insert is depicted in FIG. 22. Database searches at both the nucleic acid and protein levels reveal strong similarity between one of the ORFs contained within clone pMP628 and the zwf gene from a variety of species, which encodes the Glucose-6-Phosphate Dehydrogenase (G6PD) protein (EC 1.1.1.49). The strongest similarity is demonstrated in the Genbank entry for G6PD (Accession No. M64446; published in Lee, W. T. et al. J. Biol. Chem. 266 (1991) 13028-13034.) from Leuconostoc mesenteriodes, here abbreviated as “Lme”.

DNA sequence data: The following DNA sequence data represents the complete first-pass sequence of pMP628; the sequence below can be used to design PCR primers for the purpose of amplification from genomic DNA with subsequent DNA sequencing.

```
clone pMP628
SEQ ID NO. 3
pMP628 Length: 2494 nt
      1 AATCATTTTA AATGATTGAT CAAGATGGTA TGGCGAAAGA CCAACGTAAT
    51 CACTTAATTC TTGCAAATTG AAAGGCTCTA ATAAACGATC TTCAATATAA
  101 ACAATTGCCT GTTGTATTTG CTTGATAACG TCCAAAACTT TCACTCCAAT
  151 TAATTCAATC ATTTATTTTT ATTCTACATT ATTTCTATAA ATTATACACC
  201 CATTTGTTCA ATGATTATTA AAATAGTTTT GGGCATTGTA AAATATAATT
  251 TCATAATATA GTCTAGAAAA AAAGCGAATG ATAGAACAAT TGATTTACTT
  301 GATTCGTAAT CAATCCTTGT CATTCGCTCA TTTATTTTTG TTTAACATGT
  351 GCGTTTTAAT TCAATTATTG AATATCGTCC CACCAATGGT TACCATCACG
  401 AGCAAGTAGT AAATCACTTT CTAATGGACC ATTAGTACCT GATTCATAGT
  451 TAGGGAATTC TGGATCAACC ATATTCCATT CATCTTGGAA TTGCATCAAC
  501 AAATTTCCAT GTTGATTTTA ATTCTTCCCA GTGCGTGAAG TTAGTGGCAT
  551 CACCTTTAAG ACAATCAAAT AATAGATTTT CATATGCATC TACAGTATTC
  601 ATTTTATCTT GAGCGCTCAT TGAGTAAGAC AATTGGACAG GTTCTGTTTC
  651 GATACCTTGT GTWTTTTTCT TAGCATTTAR ATGTAAAGAT ACACCTTCAT
  701 TAGGTTGGAT ATTGATTANT AATAGGTTTG AATCTAACAG TTTATCAGTT
  751 TCATAGTATA AGTTCATTGG TACTTCTTTA AATTCAACGA CAACTTGAAT
  801 TGTTTTAGAT TTCATACGTT TACCAGTACG GATATAGAAT GGTACACCAG
  851 CCCATCTAAA GTTATCAATT GTTAATTTAC CTGAAACAAA GGTAGGTGTG
  901 TTAGAGTCAT CTGCAACGCG ATCTTCATCA CGGTATGCTT TAACTTGTTT
  951 ACCATCGATA TAGCCTTCGC CATATTGACC ACGAACAAAG TTCTTTTTAA
1001 CATCTTCAGA TTGGAAATGA CGCAGTGATT TAAGTACTTT TAACTTTCTC
1051 AGCACGGATA TCTTCACTAT TTAAACTAAT AGGTGCTTCC ATAGCTAATA
1101 ATGCAACCAT TTGTAACATG TGGTTTTGCA CCATATCTTT TAGCGCGCCA
1151 CTTGATTCAT AATAACCACC ACGATCTTCA ACACCTAGTA TTTCAGAAGA
1201 TGTAACYYGG ATGTTTGAAA TATATTTGTT ATTCCATAAT GGTTCAAACA
1251 TCGCATTCGC AAAACGTAAT ACCTCGATAT TTTGAACCAT GTCTTTTCCT
1301 AAATAGTGGT CMATACGRTA AATTTCTTCT TCTTTAAATG ATTTACGAAT
1351 TTGATTGTTT AATGCTTCGG CTGATTTTAA ATCACTACCG AATGGTTTTT
1401 CGATAACAAG GCGTTTAAAT CCTTTTGTAT CAGTAAGACC AGAAGATTTT
1451 AGATAATCAG AAATAACGCC AAAGAATTGT GGTGCCATTG CTAAATAGAA
1501 TAGTCGATTA CCTTYTAATT CAAATTGGCT ATCTAATTCA TTACTAAAAT
1551 CTAGTAATTT CTTGATAGCT TTCTTCATTA CTAACATCAT GTCTATGATA
1601 GAAGACATGT TCCATAAACG CGTCAATTTT GTTTGTATCT TTWACGTGCT
1651 TTTGAATTGA TGATTTTAAC TTGATTACGG AAATCATCAT TAGTAATGTC
1701 ACGACGTCCA ATACCGATGA TGGCAATATG TTCATCTAAA TTGTCTTGTT
1751 GGTAGAGATG GAATATTGAT GGAAACAACT TACGATGGCT TAAGTCACCA
1801 GTTGCACCAA AGATTGTGAT TAAACATGGG ATGTGTTTGT TTTTAGTACT
1851 CAAGATTAAA ACCTCAATTC WYMCATTAGA TATATSATTT ATTATKAYMM
1901 GATAATCCAT TTCAGTAGGT CATACMATAT GYTCGACTGT ATGCAGTKTC
1951 TTAAATGAAA TATCGATTCA TGTATCATGT TTAATGTGAT AATTATTAAT
2001 GATAAGTATA ACGTAATTAT CAAAATTTAT ATAGTTATGT CTAACGTTAA
2051 AGTTAGAAAA ATTAACTAGC AAAGACGAAT TTTTAACAGA TTTTGATTCA
2101 AGTATAAATT AAAACTAAAT TGATACAAAT TTTATGATAA AATGAATTGA
2151 AGAAAAGGAG GGGCATATAT GGAAGTTACA TTTTTTGGAA CGAGTGCAGG
2201 TTTGCCTACA AAAGAGAGAA ATACACAAGC AATCGCCTTA AATTTAGAAC
2251 CATATTCCAA TTCCATATGG CTTTTCGACG TTGGTGAAGG TACACAGCAC
2301 CAAATTTTAC ATCATGCAAT TAAATTAGGA AAAGTGACAC ATATATTTAT
2351 TACTCATATG CATGGCGATC ATATTTTTGG TTTGCCAGGA TTACTTTCTA
2401 GTCGTTCTTT TCAGGGCGGT GAACAGAAGC CGCTTACATT GGTTGGACCA
2451 AAAGGAATTA AAGCATATGT GGAAATGTCT ATGAATTTAT CAGA
```

Mutant: NT6
Phenotype: temperature sensitivity
Sequence map: Mutant NT6 is complemented by plasmid pMP33, which contains a 2.3 kb insert of S. aureus genomic DNA. The partial restriction map of the insert is depicted in FIG. 23; open boxes along part of the length of the clone indicate the percentage of the clone for which DNA sequence has been obtained. Database searches at both the nucleic acid and protein levels reveal identity to the S. aureus femA gene, encoding a protein involved in peptidoglycan crosslinking (Genbank Accession No. M23918; published in Berger-Baechi, B., et al., Mol. Gen. Genet. 219, (1989) 263-269) The pMP33 clone contains the complete femA ORF (denoted in relative length and direction by an arrow) as well as 5′ and 3′ flanking DNA sequences, suggesting that it is capable to direct expression of the FemA protein.

DNA sequence data: The following DNA sequence represents sequence data acquired from subclones 1006, 1007 and 1008, using standard sequencing methods and the commercially-available primers T7 and SP6:

```
subclone 1006, a 500 bp Hind III fragment
SEQ ID NO. 4
1006.sp6 Length: 400 nt
    1 AAATAATCTA AAAATTGGTA GTNCTCCTTC AGATAAAAAT CTTACTTTAA
  51 CACCATTCTT TTNAACTNNT TCCGTGTTTC TTTTTCTAAG TCCATCCATA
101 TTTTNAATGA TGTCATCTGC TGTTTTATCT TTTAAATCTA ACACTGAGTG
151 ATAACGGATT TGTAGCACAG GATCAAATCC TTTATGGAAT CCAGTATGTT
201 CAAATCCTAA GTTACTCATT TTATCAAAGA ACCAATCATT ACCAGCATTA
251 CCTGTAATCT CGCCATCATG ATTCAAGTAT TGATATGGTA AATATGGATC
301 GNTATGTAGG TATAGNCAAC GATGTTTTTT AACATATTTT GGATAATTCA
351 TTAAAGNAAA AGTGTACGAG TNCTTGATTT TCATANTCAA TCACTGGACC
subclone 1007, a 900 bp Hind III fragment
SEQ ID NO. 5
1007.sp6 Length: 398 nt
    1 TGCGTGAAAT NACTGTATGG CNTGCNATCT GTAAAGGCAC CAAACTCTTT
  51 AGCTGTTAAA TTTGTAAACT TCATTATCAT TACTCCTATT TGTCTCTCGT
101 TAATTAATTT CATTTCCGTA TTTGCAGTTT TCCTATTTCC CCTCTGCAAA
151 TGTCAAAAAT AATAAATCTA ATCTAAATAA GTATACAATA GTTAATGTTA
201 AAACTAAAAC ATAAACGCTT TAATTGCGTA TACTTTTATA GTAATATTTA
251 GATTTTNGAN TACAATTTCA AAAAAAGTAA TATGANCGTT TGGGTTTGCN
301 CATATTACTT TTTTNGAAAT TGTATTCAAT NTTATAATTC ACCGTTTTTC
351 ACTTTTTNCA AACAGTATTC GCCTANTTTT TTTAAATCAA GTAAACTT
subclone 1008, a 900 bp Hind III fragment
SEQ ID NO. 6
1008.sp6 Length: 410 nt
    1 GTAATGACAA ATNTAACTAC AATCGCTTAA AATATTACAA AGACCGTGTG
  51 TNAGTACCTT TAGCGTATAT CAACTTTAAT GAATATATTA AAGAACTAAA
101 CGAAGAGCGT GATATTTTAA ATAAAGATTT AAATAAAGCG TTAAAGGATA
151 TTGAAAAACG TCCTGAAAAT AAAAAAGCAC ATAACAAGCG AGATAACTTA
201 CAACAACAAC TTGATGCAAA TGAGCAAAAG ATTGAAGAAG GTAAACGTCT
251 ACAAGANGAA CATGGTAATG AATTACCTAT CTCTNCTGGT TTCTNCTTTA
301 TCAATCCATT TGANGTTGTT TATTATGCTG GTGGTACATC AAATGCATTC
351 CGTCATTTTN CCGGAAGTTA TGCAGTGCAA TGGGAAATGA TTAATTATGC
401 ATTAAATCAT
```

Mutant: NT8
Phenotype: temperature sensitivity
Sequence map: Mutant NT8 is complemented by plasmid pMP34, which contains a 3.5 kb insert of S. aureus genomic DNA. The partial restriction map of the insert is depicted in FIG. 24. Database searches at both the nucleic acid and protein levels reveal identity to the DNA sequence for the dfrB (dihydrofolate reductase [EC 1.5.1.3]; EMBL entry Z16422, published in Dale, G. E. et al. Antimicrob. Agents Chemother. 37 (1993) 1400-1405) and tysY (thymidylate synthase [EC 2.1.1.45]; EMBL entry X13290, published in Rouch, D. A. et al. Mol. Microbiol. 3 (1989) 161-175) genes of S. aureus. The relative size and orientations of the genes, along with sequence identities, are depicted as arrows in the restriction map:

DNA sequence data: The following DNA sequence represents data acquired from clone pMP34, starting with M13 forward and M13 reverse primers and applying primer walking strategies to complete the contig:

```
clone pMP34
SEQ ID NO. 7
pMP34 Length: 3479 nt
      1 AAGCTTCATT AAAAACTTTC TTCAATTTAT CAACATATTC AATGACGTTA
    51 GCATGTGCGA CACCAACGGA YTKSAKKTCA TGATCTCCTA TAAATTCAGC
  101 AATTTCCTTT TTCAAGTATT GGATACTAGA ATTTTGAGTT CTCGCATTGT
  151 GCACAAGCTC TAAGCGACCA TCATCTAGTG TACCAATTGG TTTAATTTTC
  201 ATAAGATTAC CAATCAAACC TTTTGTTTTA CTAATTCTGC CACCTTTAAT
  251 TAATTGATTC AATTGCCCTA TAACTACAAA TAATTTAATG TTTTCTCTTA
  301 AATGATTTAA CTTTTTAACT ATTTCAGAAG TTGAGACACC TTCTTTTACA
  351 AGCTCTACTA GGTGTTGTAT TTGATACCCT AAACCAAAAG AAATAGATTT
  401 TGAATCAATA ACAGTTACAT TAGCATCTAC CATTTGACTT GCTTGGTAAG
  451 CAGTGTTATA TGTACCACTT AATCCTGAAG AAAGATGAAT ACTTATGATT
  501 TCAGAGCCAT CTTTTCCTAG TTCTTCATAA GCAGATATAA ATTCACCTAT
  551 GGCTGGCTGA CTTGTCTTTA CATCTTCATC ATTTTCAATA TGATTAATAA
  601 ATTCTTCTGA TGTAATATCT ACTTGGTCAA CGTATGAAGC TCCTTCAATA
  651 GTTAAACTTA AAGGAATTAC ATGWATGTTG TTTGCTTCTA ARTATTCTTT
  701 AGATAAATCG GATGTTGAGT CTGTTACTAT AATCTGTTTT GTCATGGTCG
  751 TTTTCCCCCT TATTTTTTAC GAATTAAATG TAGAAAGGTA TGTGGAATTG
  801 TATTTTTCTC ATCTAGTTTA CCTTCAACTG AAGAGGCAAC TTCCCAGTCT
  851 TCAAATGTAT AAGGTGGAAA GAACGTATCA CCACGGAATT TACCTTCAAT
  901 AACAGTAATA TACATGTCGT CCACTTTATC AATCATTTCT TCAAATAATG
  951 TTTGCCCTCC AAATATGAAA ACATGGCCCG GTAGTTGGTA AATATCTTCA
1001 ATAGARTGAA TTACATCAAC GCCCTCTACG TTGAAACTTG TATCTGAAGT
1051 AAGTACAACA TTTCGACGAT TCGGTAGTGG TTTACCAATC GATTCAAATG
1101 TCTTACGACC CATTACTAAA GTATGACCTG TTGATAATTT TTTAACATGC
1151 TTCAAATCAT TTGGTAGGTG CCAAGGTAAT TGATTTTCAA AACCAATTAC
1201 TCGTTGCAAG TCATGTGCAA CTAGAATGGA TAAAGTCATA ATTATCCTCC
1251 TTCTTCTATC ATTTCATTTT TTATTACTAA GTTATCTTTA ATTTAACACA
1301 ATTTTTATCA TAAAGTGTGA TAGAAATAAT GATTTTGCAT AATTTATGAA
1351 AACGTTTAAC ACAAAAAAGT ACTTTTTTGC ACTTGAAAAT ACTATGATGT
1401 CATTTKGATG TCTATATGGT TAGCTAAYTA TGCAATGACT ACAMTGCTAT
1451 KGGAGCTTTT ATKGCTGGAT GTGATTCATA GTCAACAATT TCCAMAATCT
1501 TCATAATTTA TGTCGAAAAT AGACTTGTCA CTGTTAATTT TTAATGTTGG
1551 AGGATTGAAG CTTTCACGTG CTAATGGTGT TKCGMATCGC ATCAATATGA
1601 TTTGAATAAA TATGTGCATC TCCAAATGTA TGCACAAATT CACCCACTTC
1651 AAGTCCACAT TTCTTTGGCA ATAAGGTGTG TCAATAAAGC GTAGCYTGCG
1701 ATATTAAATG GCACACCTAA AAAGATATCT GCGCTACGTT GGTATAACTG
1751 GCAACTTAAC TTACCATCTT GGACATAAAA CTGGAACATG GTATGACAAG
1801 GCGGAAGTGC CATTGTATCA ATTTCTGTTG GATTCCATGC AGATACGATG
1851 TGTCGCCTTG AATCTGGATT ATGCTTAATT TGTTCAATTA CTGTTTTAAG
1901 TTGATCAAAA TGATTACCAT CTTTATCAAC CCAATCTCGC CMATTGTTTA
1951 CCATAAACAT TTCCTAAATC CCCGAATTGC TTCGCAAATG TATCATCTTC
2001 AAGAATACGT TGCTTAAATT GTTTCATTTG TTCTTTATAT TGTTCGTTAA
2051 ATTCAGGATC ACTCAATGCA CGATGCCCGA AATCTGTCAT ATCTGGACCT
2101 TTATACTCGT CTGATTTGAT ATAATTTTCA AAAGCCCATT CGTTCCATAT
2151 ATTATTATTA TATTTTAATA AGTATTGGAT GTTTGTATCT CCTTTAATGA
2201 ACCATAATAA TTCGGTTGCT ACTAATTTAA AAGAAACTTT CTTTGTCGTT
2251 AATAGTGGAA ATCCTTTAGA TAAGTCAAAG CGAAGTTGAT GACCAAATTT
2301 CGAAATCGTA CCTGTATTTG TGCGATCATT TCGTGTATTT CCTATTTCTA
2351 AAACTTCTTC ACAAAGACTG TGATATGCTG CATCAAATGA ATTTCAACAT
2401 ATGCGATAAC ACCTCATTTT CATTATTTAT AGTATGTATA TTTAGTTTGA
2451 TATAACTTAA CTTTATGTAG CATTTTGTTA TCACTCATTT TAGGAATATG
2501 ATATTAATAT CATGAATTCC GTTACTTTAT TTATAAAATG CTGATTAAGT
2551 ACCTACCCCA TCGTAACGTG ATATATGTTT CCAATTGGTA ATTGTTTACC
2601 CAAATCTATA ACTTTAATGC TAAAAAATTT TAAAAAAGAG GTTAACACAT
2651 GATTTGAATA TTATGTTTGA TGTCCTATTA AAACAGTTAA ATTTCTAGAA
2701 AATATAGTTG GTAAAAACGG ACTTTATTTA ACAAATAGAA TACAACTATA
2751 TTCTCTATTT TCAATGACAG ACACCATTTT TAATATTATA AAATGTGTTA
2801 ACCTTTATAT TTATTTATGT GTACTATTTA CAATTTTCGT CAAAGGCATC
2851 CTTTAAGTCC ATTGCAATGT CATTAATATC TCTACCTTCG ATAAATTCTC
2901 TAGGCATAAA ATAAACTAAA TCTTGACCTT TGAATAAAGC ATACGAAGGA
2951 CTAGATGGTG CTTGCTGAAT GAATTCTCGC ATTGTAGCAG TTGCTTCTTT
3001 ATCTTGCCCA GCAAAAACTG TAACTGTATT TGTAGGTCTA TGTTCATTTT
3051 GTGTTGCAAC TGCTACTGCA GCTGGTCTTG CTAATCCAGC TGCACAGCCG
3101 CATGTAGAGT TAATAACTAC AAAAGTAGTG TCATCAGCAT TTACTTGGTT
3151 CATATACTCC GATACTGCTT CGCTCGTTTC TAAACTTGTA AAACCATTTT
3201 GAGTTAATTC GCCACGCATT TGTTGCGCAA TTTCTTTCAT ATAAGCATCA
3251 TAYGCATTCA TATTTAATTC CTCCAATTAA ATTGTTCTGT TTGCCATTTG
3301 TYTCCATACT GAACCAAGYG CTTCAYCTCC GTTTTCAATA TCGAGATATG
3351 GCCATTTCAA TTTGTAATTT AACWTCAAAC GCMTKGTCAK KAATATGGGS
3401 WTTTAGKGCG GGAAGMTGMT YWGCATWACS WTCATSAWAG ATAWACAYAG
3451 CARCAYSCCA CYTWAYGAKT TTMWKTGGA
```

Mutant: NT12
Phenotype: temperature sensitivity
Sequence map: Mutant NT12 is complemented by pMP37, which contains a 2.9 kb insert of S. aureus genomic DNA. A partial restriction map is depicted FIG. 25. Database searches at both the nucleic acid and peptide levels reveal significant similarities to the protein encoded by the tagG gene, an integral membrane protein involved in the assembly of teichoic acid-based structures, from B. subtilis (Genbank Accession No. U13832; published in Lazarevic, et al., Mol. Microbiology, 16 (1995) 345-355).

DNA sequence data: The following DNA sequence data represents the sequence of clone pMP37, using standard M13 forward and M13 reverse sequencing primers and then completing the sequence contig via primer walking strategies. The sequences below can be used to design PCR primers for the purpose of amplification from genomic DNA with subsequent DNA sequencing.

```
clone pMP37
SEQ ID NO. 8
pMP37 Length: 2875 nt
      1 GTGGTTCCCT GTCATTYTRA TATCCATCAA ACCTTTATTA ATACACGTRG
    51 CTATCGAAGC ATTTTGTAAT TGTATTAATG AAATATGCTT GAGTYCTCTT
  101 TGTAACCGTT CAATCATAGG AATTGTTTGA TCAGTAGAAC CACCATCAAT
  151 ACAAAGGATT CTATAGTGTT CTTTACTCTC AATAGATATT AACAATTGTC
  201 GAATTGTTGC CTCATTATTA CATGTAGGTA TGATTATCGT AAACCTCATT
  251 TTGTCACCAT CTTATCTATA TATTCTGTGA GCTGATGTAA ACTTTTATCA
  301 GTATTATACT TATGCCAATC TTTAAATAAC GGACTTAATA GATGTTCTTT
  351 TTCTTGTATC GTCATTATTA AATCTTCTTC AGTATACACT TTGTAGCTAT
  401 CCGGTATTGC TTTGTAAAAT TGATTCAGGC CTCTCACCTG ATCATATGTT
  451 CCTTCATCAT ACACATAAAA TATAGTTGGA ATATCTAACA AGCTAGCTTC
  501 TATTGGCAGC GAACTATAGT CGCTAATAAT TATATCTGAC ATTAGCATTA
  551 ATGTAGACGT GTCGATTGAA GATACGTCAT CAATGTCTGA ATCTTCAATT
  601 GATGGATGTA ATTTATTAAT CAGTGTATAT CCTGGTAAAC ATTTTTCAAA
  651 ATAAGCTTTA TCAATAGCCC TATTATCTGC TTTATCTTCT CTATATGTTG
  701 GTACATATAA TACCAACTTA TTTGTAATTC CATATTTATC CTTTAACTCT
  751 GCCTTAACCG TTGCTCTATC AGCTGTGTAA TATTTATTAA TTCTCGGAAG
  801 CCCAAAATAC AGCATTTGCT CTTCTGTTGC ACCTAAAGAC TGTTTAAAAC
  851 ATTGTGACAT TTGTTCACAA CCCACTAAGT TAAAAATCCG TCGCTTGATA
  901 AACTTTACGG TACTGCTGAA CCATTGCCTT GTCAGACACA TCGACTTGAT
  951 GATCTGTTAA GCCAAAGTTT TTTAATGCAC CACTTGCATG CCACGTTTGA
1001 ACAATGTGTT TGATTAGAAK TCTTATTATA TCCACCTAGC MATAGGTAAT
1051 AATTATCGAT AATAATCATC TGCGCGCTTT TCAAAGCCTT AATTTGTTTT
1101 ACCAATGTTC GATTAGTCAT TTCTATCACA TCAACATCGT CGCTAAGTTC
1151 AGATAAATAA GGCGCTTGTT TTGGTGTTGT TAAAACAGTT TTCTGATACG
1201 ACGAATTATT TAATGCTTTG ATGATAGGCT TAATATCTTC TGGAAAAGTC
1251 ATCATAAATA CGATATGCGG TTTATCAATC ACTTGAGGSG TAWTCATTTW
1301 AGRAAGTATT CGAACTACCA AATGATAAAA TTTCTTTATT AAAAACGTTC
1351 ATAATAACAC CAACTTAATA TGTTATTTAA CTTAAATTAT AAACAAAAAT
1401 GAACCCCACT TCCATTTATT AATGGTTAGC GGGGTTTCGT CATATAAATA
1451 TATTACAAGA AGTCTGCAAA TTGATCTCTA TATTTCATGT GTWAGTACGC
1501 MCCMATTGCA AAGAAAATGG CAACAATACC GAAATTGTAT AACATTAATT
1551 TCCAATGATC CATGAAATAC CATTCGTGAT ATAAAATTGC TGCACKKTWT
1601 KATTMAKCWR TAMRGTMAAC TRGMTKATAT TTCATCATTK SATGAATTAA
1651 ACCACTGATA CCATGGTTCT TTGGTAGCCA CAAAATTGGT GAAAAGTAAA
1701 ATAATATTCT TAATATTGGC TTGCATTAAC ATTTGTGTAT CTCTAACTAA
1751 CAACACCGAG TGTTGATGTT AATAACGTCA CCGAGGCAGT TAAGAAAAAA
1801 CAAAACGGTA CATATATCAA TAATTGAATG ATATGTATTG ATGGATAAAT
1851 ACCAGTAAAC ATACATGCAA TTATCACAAG TAAAAGTAAG CCTAAATGTC
1901 CATAAAATCT ACTTGTCACA ATATATGTCG GTATTATCGA TAACGGGAAG
1951 TTCATTTTCG ATACTTGATT AAACTTTTGT GTAATTGCTT TAGTACCTTC
2001 TAAAATACCT TGGTTGATGA AGAACCACAT ACTGATACCA ACCAATAACC
2051 AATAAACAAA AGGTACACCA TGAATTGGTG CATTACTTCT TATTCCTAAT
2101 CCAAAAACCA TCCAGTAAAC CATAATTTGC ATAACAGGGT TAATTAATTC
2151 CCAAGCCACA CCTAAATAGT TACTATGATT GATAATTTTA ACTTGAAACT
2201 GAGCCAGTCT TTGAATTAAA TAAAAGTTCT WTASATGTTC TTTAAAAACT
2251 GTTCCTATTG CTGACATTCC ATTAAACCAC ACTTTCAAAT GTTTAACTAT
2301 TTCTCTAACT TAACTAAATA GTATTATAAT AATTGTTGTA AATACTATCA
2351 CTAWACATGG ATGCTATCAA AATTATTGTC TAGTTCTTTA AAATATTAGT
2401 TTATTACAAA TACATTATAG TATACAATCA TGTAAGTTGA AATAAGTTTA
2451 GTTTTTAAAT ATCATTGTTA TCATTGATGA TTAACATTTT GTGTCAAAAC
2501 ACCCACTCTG ATAATAACAA AATCTTCTAT ACACTTTACA ACAGGTTTTA
2551 AAATTTAACA ACTGTTGAGT AGTATATTAT AATCTAGATA AATGTGAATA
2601 AGGAAGGTCT ACAAATGAAC GTTTCGGTAA ACATTAAAAA TGTAACAAAA
2651 GAATATCGTA TTTATCGTAC AAATAAAGAA CGTATGAAAG ATGCGCTCAT
2701 TCCCAAACAT AAAAACAAAA CATTTTTCGC TTTAGATGAC ATTAGTTTAA
2751 AAGCATATGA AGGTGACGTC ATAGGGCTTG TTGGCATCAA TGGTTCCGGC
2801 AAATCAACGT TGAGCAATAT CATTGGCGGT TCTTTGTCGC CTACTGTTGG
2851 CAAAGTGGAT CGACCTGCAG TCATA
```

Mutant: NT14
Phenotype: temperature sensitivity
Sequence map: Mutant NT14 is complemented by plasmid pMP40, which contains a 2.3 kb insert of S. aureus genomic DNA. The partial restriction map of the insert is depicted in FIG. 26 (no Eco RI, Hind III, Bam HI or Pst I sites are apparent); open boxes along part of the length of the clone indicate the percentage of the clone for which DNA sequence has been obtained. Database searches at both the nucleic acid and protein levels reveal identity to the Staph. aureus femB gene, encoding a protein involved in peptidoglycan crosslinking (Genbank Accession No. M23918; published in Berger-Baechi, B., et al., Mol. Gen. Genet. 219, (1989) 263-269). The pMP40 clone contains the complete FemB ORF (denoted in relative length and direction by an arrow) as well as 5′ and 3′ flanking DNA sequences, suggesting that it is capable to direct expression of the FemB protein; the relation of the femA gene is also depicted to demonstrate the extent of identity between the clone and the Genbank entry.

DNA sequence data: The following DNA sequence data represents the sequences at the left-most and right-most edges of clone pMP40 obtained with the standard DNA sequencing primers T7 and SP6, and can be used to demonstrate identity to part of the published sequence (Genbank No. M23918):

```
SEQ ID NO. 9
1015.t7 LENGTH: 453 nt
    1 CTTAAAATAT TACAAAGACC GTGTGTNAGT ACCTTNAGCG TATATcAaCT
  51 TTAATGAATA TATTAAAGAA CTAAACGAAG AGCGTGATAT TTTAAATAAA
101 GATTTAAATA AAGCGTTAAA GGATATTGAA AAACGTCCTG AAAATAAAAA
151 AGCACATAAC AAGCGAGATA ACTTACAACA ACAACTTGAT GCAAATgAGC
201 AAAAGATTGA NGACGGTAAA CGTCTACAAG ANGANCATGG TAATGNTTTA
251 CCTATCTCTC CTGGTTTCTC CTTTATCAAT CCNTTTGANG TTGTTTATTA
301 TGCTGGTGGT ACATCAAATG CNTTCCGTCA TTTTNCCGGA NGTTATGCNG
351 TGCAATGGGA AATGNTTAAT TTTGCATTAA ATCATGGCAT TGNCCGTTAT
401 AATTNCTATG GTGTTAGTGG TNAATTTNCA GNAGGTGCTG AAGATGCTGG
451 TGT
SEQ ID NO. 10
1015.sp6 LENGTH: 445 nt
    1 ATGCTCAGGT CGATCATACA TCTATCATCA TTttAATTTC TAAAATACAA
  51 ACTGAATACT TTCCTAGAaT NTNaNACAGC AATCATTGCT CATGCATTTA
101 ATAAATtaCA ATTAGACAAA TATGACATTT gATATCACAC ACTTGCAAAC
151 ACACACATAT ATAATCAGAC ATAAATTGTT ATGCTAAGGT TTATTCACCA
201 AAANTATAAT ACATATTGGC TTGTTTTGAG TCATATTGNN TGANTTANAA
251 NGTATACTCA ACTCANTCAT TTNCAATTNG GTTGTGCAAT TCNTATTTNT
301 NTTTCTTGCA ATCCCTTGTT AAACTTGTCA TTTNATATAT CATTNTTCGG
351 GGCTTTATTA AAANNCATNT NNNACNGNGC CTATNGNNTC NNTNACTATN
401 NGCCCTAACA TCATTTTCNT CTNTTTCTTA TTTTTTACGG GATTT
```

Mutant: NT15
Phenotype: temperature sensitivity
Sequence map: Mutant NT15 is complemented by plasmid pMP102, which contains a 3.1 kb insert of S. aureus genomic DNA. The partial restriction map of the insert is depicted in FIG. 27; open boxes along part of the length of the clone indicate the percentage of the clone for which DNA sequence has been obtained. Database searches at both the nucleic acid and protein levels reveal strong identity at both the peptide and nucleic acid level to the SecA protein from S. carnosus (Genbank Accession No. X79725; submitted in 1994, unpublished as of 1995); the relative size and location of the secA gene predicted from similarity to the S. carnosus gene is depicted below by an arrow. The SecA protein is involved in the protein secretory pathway and serves an essential cellular function.

DNA Sequence Data:

```
clone pMP102
SEQ ID NO. 11
pMP102.forward Length: 719 nt
    1 GATCRAGGAG ATCAAGAAGT GTTTGTTGCC GAATTACAAG AAATGCAAGA
  51 AACACAAGTT GATAATGACG CTTACGATGA TAACGAGATA GAAATTATTC
101 GTTCAAAAGA ATTCAGCTTA AAACCAATGG ATTCAGAAGA AGCGGTATTA
151 CAAATGAATC TATTAGGTCA TGACTTCTTT GTATTCACAG ACAGAGAAAC
201 TGATGGAACA AGTATCGTTT ACCGCCGTAA AGACGGTAAA TATGGCTTGA
251 TTCAAACTAG TGAACAATAA ATTAAGTTTA AAGCACTTGT GTTTTTGCAC
301 AAGTGCTTTT TTATACTCCA AAAGCAAATT ATGACTATTT CATAGTTCGA
351 TAATGTAATT TGTTGAATGA AACATAGTGA CTATGCTAAT GTTAATGGAT
401 GTATATATTT GAATGTTAAG TTAATAATAG TATGTCAGTC TATTGTATAG
451 TCCGAGTTCG AAAATCGTAA AATATTTATA ATATAATTTA TTAGGAAGTT
501 ATAATTGCGT ATTGAGAATA TATTTATTAG TGATAAACTT GTTTGACACA
551 GAATGTTGAA TGAATTATGT CATAAATATA TTTATATTGA TCTACCAATG
601 AGTAAATAAN TATAATTTCC TAACTATAAA TGATAAGANA TATGTTGTNG
651 GCCCAACAGT TTTTTGCTAA AGGANCGAAC GAATGGGATT TTATCCAAAA
701 TCCTGATGGC ATAATAAGA
SEQ ID NO. 12
pMP102.reverse Length: 949 nt
    1 CTTTACCATC TTCAGCTGAA ACGTGCTTCG CTTCACCAAA CTCTGTTGTT
  51 TTTTCACGTT CAATATTATC TTCAACTTGT ACTACAGATT TTAAAATGAA
101 TTTACAAGTA TCTTCTTCAA TATTTTGCAT CATGATATCA AATAATTCAT
151 GACCTTCATT TTGATAGTCA CGTAATGGAT TTTGTTGTGC ATAAGAACGT
201 AAGTGAATAC CTTGACGTAA TTGATCCATT GTGTCGATAT GATCAGTCCA
251 ATGGCTATCA ATAGAACGAA GTAAAATCAT ACGCTCAAAC TCATTCATTT
301 GTTCTTCTAA GATATCTTTT TGACTTTGAT ATGCTGCTTC AATCTTAGCC
351 CAAACGACTT CGAAAATATC TTCAGCATCT TTACCTTTGA TATCATCCTC
401 TGTAATGTCA CCTTCTTGTA AGAAGATGTC ATTAATGTAG TCGATGAATG
451 GTTGATATTC AGGCTCGTCA TCTGCTGTAT TAATATAGTA ATTGATACTA
501 CGTTGTAACG TTGAACGTAG CATTGCATCT ACAACTTGAG AGCTGTCTTC
551 TTCATCAATA ATACTATTTC TTTCGTTATA GATAATTTCA CGTTGTTTAC
601 GTAATACTTC ATCGTATTCT AAGATACGTT TACGCGCGTC GAAGTTATTA
651 CCTTCTACAC GTTTTTGTGC TGATTCTACA GCTCTTGATA CCATTTTTGA
701 TTCAATTGGT GTAGAGTCAT CTAAACCTAG TCGGCTCATC ATTTTCTGTA
751 AACGTTCAGA ACCAAAACGA AATCATTAAT TCATCTTGTA ATGATAAATA
801 GAAGCGACTA TCCCCTTTAT CACCTTGACG TCCAGAACGA CCACGTAACT
851 GGTCATCAAT ACGACGAAGA TTCATGTCGC TCTGTACCTA TTACTGCTAA
901 ACCGCCTAAT TCCTCTACGC CTTCACCTAA TTTGATATCT GTACCACGA
SEQ ID NO. 13
pMP102.subclone Length: 594 nt
    1 GGGGATCAAT TTANAGGACG TACAATGCCA GGCCGTCGTT NCTCGGAAGG
  51 TTTACACCAA GCTATTGAAG CGAGGAAAGG CGTTCAAATT CAAAATGAAA
101 TCTAAAACTA TGGCGTCTAT TACATTCCAA AACTATTTCA GAATGTACAA
151 TAAACTTGCG GGTATGACAG GTACAGCTAA AACTGAAGAA GAAGAATTTA
201 GAAATATTTA TAACATGACA GTAACTCAAA TTCCGACAAA TAAACCTGTG
251 CAACGTAACG ATAAGTCTGA TTTAATTTAC ATTAGCCAAA AAGGTAAATT
301 TGATGCAGTA GTAGAAGATG TTGTTGAAAA ACACAAGGCA GGGCAACCMG
351 TGCTATTAGG TACTGTTGCA GTTGAGACTT CTGTATATAT TTCAAATTTA
401 CTTAAAAAAC GTGGTATCCG TCATGATGTG TTAAATGCGA RAAATCATGA
451 MCGTGAAGCT GAAATTGTTG CAGGCGCTGG RCAAAAAGGT GCCGTTACTA
501 TTGCCACTAM CATGGCTGGT CGTGGTACAG ATATCAAATT AGGTGAAGGC
551 GTTANAANGA AATTAGGCGG TTTANCCAGT AATANGTTCA GAAG
```

Mutant: NT16
Phenotype: temperature sensitivity
Sequence map: Mutant NT16 is complemented by plasmid pMP44, which contains a 2.2 kb insert of S. aureus genomic DNA. The partial restriction map of the insert is depicted in FIG. 28. Database searches at both the nucleic acid and protein levels reveal significant similarity at the peptide level to an ORF (orf3) of unknown function in the serotype “A” capsulation locus of H. influenzae (Genbank Accession No. Z37516); similarity also exists at the protein level to the tagB gene of B. subtilis (Genbank Accession No. X15200), which is involved in teichoic acid biosynthesis. Based upon the peptide level similarities noted, it is possible that the ORF(s) contained within this clone are involved in some aspect of membrane biogenesis, and should make an excellent screening target for drug development. No significant similarities are observed at the nucleic acid level, strengthening the stance that clone pMP44 represents a novel gene target(s).

DNA sequence data: The following DNA sequence data represents the sequence generated by primer walking through clone pMP44, starting with standard M13 forward and M13 reverse sequencing primers. The sequence below can be used to design PCR primers for the purpose of amplification from genomic DNA with subsequent DNA sequencing.

```
clone pMP44
SEQ ID NO. 14
pMP44 Length: 2192 nt
      1 GCATGMCTGC AGGTCGATCY SYTGAACAGT CATCAACTAC AACCACTTCA
    51 AATTCAGTTT TCGGAAAATC TTGTTTCGCA AGGCTATTAA GTAATTCTGT
  101 TATATACTTT TCTGAATTGT ATGTTGGAAC TATTACTGAA AATTTCATCA
  151 TTATACCTCT CCCACTTTGA CTACTATATA AACTTAGCTA CCAAATAAAT
  201 TTCTGACTAA ACGCTCACTT GATCGGCCAT CTTGATATTT AAAATGTTTA
  251 TCTAAGAATG GAATGACTTT TTCTCCTTCA TAATCTTCAT TGTCCAAGGC
  301 GTCCATTAAT GCGTCAAATG ATTGCACAAT TTTACCTGGA ACAAATGATT
  351 CATATGGTTC ATAAAAATCA CGCGTCGTAA TATAATCTTC TAAATCAAAT
  401 GCATAGAAAA TCATTGGCTT TTTAAATACT GCATATTCAT ATATTAAAGA
  451 TGAATAGTCA CTAATTAATA AATCTGTTAT GAACAGTATA TCATTAACTT
  501 CTCTAAAGTC AGAAACGTCA ACAAAATATT GTTTATGTTT GTCTGCAATA
  551 TTAAGTCTAT TTTTCACAAA TGGATGCATT TTAAATAATA CAACCGCGTT
  601 ATTTTTTTCG CAATATCTTG CTAAACGTTC AAAATCAATT TTGAAAAATG
  651 GGTAATGTGC TGTACCATGA CCACTACCTC TAAATGTTGG TGCGAAAAGA
  701 ATGACTTTCT TACCTTTAAT AATTGGTAAT TCATCTTCCA TCTCTTGTTT
  751 GATCTGTGTC GCATAAGCTT CATCAAATAG TACATCAGTA CGTTGGGAAC
  801 ACCTGTAGGC ACTACATTTT TCTCTTTAAT ACCAAATGCT TCAGCGTAGA
  851 ATGGAATATC GGTTTCAAGA TGATACATAA GCTTTTGTAT AAGCTACGGA
  901 TGATTTAATG AATCAATAAA TGGTCCACCC TTTTTACCAG TACGACTAAA
  951 GCCAACTGTT TTAAAGGCAC CAACGGCATG CCATACTTGA ATAACTTCTT
1001 GAGAACGTCT AAAACGCACT GTATAAATCA ATGGGTGAAA GTCATCAACA
1051 AAGATGTAGT CTGCCTTCCC AAGTAAATAT GGCAATCTAA ACTTGTCGAT
1101 GATGCCACGT CTATCTGTAA TATTCGCTTT AAAAACAGTG TGAATATCAT
1151 ACTTTTTATC TAAATTTTGA CGTAACATTT CGTTATAGAT GTATTCAAAG
1201 TTTCCAGACA TCGTTGGTCT AGAGTCTGAT GTGAACAACA CCGTATTCCC
1251 TTTTTTCAAG TGGAAAAATT TCGTCGTATT AAATATCGCT TTAAAAATAA
1301 ATTGTCTTGT ATTAAATGAT TGTTTGCGGA AATACTTACG TAATTCTTTA
1351 TATTTACGRA CGATATAAAT ACTTTTAAMT TCCCGGAGTC GTTACAACAA
1401 CATCAAGGAC AAATTCATTA ACATCGCTAG AAATTTCAGG TGTAACAGTA
1451 TAAACCGTTT TCTTTCGAAA TGCCGCCTTT TCTAAATTCT TTTAGGTAAG
1501 TCTGCAATAA GAAATTGATT TTACCATTTT GTGTTTCTAA TTCGYTGTAT
1551 TCTTCTTCTT GTTCTGGCTT TAGATTTTGA TATGCATCAT TAATCAACAT
1601 CTGGGTTTAA CTGTGCAATA TAATCAAGTT CTTGCTCATT CACTAATAAG
1651 TACTTATCTT CAGGTAAGTA ATAACCATTA TCTAAGATAG CTACATTGAA
1701 ACGACAAACG AATTGATTCC CATCTATTTT GACATCATTC GCCTTCATTG
1751 TACGTGTCTC AGTTAAATTT CTTAATACAA AATTACTATC TTCTAAATCT
1801 AGGTTTTCAC TATGTCCTTC AACGAATAAC TGAACACGTT CCCAATAGAT
1851 TTTAYCTATA TATATCTTAC TTTTAACCAA CGTTAATTCA TCCTTTTCTA
1901 TTTACATAAT CCATTTTAAT ACTGTTTTAC CCCAAGATGT AGACAGGTCT
1951 GCTTCAAAAG CTTCTGTAAG ATCATTAATT GTTGCAATTT CAAATTCTTG
2001 ACCTTTTAAA CAACGGCTAA TTTATCTAAC AATATCTGGG TATTGAATGT
2051 ATAAGTCTAA CAACATCTTG GAAATCTTTT GAACCACTTC GACTACTACC
2101 AATCAACGTT AGTCCTTTTT CCAATACTAG AACGTGTATT AACTTCTACT
2151 GGGAACTCAC TTACACCTAA CAGTGCAATG CTTCCTTCTG GT
```

Mutant: NT17
Phenotype: temperature sensitivity
Sequence map: Mutant NT17 is complemented by plasmid pMP45, which contains a 2.4 kb insert of S. aureus genomic DNA. The partial restriction map of the insert is depicted in FIG. 29. Database searches at both the nucleic acid and protein levels reveal a strong similarity to the product of the apt gene, encoding adenine phosphoribosyl transferase (EC 2.4.2.7) from E. coli (Genbank Accession No. M14040; published in Hershey, H. V. et al. Gene 43 (1986) 287-293).

DNA sequence data: The following DNA sequence data represents the sequence generated by primer walking into clone pMP45, starting with standard M13 forward and M13 reverse sequencing primers. The sequence below can be used to design PCR primers for the purpose of amplification from genomic DNA with subsequent DNA sequencing:

```
clone pMP45
SEQ ID NO. 15
pMP45 Length: 2431 nt
      1 ATGCAGGTCG ATCNCCTNGT TTATTCNGNT TCATCATTTT CCGATAAATA
    51 CTGTAAATAT GNNTAGGTCT ACCATTTATA TCGCCTTCGA TATTCATTCG
  101 GTCCATTTCA GTACGTATTC TATCAATAGC CGTTTCGATA TACGCTTCAC
  151 GTTCACTACG TTTCTTCTTC ATTAAATTGA CTATTCTAAA ATATTGCACA
  201 TTATCAATAT AACGAAGAGC CGKATCTTCT AGTTCCCATT TGATTGTATT
  251 AATACCAAGA CGATGTGCTA ATGGTGCATA AATTTCTAAT GTTTCTCGAG
  301 AAATTCTAAT TTGKTTTTCG CGCGGSATGG STTTCAAGGT ACGCATATTA
  351 TGTAATCTGT CTGCTAATTT CANCAAAATT ACGCGTACAT CTTTGGCAAT
  401 CGCAATAAAT AACTTGSGAT GATTTTCAGC TTGTTGTTCT TCTTTTGAGC
  451 GGTATTTTAC TTTTTTAAGC TTCGTCACAC CATCAACAAT TCGAGCAACT
  501 TCTTCATTGA ACATTTCTTT TACATCTTCA AATGTATACG GTGTATCTTC
  551 AATTACATCA TGCAAAAAAC CTGCGACAAT CGTCGGTCCG TCTAATCGCA
  601 TTTCTGTTAA AATACCTGCA ACTTGTATAG GATGCATAAT GTATGGTAAT
  651 CCGTTTTTTC GGAACTGACC TTTATGTGCT TCATAAGCAA TATGATAGCT
  701 TTTTAAAACA TACTCATATT CATCTGCTGA CAAATATGAT TTTGCTTTGT
  751 GAAGAACTTC GTCTGCACTA TATGGATATT CGTTGTTCAT TATATGATAC
  801 ACCCCATTCA TATTTATTAC TTCGCCTTTA AACAATGGAT TTAGGTACTC
  851 TTGTTGAATA GTATTTGTCC CACACCAATC ATACGTCCGT CGACGATAAA
  902 TATTTATCCT GTCGTGCATT AATCGTAATA TTAATTTTAC TTGAGCGAGT
  951 TTAATTTGTA TACTATTCCT ACTTTTAAAA CTTTTACAAA AATTCGACCT
1001 AAATCTACTG TTTCATTTTT TAAATATTAG TTCTATGATA CTACAATTTA
1051 TGARATAAAT AAACGAWGTT ATTAAGGTAT AATGCTCMAT CATCTATCAT
1101 TTTCAGTAAA TAAAAAATCC AACATCTCAT GTTAAGAAAA CTTAAACAAC
1151 TTTTTTAATT AAATCATTGG TYCTTGWACA TTTGATRGAA GGATTTCATT
1201 TGATAAAATT ATATTATTTA TTATTCGTCG TATGAGATTA AACTMATGGA
1251 CATYGTAATY TTTAAWAKTT TTCMAATACC AWTTAAAWKA TTTCAATTCA
1301 AATTATAAAW GCCAATACCT AAYTACGATA CCCGCCTTAA TTTTTCAACT
1351 AATTKTATKG CTGYTCAATC GTACCACCAG TAGCTAATAA ATCATCTGTA
1401 ATTRRSACAG TTGACCTGGK TTAATTGCAT CTTKGTGCAT TGTYAAAACA
1451 TTTGTACCAT ATTCTAGGTC ATAACTCATA ACGAATGACT TCACGAGGTA
1501 ATTTCCCTTC TTTTCTAACA GGTGCAAAGC CAATCCCCAT KGAATAAGCT
1551 ACAGGACAGC CAATGATAAA GCCAACGSGC TTCAGGTCCW ACAACGATAT
1601 CAAACATCTC TGTCTTTTGC GTATTCWACA ATTTTATCTG TTGCATAGCC
1651 ATATGCTTCA CCATTATCCA TAATTGTAGT AATATCCTTG AAACTAACAC
1701 CTGGTTTCGG CCAATCTTGA ACTTCTGATA CGTATTGCTT TAAATCCATT
1751 AATATTTCCT CCTAAATTGC TCACGACAAT TGTGACTTTA TCCAATTTTT
1801 TATTTCTGAA AAATCTTGAT ATAATAATTG CTTTTCAACA TCCATACGTT
1851 GTTGTCTTAA TTGATATACT TTGCTGGAAT CAATCGATCT TTTATCAGGT
1901 TGTTGATTGA TTCGAATTAA ACCATCTTCT TGTGTTACAA ATTTTAAGTC
1951 TAAGAAAACT TTCAACATGA ATTTAAGTGT ATCTGGTTTC ACACTTAAAT
2001 GTTGACACAA TAACATACCC TCTTTCTGGA TATTTGTTTC TTGTTTAGTT
2051 ATTAATGCTT TATAACACTT TTTAAAAATA TCCATATTAG GTATACCATC
2101 GAAQTAAATC GAATGATTAT GTTGCAAAAC TATAKAAAGW TGAGAAAATT
2151 GCAGTTGTTG CAAGGAATTA GACAAGTCTT CCATTGACGT TGGTAAATCT
2201 CTTAATACTA CTTTATCAGT TTGTTGTTTA ATTTCTTCAC CATAATAATA
2251 TTCATTCGCA TTTACTTTAT CACTTTTAGG ATGAATAAGC ACGACAATAT
2301 TTTCATCATT TTCTGTAAAA GGTAAACTTT TTCGCTTACT TCTATAATCT
2351 AATATTTGCT GTTCATTCAT CGCAATATCT TGAATAATTA TTTGCGGTGA
2401 TTGATTACCA TTCCATTCGT TGATTTGAAC A
```

Mutant: NT18
Phenotype: temperature sensitivity
Sequence map: Mutant NT18 is complemented by pMP48, which contains a 4.7 kb insert of S. aureus genomic DNA. A partial restriction map is depicted in FIG. 30, along with open boxes to indicate the percentage of the clone for which DNA sequence has been obtained; the sequence contig will be completed shortly. Database searches at both the nucleic acid and peptide levels reveal a strong peptide-level similarity to the ureD gene product, encoding a putative regulatory protein with strong similarities to the phosphomannomutase and the phosphoglucomutase from E. coli. The right-most sequence contig from the diagram below is responsible for complementing mutant NT102, described later; however, the full pMP48 clone described here is required for complementing mutant NT18. Based upon genomic organization and peptide-level similarities, it is highly likely that mutants NT18 and NT102 represent two different proteins in the same biochemical pathway.

DNA sequence data: The following DNA sequence data represents the sequence obtained from clone pMP48, starting with standard M13 forward and M13 reverse sequencing primers and applying primer walking strategies to augment the sequence contigs. The sequences below can be used to design PCR primers for the purpose of amplification from genomic DNA with subsequent DNA sequencing:

```
clone pMP48
SEQ ID NO. 16
pMP48.forward Length: 2018 nt
      1 GCATCAGTTG GTACTTTAAA TAAATGTGCA GTACCAGTCT TAGCAACATT
    51 TACAGTTGCT AATTCAGTAT TTTTCTTAGC ATCTTTAATA ACTAAATTTG
  101 TTGCACCTTG CTTACTATTC GTTTGCATAG TAGTAAAGTT AATAATTAAT
  151 TCTGAATCTG GTTTTACATT TACAGTTTTT GAAATACCGT TAAAGTTACC
  201 ATGATCTGTA GAATCATTTG CATTCACACG ACCTAATGCA GCCACGTTTC
  251 CTTTAGCTTG ATAGTTTTGA GGGTTATTCT TATCAAACAT ATCGCTTCGT
  301 CTTAATTCTG AGTTAACGAA ACCAATCTTA CCGTTGTTAA TTAATGAATA
  351 ACCATTTACT TTATCTGTAA CAGTTACAGT TGGATCCTGT CTATTCTCAT
  401 CTGTTGATAT GGCAGGATCA TCAAATGTTA ATGTCGTATT AATACTGCCT
  451 TCACCAGTAT TGCTAGCATT TGGATCTTGA GTTTGTGCGT TTGCTGCTAC
  501 AGGTGCTGCT GGTTGCGCTG CTGCTGGANC ATTCGCTGGC TGTGTTTGAT
  551 TTGCCGGTGT TGCATTATTA TWAGGTGTTG CTTGGTTATT TCCTTGACCT
  601 GCTTGGTWTG CCGGTGTTGC TTGATTTCCA GGTTGTGCAT GTGCAACGTT
  651 ATTCGGATCA GCTTGATCAC CTTGTCCAGC TGGTTGTGTA TTTGGTTGTG
  701 CTGCTCCTCC TGCTGGATTA GCCTGTCCAC CTTGGTTTGC TGGTTGTACT
  751 GCTGGTTGTC CTTGGTTGGC AGGTGCAGCT GGCTGTGCTG TAGGATTAGC
  801 TTGAGCACCA GCATTTGCGT TAGGCTGTGT ATTGGCATCA GCTGGTTGTG
  851 CTGGTTGATT TTGTGCAGGC TGATTTTGCT CTGCTGCAKA CGCTGTTGTC
  901 GGGTTAGTAG ATATAAAAGT AACAGTGGCA ATTAAAGCTG AAAAAATACC
  951 GACATTAAAT TTTCTGATAC TAAATTTTTG TTGTCTGAAT AAATTCATTA
1001 AGTCATCCTC CTGGTTGATT ATTCTCGCTG TTAAATGATT TCACTTAATC
1051 AACTGTTAAG ATAAGTAGTA GCATCTGCGT TAAAAACACA AAGCAACTCT
1101 ATCTAATTAA AATTAATTTT ATCATCATTA TATATTGAGT ACCAGTGTAT
1151 TTTATATTAC ATATTGATTA CTTTGTTTTT ATTTTGTTTA TATCATTTTA
1201 CGTTTGTACT ATAAATTATT TCTACAAACA CAAAAAACCG ATGCATACGC
1251 ATCGGCTCAT TTGTAATACA GTATTTATTT ATCTAATCCC ATTTTATCTT
1301 GAACCACATC AGCTATTTGT TGTGCAAATC TTTCAGCATC TTCATCAGTT
1351 GCTGCTTCAA CCATGACACG AACTAATGGT TCTGTTCCAG AAGGTCTTAC
1401 TAAAATTCGA CCTTCTCCAT TCATTTCTAC TTCTACTTTA GTCATAACTT
1451 CTTTAACGTC AACATTTTCT TCAACACGAT ATTTATCTGT TACGCGTACG
1501 TTAATTAATG ATTGTGGATA TTTTTTCATT TGTCCAGCTA ATTCACTTAG
1551 TGATTTACCA GTCATTTTTA TTACAGAAGC TAATTGAATA CCAGTTAATA
1601 AACCATCACC AGTTGTATTG TAATCCAYCA TAACGATATG TCCARATKGT
1651 TCTCCACCTA AGTTATAATT ACCGCGAMGC ATTTCTTCTA CTACATATCT
1701 GTCGCCAACT TTAGTTTTAT TAGATTTAAT TCCTTCTTGT TCAAGCGCTT
1751 TGTAAAAACC TAAATTACTC ATAACAGTAG AAAACGAATC ATGTCATTAT
1801 TCAATTCTTG ATTTTTATGC ATTTCTTGAC CAATAATAAA CATAATTTGG
1851 TCACCGTCAA CGATTTGACC ATTCTCATCT ACTGCTATGA TTCTGTCTCC
1901 ATCGCCGTCA AATGCTAACC CAAAATCACT TTCAGTTTCA ACTACTTTTT
1951 CAGCTAATTT TCAGGATGTG TAAAGCCACA TTTCTCATTG ATATTATATC
2001 CATCAGGGAC TACATCCA
SEQ ID NO. 17
pMP48.reverse Length: 2573 nt
      1 ATTCGAGCTC GGTACCCGKG GATCCTSYAG AGTCGATCCG CTTGAAACGC
    51 CAGGCACTGG TACTAGAGTT TTGGGTGGTC TTAGTTATAG AGAAAGCCAT
  101 TTTGCATTGG AATTACTGCA TCAATCACAT TTAATTTCCT CAATGGATTT
  151 AGTTGAAGTA AATCCATTGA TTGACAGTAA TAATCATACT GCTGAACAAG
  201 CGGTTTCATT AGTTGGAACA TTTTTTGGTG AAACTTTATT ATAAATAAAT
  251 GATTTGTAGT GTATAAAGTA TATTTTGCTT TTTGCACTAC TTTTTTTAAT
  301 TCACTAAAAT GATTAAGAGT AGTTATAATC TTTAAAATAA TTTTTTTCTA
  351 TTTAAATATA TGTTCGTATG ACAGTGATGT AAATGATTGG TATAATGGGT
  401 ATTATGGAAA AATATTACCC GGAGGAGATG TTATGGATTT TTCCAACTTT
  451 TTTCAAAACC TCAGTACGTT AAAAATTGTA ACGAGTATCC TTGATTTACT
  501 GATAGTTTGG TATGTACTTT ATCTTCTCAT CACGGTCTTT AAGGGAACTA
  551 AAGCGATACA ATTACTTAAA GGGATATTAG TAATTGTTAT TGGTCAGCAG
  601 ATAATTWTGA TATTGAACTT GACTGCMACA TCTAAATTAT YCRAWWYCGT
  651 TATTCMATGG GGGGTATTAG CTTTAANAGT APTATTCCAA CCAGAAATTA
  701 GACGTGCGTT AGAACAACTT GGTANAGGTA GCTTTTTAAA ACGCNATACT
  751 TCTAATACGT ATAGTAAAGA TGAAGAGAAA TTGATTCAAT CGGTTTCAAA
  801 GGCTGTGCAA TATATGGCTA AAAGACGTAT AGGTGCATTA ATTGTCTTTG
  851 AAAAAGAAAC AGGTCTTCAA GATTATATTG AAACAGGTAT TGCCAATGGA
  901 TTCAAATATT TCGCAAGAAC TTTTAATTAA TGTCTTTATA CCTAACACAC
  951 CTTTACATGA TGGTGCAAKG ATTATTCAAG GCACGAARAT TGCAGCAGCA
1001 GCAAGTTATT TGCCATTGTC TGRWAGTCCT AAGATATCTA AAAGTTGGGT
1051 ACAAGACATA GAGCTGCGGT TGGTATTTCA GAAGTTATCT GATGCATTTA
1101 CCGTTATTGT ATCTGAAGAA ACTGGTGATA TTTCGGTAAC ATTTGATGGA
1151 AAATTACGAC GAGACATTTC AAACCGAAAT TTTTGAAGAA TTGCTTGCTG
1201 AACATTGGTT TGGCACACGC TTTCAAAAGA AAGKKKTGAA ATAATATGCT
1251 AGAAAKTAAA TGGGGCTTGA GATTTATTGC CTTTCTTTTT GGCATTGTTT
1301 TTCTTTTTAT CTGTTAACAA TGTTTTTGGA AATATTCTTT AAACACTGGT
1351 AATTCTTGGT CAAAAGTCTA GTAAAACGGA TTCAAGATGT ACCCGTTGAA
1401 ATTCTTTATA ACAACTAAAG ATTTGCATTT AACAAAAGCG CCTGAAACAG
1451 TTAATGTGAC TATTTCAGGA CCACAATCAA AGATAATAAA AATTGAAAAT
1501 CCAGAAGATT TAAGAGTAGT GATTGATTTA TCAAATGCTA AAGCTGGAAA
1551 ATATCAAGAA GAAGTATCAA GTTAAAGGGT TAGCTGATGA CATTCATTAT
1601 TCTGTAAAAC CTAAATTAGC AAATATTACG CTTGAAAACA AAGTAACTAA
1651 AAAGATGACA GTTCAACCTG ATGTAAGTCA GAGTGATATT GATCCACTTT
1701 ATAAAATTAC AAAGCAAGAA GTTTCACCAC AAACAGTTAA AGTAACAGGT
1751 GGAGAAGAAC AATTGAATGA TATCGCTTAT TTAAAAGCCA CTTTTAAAAC
1801 TAATAAAAAG ATTAATGGTG ACACAAAAGA TGTCGCAGAA GTAACGGCTT
1851 TTGATAAAAA ACTGAATAAA TTAAATGTAT CGATTCAACC TAATGAAGTG
1901 AATTTACAAG TTAAAGTAGA GCCTTTTAGC AAAAAGGTTA AAGTAAATGT
1951 TAAACAGAAA GGTAGTTTRS CAGATGATAA AGAGTTAAGT TCGATTGATT
2001 TAGAAGATAA AGAAATTGAA TCTTCGGTAG TCGAGATGAC TTMCAAAATA
2051 TAAGCGAAGT TGATGCAGAA GTAGATTTAG ATGGTATTTC AGAATCAACT
2201 GAAAAGACTG TAAAAATCAA TTTACCAGAA CATGTCACTA AAGCACAACC
2151 AAGTGAAACG AAGGCTTATA TAAATGTAAA ATAAATAGCT AAATTAAAGG
2201 AGAGTAAACA ATGGGAAAAT ATTTTGGTAC AGACGGAGTA AGAGGTGTCG
2251 CAAACCAAGA ACTAACACCT GAATTGGCAT TTAAATTAGG AAGATACGGT
2301 GGCTATGTTC TAGCACATAA TAAAGGTGAA AAACACCCAC GTGTACTTGT
2351 AGGTCGCGAT ACTAGAGTTT CAGGTGAAAT GTTAGAATCA GCATTAATAG
2401 CTGGTTTGAT TTCAATTGGT GCAGAAGTGA TGCGATTAGG TATTATTTCA
2451 ACACCAGGTG TTGCATATTT AACACGCGAT ATGGGTGCAG AGTTAGGTGT
2501 ATTGATTTCA GCCTCTCATA ATCCAGTTGC AGATAATGGT ATTAAATTCT
2551 TTGSCTCGAC CNCCNNGCTN GCA
```

Mutant: NT19
Phenotype: temperature sensitivity
Sequence map: Mutant NT19 is complemented by pMP49, which contains a 1.9 kb insert of S. aureus genomic DNA. A partial restriction map is depicted FIG. 31. Database searches at both the nucleic acid and peptide levels reveal strong similarity at the nucleic acid level to the rnpA gene, which encodes the catalytic RNA component RNAse P, from the bacilli B. megaterium, B. subtilis, and B. stearothermophilus as well as from other prokaryotes. The strongest similarity observed is to the rnpA Genbank entry from B. subtilis (Genbank Accession No. M13175; published in Reich, C. et al. J. Biol. Chem., 261 (1986) 7888-7893).

DNA sequence data: The following DNA sequence data represents the sequence of clone pMP49, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP49
SEQ ID NO. 18
pMP49 Length: 1962 nt
      1 GTGCTTCCAC CAATACGTTC CACCATATGG AGGATTTCCA ATTAACGCCA
    51 CCGGTTCTTC TGTATCAATT GTTAATGTAT TGACATCTTT TACACTAAAT
  101 TTAATAATAT CAGACAACCC AACTTCTTCA GCGTTACGCT TAGCAATCTC
  151 TACCATTTCT GGATCGATAT CAGAAGCATA TACTTCGATT TCTTTATCAT
  201 AATCAGCCAT CTTATCCGCT TCATCACGGT AATCATCATA AATATTTGCT
  251 GGCATGATGT TCCATTGCTC TGATACGAAC TCGCGATTAA AACCAGGTGC
  301 GATATTTTGA GCAATTAAAC AAGCTTCTAT AGCTATTGTA CCCGAACCGC
  351 AAAATGGATC AATTAAAGGT GTATCACCTT TCCAGTTTGC AAGACGGATT
  401 AAACTTGCTG CCAACGTTTC TTTAATTGGT GCTTCACCTT GTGCTAATCT
  451 ATAACCACGT CTGTTCAAAC CAGAACCTGA TGTGTCGATA GTCAATAATA
  501 CATTATCTTT TAAAATGGCA ACTTCAACAG GGTATTTGGC ACCTGATTCA
  551 TTTAACCAAC CTTTTTCGTT ATATGCGCGA CGTAATCGTT CAACAATAGC
  601 TTTCTTAGTT ATCGCCTGAC AATCTGGCAC ACTATGTAGT GTTGATTTAA
  651 CGCTTCTACC TTGAACTGGG AAGTTACCCT CTTTATCAAT TATAGATTCC
  701 CAAGGGAGCG CTTTGGTTTG TTCGAATAAT TCGTCAAACG TTGTTGCGTW
  751 AAAACGTCCA ACAACAATTT TGATTCGGTC TGCTGTGCGC AACCATAAAT
  801 TTGCCTTTAC AATTGCACTT GCGTCTCCTT CAAAAAATAT ACGACCATTT
  851 TCAACATTTG TTTCATAGCC TAATTCTTGA ATTTCCCTAG CAACAACAGC
  901 TTCTAATCCC ATCGGACAAA CTGCAAGTAA TTGAAACATA TATGATTCTC
  951 CTTTTATACA GGTATTTTAT TCTTAGCTTG TGTTTTTTAT ACATTTCCAA
1001 CAAATTTAAT CGCTGATACA TTAACGCATC CGCTTACTAT TTTAAAACAA
1051 GGCAGTGTCA TTATATCAAG ACAAGGCGTT AATTTTAAGT GTCTTCTTTY
1101 CATGAAAAAA GCTCTCCMTC ATCTAGGAGA GCTAAACTAG TAGTGATATT
1151 TCTATAAGCC ATGTTCTGTT CCATCGTACT CATCACGTGC ACTAGTCACA
1201 CTGGTACTCA GGTGATAACC ATCTGTCTAC ACCACTTCAT TTCGCGAAGT
1251 GTGTYTCGTT TATACGTTGA ATTCCGTTAA ACAAGTGCTC CTACCAAATT
1301 TGGATTGCTC AACTCGAGGG GTTTACCGCG TTCCACCTTT TATATTTCTA
1351 TAAAAGCTAA CGTCACTGTG GCACTTTCAA ATTACTCTAT CCATATCGAA
1401 AGACTTAGGA TATTTCATTG CCGTCAAATT AATGCCTTGA TTTATTGTTT
1451 CAYCAAGCRC GAACACTACA ATCATCTCAG ACTGTGTGAG CATGGACTTT
1501 CCTCTATATA ATATAGCGAT TACCCAAAAT ATCACTTTTA AAATTATAAC
1551 ATAGTCATTA TTAGTAAGAC AGTTAAACTT TTGTATTTAG TAATTATTTA
1601 CCAAATACAG CTTTTTCTAA GTTTGAAATA CGTTTTAAAA TATCTACATT
1651 ATTTGAAGAT GTATTTGTTG TTGTATTATT CGAAGAAAAA CTTTTATTGT
1701 CCTGAGGTCT TGATGTTGCT ACACGTAGTC TTAATTCTTC TAATTCTTTT
1751 TTAAGTTTAT GATTCTCTTC TGATAATTTT ACAACTTCAT TATTCATATC
1801 GGCCATTTTT TGATAATCAG CAATAATGTC ATCTAAAAAT GCATCTACTT
1851 CTTCTCTTCT ATAGCCACGA GCCATCGTTT TTTCAAAATC TTTTTCATAA
1901 ATATCTTTTG CTGATAATTT CAATGAAACA TCTGACATTT TTTCCACCTC
1951 ATTAGAAACT TT
```

Mutant: NT23
Phenotype: temperature sensitivity
Sequence map: Mutant NT23 is complemented by pMP5.5, which contains a 5.2 kb insert of S. aureus genomic DNA. A partial restriction map is depicted FIG. 32. Database searches at both the nucleic acid and peptide levels reveal limited similarity at the protein level only to S. aureus proteins FemA and FemB, suggesting that clone pMP55 contains a new Fem-like protein. Since the Fem proteins are involved in peptidoglycan formation, this new Fem-like protein is likely to make an attractive candidate for screening antibacterial agents. Since clone pMP55 does not map to the same location as the femAB locus (data not shown here), the protein is neither FemA nor FemB and represents a novel gene.

DNA sequence data: The following DNA sequence data represents the sequence of clone pMP55, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP55, a 5000 bp genomic fragment
SEQ ID NO. 19
pMP55 Length: 5253 nt
      1 TAACTGGACT ACWACCGCCA ACTRAGTATT GAATTGTTTT AACATGCTTT
    51 TCCTGTTTTA AATATTTTTA AACATCTTTC GCATGATTCA ACACTGCTTG
  101 CTCCGTTTCA CCAGGCTTCG GTGTATAAGT AATAGCTAAA AATTTATCGT
  151 CACCTGCTGA AATAAAGCTA GTGCCTAGTC TCGGTCCTCC AAATACAATA
  201 GTTGCAACCA AAATTAATGT ACTTAATATA ATTWCAATCC ACTTATGATT
  251 TAATGACCAA TGTAATACTT TTTTATAAGT TGTACTAACA ACACCTAATC
  301 CTTCTTGATG TTGTTTATTA CGACGTTTAA CGCCTTTTTT AAATAGTGTA
  351 GCTGCCAACG CTGGAACGAG TGTAATTGAC ACTAATAACG ATGCTAATAA
  401 ACTAAATGCA ATAGCCAATG CAAAAGGTCT AAACATTTCG CCTACTGAAC
  451 CTGATACAAA CACAAGTGGT AAGAAGACGA TAATAGKAAC TAGTGTCGAT
  501 GRCATTATTG GTTTAAATAC TTCAGTTGTC GCACTGATAA TTAAATTTTC
  551 ACCTTTTAGT TGGTTCTTCT GAATCTGTTA AGCGTCGATA AATATTTTCA
  601 MCAACTACAA TCGAATCGTC TATCACACGT CCAATCGCTA CTGTTAATGC
  651 ACCTAACGTT AGTATATTCA ATGAMACATC ACTCAATTTC AGAGCAATAA
  701 GCGSCATAAG AAGTGATAAC GGMATCGATA TMATAGAAAT TGCCGTCGTA
  751 CGAATGTTTC TTAAAAACAG CAAAATAACT ATAATTGCCA CGRATTGTAC
  801 CTAATGATGC TTTTTCAACC ATCGTATAAA GTGATTTCTC AACAGGCTTT
  851 GCAGTATCCA TTGTTTTTGT GACATTAAAA TCTTTATTTT CATCAACGAA
  901 TGTATCAATT TTACGTTGTA CATCTTTGGC TACTTGAACT GTATTGGCAT
  951 CTTGAGCTTT AGTTATTTGT AGATTAACCG CATCCTTTCC ATTCGTTTTA
1001 GAAATAGAAG TACGCACATC ACCAACTGTA ATATCAGCTA AATCTCCTAG
1051 TTTCGCTGTC GGCATACCAC TTATATTATT TGGTGCTGAC GCTTTTGAAT
1101 TTTGCTGTGG TGATGCCTGA TTAACGTCTG ACATGGCTGA AATTTTGTTT
1151 ATTGTCACTT TGGGATTGAG ATTGCCCTTG TCCTCCTGCC AACGTTAATG
1201 GAATATTTAT GTTTTTAAAA GCATCAACAG ATTGATATTG ACCATCAACA
1251 ACAATTGATT TATCTTTATC ACCAAATTGG AACAATCCAA GTGGCGTTGT
1301 TCTTGTTGCC GTTTTTAGAT AGTTTTCTAC ATCATCAGCA GTCAACCCAT
1351 ATTTTCAAGT TCATTTTGCT TAAATTTAAG GGTGATTTCA CGGTTCGTCT
1401 GCCCATTTAA TTGCGCATTT TGNACACCAT CTACCGTTTG CAATTTTGGT
1451 ATNAATTGTT CATTCAGTAC TTTCGTTACT TTTTTCAAGT CATTCNCTTT
1501 ATTTGAAAAT GAATATGCTA AAACCGGAAA AGCATCCATC GAATTACGTC
1551 NTANTTCTGG TTGACCAACT TCATCTTTAA ATTTAATTTT NTNTATTTCT
1601 NTTNTAAGCT GTTCTTCTGC TTTATCCAAA TCTGTATTMT TTTCATATTC
1651 AACTGTTACA ATTGAAGCAT TTTGTATGGA TTGCGTTTTA ACATTTTTCA
1701 CATATGCCAA TGATCTTACY TGAWTGTCAA TTTTACTACT TATTTCATCT
1751 TGGGTACTTT GTGGCGTTGC ACCCGGCATT GTTGTTGTAA CTGAAATAAC
1801 TGGATKTTGT ACATTTGGTA KTAATTCTMA TTTCAATTTA GCACTCGCAT
1851 ATACACCGCC CAAGACAACT WAAACAACCA TTAMAAAGAT AGCAAACYTA
1901 TTCCCTAAAA RGAAAATTGT AATAGCTTTT TTAWCAACAG TMCTYCCCCC
1951 TCTTTCACTA WAATTCAAAA AATTATTTTA CTCAACCATY CTAWWWTGTG
2001 TAAAAAAAAT CTGAACGCAA ATGACAGYCT TATGAGCGTT CAGATTTCAG
2051 YCGTTAATCT ATTTYCGTTT TAATTTACGA GATATTTTAA TTTTAGCTTT
2101 TGTTAAACGC GGTTTAACTT GCTCAATTAA TTGGYACAAT GGCTGATTCA
2151 ATACATAATC AAATTCACCA ATCTTTTCAC TTAAGTATGT TCCCCACACT
2201 TTTTTAAATG CCCATAATCC ATAATGTTCT GAGTCTTTAT CTGGATCATT
2251 ATCTGTACCA CCGAAATCGT AAGTTGTTGC ACCATGTTCA CGTGCATACT
2301 TCATCATCGT ATACTGCATA TGATGATTTG GTAAAAAATC TCTAAATTCA
2351 TTAGAAGACG CACCATATAA GTAATATGAT TTTGAGCCAG CAAACATTAA
2401 TAGTGCACCA GAAAGATAAA TACCTTCAGG ATGTTCCTTT TCTAAAGCTT
2451 CTAGGTCTCG TTTTAAATCT TCATTTTTAG CAATTTTATT TTGCGCATCA
2501 TTAATCATAT TTTGCGCTTT TTTAGCTTGC TTTTCAGATG TTTTCATCTT
2551 CTGCTGCCAT TTAGCAATTT CGGCATGAAG TTCATTCAAT TCTTGATTTA
2601 CTTTCGCTAT ATTTTCTTTT GGATCCAACT TTACTAAAAA TAGTTCAGCA
2651 TCTCCATCTT CATGCAACGC ATCATAAATA TTTTCAAAGT AACTAATATC
2701 ACGCGTTAAG AAGCCATCGC GTTCCCCAGT GATTTTCATT AACTCAGCAA
2751 ATGTTTTTAA ACCTTCTCTA TCAGATCGTT CTACTGTCGT ACCTCGCTTT
2801 AAAGCCAAGC GCACTTTTGA ACGATTTCGG CGTTCAAAAC TATTTAATAA
2851 CTCATCATCA TTTTTATCAA TTGGTGTAAT CATAGTCATA CGTGGTTGGA
2901 TGTAGTCTTT TGATAAACCT TCTTTAAATC CTTTATGTTT AAAACCAAGC
2951 GCTTTCAAAT TTTGCAAAGC ATCTGTRCCT TTATCAACTT CAACATCAGG
3001 ATCGRTTTTA ATTGCATACG CTTTCTCAGC TTTAGCAATT TCTTTTGCAC
3051 TGTCTAACMA TGSMTTTAAC GYTTCTTTAT TACTATTAAT CAACAACCAA
3101 AACCMCGCGR RAWTATWACM TAGSGTATAA GGTAATTTAG GTACTTTTTT
3151 AAAAAGTAAC TGCGCAACAC CCTGGAACTT SMCCGTCACG ACCTACAGCG
3201 ATTCTTCGCG CGTACCATCC AGTTAATTTC TTTGTTTCTG CCCATTTCGT
3251 TAATTGTAAT AAATCTCCAT TTGGGTGGGR WTTWACAAAT GCGTCATGTT
3301 CCTGATTAGG KGATATGCAT CTTTTCCATG ATTTATGATA TCTCCTTCTA
3351 TTTAACAATA CCTTTAATTA TACAGTTTGT ATCTTATAGT GTCGATTCAG
3401 AGCTTGTGTA AGATTTGAAC TCTTATTTTT GGAAATGTCC ATGCTCCAAT
3451 TAATAGTTTA GCAAGTTCAA ATTTACCCAT TTTAATTGTG AATCATTTTA
3501 TATCTATGTT TCGTGTTAAA TTTAATGTTA TCGTACARTT AATACTTTTC
3551 AACTAGTTAC CTATACTTCA ATATACTTTC ATCATCTAAC ACGATATTCA
3601 TTTCTAARAA TGAACCAACT TGACTTCAAT GAATAAATTT TTCCTCAAGC
3651 AACCACATTA ATGTTCATAT ACAATTACCC CTGTTATAAT GTCAATAATC
3701 TAACAATGAG GTGTTTGATA TGAGAACAAT TATTTTAAGT CTATTTATAA
3751 TTATGRACAT CGTTGCAATC ATTATGACAT TGAGTCAACC TCTCCACCGT
3801 GAATTACTTT AGTTTACGGG TTATACTTAT CTTTTTCACA TTTATATTAT
3851 CAATCTTTTT CATTTTAATT AAGTCATCAC GATTAAATAA TATATTAACG
3901 ATTMWWTCCA TTGTGCTTGT CATTATTCAT ATGGGCATTC TCGCTCATAG
3951 CACTTACGTA TATTTATACT AATGGTTCAA AGCGATAAAT AGCACCTCTG
4001 ATAAAAATTG AATATGGTGA AGTTGCTTGT GCGTCTTTTA TGATAACCGA
4051 ATGATATTTT GAAACTTTAC CATCTTCAAT TCTAAAATAA ATATCATCAT
4101 TTTTTAAAAT CAAATCTGTG TAATGGTCAT TTYKTCHACA ATGTCCATAT
4151 CAARCCATTT CAACCAATTC GATACTGTWK GTGATCGGTT TTTACTTTTC
4201 ACAATAACAG TTTCAAWTGA AAATTGTTTT TGAAAATATT TTTGCAATTT
4251 TTTAGTACGC ATGGAATCAC TTTCTTCCCA TTGAATAAAA AATGGTGGCT
4301 TAATTTCATC ATCATCCTGA TTCATTATAT AAAGCAATTG CCACTTTACC
4351 TWCACCATCT TTATGTGTAT CTCTTTCCAT TTGAATCGGC CCTACTACTT
4401 CAACCTGCTC ACTNTGTAGT TTATTTTTAA CTGCCTCTAT ATCATTTGTA
4451 CGCAAACAAA TATTTATTAA AGCCTTGCTC ATACTTCTCT TGAACAATTT
4501 GAGTAGCAAA AGCGACTCCG CCTTCTATCG TTTTTGCCAT CTTTTTCAAC
4551 TTTTCATTAT TTTACTACAT CTAGTAGCTC AAGATAATTT CATTGATATW
4601 ACCTAAKKTA TTGAATGTTC CATATTTATG ATGATACCCA CCTGAATGTA
4651 ATTTTATAAC ATCCTCCTGG AAAACTAAAC CGATCTAACT GATCTATATA
4701 ATGAATGATG TGATCANATT TCAATATCAT TAGTATCCCC CTATTTACAT
4751 GTAATTACGC TTATTTTAAA CAAAGTAWAA TTATTTTTGC YCTTAATAAT
4801 TATATAKTGA YYYCWAATTG CTCCCGTTTT ATAATTACTA TTGTTGTAAA
4851 ARGGTTAGCT AAGCTAACTA TTTTGCCTTA GGAGATGTCA CTATGCTATC
4901 ACAAGAATTT TTCAATAGTT TTATAACAAT ATAYCGCCCC TATTTAAAAT
4951 TAGCCGAGCC GATTTTAGRA AAACACAATA TATATTATGG CCAATGGTTA
5001 ATCTTACGCG ATATCGCTAA ACATCAGCCC ACTACTCTCA TTGNAATTTC
5051 ACATAGACGG GCAATTGAAA AGCCTACTGC AAGAAAAACT TTAAAAGCTC
5101 TAATAGGAAA TGACCTTATW ACAGTAGAAA ACAGNTTAGA GGATAAACNA
5151 CAAAAGNTTT TAACTTTAAC ACCTAAAGGG CATKAATTAT ATGAGATTGT
5201 TTGTCTTGAT GNACAAAAGC TCCNACAAGC AGNNAGTTGC CAAAACAAAG
5251 ATT
```

Mutant: NT27
Phenotype: temperature sensitivity
Sequence map: Mutant NT27 is complemented by pMP59, which contains a 3.2 kb insert of S. aureus genomic DNA. A partial restriction map is depicted FIG. 33. Database searches at both the nucleic acid and peptide levels reveal strong peptide-level similarities to two hypothetical ORFs from B. subtilis. These hypothetical ORFs are also found in other bacteria, but in all cases, nothing has been reported in the literature about the functions of the corresponding gene products.

DNA sequence data: The following DNA sequence data represents the sequence of clone pMP59, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP59
SEQ ID NO. 20
pMP59 Length: 3263 nt
      1 ACATTGAMAA AGATCACCCA TTACAACCAC ATACAGATGC AGTAGAAGTT
    51 TAAAACACAT TTTTCTAATT ATCAAAGCTT AGGATAAATA TGATGTCCTA
  101 AGCTTTTCCT TTTACAACTT TTTCGAATAA ACAACAGTTA AATATATTCA
  151 CCTTTCTACC AAACTTTTTA TCCCCTCATT TAAATTTTAC CGGKYTCATA
  201 TAAAATCCTT TAATTCTTTC TTAACATTAW TTTWTWATCT CTACATYTAT
  251 TTTAATAAAT AGAACTGCAC ATTTATTCGA AATACTTAGA TTTCTAGTGA
  301 GATAAACTGC TTTATTTATT ATCATTCATC ATGTAAAATA AGATTTAACT
  351 GAAATTTTAG TGTTATTTCA CTAATTTTTT AAAATGAACG ACATGATGAA
  401 CCTAGTTATT AACCAAATCG TTATTAAGTT ACATTATAGA GATGATTGGA
  451 ATGAATTTAT CGATATATAC TCCAATACGA TTTTACTAGG GTTAACAATA
  501 AATTAAACAA ACATTCTTAG GAGGRATTTT TAACATGGCA GTATTTAAAG
  551 TTTTTTATCA ACATAACAGA GTACGAGGTR RTTGTGCGTG AAAATACACA
  601 ATCACTTTAT GTTGAAGCTC ARACAGAAGA ACAAGTAGCG TCGTTACTTG
  651 AAAGATCGTA ATTTTAATAT CGAATTTATC ACTAAATTAG AGGGCGCACA
  701 TTTAGATTAC GAAAAAGAAA ACTCAGCAAC ACTTTAATGT GGAGATTGCT
  751 AAATAATGAA ACAATTACAT CCAAATGAAG TAGGTGTATA TGCACTTGGA
  801 GGTCTAGGTG AAATCGGTAA AAATACTTAT GCAGTTGAGT ATAAAGACGA
  851 AATTGTCATT ATCGATGCCG GTATCAAATT CCCTGATGAT AACTTATTAG
  901 GGATTGATTA TGTTATACCT GACTACACAT ATCTAGTTCA AAACCAAGAT
  951 AAAATTGTTG GCCTATTTAT AACACATGGT CACGAAGACC ATATAGGCGG
1001 TGTGCCCTTC CTATTAAAAC AACTTAATAT ACCTATTTAT GGTGGTCCTT
1051 TAGCATTAGG TTTAATCCGT AATAAACTTG AAGAAACATC ATTTATTACG
1101 TACTGCTAAA CTAAATGAAA TCAATGAGGA CAGTGTGATT AAATCTAAGC
1151 ACTTTACGAT TTCTTTCTAC TTAACTACAC ATAGTATTCC TGAAACTTAT
1201 GGCGTCATCG TAGATACACC TGAAGGAAAA KTAGTTCATA CCGGTGACTT
1251 TAAATTTGAT TTTACACCTG TAGGCAAACC AGCAAACATT GCTAAAATGG
1301 CTCAATTAGG CGAAGAAGGC GTTCTATGTT TACTTTCAGA CTCAACAAAT
1351 TCACTTGTGC CTGATTTTAC TTTAAGCGAA CGTTGAAGTT GGTCAAAACG
1401 TTAGATAAGA TCTTCCGTAA TTGTAAAGGT CCGTATTATA TTTGCTACCT
1451 TCGCTTCTAA TATTTACCGA GTTCAACAAG CAGTTGAAGC TGCTATCAAA
1501 AATAACCGTA AAATTGTTAC KTTCGGTCCG TTCGATGGAA AACAATATTA
1552 AAATAGKTAT GGAACTTGGT TATATTAAAG CACCACCTGA AACATTTATT
1601 GAACCTAATA AAATTAATAC CGTACCGAAG CATGAGTTAT TGATACTATG
1651 TACTGGTTCA CAAGGTGAAC CAATGGCAGC ATTATCTAGA ATTGCTAATG
1701 GTACTCATAA GCAAATTAAA ATTATACCTG AAGATACCGT TGTATTTAGT
1751 TCATCACCTA TCCCAGGTAA TACAAAAAGT TATTAACAGA ACTATTAATT
1801 CCTTGTATAA AGCTGGTGCA GATGTTATCC ATAGCAAGAT TTCTAACATC
1851 CATACTTCAG GGCATGGTTC TCAAGGGTGA TCAACAATTA ATGCTTCCGA
1901 TTAATCAAGC CGAAATATTT CTTACCTATT CATGGTGAAT ACCGTATGTT
1951 AAAAGCACAT GGTGAGACTG GTGTTGAATG CGSSKTTGAA GAAGATAATG
2001 TCTTCATCTT TGATATTGGA GATGTCTTAG CTTTAACACM CGATTCAGCA
2051 CGTAAAGCTG KTCGCATTCC ATCTGGTAAT GWACTTGTTG ATGGTAGTGG
2101 TATCGGTGAT ATCGGTAATG TTGTAATAAG AGACCGTAAG CTATTATCTG
2151 AAGAAGGTTT AGTTATCGTT GTTGTTAGTA TTGATTTTAA TACAAATAAA
2201 TTACTTTCTG GTCCAGACAT TATTTCTCGA GGATTTGTAT ATATGAGGGA
2251 ATCAGGTCAA TTAATTTATG ATGCACAACG CMAAAWCMAA ACTGATGTTT
2301 ATTAGTWAGT TWAATCCAAA ATAAAGAWAT TCAATGGCAT CAGATTAAAT
2351 CTTCTATCAT TGAAACATTA CAACCTTATT TATTKGAAAA AACAGCTAGR
2401 AAACCAATGA TTTTACCAGT CATTATGGAA GGTAAACGAA CAAAARGAAT
2451 CAAACAATAA ATAATCAAAA AGCTACTAAC TTTGAAGTGA AGTTTTAATT
2501 AAACTCACCC ACCCATTGTT AGTAGCTTTT TCTTTATATA TGATGAGCTT
2551 GAGACATAAA TCAATGTTCA ATGCTCTACA AAGTTATATT GGCAGTAGTT
2601 GACTGAACGA AAATGCGCTT GTWACAWGCT TTTTTCAATT STASTCAGGG
2651 GCCCCWACAT AGAGAATTTC GAAAAGAAAT TCTACAGGCA ATGCGAGTTG
2701 GGGTGTGGGC CCCAACAAAG AGAAATTGGA TTCCCCAATT TCTACAGACA
2751 ATGTAAGTTG GGGTGGGACG ACGGAAATAA ATTTTGAGAA AATATCATTT
2801 CTGTCCCCAC TCCCGATTAT CTCGTCGCAA TATTTTTTTC AAAGCGATTT
2851 AAATCATTAT CCATGTCCCA ATCATGATTA AAATATCACC TATTTCTAAA
2901 TTAATATTTG GATTTGGTGA AATGATGAAC TCTTTGCCTC GTTTAATTGC
2951 AATAATGTTA ATTCCATATT GTGCTCTTAT ATCTAAATCA ATGATAGACT
3001 GCCCCGCCAT CTTTTCAGTT GCTTTCAATT CTACAATAGA ATGCTCGTCT
3051 GCCAACTCAA GATAATCAAG TACACTTGCA CTCGCAACAT TATGCGCNAT
3101 ACGTCTACCC ATATCACGCT CAGGGTGCAC AACCGTATCT GCTCCAATTT
3151 TATTTAAAAT CTTTGCNTGA TAATCATTTT GTGCTCTTAG CAGTTACTTT
3201 TTTTACACCT AACTCTTTTA AAATTAAAGT CGTCAACGTA CTTGNTTGAA
3251 TATTTTCACC AAT
```

Mutant: NT28
Phenotype: temperature sensitivity
Sequence map: Mutant NT28 is complemented by pMP60, which contains a 4.7 kb insert of S. aureus genomic DNA. A partial restriction map is depicted FIG. 34, along with open boxes to indicate the percentage of the clone for which DNA sequence has been obtained. Database searches at both the nucleic acid and peptide levels reveal identity of clone pMP60 at both the nucleic acid and peptide levels to the polC gene, encoding DNA Polymerase III alpha subunit, from S. aureus(Genbank Accession No. Z48003; unpublished as of 1995). The relative size and orientation of the complete ORF encoding Pol III is depicted by an arrow in the map.

DNA sequence data: The following DNA sequence data was generated by using the standard sequencing primers SP6 and T7, and can be used to demonstrate identity between clone pMP60 and Genbank entry Z48003:

```
subclone 1022, a 900 bp EcoR I fragment
SEQ ID NO. 21
1022.sp6 Length: 510 nt
    1 GGGTACCGAG CTCGAATTCG AGGTGTACGG TAGAAATACT TCACCAATGA
  51 TGCACTTACA ATTTTAAATA GATTTTNAAG ACCTTGTTGG TTTTGTACAA
101 TTAATGTGAC ATGACTAGGT CTTGCACGTT TATATGCATC TNCATTACTG
151 AGTTTTTTGT TGATTTCGTT ATGATTTAAT ACGCCTAATT CTTTCATTTG
201 TTGAACCATT TTNATGAAAA TGTAAGCTGT TGCTTCTGTA TCATAAATGG
251 CACGGTGATG TTGCGTTAAT TCTACGCCAT ATTTTTTAGC CAAGAAATTC
301 AAACCATGTT TACCATATTC AGTATTAATC GTACGNGATA ATTCTAAAGT
351 ATCGNTAACA CCATTCGTTG ATGGTCCAAA CCCAAGACGT TCATATCCCG
401 TATCGATGNN GCCCATATCA AACGGAGCAT TATGCGTTAC GGTTTTCGNA
451 TCGGCAACCC TTCTTAAACT CTGTAAGNAC TTCTTCATTT CAGGGGATCT
501 NCTANCATAT
subclone 1023, a 1200 bp EcoR I fragment
SEQ ID NO. 22
1023.sp6 Length: 278 nt
    1 GGGTACCGAG CTCGAATTCT ACACGCTTTT CTTCAGCCTT ATCTTTTTTT
  51 GTCGCTTTTT TAATCTCTTC AATATCAGAC ATCATCATAA CTAAATCTCT
101 AATAAATGTA TCTCCTTCAA TACGNCCTTG AGCCCTAACC CATTTACCAA
151 CANTTAGNGC TTTAAAATGT TCTAAATCAT CTTTGTTTTT ACGAGTAAAC
201 ATTTTTAAAA CTAAAGNGTC CGTATAGTCA GTCACTTTAA TTTCTACGGT
251 ATGGNGGCCA CTTTTAAGTT CTTTTAAG
subclone 1024, a 1400 bp EcoR I fragment
SEQ ID NO. 23
1024.sp6 Length: 400 nt
    1 GGGTACCGAG CTCGAATTCT GGTACCCCAA ATGTACCTGT TTTACATAAA
  51 ATTTCATCTT CAGTAACACC CAAACTTTCA GGTGTACTAA ATATCTGCAT
101 AACTNCTTTA TCATCTACAG GTATTGTTTT TGGNTCAATT CCTGATAAAT
151 CTTGAAGCAT ACGAATCATT GTTGGNTCAT CGTGTCCAAG TATATCANGT
201 TTTAATACAT TATCATGAAT AGAATGGAAA TCAAAATGTG TCGTCATCCA
251 TGCTGAATTT TGATCATCGG CAGGATATTG TATCGGCGTA AAATCATAAA
301 TATCCATGTA ATCAGGTACT ACAATAATAC CCCCTGGNTG CTGTCCAGTT
351 GTACGTTTAA CACCTGTACA TCCTTTAACG NGTCGATCTA TTTCAGCACC
subclone 1025, a 1200 bp EcoR I/Hind III fragment
SEQ ID NO. 24
1025.sp6 Length: 528 nt
    1 GATCATTTGC ATCCATAGCT TCACTTATTT NTCCAGAAGC TAGCGTACAA
  51 TCATTTAAAT CTACGCCACC TTCTTTATCA ATAGAGATTC TAAGAAAATN
101 ATCTCTACCC TCTTTGACAT ATTCAACGTC TACAAGTTCA AAATTCAAGT
151 CTTCCATAAT TGGTTTAACA ATCACTTCTA CTTGTCCTGT AATTTTNCTC
201 ATACAGGCCT CCCTTTTTGG CAAATAGAAA AGAGCGGGAA TCTCCCACTC
251 TTCTGCCTGA GTTCACTAAT TTTTAAGCAA CTTAATTATA GCATAAGTTT
301 ATGCTTGAAA CAAATGACTT CACTATTAAT CAGAGATTCT TGTAAAAGTT
351 TGTCCCTTTA TTTCACCATT ACATTTGAAT NGNCTCGTNA GNCATTGTAA
401 AGAGATNCGG GCATAATTTT GTGTCCAGCA TCAATTTTGG TATTTCTTGT
451 CTTACGGCTT ACGGTTNATT AAATACCThG GNTTTTTNTC TTTTACCTNT
501 NATATNTCGN ANGNTGGGNT TTTTCNNG
```

Mutant: NT29
Phenotype: temperature sensitivity
Sequence map: Mutant NT29 is complemented by pMP62, which contains a 5.5 kb insert of S. aureus genomic DNA. A partial restriction map is depicted FIG. 35, along with open boxes to indicate the percentage of the clone for which DNA sequence has been obtained. Database searches at both the nucleic acid and peptide levels reveal identity between clone pMP62 and the gyrBA locus of S. aureus (Genbank Accession No. M86227; published in Margerrison, E. E., et al. J. Bacteriology, 174 (1992) 1596-1603), which encodes DNA gyrase (EC 5.99.1.3). Arrows above the restriction map indicate relative size and position of the ORFs, demonstrating that both gyrB and gyrA genes are fully contained within clone pMP62 and are likely to be expressed.

DNA sequence data: The following DNA sequence data are those obtained from subclones of clone pMP62, using standard sequencing conditions and the primers T7 or SP6. These data can be used to demonstrate identity between the pMP62 clone and Genbank entry M86227.

```
subclone 29.2e.a, a 550 bp EcoR I fragment
SEQ ID NO. 25
29.2e.a.sp6 LENGTH: 557 nt
      1 CAGCCGACAG TTNACAACCA GCNTCACCGT NAGACAGCAA ACGCCACAAA
    51 CTACAAGGNT CCAAATGNCT AGACAATACT GGTGNAAGGC ANGTAATAAT
  101 ACGACATTAA CATTTGATGA TCCTGCCATA TCAACAGNTC AGAATAGACA
  151 GGATCCAACT GTAACTGTTA CAGATAAAGT AAATGGTTAT TCATTAATTA
  201 ACAACGGTAA GATTGGTTTC GTTAACTCAG AATTAAGACG AAGCGATATG
  251 TTTGATAAGA ATAACCCTCA AAACTATCAA GCTAAAGGAA ACGTGGCTGC
  301 ATTAGGTCGT GTGAATGCAA ATGATTCTAC AGATCATGGT AACTTTAACG
  351 GTATTTCAAA AACTGTAAAT GTAAAACCAG NTTCAGAATT AATTATTAAC
  401 TTTACTACTA TGCAAACCGG ATAGTNAGCA AGGTGCAACA AATTTAGTTA
  451 TTAAAGGATG CTAAGGAANW TACTGNNTTA GCACCTGTAA AATGTTGCTT
  501 AGGCTGGTCC TGCACATTTA TTTTAAGGTC CNNCTTGTNC TGNTNGGCTC
  551 TNGGGGG
SEQ ID NO. 26
29.2e.a.t7 LENGTH: 527 nt
      1 GTCGATCAGC ATCATTGGTA CTTTAAATAA ATGTGCAGTA CCAGTCTTAG
    51 CAACATTTAC AGTTGCTAAT TCAGTATTTT CNTTAGCATC TTTAATAACT
  101 AANTTTNTNG CACCTTGCNT ACTATTCGTT TGCATAGTAG TAAAGTTAAT
  151 AATTAATTCT GANTCTGGTT TTACATTTAC AGTTTTTGAA ATACCGTTAA
  201 AGTTACCATG ANCTGTAGNA TCATTTGCNT TCACACGGCC TAATGCAGCC
  251 NCGGTTCCTT TAGCTTGATA GTTTTGAGGG GTATTCTTAT CAAACATATC
  301 GNTTCGGCTT AATTCTGAGG TAACTGGNAC CNATCTTTAC CNTTGTTAAT
  351 TAATGGNTTC CCCTTTACNT TAATCTGTAA CAGTTACAGT TGGGTCCCCG
  401 TCTATTCTCA TCTGTTGGTA TGGCAGGGTC ACCACAATGN TAATGTCGGT
  451 TTATACTGGN NTCNCCCGNA TTGCTTAGGT TTGGNGCTTG NGGTGTGCGN
  501 TTNCTNGCTT CAGGGGNCTG CTGGGTT
subclone 29.2h.2a, a 1800 bp Hind III fragment
SEQ ID NO. 27
29.2h.2a.sp6 LENGTH: 578 nt
      1 TGTGAGCTCC CAThACCACC AGTGCGNNCA TTGCCTGGGC TACCGATTGT
    51 CAATTTAAAG TCTTCATCTT TAAAGAAAAT TTCAGTACCA TGTTTTTTAA
  101 GTACAACAGT TGCACCTAAA CGATCAACTG CTTCACGATT ACGCTCATAT
  151 GTCTGTTCCT CAATAGGAAT ACCACTTAAT CGTTCCCATT CTTTGAGGTG
  201 TGGTGTAAAG ATCACACGAC ATGTAGGTAA TTGCGGTTTC AGTTTACTAA
  251 AGATTGTAAT CGCATCGCCG TCTACGATTA AATTTTGATG CGGTTGTATA
  301 TTTTGTAGTA GGAATGTAAT GGCATTATTT CCTTTGTAAT CAACGCCAAG
  351 ACCTGGACCA ATTAGTATAC TGTCAGTCAT TTCAATCATT TTCGTCAACA
  401 TTTTCGTATC ATTAATATCA ATAACCATCG CTTCTGGGCA ACGAGAAAGT
  451 AATGCTGAAT GATTTGTTGG ATGTGTAGTA CAGTGATTAA ACCACTACCG
  501 CTAAATACAC ATGCACCGAG CCGCTAACAT AATGGCACCA CCTAAGTTAG
  551 CAGATCGGCC CTCAGGATGA AGTTGCAT
SEQ ID NO. 28
29.2h.2a.t7 LENGTH: 534 nt
      1 CGAGCCAGCA GNTTGCAGCG GCGTGTCCCA TAACTAAGGT GGTGCCATTA
    51 TGTNAGCGGC TCGTCCATGT NTATTTGGCG GTAGTGGTTT AATCACTGTA
  101 GCTACACATC CAACAAATCA TTCAGCATTA CATTCTCGTN GCCCAGAAGC
  151 GATGGTTATT GATATTAATG ATACGAAAAT NTTGACGAAA ATNATTGAAA
  201 TGACTGACAG TATACTAATN GGNCCAGGTC TTGGCGTTGA TTTCAAAGGA
  251 AATAATGCCA TTNCATTCCT ACTACAAAAT ATACAACCGC ATCAAAATTT
  301 AANCGTAGAC GGCGNTGCGA TTNCAATCTT TNGTAAACTG NAACCGCAAT
  351 TACCTACATG TNGTGTGNNC TTNACACCAC ACCTCAAAGG NNTGGGNCGG
  401 TTANGTGGTA TTCCNNTTGN GGACAGGCAT ATGGNGCGTA ATCGTGNAGC
  451 AGTTGNTCGT TTAGGNGCAC TNTNGTCCTT AAAAAACATG GTCTGNATNT
  501 CCTTTAANGN NGNNGCTTTA AATTGGCAAT CGGT
subclone 29.2he, 2400 bp Hind III, EcoR I fragment
SEQ ID NO. 29
29.2he.1.sp6 LENGTH: 565 nt
      1 ACCATTCACA GTGNCATGCA TCATTGCACA CCAAATGNTG TTTGAAGAGG
    51 TGTTTGTTTG TATAAGTTAT TTAAAATGAC ACTAGNCATT TGCATCCTTA
  101 CGCACATCAA TAACGACACG CACACCAGTA CGTAAACTTG TTTCATCACG
  151 TAAATCAGTG ATACCGTCAA TTTTCTTGTC ACGAACGAGC TCTGCAATTT
  201 TTTCAATCAT ACGAGCCTTA TTCACTTGGA AAGGAATTTC AGTGACAACA
  251 ATACGTTGAC GTCCGCCTCC ACGTTCTTCA ATAACTGCAC GAGAACGCAT
  301 TTGAATTGAA CCACGNCCTG TTTCATATGC ACGTCTAATA CCACTCTTAC
  351 CTAAAATAAG TCCNGCAGTT GGGGAATCAG GACCTTCAAT ATCCTCCATT
  401 AACTCAGCAA ATTGNAATNT CAAGGGGTCT TTACTTTAAG GCTNAGNNCA
  451 CCCTTGGTTA ATTCTGTTAA GTTATTGTGG TGGGATATTT CGGTTGCCAT
  501 NCCTNCCNCG GGTACCCNNA TGCACCCNTT GGGTAATNAG GNTTGGGGGT
  551 TTGTGCCCGG TAAGC
SEQ ID NO. 30
29.2he.1.t7 Length: 558 nt
      1 CGCAAAACGT CANCAGAANG NACTNCCTAA TGCACTAATG AAGGGCGGTA
    51 TTAAATCGTA CGTTGAGTTA TTGANCGNAA AATAAAGGAA CCTATTCATG
  101 AATGAGCCAA TTTATATTCA TCAATCTAAA GATGATATTG ANGTAGAAAT
  151 TGCNATTCAN TATAACTCAG GATATGCCAC AAATCTTTTA ACTTACGCAA
  201 ATAACATTCA TACGTATGAN GGTGGTACGC ATGANGACGG ATTCAAACGT
  251 GCATTTACGC GTGTCTTAAA TAGTTATGGT TTAAGTAGCA AGATTNTGTA
2301 AGANGGAAAA GNTAGNCTTT CTGGTGAAGN TACACGTGAA GGTATNNCNG
  351 CNNTTNTATC TNTCAAACNT GGGGNTCCNC AATTNGGAGG TCAAACGGGG
  401 CAAAAATTTG GGNNTTCTGT AGTGCGTCAN GTTGTNGGTN AATTATTCNN
  451 NGNGNCTTTT TACNGTTTTN CTTTGNAAAT CCNCNAGTCG GNCGTNCNGT
  501 GGTTTNNAAA AGGGTTTTTT GNGGCACGTG NACGTGTTNT TCGGAAAAAA
  551 AGCGGGTT
```

Mutant: NT31
Phenotype: temperature sensitivity
Sequence map: Mutant NT31 is complemented by pMP64, which contains a 1.4 kb insert of S. aureus genomic DNA. A partial restriction map is depicted FIG. 36. Database searches at both the nucleic acid and peptide levels reveal strong similarity at the nucleic acid and peptide levels to the aroE gene of B. aphidicola (Genbank Accession No. U09230; unpublished as of 1995), which encodes the shikimate-5-dehydrogenase protein (EC 1.1.1.25). Strong similarities also exist at the peptide level to the aroE genes from E. coli and P. aeruginosa. The size and relative position of the predicted AroE ORF within the pMP64 clone is depicted in the restriction map by an arrow.

DNA sequence data: The following DNA sequence data represents the sequence of clone pMP64, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP64
SEQ ID NO. 31
pMP64 Length: 1508 nt
      1 AGTSGWTCCG TGTGCATAGG TRTGAACTTT GAACCACCAC GTTTAATTTC
    51 ATCGTCACAA ATATCTCCAA AACCAAGCTC GTCGATAATC ATCTGTATCA
  101 TTGTTAATCT GTGCTGAACG TCTATAAAAT CATGGTGCTT TTTCAATGGA
  151 GACATAAAAC TAGGTAAAAA ATAAAATTCA TCTGGCTGTA ATTCATGAAA
  201 TACTTCGCTA GCTACTATCA TATGTGCAGT ATGGATAGGG TTAAACTGAC
  251 CGCCGTAAAG TACTATCTTT TTCATTATTA TGGCAATTCA ATTTCTTTAT
  301 TATCTTTAGA TTCTCTATAA ATCACTATCA TAGATCCAAT CACTTGCACT
  351 AATTCACTAT GAGTAGCTTC GCTTAATGTT TCAGCTAATT CTTTTTTATC
  401 ATCAAAGTTA TTTTGTAGTA CATGTACTTT AATCAATTCT CTGTTTTCTA
  451 ACGTATCATC TATTTGTTTA ATCATATTTT CGTTGATACC GCCTTTTCCA
  501 ATTTGAAAAA TCGGATCAAT ATTGTGTGCT AAACTTCTTA AGTATCTTTT
  551 TTGTTTGCCA GTAAGCATAT GTTATTCTCC TTTTAATTGT TGTAAAACTG
  601 CTGTTTTCAT AGAATTAATA TCAGCATCTT TATTAGTCCA AATTTTAAAG
  651 CTTTCCGCAC CCCTGGTAAA CAAACATATC TAAGCCATTA TAAATATGGT
  701 TTCCCTTGCG CTCTGCTTCC TCTAAAATAG GTGTTTTATA CGGTATATAA
  751 ACAATATCAC TCATTAAAGT ATTGGGAGAA AGATGCTTTA AATTAATAAT
  801 ACTTTCGTTA TTTCCAGCCA TACCCGCTGG TGTTGTATTA ATAACGATAT
  851 CGAATTCAGC TAAATAACTT TTCAGCATCT GCTAATGAAA TTTGGTTTAT
  901 ATTTAAATTC CAAGATTCAA AACGAGCCAT CGTTCTATTC GCAACAGTTA
  951 ATTTGGGCTT TACAAATTTT GCTAATTCAT AAGCAATACC TTTACTTGCA
1001 CCACCTGCGC CCAAAATTAA AATGTATGCA TTTTCTAAAT CTGGATAAAC
1051 GCTGTGCAAT CCTTTAACAT AACCAATACC ATCTGTATTA TACCCTATCC
1101 ACTTGCCATC TTTTATCAAA ACAGTGTTAA CTGCACCTGC ATTAATCGCT
1151 TGTTCATCAA CATAATCTAA ATACGGTATG ATACGTTCTT TATGAGGAAT
1201 TGTGATATTA AAGCCTTCTA ATTCTTTTTT CGAAATAATT TCTTTAATTA
1251 AATGAAAATC TTCAATTGGA ATATTTAAAG CTTCATAAGT ATCATCTAAT
1301 CCTAAAGAAT TAAAATTTGC TCTATGCATA ACGGGCGACA AGGAATGTGA
1351 AATAGGATTT CCTATAACTG CAAATTTCAT TTTTTTAATC ACCTTATAAA
1401 ATAGAATTTC TTAATACAAC ATCAACATTT TTAGGAACAC GAACGATTAC
1451 TTTAGCCCCT GGTCCTATAG TTATAAAGCC TAGACCAGAG ATCGACCTGC
1501 AGGCAGCA
```

Mutant: NT33a
Phenotype: temperature sensitivity
Sequence map: Mutant NT33a is complemented by pMP67, which contains a 1.8 kb insert of S. aureus genomic DNA. A partial restriction map is depicted FIG. 37. Database searches at both the nucleic acid and peptide levels reveal strong peptide-level similarities to ORFs of unknown function in Synechoccocus sp. (identified as “orf2” in Genbank Accession No. L19521), M. tuberculosis (Genbank Accession No. U00024) and E. coli (Genbank Accession No. M86305).

DNA sequence data: The following DNA sequence data represents the sequence of clone pMP59, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP67
SEQ ID NO. 32
pMP67 Length: 1810 nt
      1 CGCGTCTTCC AAATTTCNAA AGCTGTAAAA AGTTATTAAA TCAAATCTTG
    51 CGAATTTGGA TNTAGAGGCA CAATCTGANG TTTATAAAAN TAATGCAGAT
  101 AGAGCTTTAA AAGCNTTGTC AAAACGTGAT ATTCAATTTG ATNTCATTTT
  151 CTTAGATCCA CCTTATAATA AAGGTCTCAT TGATAAAGCT TTAAAACTAA
  201 TTTCAGAGTT TAATTTATTG AAAGAAAATG GTATCATCGT TTGTGAATTT
  251 AGCAATCATG AAGAAATAGA TTATCAACCG TTTAATATGA TTAAACGTTA
  301 CCATTATGGG TTGACAGACA CATTGTTATT AGAAAAGGGA GAATAGCATG
  351 GAACATACAA TAGCGGTCAT TCCGGGTAGT TTTGACCCCA TTACTTATGG
  401 TCATTTAGAC ATTATTGAGA GAAGTACAGA TAGATTTGAT GAAATTCATG
  451 TCTGTGTTCT TAAAAATAGT AAAAAAGAAG GTACGTTTAG TTTAGAAGAG
  501 CGTATGGATT TAATTGAACA ATCTGTTAAA CATTTACCTA ATGTCAAGGT
  551 TCATCAATTT AGTGGTTTAC TAGTCGATTA TTGTGAACAA GTAGGAGCTA
  601 AAACAATCAT ACGTGGTTTA AGAGCAGTCA GTGATTTTGA ATATGAATTA
  651 CGCTTAACTT CMATGAATAA AAAGTTGAAC AATGAAATTG AAACGTTATA
  701 TATGATGTCT AGTACTAATT ATTCATTTAT AAGTTCAAGT ATTGTTAAAG
  751 AAGTTGCAGC TTATCGAGCA GATATTTCTG AATTCGTTCC ACCTTATGTT
  801 GAAAAGGCAT TGAAGAAGAA ATTTAAGTAA TAAAAATAAC AGTATTTTAG
  851 GTTTATCATG GTTTACAATC CTAAAATACT GTTTTCATTT GTTAACGATA
  901 TTGCTGTATG ACAGGCGTGT TGAAATCTGT TTGTTGTTGC CCGCTTATTG
  951 CATTGTATAT GTGTGTTGCT TTGATTTCAT TTGTGAAGTA ATGTGCATTG
1001 CTTTTGTTAA TATTGGTTAT ATATTGTCTT TCTGGGAACG CTGTTTTTAA
1051 ATGCTTTAAA TATTGTCTGC CACGGTCGTT CATCGCTAAT ACTTTAACTG
1101 CGTGAATGTT ACTCGTAACA TCTGTAGGTT TAATGTTTAA TAATACATTC
1151 ATTAACAGTC TTTGGATATG CGTATATGTA TAACGCTTTG TTTTTAGTAA
1201 TTTTACAAAA TGATGAAAAT CAGTTGCTTC ATAAATGTTA GATTTCAAAC
1251 GATTTTCAAA ACCTTCAGTA ACAGTATAAA TATTTTTTAA TGAATCTGTA
1301 GTCATAGCTA TGATTTGATA TTTCAAATAT GGAAATATTT GATTTAATGT
1351 WATATGAGGT GTTACGTACA AGTGTTGAAT ATCTTTAGGT ACCACATGAT
1401 GCCAATGATC ATCTTGACTA ATGATTGATG TTCTAATAGA TGTACCACTT
1451 SCAAACTGAT GGTGTTGAAT TAATGAATCA TGATGTTGAG CATTTTCTCG
1501 TTTGATAGAA ATTGCATTGA TGTTTTTAGC ATTTTTAGCA ATTGCTTTCA
1551 GGTAACTAAT ACCAAGTATG TTGTTAGGAC TTGCTAGTGC TTCATGATGC
1601 TCTAATAATT CGCTAATGAT ACGAGGGTAG CTTTTACCTT CTTTTACTTT
1651 TNGTGAAAAG GATTCAGATN GTTCAATTTC ATTAATNCTG NGTGCTAATT
1701 GCTTTAANGT TTNGATATCA TTATTTTCAC TACCAAATGC AATGGTATCG
1751 ACACTCATAT AATCNGCGAC TTNAACGGCT AGTTCGGCCA AGGGATCGAC
1801 CGGCAGGCAG
```

Mutant: NT33b
Phenotype: temperature sensitivity
Sequence map: Mutant NT33b is complemented by pMP636, which contains a 1.8 kb insert of S. aureus genomic DNA. A partial restriction map is depicted FIG. 38. Database searches at both the nucleic acid and peptide levels reveal strong peptide-level similarities to the lepC gene product, encoding signal peptidase I (EC 3.4.99.36) from B. caldolyticus (abbreviated as “Bca” in the sequence map).

DNA sequence data: The following DNA sequence data represents the sequence of clone pMP636, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP636
SEQ ID NO. 33
pMP636 Length: 1876 nt
      1 TCTGAATGAT CTARACGGAT TAAATTATTT AGCTGGTAAA ACAATCGACG
    51 AAGTTAACAC AAAAGCATTC GAAGGTACAT TATTAGCGCA TACTGATGGT
  101 GGTGTTCCTA ACATGGTAGT GAACATTCCA CAATTAGATG AAGAAACTTT
  151 CGGTTACGTC GTATACTTCT TCGAACTTGC TTGTGCAATG AGTGGATACC
  201 AATTAGGCGT AAATCCATTT AACCAACCTG GTGTAGAAGC ATATAAACAA
  251 AACATGTTCG CATTATTAGG TAAACCTGGT TTTGAAGACT TGAAAAAAGA
  301 ATTAGAAGAA CGTTTATAAA ATACATTACT TCAAAGATTA GTGAAGTTTG
  351 AAAAGATAGA ACTAGACGTT AACTATTTAA AGCATATTTT CGAGGTTGTC
  401 ATTACAAATG TAAAAATGTA ATGACAACCT CGTTTTTATT TATATGCAAG
  451 AACTAGGTTA CTAGCTAATG TGACAAGATG TTWAGAGAAA ATTAAAGATA
  501 AAATAATATC TGCCTTACAA TAATATTGTT ATACTACTAG AGACTGATTT
  551 ATTAGCATGA TTACATGTTA ATGTTTCTTT ACTTAGTAAT TAACTTTRTA
  601 ATGTAARAHT AATTATCTTC ADCCAHAGAA AGGGATTGAT GATTTGTCGT
  651 WTCMTCAATT AGAAGAATGG TTTGAGATAT KTCGACAGTT TGGTTWTTTA
  701 CCTGGATTTA TATTGTTATA TATTAGAGCT NTAATTCCAG TATTTCCTTT
  751 ARCACTCTAT ATTTTAATTA ACATTCAAGC TTATGGACCT ATTTTAGGTA
  801 TATTGATTAG TTGGCTTGGA TTAATTTCTG GAACATTTAC AGTCTATTTG
  851 ATCTGTAAAC GATTGGTGAA CACTGAGAGG ATGCAGCGAA TTAAACAACG
  901 TACTGCTGTT CAACGCTTGA TTAGTTTTAT TGATCGCCAA GGATTAATCC
  951 CATTGTTTAT TTTACTTTGT TTTCCTTTTA CGCCAAATAC ATTAATAAAT
1001 TTTGTAGCGA GTCTATCTCA TATTAGACCT AAATATTATT TCATTGTTTT
1051 GGCATCATCA AAGTTAGTTT CAACAATTAT TTTAGGTTAT TTAGGTAAGG
1101 AAATTACTAC AATTTTAACG CATCCTTTAA GARGGATATT AATGTTAGTT
1151 GGTGTTGGTT GTATTTTGGA TTGTTGGAAA AAAGTTAGAA CAGCATTTTA
1201 TGGGATCGAA AAAGGAGTGA CATCGTGAAA AAAGTTGTAA AATATTTGAT
1251 TTCATTGATA CTTGCTATTA TCATTGTACT GTTCGTACAA ACTTTTGTAA
1301 TAGTTGGTCA TGTCATTCCG AATAATGATA TGYMCCCAAC CCTTAACAAA
1351 GGGGATCGTG TTATTGTWAA TAAAATTAAA GTAACATTTA ATCAATTGAA
1401 TAATGGTGAT ATCATAACAT ATAGGCGTGG TAACGGAGAT ATATACTAGT
1451 CGAATTATTG CCAAACCTGG TCAATCAATG GCGTTTCGTC AGGGACAATT
1501 ATACCGTGAT GACCGACCGG TTGACGCATC TTATGCCAAG AACAGAAAAA
1551 TTAAAGATTT TAGTTTGCGC AATTTTAAAG AATTAGGATG GTGATATTAT
1601 TCCGCCAAAC AATTTTGTTG TGCTAAATGA TCAAGATAAT AACAAGCACG
1651 ATTCAAGACA ATTTGGTTTA ATCGATAAAA AGGATATTAT TGGTAATGTT
1701 AGTTTACGAT ACTATCCTTT TTCAAAATGG ACTGTTCAGT TCAAATCTTA
1751 AAAAGAGGTG TCAAAATTGA AAAAAGAAAT ATTGGAATGG ATTATTTCAA
1801 TTGCAGTCGC TTTTGTCATT TTATTTATAG TAGGTAAATT TATTGTTACG
1851 CCATATACAA TTAAAGGTGA ATCAAT
```

Mutant: NT36
Phenotype: temperature sensitivity
Sequence map: Mutant NT36 is complemented by pMP109, which contains a 2.7 kb insert of S. aureus genomic DNA. A partial restriction map is depicted FIG. 39. Database searches at both the nucleic acid and peptide levels reveal identity at one end of the pMP109 clone to the plaC gene from S. aureus (Genbank Accession No. M63177), encoding a DNA-directed RNA polymerase (EC 2.7.7.6). Since clone pMP109 does not contain the entire plaC ORF, the complementation of mutant NT36 by clone pMP109 is not likely to be due to the presence of this gene. Further analysis of clone pMP109 reveals strong similarity at the peptide level to the dnaG gene of L. monocytogenes (Genbank Accession No. U13165; published in Lupski et al., 1994, Gene 151:161-166), encoding DNA primase (EC 2.7.7.-); these similarities also extend to the dnaG genes of L. lactis, B. subtilis, and E. coli. The relative size and location of the dnaG ORF within clone pMP109 is denoted by an arrow in the sequence map.

DNA sequence data: The following DNA sequence data represents the sequence of clone pMP109, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP109
SEQ ID NO. 34
pMP109 Length: 2687 nt
      1 TATGATGATG GTAAAGATCC TAAAGGATTA CCTAAAGCTG ATATTGTTTT
    51 ACTTGGTATT TCGAGAACTT CAAAGACACC ATTATCTCAG TATTTAGCGC
  101 ATAAGAGTTA CAAAGTTATG AATGTACCGA TTGTACCAGA AGTGACACCG
  151 CCAGATGGCT TATATGATAT TAATCCAAAG AAATGTATCG CACTTAAAAT
  201 AAGTGAAGAA AAATTAAATC GCATTAGAAA AGAGCGACTA AAACAATTAG
  251 GACTAGGTGA CACAGCTCGA TATGCAACAG AAGCACGAAT TCAAGAAGAA
  301 TTGAATTACT TTGAAGAAAT CGTAAGTGAA ATTGGATGTC CTGTCATTGA
  351 TGTTTCTCAA AAAGCAATCG AAGAAACAGC AAACGATATA ATCCATTATA
  401 TTGAACAAAA TAAATCGAAA TGATTTCATT TTTGTCGAAA ATTAGGTATA
  451 ATAGTATAAC TAATGCTTAA TAGGTGATTT AATTTGCGAA TAGATCAATC
  501 GATCATTAAT GAAATAAAAG ATAAAACCGA CATTTTAGAC TTGGTAAGTG
  551 AATATGTWAA ATTAGAAAAG AGAGGACGCA ATTATATAGG TTTGTGTCCT
  601 TTTCATGATG AAAAGACACC TTCATTTACA GTTTCTGAAG ATAAACAAAT
  651 TTGTCATTGT TTTGGTTGTA AAAAAGGTGG CAATGTTTTC CAATTTACTC
  701 AAGAAATTAA AGACATATTC ATTTGTTGAM GCGGTThAAG AATTAGGTGG
  751 WTAGRGTTAA TGTTTGCTGT AGRTATTGAG GCAMCACAAT CTTWACTCAA
  801 ATGTYCAAAT TSCTTCTSRY GRTTTACAAA TGATTGACAW TGCATGGRGT
  851 TAWTACAAGR ATTTTATTAT TACGCTTTAA CAAAGACAGT CGAAGGCGAA
  901 CAAGCATTAA CGTACTTACA AGAACGTGGT TTTACAGATG CGCTTATTAA
  951 AGAGCGAGGC ATTGGCTTTG CACCCGATAG CTCACATTTT TGTCATGATT
1001 TTCTTCAAAA AAAGGGTTAC GATATTGAAT TAGCATATGA AGCCGGATTA
1051 TWATCACGTA ACGAAGAAAA TTTCAGTTAT TTACGATAGA TTYCGAAAYC
1101 GTATTATGTT YCCTTTGAAA AATGCGCAAG GAAGAATTGT TGGATATTCA
1151 GGTCGAACAT ATACCGGTCA AGAACCAAAA TACTTAAATA GTCCTGAAAC
1201 ACCTATCTTT CAAAAAAGAA AGTTGTTATA CAACTTAGAT AAAGCGCGTA
1251 AATCAATTAG AAAATTAGAT GAAATCGTAT TACTAGAAGG TTTTATGGAT
1301 GTTATAAAAT CTGATACTGC TGGCTTGAAA AACGTTGTTG CAACAATGGG
1351 TACACAGTTG TCAGATGAAC ATATTACTTT TATACGAAAG TTAACATCAA
1401 ATATAACATT AATGTTTGAT GGGGATTTTG CGGGTAGTGA AGCAACACTT
1451 AAAACAGGTY CAAAATTTGT TACAGCAAGG GCTAAATGTR TTTKTTATAC
1501 AATTGCCATC AGGCATGGAT CCGGATGAAT ACATTGGTAA GTATGGCAAC
1551 GATGCATTTM CTGCTTTTST AAAAAATGAC AAAAAGTCAT TTSCACATTA
1601 TAAAGTGAGT ATATTAAAAG ATGAAATTGC ACATAATGAC CTTTCATATG
1651 AACGTTATTT GAAAGANCTA AGTCATGATA TTTCGCTTAT GAAATCATCG
1701 ATTTTGCAAC AAAAGGCTTT AAATGATGTT GCACCATTTT TCAATGTTAG
1751 TCCTGAGCAA TTAGCTAACG AAATACAATT CAATCAAGCA CCAGCCAATT
1801 ATTATCCAGA AGATGAGTAT GGCGGTTACA TTGAACCTGA GCCAATTGGT
1851 ATGGCACAAT TTGACAATTT GAGCCGTCAA GAAAAAGCGG AGCGAGCATT
1901 TTTAAAACAT TTAATGAGAG ATAAAGATAC ATTTTTAAAT TATTATGAAA
1951 GTGTTGATAA GGATAACTTC ACAAATCAGC ATTTTAAATA TGTATTCGAA
2001 GTCTTACATG ATTTTTATGC GGAAAATGAT CAATATAATA TCAGTGATGC
2051 TGTGCAGTAT GTTAATTCAA ATGAGTTGAG AGAAACACTA ATTAGCTTAG
2101 AACAATATAA TTTGAATGAC GAACCATATG AAAATGAAAT TGATGATTAT
2151 GTCAATGTTA TTAATGAAAA AGGACAAGAA ACAATTGAGT CATTGAATCA
2201 TAAATTAAGG GAAGCTACAA GGATTGGCGA TGTAGAATTA CAAAkATACT
2251 ATTTACAGCA AATTGTTGCT AAGAATAAAG AACGCATGTA GCATGTGATT
2301 TTAAAGAATA ATACGAATAA TGATTATGTC AAAATGTATA AGGGTAAATG
2351 ATAGTTACCG CATTTAAACA ACACTATTGA AAAATAAATA TTGGGATTAG
2401 TTCCAATTTG TAAAATAAAA TTAAAAATAT GGATGAATTA ATTAAGAATT
2451 TAGTTTAAAA TAGCAATATT GAATAAATTT CGAATGTTCA TATTTAAAAT
2501 CGGGAGGCCG TTTCATGTCT GATAACACAG TTAAAATTAA AAAACAAACA
2551 ATTGATCCGA CATTAACATT AGAAGATGTT AAGAAGCAAT TAATTGAAAA
2601 AGGTAAAAAA GAGGGTCATT TAAGTCATGA AGAAATTGCT GAAAAACTTC
2651 AGAATTTTGA TATCGACTCT GATCAAATGG ATGATTT
```

Mutant: NT37
Phenotype: temperature sensitivity
Sequence map: Mutant NT37 is complemented by pMP72, which contains a 2.8 kb insert of S. aureus genomic DNA. A partial restriction map is depicted 40. Database searches at both the nucleic acid and peptide levels reveal a strong similarity at the peptide level to the glmS gene of B. subtilis (Genbank Accession No. U21932; published in Morohoshi, F. et al. J. Bacteriol. 175 (1993) 6010-6017), which encodes the protein L-glutamine-D-fructose-6-phosphate amidotransferase (EC 2.6.1.16). The relative location and predicted size of this ORF is designated by an arrow in the sequence map.

DNA sequence data: The following DNA sequence data represents the sequence of clone pMP72, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP72
SEQ ID NO. 35
pMP72 Length: 2800 nt
      1 NTNAATTAAC ATGCGAGGNC ACCCCTTTAT TGCTACTCCA TACTTCTCAT
    51 AAAATCATAT TAACATAACA CCCTTAATTG TCAGACTATT NAAATAAATA
  101 AAACACTTCA TTTTTACGCA TTTCTGCCAA ATTAAGATGA AGTAAAAGCT
  151 AAGTCGACCT AAAAAAGCAC CCTTCTAGTC GATTAATCTA AAAGGGGTGC
  201 CATATACTTT AATTTTAATA CATGATTGAT TCTAAAAAAG TGAATTATTC
  251 CACAGTAACT GATTTAGCAA GGTTACGTGG TTTATCAACA TCTAAATCTC
  301 TGTGTAATGC TGCATAGTAT GAAATTAATT GTAATGCAAC CACTGATACT
  351 AATGGCGTTA ACAATTCATG TACATGAGGA ATGACATAAG TGTCGCCTTC
  401 TTTTTCAAGA CCCTCCATAG AAATAATACA TGGATGTGCA CCACGTGCTA
  451 CTACCTCTTT AACGTTACCA CGAATTGATA AATTAACTTT CTCTTGTGTT
  501 GCTAAACCTA CAACTGGTGT ACCTTCTTCG ATTAAGGCAA TTGTACCATG
  551 TTTAAGTTCT CCACCAGCAA AACCTTCTGC TTGAATGTAA GAAATTTCTT
  601 TAAGTTTTAA CGCACCTTCT AAACTTACGT TATAGTCAAT AGTACGTCCG
  651 ATAAANAATG CATTGCGTGT TGTTTCTAAG AAATCTGTAG CAATTTGTTC
  701 CATAATTGGT GCATCGTCAA CAATTGCTTC TATTGCTGTT GTTACTTTTG
  751 CTAATTCTCT CAATAAATCA ATATCTGCTT CACGACCATG CTCTTTTGCA
  801 ACGATTTGAG ACAAGAWTGA TAATACTGCA ATTTGTGCAG WATAWGCTTT
  851 TGTAGATGCA ACTGCGAWTT CAGGGACCCG CGTGTAATAA CAATGTGTGG
  901 TCTGCTTCAC GTTGATAAAG TTGAACCTGC AACATTAGTG ATTGTTAATG
  951 AWTTATGAMC TAATTTATTA GTTWCAACTA AATACGGCGC GGCTATCTGG
1001 CAGTTTCACC TGATTGAGAA ATATAAACGA ACAATGGTTT TTAAGATAAT
1051 AATGGCATGT TGTAGACAAA CTCTGATGCA ACGTGTACTT CAGTTGGTAC
1101 GCCAGCCCAT TTTTCTAAAA ATTCTTTACC TACTAAACCT GCATGGTAGC
1151 TTGTACCTGC TGCAATAACG TAAATGCGGT CTGCTTCTTT AACATCATTG
1201 ATGATGTCTT GATCAATTTT CAAGTTACCT TCTGCATCTT GATATTCTTG
1251 AATAATACGA CGCATTACTG CTGGTTGTTC ATGAATTTCT TTTAACATGT
1301 AGTGTGCATA AACACCTTTT TCAGCATCTG ATGCATCAAT TTCAGCAATA
1351 TATGAATCAC GTTCTACAAC GTTTCCATCT GCATCTTTAA TAATAACTTC
1401 ATCTTTTTTA ACAATAACGA TTTCATGGTC ATGGRTTTCT TTATATTCGC
1451 TTGTCACTTG TAACATTGCA AGTGCGTCTG ATGCGATAAC ATTGAAACCT
1501 TCACCAACAC CTAATAATAA TGGTGATTTA TTTTTAGCAA CATAGATTGT
1551 GCCTTTGHCT TCAGCATCTA ATAAACCTAA TGCATATGAA CCATGTAATA
1601 ATGACACAAC TTTTGTAAAT GCTTCTTCAG TTGAAAGTCC TTGATTTGAA
1651 AAGTATTCAA CTAATTGAAC GATAACTTCT GTATCTGTTT CTGAAATGAA
1701 TGATACACCT TGTAAGTATT CACCTTTTAA CTCTTCATAG TTTTCAATAA
1751 CACCGTTATG AACTAGAGTA AAACGGCCAT TTGATGATTG ATGTGGATGA
1801 GAGTTTTCAT GATTCGGTAC ACCGTGTGTT GCCCAACGTG TGTGACCGAT
1851 TCCAACAGGT CCATTCAAAA TCGCTACTAT CAGCAACTTT ACGTAATTCT
1901 GCAATACGAC CTTTTTCTTT AAATACAGTT GTATTATCAT YATTTACTAC
1951 TGCGATACCT GCAGAGTCAT AACCTCTGTA TTCTAATTTT TCTACAACCT
2001 TTTAATAATA ATTTCTTTGG CATTATCATA GCCAATATAA CCAACAATTC
2051 CACACATAAC GACATTTTCC TCCATATTGG AATAGTACGS GTAAATTATG
2101 ATTTATTGCC GATAATTTAG ATTGACAATC TGCTTTCATA ATATAAATAG
2151 GAACATGCTA TCATCGCATT CATCCATAAC AAATTAAGCA TAGTTATTTT
2201 TACAACTATA CAAATTGCTC ACACTGTACT TTCCATATTA ATATTTTTTA
2251 TATTCAATTT CTGGCGATCT TATTAACTTT GTCCATTAAG TCACCCTAAT
2301 GTTTTACTTA ATAAGCTAAC GAATGAGCCA CATCCGGGAT AGCATCCGCC
2351 GATCTATTCG ATCACTATCC TCTTCGTCTA CAAATACATA TATTGCACTC
2401 TATAAAGGCC ACTCATATAT TAACCTTTAA TCTTCAAATA CAAATATTTA
2451 TTTGCACAGG CGCTTTAACT GTACTGCCGA ACTTTCCCCC TTTCCATTAA
2501 TCATTATTGT ACAACGGTGT TGTTTTGTTT TGCAAATATT TTCACAATAA
2551 AATTTTAAAA ATCCTAAAAC AATTTTTTTG TTTTACTTTT TCAAAATATC
2601 TATACTGTCA CATTGATGAC ACTTTATTTA ATTTTGTCAC ATTTATTTTG
2651 ACAAAGTTGA TTTTTGTTTA TATTGAGTAA CAAGTAACCT CTCTATACAC
2701 TATATATAGT CACATATATT AAAAAAGAGG TGTAAACATG TCACAAACTG
2751 AAGAGAAAAA AGGAATTGGT CGTCGTGTTC AAGCATTTGG ATCGACCGCA
```

Mutant: NT41/64
Phenotype: temperature sensitivity
Sequence map: Mutants NT41 and NT64 are complemented by pMP98, which contains a 2.9 kb insert of S. aureus genomic DNA. A partial restriction map is depicted FIG. 41. Database searches at both the nucleic acid and peptide levels reveal identity at both the peptide and nucleic acid levels to the C-terminal fragment of the pcrA gene from S. aureus (Genbank Accession No. M63176; published in Iordanescu, S. M. et al. J. Bacteriol. 171 (1989) 4501-4503), encoding DNA helicase (EC 3.6.1.-). Since only a small portion of the C-terminal fragment of the helicase protein is contained within clone pMP98, the pcrA gene is unlikely to be responsible for restoring a wild-type phenotype to mutants NT41 and 64. Further analysis reveals strong peptide level similarity to the lig gene of E. coli(Genbank Accession No. M30255; published in Ishino, Y. et al., Mol. Gen. Genet. 204 (1986) 1-7), encoding the protein DNA ligase (EC 6.5.1.2). The relative location and predicted size of the ORF encoding the putative S. aureus lig gene is depicted by an arrow in the sequence map.

DNA sequence data: The following DNA sequence data represents the sequence of clone pMP98, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP98
SEQ ID NO. 36
pMP98 Length: 2934 nt
      1 CATGAAATGC AAGAAGAACG TCGTATTTGT TATGTAGCAA TTACAAGGGC
    51 TGAAGAGGTG TTATATATCA CTCATGCGAC ATCAAGAATG TTATTTGGTC
  101 GCCCTCAGTC AAATATGCCA TCCAGATTTT TAAAGGAAAT TCCAGAATCA
  151 CTATTAGAAA ATCATTCAAG TGGCAAACGA CAAACGATAC AACCTAAGGC
  201 AAAACCTTTT GCTAAACGCG GATTTAGTCA ACGAACAACG TCAACGAAAA
  251 AACAAGTATT GTCATCTGAT TGGAATGTAG GTGACAAAGT GATGCATAAA
  301 GCCTGGGGAG AAGGCATGGT GAGTAATGTA AACGAGAAAA ATGGCTCAAT
  351 CGAACTAGAT ATTATCTTTA AATCACAAGG GCCAAAACGT TTGTTAGCGC
  401 AATTTGCACC AATTGAAAAA AAGGAGGATT AAGGGATGGC TGATTTATCG
  451 TCTCGTGTGA ACGRDTTACA TGATTTATTA AATCAATACA GTTATGAATA
  501 CTATGTAGAG GATAATCCAT CTGTACCAGA TAGTGAATAT GACAAATTAC
  551 TTCATGAACT GATTAAAATA GAAGAGGAGC ATCCTGAGTA TAAGACTGTA
  601 GATTCTCCAA CAGTTAGAGT TGGCGGTGAA GCCCAAGCCT CTTTCAATAA
  651 AGTCAACCAT GACACGCCAA TGTTAAGTTT AGGGAATGCA TTTAATGAGG
  701 ATGATTTGAG AAAATTCGAC CAACGCATAC GTGAACAAAT TCGCAACGTT
  751 GAATATATGT GCGAATTAAA AATTGATGGC TTAGCAGTAT CATTGAAATA
  801 TGTTGATGGA TACTTCGTTC AAGGTTTAAC ACGTGGTGAT GGAACAACAG
  851 GTTGAAGATA TTACCGRAAA TTTAAAAACA ATTCATGCGA TACCTTTGAA
  901 AATGAAAGAA CCATTAAATG TAGAAKTYCG TGGTGAAGCA TATATGCCGA
  951 GACGTTCATT TTTACGATTA AATGAAGAAA AAGAAAAAAA TGATGAGCAG
1001 TTATTTGCAA ATCCAAGAAA CGCTGCTGCG GGATCATTAA GACAGTTAGA
1051 TTCTAAATTA ACGGCAAAAC GAAAGCTAAG CGTATTTATA TATAGTGTCA
1101 ATGATTTCAC TGATTTCAAT GCGCGTTCGC AAAGTGAAGC ATTAGATGAG
1151 TTAGATAAAT TAGGTTTTAC AACGAATAAA AATAGAGCGC GTGTAAATAA
1201 TATCGATGGT GTTTTAGAGT ATATTGAAAA ATGGACAAGC CAAAGAAGAG
1251 TTCATTACCT TATGATATTG ATGGGATTGT TATTAAGGTT AATGATTTAG
1301 ATCAACAGGA TGAGATGGGA TTCACACAAA AATCTCCTAG ATGGGCCATT
1351 GCTTATAAAT TTCCAGCTGA GGAAGTAGTA ACTAAATTAT TAGATATTGA
1401 ATTAAGTATT GGACGAACAG GTGTAGTCAC ACCTACTGCT ATTTTAGAAC
1451 CAGTAAAAGT AGCTGGTACA ACTGTATCAA GAGCATCTTT GCACAATGAG
1501 GATTTAATTC ATGACAGAGA TATTCGAATT GGTGATAGTG TTGTAGTGAA
1551 AAAAGCAGGT GACATCATAC CTGAAGTTGT ACGTAGTATT CCAGAACGTA
1601 GACCTGAGGA TGCTGTCACA TATCATATGC CAACCCATTG TCCAAGTTGT
1651 GGACATGAAT TAGTACGTAT TGAAGGCGAA GTTAGCACTT CGTTGCATTA
1701 ATCCAAAATG CCAAGCACAA CTTGTTGAAG GATTGATTCA CTTTGTATCA
1751 AGACAAGCCA TGAATATTGA TGGTTTAGGC ACTAAAATTA TTCAACAGCT
1801 TTATCAAAGC GAATTAATTA AAGATGTTGC TGATATTTTC TATTTAACAG
1851 AAGAAGATTT ATTACCTTTA GACAGAATGG GGCAGAAAAA AGTTGATAAT
1901 TTATTAGCTG CCATTCAACA AGCTAAGGAC AACTCTTTAG AAAATTTATT
1951 ATTTGGTCTA GGTATTAGGC ATTTAGGTGT TAAAGCGAGC CAAGTGTKAG
2001 CAGAAAAATA TGAAACGATA GATCGATTAC TAACGGTAAC TGAAGCGGAA
2051 TTAGTAGAAT TCATGATATA GGTGATAAAG TAGCGCAATC TGTAGTTACT
2101 TATTTAGCAA ATGAAGATAT TCGTGCTTTA ATTCCATAGG ATTAAAAGAT
2151 AAACATGTTA ATATGATTTA TGAAGGTATC CAAAACATCA GATATTGAAG
2201 GACATCCTGA ATTTAGTGGT AAAACGATAG TACTGACTGG TAAGCTACAT
2251 CCAAATGACA CGCAATGAAG CATCTAAATG GCTTGCATCA CCAAGGTGCT
2301 AAAGTTACAA GTAGCGTTAC TAAAAATACA GATGTCGTTA TTGCTGGTGA
2351 AGATGCAGGT TCAAAATTAA CAAAAGCACA AAGTTTAGGT ATTGAAATTT
2401 GGACAGAGCA ACAATTTGTA GATAAGCAAA ATGAATTAAA TAGTTAGAGG
2451 GGTATGTCGA TGAAGCGTAC ATTAGTATTA TTGATTACAG CTATCTTTAT
2501 ACTCGCTGCT TGTGGTAACC ATAAGGATGA CCAGGCTGGA AAAGATAATC
2551 AAAAACATAA CAATAGTTCA AATCAAGTAA AAGAAATTGC AACGGATAAA
2601 AATGTACAAG GTGATAACTA TCGTACATTG TTACCATTTA AAGAAAGCCA
2651 GGCAAGAGGA CTTTTACAAG ATAACATGGC AAATAGTTAT AATGGCGGCG
2701 ACTTTGAAGA TGGTTTATTG AACTTAAGTA AAGAAGTATT TCCAACAGAT
2751 AAATATTTGT ATCAAGATGG TCAATTTTTG GACAAGAAAA CAATTAATGC
2801 CTATTTAAAT CCTAAGTATA CAAAACGTGA AATCGATAAA ATGTCTGAAA
2851 AAGATAAAAA AGACAAGAAA GCGAATGAAA ATTTAGGACT TAATCCATCA
2901 CACGAAGGTG AAACAGATCG ACCTGCAGKC ATGC
```

Mutant: NT42
Phenotype: temperature sensitivity
Sequence map: Mutant NT42 is complemented by pMP76, which contains a 2.5 kb insert of S. aureus genomic DNA. A partial restriction map is depicted FIG. 42. Database searches at both the nucleic acid and peptide levels reveal strong similarity at the peptide level to ORFs of unknown function in B. subtilis (Genbank Accession No. Z38002; characterization of the Ipc29D polypeptide is unpublished as of 1995). Strong similarity is also noted to the SUA5 protein from the yeast S. cerevisiae, which is described as being essential for normal growth (published in Na, J. G. et al. Genetics 131 (1992) 791-801).

DNA sequence data: The following DNA sequence data represents the sequence of clone pMP76, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP76
SEQ ID NO. 37
pMP76 Length: 2515 nt
      1 CSYCGGWACC CGGGGATCCT CTAGAGTCGA TCGTTCCAGA ACGTATTCGA
    51 ACTTATAATT ATCCACAAAG CCGTGTAACA GACCATCGTA TAGGTCTAAC
  101 GCTTCAAAAA TTAGGGCAAA TTATGGAAGG CCATTTAGAA GAAATTATAG
  151 ATGCACTGAC TTTATCAGAG CAGACAGATA AATTGAAAGA ACTTAATAAT
  201 GGTGAATTAT AAAGAAAAGT TAGATGAAGC AATTCATTTA ACACAACAAA
  251 AAGGGTTTGA ACAAACACGA GCTGAATGGT TAATGTTAGA TGTATTTCAA
  301 TGGACGCGTA CGGACTTTGT AGTCCACATG CATGATGATA TGCCGAAAGC
  351 GATGATTATG AAGTTCGACT TAGCATTACA ACGTATGTTA TTAGGGAGAG
  401 CCTATACAGT ATATAGTTGG CTTTGCCTCA TTTTATGGTA GAACGTTTGA
  451 TGTAAACTCA AATTGTTTGA TACCAAGACC TGAAACTGAA GAAGTAATGT
  501 TGCATTTCTT ACAACAGTTA GAAGATGATG CAACAATCGT AGATATCGGA
  551 ACGGGTAGTG GTGTACTTGC AATTACTTTG AAATGTTGAA AAGCCGGATT
  601 TAAATGTTAT TGCTACTGAT ATTTCACTTG AAGCAATGAA TATGGCTCCG
  651 TAATAATGCT GAGAAGCATC AATCACAAAT ACAATTTTTA ACAGGGGATG
  701 CATTAAAGCC CTTAATTAAT GAAGGTATCA AKTTGAACGG CTTTGATATC
  751 TAATCCMCCA TATATAGATG AAAAAGATAT GGTTACGATG TCTCCMACGG
  801 TTACGARATT CGAACCACAT CAGGCATTGT TTGCAGATAA CCATGGATAT
  851 GCTATTTATG AATCAATCAT GGAAGATTTA CCTCACGTTA TGGAAAAAGG
  901 CAGCCCAGTT GTTTTTGAAA TTGGTTACAA TCAAGGTGAG GCACTTAAAT
  951 CAATAATTTT AAATAAATTT CCTGACAAAA AAATCGACAT TATTAAAGAT
1001 ATAAATGGCC ACGATCGAAT CGTCTCATTT AAATGGTAAT TAGAAGTTAT
1051 GCCTTTGCTA TGATTAGTTA AGTGCATAGC TTTTTGCTTT ATATTATGAT
1101 AAATAAGAAA GGCGTGATTA AGTTGGATAC TAAAATTTGG GATGTTAGAG
1151 AATATAATGA AGATTTACAG CAATATCCTA AAATTAATGA AATAAAAGAC
1201 ATTGTTTTAA ACGGTGGTTT AATAGGTTTA CCAACTGAAA CAGTTTATGG
1251 ACTTGCAGCA AATGCGACAG ATGAAGAAGC TGTAGCTAAA ATATATGAAG
1301 CTAAAGGCCG TCCATCTGAC AATCCGCTTA TTGTTCATAT ACACAGTAAA
1351 GGTCAATTAA AAGATTTTAC ATATACTTTG GATCCACGCG TAGAAAAGTT
1401 AATGCAGGCA TTCTGGCCGG GCCCTATTTC GTTTATATTG CCGTTAAAGC
1451 TAGGCTATCT ATGTCGAAAA GTTTCTGGAG GTTTATCATC AGTTGCTGTT
1501 AGAATGCCAA GCCATTCTGT AGGTAGACAA TTATTACAAA TCATAAATGA
1551 ACCTCTAGCT GCTCCAAGTG CTAATTTAAG TGGTAGACCT TCACCAACAA
1601 CTTTCAATCA TGTATATCAA GATTTGAATG GCCGTATCGA TGGTATTGTT
1651 CAAGCTGAAC AAAGTGAAGA AGGATTAGAA AGTACGGTTT TAGATTGCAC
1701 ATCTTTTCCT TATAAAATTG CAAGACCTGG TTCTATAACA GCAGCAATGA
1751 TTACAGAAAT AMTTCCGAAT AGTATCGCCC ATGCTGATTA TAATGATACT
1801 GAACAGCCAA TTGCACCAGG TATGAAGTAT AAGCATTACT CAACCCAATA
1851 CACCACTTAC AATTATTACA GATATTGAGA GCAAAATTGG AAATGACGGT
1901 AAAGATTRKW MTTCTATAGC TTTTATTGTG CCGAGTAATA AGGTGGCGTT
1951 TATACCAAGT GARSCGCAAT TCATTCAATT ATGTCAGGAT GMCAATGATG
2001 TTAAACAAGC AAGTCATAAT CTTTATGATG TGTTACATTC ACTTGATGAA
2051 AATGAAAATA TTTCAGCGGC GTATATATAC GGCTTTGAGC TGAATGATAA
2101 TACAGAAGCA ATTATGAATC GCATGTTAAA AGCTGCAGGT AATCACATTA
2151 TTAAAGGATG TGAACTATGA AGATTTTATT CGTTTGTACA GGTAACACAT
2201 GTCGTAGCCC ATTAGCGGGA AGTATTGCAA AAGAGGTTAT GCCAAATCAT
2251 CAATTTGAAT CAAGAGGTAT ATTCGCTGTG AACAATCAAG GTGTTTCGAA
2301 TTATGTTGAA GACTTAGTTG AAGAACATCA TTTAGCTGAA ACGACCTTAT
2351 CGCAACAATT TACTGAAGCA GATTTGAAAG CAGATATTAT TTTGACGATG
2401 TCGTATTCGC ACAAAGAATT AATAGAGGCA CACTTTGGTT TGCAAAATCA
2451 TGTTTTCACA TTGCATGAAT ATGTAAAAGA AGCAGGAGAA GTTATAGATC
2501 GACCTGCAGG CATGC
```

Mutant: NT47
Phenotype: temperature sensitivity
Sequence map: Mutant NT47 is complemented by pMP639, which contains a 2.6 kb insert of S. aureus genomic DNA. A partial restriction map is depicted FIG. 43, along with open boxes to indicate the percentage of the clone for which DNA sequence has been obtained. Database searches at both the nucleic acid and peptide levels reveal strong similarity at the peptide level to two hypothetical ORFs of unknown function, one from K. pneumonia and one from Synechocystis spp. (abbreviated as “Kpn” and “Scy” in the diagram below. Experiments are currently underway to determine which ORF (or both) is an essential gene. The relative orientation and predicted size of these uncharacterized ORFs with respect to the partial restriction map of clone pMP639 are depicted by arrows in the map.

DNA sequence data: The following DNA sequence data represents the sequence of clone pMP639, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP639
SEQ ID NO. 38
pMP639 Length: 2635 nt
      1 ATTCTCTGTG TTGGGGCCCC TGACTAGAGT TGAAAAAAGC TTGTTGCAAG
    51 CGCATTTTCA TTCAGTCAAC TACTAGCAAT ATAATATTAT AGACCCTAGG
  101 ACATTGATTT ATGTCCCAAG CTCCTTTTAA ATGATGTATA TTTTTAGAAA
  151 TTTAATCTAG ACATAGTTGG AAATAAATAT AAAACATCGT TGCTTAATTT
  201 TGTCATAGAA CATTTAAATT AACATCATGA AATTCGTTTT GGCGGTGAAA
  251 AAATAATGGA TAATAATGAA AAAGAAAAAA GTAAAAGTGA ACTATTAGTT
  301 GTAACAGGTT TATCTGGCGC AGGTAAATCT TTGGTTATTC AATGTTTAGA
  351 AGACATGGGA TATTTTTGTG TAGATAATCT ACCACCAGTG TTATTGCCTA
  401 AATTTGTAGA GTTGATGGAA CAAGGGAAAT CCATCCTTAA GAAAAAGTGG
  451 CAATTGCAAT TGATTTAAGA RGTAAGGAAC TATTTAATTC ATTAGTTGCA
  501 GTAGTGGATA AAGTTCAAAA GTTGAAAGTG ACGTCATCAT TGATGTTATG
  551 TTTTTAGAAG CAAGTACTGA AAAATTAATT TCAAGATATA AGGAAACGCG
  602 TCCKTGCACA TCCTTTGATG GAACAAGGTT AAAAGATCGT TAATCAATGC
  651 MATTAATGAT GAGCGAGAGC ATTTGTCTCA AATTAGAAGT ATAGCTAATT
  701 TTGTTATAGA TAACTACAAA GTTATCACCT AAAGAATTAA AAGAACGCAT
  751 TCGTCGATAC TATGAAGATG AAGAGTTTGA AACTTTTACA ATTAATGTCA
  801 CAAGTTTCGG TTTTAAACAT GGGATTCAGA TGGATGCAGA TTTAGTATTT
  851 GATGTACGAT TTTTACCAPA TCCATATTAT GTAGTAGATT TAAGACCTTT
  902 AACAGGATTA GATAAAGACG TTTATAATTA TGTTATGAAA TGGAAAGAGA
  951 CGGAGATTTT TCTTTGAAAA ATTAACTGAT TTGTTAGATT TTATGATACC
1001 CGGGTWTAAA AAAGAAGGGA AATCTCAATT AGTAATTGCC ATCGGTTGTA
1051 CGGGTGGGAC AACATCGATC TGTAGCATTA GCAGAACGAC TAGGTWATTA
1101 TCTAAATGAA GTWTTTGAAT ATAATGTTTA TGTGCATCAT AGGGACGCAC
1151 ATATTGAAAG TGGCGAGAAA AAATGAGACA AATAAAAGTT GTACTTATCG
1201 GGTGGTGGCA CTGGCTTATC AGTTATGGCT AGGGGATTAA GAGAATTCCC
1251 AATTGATATT ACGGCGATTG TAACAGTTGC TGATAATGGT GGGAGTACAG
1301 GGAAAATCAG AGATGAPATG GATATACCAG CACCAGGAGA CATCAGAAAT
1351 GTGATTGCAG CTTTAAGTGA TTCTGAGTCA GTTTTAAGCC AACTTTTTCA
1401 GTATCGCTTT GAAGAAAATC AAATTAGCGG TCACTCATTA GGTAATTTAT
1451 TAATCGCAGG TATGACTAAT ATTACGAATG ATTTCGGACA TGCCATTAAA
1501 GCATTAAGTA AAATTTTAAA TATTAAAGGT AGAGTCATTC CATCTACAAA
1551 TACAAGTGTG CAATTAAATG CTGTTATGGA AGATGGAGAA ATTGTTTTTG
1601 GAGAAACAAA TATTCCTAAA AAACATAAAA AAATTGATCG TGTGTTTTTA
1651 GAACCTAACG ATGTGCAACC AATGGAAGAA GCAATCGATG CTTTAAGGGA
1701 AGCAGATTTA ATCGTTCTTG GACCAGGGTC ATTATATACG AGCGTTATTT
1751 CTAACTTATG TTKTGAATGG TATTTCAGAT GCGTTWATTC ATTCTGATGC
1801 GCCTAAGCTA TATGTTTCTA ATGTGATGAC GCAACCTGGG GAAACAGATG
1851 GTTATAGCGT GAAAGATCAT ATCGATGCGA TTCATAGACA AGCTGGACAA
1901 CCGTTTATTG ATTATGTCAT TTGTAGTACA CAAACTTTCA ATGCTCAAGT
1951 TTTGAAAAAA TATGAAGAAA AACATTCTAA ACCAGTTGAA GTTAATAAGG
2001 CTGAACTKGA AAAAGAAAGC ATAAATGTAA AAACATCTTC AAATTTAGTT
2051 GAAATTTCTG AAAATCATTT AGTAAGACAT AATACTAAAG TGTTATCGAC
2101 AATGATTTAT GACATAGCTT TAGAATTAAT TAGTACTATT CCTTTCGTAC
2151 CAAGTGATAA ACGTAAATAA TATAGAACGT AATCATATTA TGATATGATA
2201 ATAGAGCTGT GAAAAAAATG AAAATAGACA GTGGTTCTAA GGTGAATCAT
2251 GTTTTAAATA AGAAAGGAAT GACTGTACGA TGAGCTTTGC ATCAGAAATG
2301 AAAAATGAAT TAACTAGAAT AGACGTCGAT GAAATGAATG CAAAAGCAGA
2351 GCTCAGTGCA CTGATTCGAA TGAATGGTGC ACTTAGTCTT TCAAATCAAC
2401 AATTTGTTAT AAATGTTCAA ACGGAAAATG CAACAACGGC AAGACGTATT
2451 TATTCGTTGA TTAAACGTGT CTTTAATGTG GAAGTTGAAA TATTAGTCCG
2501 TAAAAAAATG AAACTTAAAA AAAATAATAT TTATATTTGT CGTACAAAGA
2551 TGAAAGCGAA AGAAATTCTT GATGAATTAG GAATTTTAAA AGACGGCATT
2601 TTTACGCATG AAATTGATCG ACCTGCAGGC ATGCA
```

Mutant: NT51
Phenotype: temperature sensitivity
Sequence map: Mutant NT51 is complemented by pMP86, which contains a 1.9 kb insert of S. aureus genomic DNA. A partial restriction map is depicted FIG. 44 (there are no apparent restriction sites for EcoR I, Hind III, or BamH I). Database searches at both the nucleic acid and peptide levels reveal strong similarity at the peptide level to an ORF of undetermined function in H. influenzae (Genbank Accession No. U32702):

DNA sequence data: The following DNA sequence data represents the sequence of clone pMP86, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP86
SEQ ID NO. 39
pMP86 Length: 1952 nt
      1 TGCATGTACA GCAGGCTCTA CACAACCGTC GCATGTTTTA GATGCAATGT
    51 TCGAAGATGA GGAGCGATCA AATCATTCGA TTCGATTTAG TTTTAACGAA
  101 TTGACTACTG AAAATGAAAT TAATGCAATT GTAGCTGAAA TTCATAAAAT
  151 ATATTTTAAA TTTAAGGAGG AGTCATAATT GTCAAATAAA GATATAACGT
  201 GTTGTCGTTG GTATGTCAGG CGGTGTAGAT AGTTCTGTAA CAGCCCACGT
  251 CTTAAAAGAA CAAGGTTATG ATGTCATTGG CATATTTATG AAAAACTGGG
  301 ATGACACTGA CGAAAATGGC GTATGTACTG CAACTGAAGA TTACAACGAT
  351 GTTATTGAAG TGTGTAATCA AATTGGCATT CCGTATTACG CTGTTAATTT
  401 TGAAAAAGAA TATTGGGATA AAGTCTTTAC GTATTTCTTA GATGAATACA
  451 AAAAAGGTCG TACTCCAAAT CCAGACGTTA TGTGTAATAA AGAAATTAAG
  501 TTTAAAGCCT TTTTAGATCA TGCGATGAAT TTAGGTGCAG ATTATGTAGC
  551 AACAGGACAT TACGCACGCA TACATCGTCA TGAASRTGGT CATGTTGAAA
  601 TGTTACGTGG TGTAGATAAT AATAAAGATC ARACATACTK CWKGMATGCA
  651 AKTATCTCAA CAACAACTTT CAAAAGTGAT GTTCCCAATT GGCGACATCG
  701 AAAAGAGTGA AGTGCGTCGA ATTGCTGAAG AACAAGGACT TGTTACTGCT
  751 AAGAAAAAAG ATTCTACAGG CATTTGTTTT ATCGGCGAAA AAAACTTTAA
  801 AACATTTTTA TCACAATATT TACCTGCACA ACCGGGTGAT ATGATAACAC
  851 TTGATGGTAA GAAAATGGGT AAACATAGTG GTTTGATGTA TTACACAATA
  901 GGACAAAGAC ATGGATTAGG TATAGGTGGG AGATGGCGAT CCTTGGTTTG
  951 TTGTCGGTAA AAACCTAAAA GATAATGTTT TATATGTWGA ACAAGGATCC
1001 ATCACGATGC ATTATACAGT GATTACTTAA TTGCTTCAGA CTATTCATTT
1051 GTAAATCCCA GAAGATAATG ACTTAGATCA AGGTTTTGAA TGTACAGCTA
1101 AATTTAGATA TCGCCAAAAA GATACGAAAG TTTTTGTGAA ACGTGAAAAA
1151 CGACCATGCA CTACGTGTTA CTTTTGCTGA GCCAGTAAGA GCAATCACAC
1201 CTGGACAAGC AGTTGTTTTT TATCAAGGTG ATGTGTTGTC TTGGTGGTGC
1251 AACAATTGAC GATGTKTTCA AAAATGAAGG TCAATTAAAT TATGTTGTAT
1301 ANACAATGGC AACAATAAAT TACTTATTTG AAGTTTCNAC GTTGAAAATG
1351 ACGAAAGACA GTTTTTGATG AGAATAATTC ATGAGGATAG AGTCTGGGAC
1401 ATCACAATGT CCTAGGCTCT ACAATGTTAT ATKGGCGGGA CCACAACATA
1451 GAGAATTTCG TAAAGAAATT CWACAGGCAA TGCCAGTTGG GGATAACGAA
1501 TTTAATTTTG TTAAAATATC ATTTCTGTCC CACTCCCTAT GCATGAATCT
1551 AATTATGTAT TCTTATTTTT AAGTACATAA TAGTGGTGGC TAATGTGGAA
1601 GAACCATTAC ATAATAAACC GTTAATGGTT CTTAAGCATT TYTATTCCAT
1651 TCCCGCTTTT TCATGAATGA AGATGATATT AGATTATATT TTATTCGTTG
1701 TTAAGTGATT CGAGACATAC AATTTATCAA GATGTTTATA ATTGATGAGA
1751 AATGAGGTTC GTAAATGATA GATCAACAAA CAATTTATCA ATACATACAA
1801 AATGGAAAAA TAGAAGAAGC GTTACAAGCA TTGTTCGGAA ATATCGAAGA
1851 AAATCCTACA ATTATTGAAA ATTATATTAA TGCTGGTATC GTACTTGCTG
1901 ATGCGAATGA GATTGAAAAG GCAGAGCGTT TTTTCCAAAA AGCTTTAACA
1951 AT
```

Mutant: NT52
Phenotype: temperature sensitivity
Sequence map: Mutant NT52 is complemented by pMP87, which contains a 2.3 kb insert of S. aureus genomic DNA. A partial restriction map is depicted FIG. 45. Database searches at both the nucleic acid and peptide levels strong peptide-level similarity to the kimE gene product, encoding mevalonate kinase (EC 2.7.1.36), from M. thermoautotrophicum (abbreviated as “Mth” in the sequence map.

DNA sequence data: The following DNA sequence data represents the sequence of clone pMP87, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP87
SEQ ID NO. 40
pMP87 Length: 2273 nt
      1 TAACCAATAT TGATAAAACC TTGATGTGTT TCGTGTCAAT GACATACCAT
    51 ATCGACTAGG TACCTTTTTA GAATGTTGAT TAATCACAAC AAATATCATG
  101 GCAAGGTCAT CTTCAAAATG ATTCGATTCA AGTGGAACGG CATATGACGT
  151 CTCATCACTA TACCCTTTTT CCCATTCTGC AAATCCACCA TAAATACTAC
  201 GCGACGCAGA ACCCGAACCA ATTCGCGCCA ATCTCGATAA ATCCTTATCT
  251 GACAGCTGCA TGTCTAGCGC TTGATTACAA GCTGCTGCTA AAGCTGCATA
  301 TGCGCTTGCC GATGAAGCCA ACCCTGCTGC TGTTGGTACA AAATTGTCGC
  351 TTTCAATTTC TGCATACCAA TCGATGCCAG CTCTATTTCT GACAATATCC
  401 ATATATTTTG AAATTTTCTC TAATTCTTTG CCACTAACCT TTTCACCATT
  451 CAACCAAAAT TGATCCTGTG TTAACTGGTC GTTAAAAGTG ACTTTCGTTT
  501 CAGTGTWAAA TTTTTCTAAT GTWACAGATA TGCTATTATT CATTGGAATG
  551 ATTAGTGCTT CATCTTTTTT ACCCCAATAT TTTATAAGTG CAATATTCGT
  601 ATGTGCACGT GCTTTGCCAC TTTTAATCAA CGCATTAACC TCCTAAATTC
  651 TCAATCCAAG TATGTGCTGC ACCAGCTTTT TCTACAGCTT TTACAATATT
  701 TTTCGCTGTT GGTAAATCTT TGGCAAGCAA TAACATACTT CCACCACGAC
  751 CAGCGCCAGT AAGTTTTCCA GCAATCGCAC CATTTTCTTT ACCAATTTTC
  801 ATTAATTGTT CTATTTTATC ATGACTAACT GTCAACGCCT TTAAATCCGC
  851 ATGACATTCA TTAAAAATAT CCGCTAAGGS TTCAAAGTTA TGATGTTCAA
  901 TCACATCACT CGCACGTAAA ACTAACTTAC CGATATGTTT TACATGTGAC
  951 ATGTACTGAG GGTCCTCACA AAGTTTATGA ACATCTTCTA CTGCTTGTCT
1001 TGTTGAACCT TTCACACCAG TATCTATAAC AACCATATAG CCGTCTAAAC
1051 TTAACGTTTT CAACGTTTCA GCATGACCTT TTTGGAACCA AACTGGTTTG
1101 CCTGATACAA TCGTTTGCGT ATCAATACCA CTTGGTTTAC CATGTGCAAT
1151 TTGCTCTGCC CAATTAGCCT TTTCAATGAG TTCTTCTTTC GTTAATGATT
1201 TCCCTAAAAA ATCATAACTT GCACGAACAA AAGCAACCGC GACAGCTGCA
1251 CTCGATCCTA ATCCACGTGA TGGTGGTAAA TTCGTTTGGA TCGTTACTGC
1301 TAGCGGCTCT GTAATATTAT TTAATTCTAC AAAACGGTTC ACCAAAGAMT
1351 TAAGATGGTC AGGCGCATCA TATAAACATA CCATCGTAAA ACATCGCTTT
1401 TAATAGAGGA ATAGTTCCCG CTCTCTAAGG TTCTATTAAA ACTTTGATTT
1451 TAACCGGCGT TAAACGGTAC TGCAATAGCA GGCTCTCCAA ATGTAACAGC
1501 ATGTTCTCCT ATTAAAATAA TCTTACCTGT CGATTCCCCA TATCCTTTTC
1551 TTGTCATGTC AATATCACCT TTTATATTTA TCCTAWACTT GATTCATTAT
1601 TTTTATTTAT TAGTAAAAGA CATCATATTC TAAGTKGCAW ACGCATTCGC
1651 GTTAAATTTC ATTGCAGTCT TTATCTCACA TTATTCATAT TATGTATAAT
1701 CTTTATTTTG AATTTATATT TGACTTAACT TGATTAGTAT AAAACTAACT
1751 TTCGTTTACT TCAAAGTTTA AATCTTATCG AGTGATATTT CAGATTCTTT
1801 ATCTTTTTAT AAAATAGCCC TACAATTTAT AATTTTCCAC CCTAACTATA
1851 ATACTACAAA TAATAATTGG AATATATAGA TTTACTACTA AAGTATTAGA
1901 ACATTTCAAT AGAAGGTCGT TTCTTTCATA GTCATACGCA ITATATATAC
1951 CCTATTCTCA ATCTATTTAA TACGTAAAAC ATGAAATTTT CTTATTAAAT
2001 TTATTATTTC CATCATATCA TTACTTTTAA TTTAATGATG TTCAATTTAA
2051 ATATTAGGTC AATAACATAT TTATGCTTTT TATGGATACT TTCAAAAATA
2101 ACAGCCCCAA ACGATAACTT GAAAGGGGCT GTTAAATATT TAACTATTGC
2151 ATTTGATCKA TCATTYTMKW GKWTCYYYSR RTMMYKWKMT CRAAATACGT
2201 ATCGTATCTT TGCCATTCTT CTTGAGTAAT TGGCGTCATA TTTAATACAC
2251 CGCCAAGATC GACCTGCAGG CAT
```

Mutant: NT53
Phenotype: temperature sensitivity
Sequence map: Mutant NT53 is complemented by pMP143, which contains a 3.0 kb insert of S. aureus genomic DNA. A partial restriction map is depicted FIG. 46, along with open boxes to indicate the percentage of the clone for which DNA sequence has been obtained. Database searches at both the nucleic acid and peptide levels reveal strong similarity at the peptide level to papS, encoding poly-A polymerase (EC 2.7.7.19) from B. subtilis (Genbank Accession No. L38424; published in Bower, S. et al. J. Bacteriol. 9 (1995) 2572-2575). Also included in this clone is the gene homolog for birA, which encodes biotin [acetyl-CoA-carboxylase] ligase and functions as a biotin operon repressor protein.

DNA sequence data: The following DNA sequence data represents the sequence of clone pMP143, starting with the standard M13 forward and M13 reverse sequencing primers and applying primer walking strategies to augment the sequence contigs. The sequences below can be used to design PCR primers for the purpose of amplification from genomic DNA with subsequent DNA sequencing:

```
clone pMP143
SEQ ID NO. 41
pMP143.forward Length: 928 nt
      1 TCCTCTAGAG TCGATCAATA TGAGTATTAT TATCAAAAAA TGCTAAATNA
    51 GCATAACAAA AGTAAAGGCG AGTAATAATA TGGATAAATC ATTATTTGAA
  101 YAGGCAAGGC CTATATTAGA ACAAATTCAA GACAAT6GTT TTNAAGCATA
  151 TTATGTAGGT GGCTCTGTAA GAGATTATGT CATGGGAAGA AATATTCATG
  201 ATATAGATAT CACAACAAGT GCAACGNCGG ATGAAATAGA ATCTATCTTT
  251 AGTCATACGA TACCTGTAGG TAAAGAACAT GGCACGATAA ATGTAGTTTT
  301 TAATGATGAA AATTATGAAG TGACAACATT CCGGGCTGAA GAAGATTATG
  351 TCGATCACCG TAGACCAAGT GGTGTTACAT TTGTYCGTGA TTTATACGAR
  401 GATTTGCAAC GACGAGATTT CACGATGAAT GCGATAGAAT GGATACAGCA
  451 TACAAATTGT ATGATTATTT TGATGGTCAA CAAGATATTA ATAATCGAWT
  501 AATAAGAACT GTAGGTATAG CTGAGGAACG TTCCAAGAAG ATGCTTTACG
  551 TATGATTCGA TGTTTAAGGT TCCAGTCACA ATTATCATTT GATATTGCAA
  601 CGGAAACATT CGAAGCGATG CGTATACAAA TGGCAGATAT TAAATTTTTA
  651 TCAATTGAGC GTATAGTGAT TGAACTAACT AAATTAATGC GAGGTATTAA
  701 TGTTGAAAAG AGTTTTAATC ATTTAAAATC GCTGAAAGCA TTTAATTATA
  751 TGCCGTATTT CGAACATCTT GATATGAATC AAATTAATGT AACTGAAGCA
  801 ATTGATTTAG AATTGTTGAT TGCTATAGTA TCAGTTAAAT TTGATATTAA
  851 TTACTCATTG AAGCCTTTAA AGCTAAGTTA ACCGACAAGT TAAAAGATAT
  901 CAATCAATAT ATTCAAATTA TGAATGCA
SEQ ID NO. 42
pMP143.reverse Length: 2119 nt
      1 TGCATGCCTG CAGGTCGATC TAATATAGTT TCCGCTAAAT ATAATTGTTG
    51 CGGTCGATAT GTTAAGCCAR GTYGATCTAC AGCTTTGCTA TATAAAGACT
  101 TCAAGCTGCC ATTATAATTT GTTGTCGGCT TTTTAAAATC AACTTGCTTA
  151 CGATAGATAA TCTGTTCGAA CTTTTCGTAC GATTTATCCA ATGGCTTTGC
  201 ATCATATTGC CTAACCATCT CAAAGAAAAT ATCATACAAA TCGTATTTCA
  251 ACTGTTTACT TAAATAATAT AATTGCTTCA AAGTATCTAA CGGTAACTTT
  301 TCAAATTTTT CAAAAGCTAA TATCATCAAT TTAGCAGTAG TAGCGGCATC
  351 TTCGTCAGCT CGATGGGCAT TTGCTAAGGT AATACCATGT GCCTCTGCTA
  401 ATTCACTTAA TTGATAGCTT TTATCTGTAG GAAAAGCTAT TTTAAAGATT
  451 TCTAGTGTAT CTATAACTTT TTTGGGACGA TATTGAATAT TACAATCTTT
  501 AAATGCCTTT TTAATAAAAT TCAAATCAAA ATCTACATTA TGAGCTACAA
  551 AAATGCAATC TTTWATCTTA TCGTAGATTT CTTGTGCAAC TTGATTAAAA
  601 TATGGCGCTT GTTGTAGCAT ATTTKCTTCA ATGGATGTTA ACGCWTGAAT
  651 GAACGGCGGA AWCTCTAAAT TTGTTCTAAT CATAGAATGA TATGTATCAA
  701 TAATTTGGTT ATTGCGSACA AACGTTATAC CAATTTGAAT GATATCGTCA
  751 AAATCTAATT GGTTGCCTGT TGTTTCCAAA TCCACAACGG CATAGGTTGC
  801 CATACCCATA GCTATCTCTC CTTGCTTTAG TGTTAAAAAT CTATATCTGC
  851 ACTAATTAAA CGGTGTGATT CACCCGCTTC ATCTCTAACA ATTAGATAGC
  901 CATCGTAATC TAAATCAATT GCTTGTCCTT TAAACTGTTT ATCATTTTCT
  951 GTAAATAGCA ACGTTCTATT CCAAATATTA GAAGCTGCAG TATATTCTTC
1001 ACGAATTTCA GAAAAAGGTA ACGTTAAAAA TTGATTATAT CTTTTTYCAA
1051 TTTCTTGAAG TAATATCTCT AAAAATTGAT ATCTATCTAA TTWATTTTTA
1101 TCATGTAATT GTATACTTGT TGCTCTATGT CTAATACTTY CATCAAAGTT
1151 TTCTAGTTGT TTGCGTTCAA ATTAATACCT ATACCACATA TTATTGCTTC
1201 TATACCATCC ATTATTAGCA ACCATTTCAG TTAAGAAACC ACACACTTTA
1251 CCATTATCAA TAAATATATC ATTCGGCCAT TTCACTTTGA CTTCATCTTG
1301 ACTAAAATGT TGAATCGCAT CTCTTATCCC TAATGCAATA AATAAATTAA
1351 ATTTAGATAT CATTGAGAAT GCAACGTTAG GTCTTAACAC GACAGACATC
1401 CAAAGTCCTT GCCCTTTTGA AGAACTCCAA TGTCTATTAA ATCGCCCACG
1451 ACCTTTCGTT TGTTCATCAC TCAAGATAAA AAATGAAGAT TGATTTCCAA
1501 CAAGTGACTT TTTCGCAGCA AGTTGTGTAG AATCTATTGA ATCGTATACT
1551 TCACTAAAAT CAAACAAAGC AGAACTTTTT GTATATTGGT CTATTATACC
1601 TTGATACCAA ATATCTGGGA GCTGTTGTAA TAAATGCCCT TTATGATTTA
1651 CTGAATCTAT TTTACATCCC TCTAACTTTA ATTGGTCAAT CACTTTTTTT
1701 ACTGCAGTGC GTGGAAATAT TAAGTTGATT CCGCAATGCT TTGTCCAGAA
1751 TATATAATTC GGTTTATTTT TATAGAGTAA TTGAAGTTAC ATCTTGACTA
1801 TATTTTNACA TGATTATCCA CCCATTTCAA AATTNCAGTT TCTNCGTTGC
1851 TTACTTTACC TGTNACAATC GCTATCTCAA TTTGTCTTAG CACATCTTTT
1901 AACCACGGAC CACTTTTGGC ATTTAAATGT GCCATAAGTA CACCGCCATT
1951 AACCATCATG TCTTTNCTAT TATGCATAGG TAAACGATGT AATGTTTCAT
2001 CAATCGTTTG AAGGTTAACG CTTAATGGTT CATGTCCTTG GTATCATAAC
2051 GCCTGTNTCA AGCGTTCTNC AANCATGTAC AGTTNTTCAA TGTGGNGTGT
2101 CCGNATTAAC GCTATTCAA
```

Mutant: NT54
Phenotype: temperature sensitivity
Sequence map: Mutant NT54 is complemented by pMP145, which contains a 3.1 kb insert of S. aureus genomic DNA. A partial restriction map is depicted FIG. 47, along with open boxes to indicate the percentage of the clone for which DNA sequence has been obtained. Database searches at both the nucleic acid and peptide levels reveal identity at the nucleic acid level and peptide level to the C-terminal portion of the pbp4 gene, encoding D,D-carboxy peptidase (EC 3.4.16.4) from S. aureus(Genbank Accession No. U29454; unpublished as of July, 1995). Since clone pMP146 does not contain the complete Pbp4 ORF, this gene is unlikely to be responsible for restoring mutant NT54 to a wild-type phenotype. Cross complementation with clone pMP91, which contains a 5.2 kb insert of S. aureus genomic DNA, reveals that only 800 additional base pairs downstream (3′ to) the Pbp4 ORF are necessary for complementation (data not shown). DNA sequence of this region reveals strong similarity at the nucleic acid and peptide levels to the tagD gene, encoding glycerol-3-phosphate cytidylyl transferase (EC 2.7.7.39), from B. subtilis (Genbank Accession No. M57497; published in Mauel, C. et al., J. Gen. Microbiol. 137 (1991) 929-941). The tagD gene of B. subtilis has been reported to be an essential gene and is therefore likely to be a good candidate for screen development. The relative size and location of the TagD ORF with respect to clone pMP145 is depicted by an arrow in the restriction map.

DNA sequence data: The following DNA sequence data represents the sequence of the right-most portion of clone pMP145, starting with the standard M13 reverse sequencing primer and applying primer walking strategies to complete the sequence contig. The sequence below can be used to design PCR primers for the purpose of amplification from genomic DNA with subsequent DNA sequencing:

```
clone pMP145
SEQ ID NO. 43
pMP145 Length: 1407 nt
      1 TTCACAGTGT TGTCGGGATA CGATATAGTA CACTGTACAG TACGNTGGAG
    51 ATTTATTAGA TTTTCACAGA ATTNTGAAAA TAAGACNACG GGTCATGGAA
  101 ATGTTACTAT TACCTGAACA AAGGCTATTA TATAGTGATA TGGTTGNTCG
  151 TATTTTATTC AATAATTCAT TAAAATATTA TATGAACGAA CACCCAGCAG
  201 TAACGCACAC GACAATTCAA CTCGTAAAAG ACTATATTAT GTCTATGCAG
  251 CATTCTGATT ATGTATCGCA AAACATGTTT GACATTATAA ATACAGTTGA
  301 ATTTATTGGT GAGAATTGGG ATAGAGAAAT ATACGAATTG TGGCGACCAA
  351 CATTAATTCA AGTGGGCATT AATAGGCCGA CTTATAAAAA ATTCTTGATA
  401 CAACTTAAAG GGAGAAAGTT TGCACATCGA ACAAAATCAA TGTTAAAACG
  451 ATAACGTGTA CATTGATGAC CATAAACTGC AATCCTATGA TGTGACAATA
  501 TGAGGAGGAT AACTTAATGA AACGTGTAAT AACATATGGC ACATATGACT
  551 TACTTCACTA TGGTCATATC GAATTGCTTC GTCGTGCAAG AGAGATGGGC
  601 GATTATTTAA TAGTAGCATT ATCAACAGAT GAATTTAATC AAATTAAACA
  651 TAAAAAATCT TATTATGATT ATGAACAACG AAAAATGATG CTTGAATCAA
  701 TACGCTATGT CRTATTTAGT CATTCCAGAA AAGGGCTGGG GACAAAAAGA
  751 AGACGATGTC GAAAAATTTG ATGTAGATGT TTTTGTTATG GGACATGACT
  801 GGGAAGGTGA ATTCGACTTC TTAAAGGATA AATGTGAAGT CATTTATTTA
  851 AAACGTACAG AAGGCATTTC GACGACTAAA ATCAAACAAG AATTATATGG
  901 TAAAGATGCT AAATAAATTA TATAGAACTA TCGATACTAA ACGATAAATT
  951 AACTTAGGTT ATTATAAAAT AAATATAAAA CGGACAAGTT TCGCAGCTTT
1001 ATAATGTGCA ACTTGTCCGT TTTTAGTATG TTTTATTTTC TTTTTCTAAA
1051 TAAACGATTG ATTATCATAT GAACAATAAG TGCTAATCCA GCGACAAGGC
1101 ATGTACCACC AATGATAGTG AATAATGGAT GTTCTrCCCA CATACTTTTA
1151 GCAACAGTAT TTGCCTTTTG AATAATTGGC TGATGAACTT CTACAGTTGG
1201 AGGTCCATAA TCTTTATTAA TAAATTCTCT TGGATAGTCC GCGTGTACTT
1251 TACCATCTTC GACTACAAGT TTATAATCTT TTTTACTAAA ATCACTTGGT
1301 AAAACATCGT AAAGATCATT TTCAACATAA TATTTCTTAC CATTTATCCT
1351 TTGCTCACCT TTAGACAATA TTTTTACATA TTTATACTGA TCAAATGAVC
1401 GTTCCAT
```

Mutant: NT55
Phenotype: temperature sensitivity
Sequence map: Mutant NT55 is complemented by pMP92, which contains a 2.0 kb insert of S. aureus genomic DNA. A partial restriction map is depicted FIG. 48. Database searches at both the nucleic acid and peptide levels reveal strong peptide-level similarity to the nadE gene product, encoding the nitrogen regulatory protein NH3-dependent NAD synthetase (EC 6.3.5.1), from E. coli (Genbank Accession No. M15328; published in Allibert, P. et al. J. Bacteriol. 169 (1987) 260-271).

DNA sequence data: The following DNA sequence data represents the sequence of clone pMP92, starting with the standard M13 forward and M13 reverse sequencing primers and applying primer walking strategies to complete the sequence contig. The sequences below can be used to design PCR primers for the purpose of amplification from genomic DNA with subsequent DNA sequencing:

```
clone pMP92
SEQ ID NO. 44
pMP92 Length: 1996 nt
      1 TCCTCTAGAG TCGATCGTAT TAAATTATCA AATAACGCTG AAAAGGTTAC
    51 GACGCCAGGT AAGAAAAATG TATATCGCAT TATAAACAAG AAAACAGGTA
  101 AGGCAGAAGG CGATTATATT ACTTTGGAAA ATGAAAATCC ATACGATGAA
  151 CAACCTTTAA AATTATTCCA TCCAGTGCAT ACTTATAAAA TGAAATTTAT
  201 AAAATCTTTC GAAGCCATTG ATTTGCATCA TAATATTTAT GAAAATGGTA
  251 AATTAGTATA TCAAATGCCA ACAGAAGATG AATCACGTGA ATATTTAGCA
  301 CTAGGATTAC AATCTATTTG GGATGAAAAT AAGCGTTTCC TGAATCCACA
  351 AGAATATCCA GTCGATTTAA GCAAGGCATG TTGGGATAAT AAACATAAAC
  401 GTATTTTTGA AGTTGCGGAA CACGTTAAGG AGATGGAAGA AGATAATGAG
  451 TAAATTACAA GACGTTATTG TACAAGAAAT GAAAGTGAAA AAGCGTATCG
  501 ATAGTGCTGA AGAAATTATG GAATTAAAGC AATTTATAAA AAATTATGTA
  551 CAATCACATT CATTTATAAA ATCTTTAGTG TTAGGTATTT CAGGAGGACA
  601 GGATTCTACA TTAGTTGGAA AACTAGTACA AATGTCTGTT AACGAATTAC
  651 GTGAAGAAGG CATTGATTGT ACGTTTATTG CAGTTAAATT ACCTTATGGA
  701 GTTCAAAAAG ATGCTGATGA AGTTGAGCAA GCTTTGCGAT TCATTGAACC
  751 AGATGAAATA GTAACAGTCA ATATTAAGCC TGCAGTTGAT CAAAGTGTGC
  801 AATCATTAAA AGAAGCCGGT ATTGTTCTTA CAGATTTCCA AAAAGGAAAT
  851 GAAAAAGCGC GTGAACGTAT GAAAGTACAA TTTTCAATTG CTTCAAACCG
  901 ACAAGGTATT GTAGTAGGAA CAGATCATTC AGCTGAAAAT ATAACTGGGT
  951 TTTATACGAA GTACGGTGAT GGTGCTGCAG ATATCGCACC TATATTTGGT
1001 TTGAATAAAC GACAAGGTCG TCAATTATTA GCGTATCTTG GTGCGCCAAA
1051 GGAATTATAT GAAAAAACGC CAACTGCTGA TTTAGAAGAT GATAAACCAC
1101 AGCTTCCAGA TGAAGATGCA TTAGGTGTAA CTTATGAGGC GATTGATAAT
1151 TATTTAGAAG GTAAGCCAGT TACGCCAGAA GAACAAAAAG TAATTGAAAA
1201 TCATTATATA CGAAATGCAC ACAAACGTGA ACTTGCATAT ACAAGATACA
1251 CGTGGCCAAA ATCCTAATTT AATTTTTTCT TCTAACGTGT GACTTAAATT
1301 AAATATGAGT TAGAATTAAT AACATTAAAC CACATTCAGC TAGACTACTT
1351 CAGTGTATAA ATTGAAAGTG TATGAACTAA AGTAAGTATG TTCATTTGAG
1401 AATAAATTTT TATTTATGAC AAATTCGCTA TTTATTTATG AGAGTTTTCG
1451 TACTATATTA TATTAATATG CATTCATTAA GGTTAGGTTG AAGCAGTTTG
1501 GTATTTAAAG TGTAATTGAA AGAGAGTGGG GCGCCTTATG TCATTCGTAA
1551 CAGAAAATCC ATGGTTAATG GTACTAACTA TATTTATCAT TAACGTTTGT
1601 TATGTAACGT TTTTAACGAT GCGAACAATT TTAACGTTGA AAGGTTATCG
1651 TTATATTGCT GCATCAGTTA GTTTTTTAGA AGTATTAGTT TATATCGTTG
1701 GTTTAGGTTT GGTTATGTCT AATTTAGACC ATATTCAAAA TATTATTGCC
1751 TACGCATTTG GTTTTTCAAT AGGTATCATT GTTGGTATGA AAATAGAAGA
1801 AAAACTGGCA TTAGGTTATA CAGTTGTAAA TGTAACTTCA GCAGAATATG
1851 AGTTAGATTT ACCGAATGAA CTTCGAAATT TAGGATATGG CGTTACGCAC
1901 TATGCTGCGT TTGGTAGAGA TGGTAGTCGT ATGGTGATGC AAATTTTAAC
1951 ACCAAGAAAA TATGAACGTA AATTGATGGA TACGATAAAA AATTTA
```

Mutant: NT57
Phenotype: temperature sensitivity
Sequence map: Mutant NT57 is complemented by pMP94, which contains a 3.6 kb insert of S. aureus genomic DNA. A partial restriction map is depicted FIG. 49, along with open boxes to indicate the percentage of the clone for which DNA sequence has been obtained. Database searches at both the nucleic acid and peptide levels reveal significant similarity at the peptide level to the gap gene, encoding glyceraldehyde-3-phosphate dehydrogenase (EC 1.2.1.12), from a number of prokaryotes and eukaryotes (e.g. Genbank Accession No. M24493, for the corresponding gene from B. stearothermophilus; published in Branlandt, C. et al., 1989, Gene 75:145-155). From the opposite sequence contig, a strong peptide-level similarity is noted to the dnaB gene product, encoding an essential protein involved in the initiation of DNA replication, from B. subtilis (Genbank Accession No. M15183; published in Hoshino, T. et al. Proc. Natl. Acad. Sci. USA 84 (1987) 653-657). Also of significance is the similarity of a subclone sequence to an ORF of unknown function, conserved among prokaryotes including E. coli, M. leprae, C. acetobutylicum, H. influenzae and B. subtilis (e.g. “orf 168” from Genbank Accession No. D28752). The relative orientations and predicted sizes of the ORFs identified in this entry are denoted by arrows in the restriction map.

DNA sequence data: The following DNA sequence data represents the partial sequence of clone pMP94, starting with the standard M13 forward and M13 reverse sequencing primers and applying primer walking strategies to augment the sequence contigs as well as obtain subclone sequence data. The sequences below can be used to design PCR primers for the purpose of amplification from genomic DNA with subsequent DNA sequencing:

```
clone pMP94
SEQ ID NO. 45
pMP94.forward Length: 1017 nt
      1 CTTYGARCTC GGTACCCGGG GMTCCTCTAR AGTCGATCTT TATACTCTTG
    51 TAACACATTT AAGTCTTCAT CAATCATAGC ATTCGTTAAT TCAGCTCGAT
  101 GCGCTTCCAA AAATTGCTTA ACATCTGGGT CATWGATGTC TCCTGATTTT
  151 ATCTTTTCTA TTCTTTTTTC AAAGTCCTGC GACGTGTTAA TTATACTTTT
  201 AAATTGCTTC ATTATTGACT GTCCTCCTCC CATTTTTTAG ATAATTTATC
  251 TAGAAATGCT TGTCGATCTT GCTCTAATTG TTGATCATCT ACGCTATTAT
  301 CTTTAGCCGA ATCTTCTTCA CTAGGTTTAT CTCTATTTTC TAACCATTTA
  351 GGTGTTTTTT CTTTTGAAAT ACGATTACGC TGCCCATAGT ATGAACCACG
  401 CTTTTGGTAA TTTCCGCTAG AACCCTCATT TTTAGGTTGA TTAACTTTTT
  451 TAGCGTAATT ATATGCTTCT TTAGCTGTCT TAATACCTTT TTTCTTCCAA
  501 TTTGATGCTA TTTCCAAAAT ATACGCTTTA GGAAGTTTCA TATCTTCTTT
  551 TAACATGACA AATTGCAACA AAATATTAAT GACGCCAAAA GACATTTTTT
  601 CACGTTTCAA TTAATTCTTC AACCATTGTC TTTTGCGATA TAGTTGGTYC
  651 TGATTCAGAM CAAGAAGCTA ACATATCAAT TGGACTCGTT TGTTCAAGTA
  701 ACTCAAACCA TTCATCACTT TGTGGCTTTG GATTCACTTC TGAAGATTTG
  751 CCCGCCGAAG ATGATGTAGC AGGAGATTTC ACCTGTAATT TAGGCATTTG
  801 ATTTTCGTGT TCCATTAAGT AATACGAGCG TGCTTGTTTA CGCATTTCTT
  851 CAAAGGATAA CTGTTGTCCA CTTGTAATTG AATTTAAAAT AACATGCTTC
  901 ATGCCATCTG CTGTTAAACC ATATAAATCN CGAATTGTGT TATTAAACCC
  951 TTGCATCTTG GTAACAATGT CTTGACTAAT AAATGTTTAC CTAACATTGT
1001 CTCCACATTT CNANTCC
SEQ ID NO. 46
pMP94.reverse Length: 1035 nt
      1 TGCATGCCTG CAGGTCGATC AAGGGGTGCT TTTAATGTCA AMGAATATTG
    51 CAATTRATGG TATGGGTAGA ATTGGAAGAA TGGTATTACG TATTGCATTA
  101 CAAAATAAAA ATTTAAATGT AGTAGCGATA AATGCTAGTT ATCCACCCGA
  151 AACAATTGCA CATTTAATCA ATTACGATAC GACACATGGA AAATATAATC
  201 TAAAAGTTGA ACCGATTGAA AATGGATTGC AAGTTGGAGA TCATAAAATT
  251 AAATTGGTTG CTGATCGCAA TCCTGAAAAC TTGCCATGGA AAGAATTAGA
  301 TATCGATATT GCTATAGATG CAACTGGTAA ATTTAATCAT GGTGATAAAG
  351 CCATCGCACA TATTAAAGCA GGTGCCAAAA AAGTTTTGTT AACTGGTCCT
  401 TCAAAAGGTG GACATGTTCA AATGGTAGTT AAAGGCGTAA ATGATAACCA
  451 ATTAGATATA GAAGCATTTG ACATTTTTAG TAATGCTTCA TGTACTACTA
  501 ATTGCATTGG TCCAGTTGCA AAAGTTTTAA ATAATCAGTT TGGGAATAGT
  551 TAATGGTTTA ATGACTACTG TTCACGCTAT TACAAATGAC CAAAAAAATA
  601 TTGATAATCC MCATAAAGAT TTAAGACGTG CACGTTCATG TWATGAAAGC
  651 ATTATTCCTA CTTCTACTGG TGCGGCGAAA GCTTTAAAAG AAGTATTACC
  701 AGAATTAGAA GGTAAATTAC ACGGCATGGC ATTACGTTGT ACCAACAAAG
  751 AATGTATCGC TCGTTGATTT AGTTGTTGAT TTAGAAAAAG AAGTAACTGC
  801 AGAAGAANTA AACCAAGCTT TTGAAAATGC AGGTTTAGAA GGTATCATAG
  851 AANTCGAACA TCACCACTAG TGTCTGTTGA TTTTAATACT AATCCCAATT
  901 CAGCTATTAT TGATGCCAAA CCACNATGTC ATGTTCCGGG AAATAAGTAA
  951 ANTTATTGCT TGGTATGAAN ATGAATGGGG TTATTCCAAT AAATTGTTAA
1001 NNTTGCNGAA CAAATTGGAC NCTTTGGANT CCAAA
SEQ ID NO. 47
pMP94.subclone Length: 483 nt
      1 CTCCGTTTGT TTTCGCTTAA AATCCCTTGC ATCGATGCTA ACAATTGATC
    51 AACATCTTTA AATTCTTTAT AGACTGATGC AAATCTAACA TATGAAACTT
  101 GATCAACATG CATTAACAAG TTCATAACGT GTTCACCTAT ATCTCGTGAA
  151 GACACTTCCG TATGACCTTC ATCTCGTAAT TGCCATTCAA CCTTGTTAGT
  201 TATGACTTCA AGTTGTTGAT ATCTAACTGG TCGTTTCTCA CAAGAACGCA
  251 CAAGTCCATT AAGTTATCTT TTCTCTTGAA AACTGCTCTC TTGTGCCATC
  301 TTTTTTCACA ACTATAAGCT GACTAACTTC GATATGNTTC AAATGTTAGT
  351 GGAAACGTTG TTTCCACAAT TTTCACATTC TCTTCGTCTT CCGAAATGGC
  401 ATTTAATTCA TCGGGCATGC CTTGAATCTA CAACTTTAGA ATTGTGTTAG
  451 AATTACATTT CGGGCATTTC ATTACATCAC CTC
```

Mutant: NT68
Phenotype: temperature sensitivity
Sequence map: Mutant NT68 is complemented by pMP163, which contains a 5.8 kb insert of S. aureus genomic DNA. A partial restriction map is depicted FIG. 50. Database searches at both the nucleic acid and peptide levels reveal strong peptide-level similarities to the dnaE gene, encoding DNA polymerase III alpha subunit (EC 2.7.7.7), from Gram-negative bacteria such as S. typhimurium (Genbank Accession No. M29701; published in Lancey, E. D., et al. J. Bacteriol. 171 (1989) 5581-5586). This mutant is distinct from NT28, described previously as having a mutation in the polC gene which also encodes an alpha subunit of DNA polymerase III (found so far in Gram-positive bacteria). Although dnaE and polC putatively encode proteins of the same enzymatic function, in S. aureus these two genes are quite distinct and may or may not encode proteins of redundant function; since the DNA sequences of each are less than 65% identical, they are confirmed as being two distinct essential genes.

DNA sequence data: The following DNA sequence data represents the sequence generated by primer walking through clone pMP163, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP163
SEQ ID NO. 48
pMP163 Length: 5718 nt
      1 CTCGGTACCC GGGGATCGTC ATGGAATACC GGAATATTAG TTTCTTTTTT
    51 CAATCGTTCT TCAATTTCAA AACAACGTGG TGCCGAAATA TCCTCTAAAT
  101 TAATACCACC ATAATTAGGT TCTAACAACT TAACTGTTTT AATGATTTCT
  151 TCGGTATCAG TTGTATTTAA CGCAATAGGC ACCCCATTGA TACCAGCGAA
  201 GCTTTTGAAT AATACTGCTT TACCTTCCAT TACAGGAATA CTTGCTTCAG
  251 GTCCAATGTT ACCTAAACCT AATACCGCTG TTCCATCAGT AATAACTGCA
  301 ACTGTATTTC CTTTAATTGT GTAATCATAT ACTTTTCTTT TATCTTCATA
  351 AATATCTTTA CACGGTTCAG CAACGCCAGG TGAGTATGCT AAACTTAATT
  401 CCTCTTTATT AGTAACTTTT ACATTTGGTT TAACTTCTAA TTTACCTTGA
  451 TTACGTTTGT GCATTTCCAA TGCTTCATCT CTTAATGACA TGAAATCAGC
  501 CCCTAATTCA ATATTTATTT TTAAAAAATA ACTTGGATAA AACGCATTAC
  551 ATTATAAAAG TAAAAATATT GGGTAATCTG AATGARTAAG AATTTATGGT
  601 TTTGATTATG TAACACAAAT AGCGATAAAC GATAATAAAA TAATATTTAT
  651 AAAGATACAT TAAACCATAC TATCTAAAGA TATACCTTTA ATTATTATAA
  701 TGGATAGCAA AAACCAATAT ATCAAAAAGT TATTATTTTT CCGCACGATA
  751 TATCGACAAA ATTCTTTACT CAATTTATGT ATACTGCTTT TTGTGCTAAT
  801 TATTCTTATG GATTAATCAA TAATGTAAAG TGAAACTCAT AAAAATAATA
  851 AGCATAAAAA ACTAATATAA ACGCAAACTG ATGGTTAAAA AATATCTAAC
  901 CATCAGTTTA CTATATCATA ATTTATTAGT TGATAAAAGT TATATAAGCC
  951 TAATATCACT AGGGTTAAAG GGATTGTATA AAATTATTAA ACATACTATC
1001 TTTTTGATTA ATATAGCCTA AAGTAGTCAT TTGTTTAATC GTTTCATCAT
1051 AAAAGGATAA CACAACATCA TTAGCATTCT CTTTCGTAGC TTTAATCATC
1101 TCTTCAAACA TATCTATTTG TGATTTATTT CTAATTATAA TTTGTTTGGC
1151 AAATGCTAAT TTTTGTTCTT CAAAAGTGGC TAATGTCTGA ATCTCATTTA
1201 TAATTAGTTG ACGTTGTTGC TTTCTATGGT CAAATTTCCC GCTAACTATA
1251 AACAAGTCAT TATGTGATAA CAACTCTTCG TACTTTTTAA ACTGATTAGG
1301 GAAAATCACA CCATCTAAAG TTTCAATGCC ATCATTTAAT GTTGACGAAT
1351 GCCATATTTT GACCATTTTT AGTTCGAATT TGTTTAACTT TATCAAACTG
1401 TACTAATATA GGTTTATAAT TCTGCGCGTT ACTCAATTTA AATATCGTTA
1451 AATATTGTTT GGCAACAAAC TTTTTATCTA CTGGGTGTTG CGAAACATAA
1501 AATCCTAAAT ATTCTTTTTC GTACTGACTA ATAAGTGCAT CAGGCAATTC
1551 TTCTTTATCT TCATACATCT GTTTTGGCGT TAAAATATCA AATAAAAAAC
1601 CATCTTGTTC AATGTTTAAA TCGCCATCCA ACACTTGATC AATAGCTTGC
1651 AACAACGTTG AACGTGTTTT ACCAAAAGCA TCAAACGCTC CCACTAAAAT
1701 CAGTGCTTCA AGTAACTTTC TCGTTWTGAM YCTCTTCGGT ATACGTCTAG
1751 CAWAATCAAA GAAATCTTTA AATTTGCCGT TCTGATAACG TTCATCAACA
1801 ATCACTTTCA CACTTTGATA ACCAACACCT TTAATTGTAC CAATTGATAA
1851 ATAAATGCCT TCTTGGGAAG GTTTATAAAA CCAATGACTT TCGTTAATGT
1901 TCGGTGGCAA TATAGTGATA CCTTGTTTTT TTGCTTCTTC TATCATTTGA
1951 GCAGTTTTCT TCTCACTTCC AATAACATTA CTTAAAATAT TTGCGTAAAA
2001 ATAATTTGGA TAATGGACTT TTAAAAAGCT CATAATGTAT GCAATTTTAG
2051 AATAGCTGAC AGCATGTGCT CTAGGAAAAC CATAATCAGC AAATTTCAGA
2101 ATCAAATCAA ATATTTGCTT ACTAATGTCT TCGTGATAAC CATTTTGCTT
2151 TGSMCCTTCT ATAAAATGTT GACGCTCACT TTCAAGAACA GCTCTATTTT
2201 TTTTACTCAT TGCTCTTCTT AAAATATCCG CTTCACCATA ACTGAAGTTT
2251 GCAAATGTGC TCGCTATTTG CATAATTTGC TCTTGATAAA TAATAACACC
2301 GTAAGTATTT TTTAATATAG GTTCTAAATG CGGATGTAAA TATTGAACTT
2351 TGCTTGGATC ATGTCTTCTT GTAATGTAAG TTGGAATTTC TTCCATTGGA
2401 CCTGGTCTAT ACAAAGAAGT TACAGCAACA ATATCTTCAA AGTGTTCCGG
2451 CTTTAATTTT TTTAATACAC TTCTTACACC GTCAGACTCT AATTGGAATA
2501 TGCCAGTCGT ATCTCCTTGC GACAACAATT CAAACACTTT TTGATCATCA
2551 AACGGAATCT TTTCGATATC AATATTAATA CCTAAATCTT TTTTGACTTG
2601 TGTTAAGATT TGATGAATAA TCGATAAGTT TCTCAACCCT AGAAAATCTA
2651 TTTTTAATAA CCCAATACGT YCGGCTTCAG TCATTGTCCA TTGCGTTAAT
2701 AATCCTGTAT CCCCTTTCGT TAAAGGGGCA TATTCATATA ATGGATGGTC
2751 ATTAATAATA ATYCCTGCCG CATGTGTAGA TGTATGTCTT GGTAAACCTT
2801 CTAACTTTTT ACAAATACTG AACCAGCGTT CATGTCGATG GTTTCGATGT
2851 ACAAACTCTT TAAAATCGTC AATTTGATAT GCTTCATCAA GTGTAATTCC
2901 TAATTTATGT GGGATTAAAC TTGAAAATTT CATTTAATGT AACTTCATCA
2951 AACCCCATAA TTCTTCCAAC ATCTCTAGCA ACTGCTCTTG CAAGCAGATG
3001 AMCGAAAGTC ACAATTCCAG ATACATGTAG CTCGCCATAT TTTTCTTGGA
3051 CGTACTGAAT GACCCTTTCT CGGCGTGTAT CTTCAAAGTC AATATCAATA
3101 TCAGGCATTG TTACACKTTC TGGGTTTAAA AAACGTTCAA ATAATAGATT
3151 GAATTTAATA GGATCAATCG TTGTAATTCC CAATAAATAA CTGACCAGTG
3201 AGCCAGCTGA AGAACCACGA CCAGGACCTA CCATCACATC ATTCGTTTTC
3251 GCATAATGGA TTAAATCACT WACTATTAAG AAATAATCTT CAAAACCCAT
3301 ATTAGTAATA ACTTTATACT CATATTTCAA TCGCTCTAAA TAGACGTCAT
3351 AATTAAGTTC TAATTTTTTC AATTGTGTAA CTAAGACACG CCACAAATAT
3401 TTTTTAGCTG ATTCATCATT AGGTGTCTCA TATTGAGGAA GTAGAGATTG
3451 ATGATATTTT AATTCTGCAT CACACTTTTG AGCTATAACA TCAACCTGCG
3501 TTAAATATTT CTTGGTTAAT ATCTAATTGA TTAATTTCCT TTTTCAGTTA
3551 AAAAATGTGC ACCAAAATCT TTCTTGATCA TGAATTAAGT CTAATTTTGT
3601 ATTGTCTCTA ATAGCTGCTA ATGCAGAAAT CGTATCGGCA TCTTGACGTG
3651 TTTGGTAACA AACATTTTGA ATCCAAACAT GTTTTCTACC TTGAATCGAA
3701 ATACTAAGGT GGTCCATATA TGTGTCATTA TGGGTTTCAA ACACTTGTAC
3751 AATATCACGA TGTTGATCAC CGACTTTTTT AAAAATGATA ATCATATTGT
3801 TAGAAAATCG TTTTAATAAT TCAAACGACA CATGTTCTAA TGCATTCATT
3851 TTTATTTCCG ATGATAGTTG ATACAAATCT TTTAATCCAT CATTATTTTT
3901 AGCTAGAACA ACTGTTTCGA CTGTATTTAA TCCATTTGTC ACATATATTG
3951 TCATACCAAA AATCGGTTTA ATGTTATTTG CTATACATGC ATCATAAAAT
4001 TTAGGAAAAC CATACAATAC ATTGGTGTCA GTTATGGCAA GTGCATCAAC
4051 ATTTTCAGAC ACAGCAAGTC TTACGGCATC TTCTATTTTT AAGCTTGAAT
4101 TTAACAAATC ATAAGCCGTA TGAATATTTA AATATGCCAC CATGATTGAA
4151 TGGCCCCTTT CTATTAGTTA AGTTTTGTGC GTAAAGCTGT AGCAAGTTGC
4201 TCAAATTCAT CCCAGCTGTC CAACTGAAAY TCCTGACGCA TTCGGATGAC
4251 CACCGCCACC AAAATCTTGC GCAATATCAT TAATAATCAA TTGCCCTTTA
4301 GAACGTAATC GACATCTGAT TTCATTACCT TCATCGACTG CAAATACCCA
4351 TATTTTCAAG CCTTTGATGT CAGCAATTGT ATTAACAAAC TGAGATGCTT
4401 CATTTGGCTG AATACCGAAT TGCTCCAATA CATCTTCAGT TATTTTAACT
4451 KGGCAGAATC CATCATCCAT AAGTTCGAAA TGTTGYAAAA CATAACCTTG
4501 AAACGGCAAC ATTKYTGGGT CCTTCTCCAT CATTTTATTT AAAAGCGCAT
4551 TATGATCAAT ATCATGCCCA ATTAACTTTC CAGCAATTTC CATAGTATGT
4601 TCWGAGGTAT TGTTAAAAAG GRGATCGCCC AGTATCACCG ACGATACCAA
4651 GATATAAAAC GCTCGCGATA TCTTTATTAA CAATTGCTTC ATCATTAAAA
4701 TGTGAGATTA AATCGTAAAT GATTTCACTT GTAGATGACG CGTTCGTATT
4751 AACTAAATTA ATATCACCAT ACTGATCAAC TGCAGGATGA TGATCTATTT
4801 TAATAAGTYT ACGACCTGTA CTATAACGTT CATCGTCAAT TCGTGGAGCA
4851 TTGGCAGTAT CACATACAAT TACAAGCGCA TCTTGATATG TTTTATCATC
4901 AATGTTATCT AACTCTCCAA TAAAACTTAA TGATGATTCC GCTTCACCCA
4951 CTGCAAATAC TTGCTTTTGC GGAAATTTCT GCTGAATATA GTATTTTAAA
5001 CCAAGTTGTG AACCATATGC ATCAGGATCK RSTYTARMRK RTCYSYGKMT
5051 AMYRATTGYA TCGTTGTCTT CGATACATTT CATAATTTCA TTCAAAGTAC
5101 TAATCATTTT CAWACTCCCT TTTTTAGAAA AGTGGCTTAA TTTAAGCATT
5151 AGTCTATATC AAAATATCTA AATTATAAAA ATTGTTACTA CCATATTAAA
5201 CTATTTGCCC GTTTTAATTA TTTAGATATA TATATTTTCA TACTATTTAG
5251 TTCAGGGGCC CCAACACAGA GAAATTGGAC CCCTAATTTC TACAAACAAT
5301 GCAAGTTGGG GTGGGGCCCC AACGTTTGTG CGAAATCTAT CTTATGCCTA
5351 TTTTCTCTGC TAAGTTCCTA TACTTCGTCA AACATTTGGC ATATCACGAG
5401 AGCGCTCGCT ACTTTGTCGT TTTGACTATG CATGTTCACT TCTATTTTGG
5451 CGAAGTTTCT TCCGACGTCT AGTATGCCAA AGCGCACTGT TATATGTGAT
5501 TCAATAGGTA CTGTTTTAAT ATACACGATA TTTAAGTTCT CTATCATGAC
5551 ATTACCTTTT TTAAATTTAC GCATTTCATA TTGTATTGTT TCTTCTATAA
5601 TACTTACAAA TGCCGCTTTA CTTACTGTTC CGTAATGATT GATTAAAAGT
5651 GGTGAAACTT CTACTGTAAT TCCATCTTGA TTCATTGTTA TATATTTGGC
5701 GATTTGATCC TCTAGAGT
```

Mutant: NT78
Phenotype: temperature sensitivity
Sequence map: Mutant NT78 is complemented by pMP115, which contains a 5.3 kb insert of S. aureus genomic DNA. A partial restriction map is depicted FIG. 51, along with open boxes to indicate the percentage of the clone for which DNA sequence has been obtained. Database searches at both the nucleic acid and peptide levels reveal no significant similarities between the sequences obtained at the left-most and right-most edges and any published sequences. The sequence generated from a Msp I subclone, however, matches at both the nucleic acid and peptide level to hsp60, encoding the GroEL protein from S. aureus (Genbank Accession No. D14711). The relative size and orientation of the GroEL ORF is depicted by an arrow; other proteins (i.e. GroES) are known to reside near the identified ORF and will be confirmed by further DNA sequencing.

DNA sequence data: The following DNA sequence data represents the sequence generated bye sequencing the left-most and rightmost edges of pMP115 and its subclone 78.3, starting with standard M13 forward and M13 reverse sequencing primers. The sequence below can be used to design PCR primers for the purpose of amplification from genomic DNA with subsequent DNA sequencing.

```
clone pMP115, a 5,300 bp genomic fragment
SEQ ID NO. 49
pMP115.m13f Length: 513 nt
    1 TTCTTGCCTC CCAATCGCCT AATAGCCCTN AAAACTACTT TTTTTAATCT
  51 ATAGGCGATG TAAAAATACC ATATATTGAN GGTGCTATAC CTCCTAAAAT
101 AGCAGTTCCC AAAGTTGTCA TTACTGAAAT TACTGCGAAA GTATCATCCG
151 AAAGCAATAA ATTCAAACTA ATGCATTGTT TATTACCCAT CGAATTTATT
201 GACCAAATAG CTAGAGAAAT AAACAACCCA AAATTTAAAA TAAATGATAT
251 AGTAATAGCA ATTGTTTACA AAACACGGAA TTTTTCATTT TTATTTATAT
301 TATCCATTTT NCTCCCTTTT NCTTAAATCA TTTTATTATA TATTNCAATA
351 ATCAATCTGA AATGTTGATG TAATTTGNNA AAAATATCAT ACTTTTNCTC
401 CTGAAAACCT CCCTAAATCA TCAATATGGN AATCNGTNTT NGGGTATTGC
451 GNTTNCAACT CTTTTAAANC TCACTCNTTC TTCTCATCGN CTTAACCGTA
501 CTATCANTAA AAT
SEQ ID NO. 50
pMP115.m13r Length: 533 nt
    1 CTGAGCTGCT TNCANNNCCA NTNTGAAAAA GCCCCCAGNN CAGCCCGNTT
  51 NCAAAACAAC GNCTNCATTT GAANCCCCAT GAAAAAGAAC GAATTTTGAC
101 AATGGNTTAA AAAACANGNA AGATAATAAG AAAAAGTGCC GTCAACTGCA
151 TATAGTAAAA GTTGGCTAGC AATTGTATGT NCTATGATGG TGGTATTTTC
201 AATCATGCTA TTCTTATTTG TAAAGCGAAA TAAAAAGAAA AATAAAAACG
251 AATCACAGCG ACGNTAATCC GTGTGTGAAT TCGTTTTTTT TATTATGGAA
301 TAAAAATGTG ATATATAAAA TTCGCTTGTC CCGTGGCTTT TTTCAAAGCC
351 TCAGGNTTAA GTAATTGGAA TATAACGNCA AATCCGTTTT GTAACATATG
401 GGTAATAATT GGGAACAGCA AGCCGTTTTG TCCAAACCAT ATGCTAATGN
451 AAAAATGNCA CCCATACCAA AATAAACTGG GATAAATTTG GNATCCATTA
501 TGTGCCTAAT GCAAATNCCT NATGACCTTC CTT
```

The following DNA sequence data were acquired using standard sequencing methods and the commercially-available T7 and SP6 primers and can be used to demonstrate identity to the GroEL protein from S. aureus:

```
subclone 78.3, a 2000 bp Msp I fragment
SEQ ID NO. 51
78.3.sp6 Length: 568 nt
    1 CCGACAGTCG TTCCCNTCAT GCAAAATATG GGGGCTAAAC TCAGTTCAAG
  51 AAGTCGGCAA ATAAGACAAA TGAAATTGCC TGGTGACGGT AGNACAACTG
101 CAACAGTATT AGCTCAAGCA ATGATTCAAG AAGGCTTGAA AAATGTTACA
151 AGTGGTGCGA ACCCAGTTGG TTTACGACAA GGTATCGACA AAGCAGTTAA
201 AGTTGCTGTT GAAGCGTTAC ATGAAAATTC TCAAAAAGTT GAAAATAAAA
251 ATGAAATTNC GCAAGTAGGT GCGNTTTCAG CAGCAGATGN AGNAATTNGA
301 CGTTATATTT CTGAAGCTAT NGGNAAAGTA GGTAACGNTG GTGTCATTAC
351 ANTTNTNGGG TCAAATGGGC TNTNCACTNN NCTNGANGTG GTTGNNGGTG
401 TNCNATTTGA TCNNNGTTAT CANTCACCNN CTATNGTTAC TGCTTCNGCT
451 AAAATGGTTG CTGCNTTTGG NCGCCCCTAC ATTTTTGTNA CNGCTTNGGG
501 ANTCTCGTCT TTNCNCGATT CTTTCCCCTT TTTGGCCCNT GGGNAATCTT
551 TTNGGNCNCC CTTTATTT
```

Mutant: NT81
Phenotype: temperature sensitivity
Sequence map: Mutant NT81 is complemented by clone 81-3, which contains a 1.7 kb insert of S. aureus genomic DNA. A partial restriction map is depicted FIG. 52, along with open boxes to indicate the percentage of the clone for which DNA sequence has been obtained. Database searches at both the nucleic acid and peptide levels reveal identity to the fib locus, encoding a fibrinogen binding protein, from S. aureus (Genbank Accession No. X72013; published in Boden, M. K. et al., Mol. Microbiol. 12 (1994) 599-606.) The relative size and orientation of the Fib ORF with respect to the restriction map is depicted by an arrow; also identified in this analysis is an ORF of unknown function downstream from (3′ to) the Fib ORF.

DNA sequence data: The following DNA sequence data represent the sequences at the left-most and right-most edges of subclones pMP1043 and pMP1042, using standard SP6 and T7 sequencing primers. The sequences below can be used to design PCR primers for the purpose of amplification from genomic DNA with subsequent DNA sequencing:

```
subclone 1042, a 400 bp Hind III fragment
SEQ ID NO. 52
1042.con Length: 437 nt
    1 CAAYTTAGYC AACTACTACC AATATAGCAC TAGAACTGGA AATGATAATT
  51 TAATATTGKG CACTTTTTSA TTGKTTAAAC ATGTACATAT TTNAAAAAAT
101 AGGAGAGCAA AGKAAATAAT TGATATAGTT ATTTTSAGAG TAATCCTAGG
151 AACTATTGTA TTTATATTTS TCTCCCCTAC TTTTAAATGT CATTCATTAT
201 ACATAAGCAT TTTGATATAG AATTTATCAC ATATGCAAAT TGAAAACAGG
251 TTAAGACCAT TTTTTGTCTC AACCTGTTTT ATTTATTATC TATTTMTAAT
301 TTCATCAATT TCTTTGTATA TTTTTYCTAA TGCAACTTTA GCATCAGCCA
351 TTGATACGAA ATCATTTTYC TTAAGTGCCG CTTTAGCTCT ATATTCATTC
401 ATYATAATCG TACGTTTATA ATATGGATTT ACGTTGA
subclone 1043, a 1300 bp EcoR I/Hind III fragment
SEQ ID NO. 53
1043.t7 Length: 659 nt
    1 CCCGATTCGA GCTCGGTACC GGNGATCCTC TAGAGTCGAT CTATCAAGCA
  51 GTPAATGAAA AAATGGACAT TAATGATATT AATATCGACA ATTTCCAATC
101 TGTCTTTTTT GACGTGTCTA ATTTGAATTT AGTAATTCTA CCAACGTTAA
151 TCATTAGCTG GGTCACAATA TTTAACTATA GAATGAGAAG TTACAAATAA
201 AATCTATGAG ATTATACCTN CAGACACCAA CATTCAAATG GTGTCTTTTN
251 TGTTGTGTGG TTTTATTTNT GAAATNCGAA AAAGTAGAGG CATGAATTTT
301 GTGACTAGTG TATAAGTGCT GATGAGTCAC AAGATAGATA GCTATATTTT
351 GTCTATATTA TAAAGTGTTT ATAGNTAATT AATAATTAGT TAATTTCAAA
401 AGTTGTATAA ATAGGATAAC TTAATAAATG TAAGATAATA ATTTGGAGGA
451 TAATTAACAT GAAAAATAAA TTGATAGCAA AATCTTNATT AACATTAGGG
501 GCAATAGGTA TTACTACAAC TACAATTGCG TCAACAGCAG ATGCGAGCGA
551 AGGATACGGT CCAAGAGAAA AGAAACCAGT GAGTATTAAT CACAATATCG
601 NAGAGTACAA TGATGGTACT TTTAATATCA ATCTTGANCA AAATTACTCA
651 ACAACCTAA
SEQ ID NO. 54
1043.sp6 Length: 298 nt
    1 AATNCTCCTC CNATGNTTTA TNATGAAACT AACTTTAAGT NAAATATTTN
  51 TCCAGACTAC TTGCATCTCC NTTATNCCCT TCTATAGTTN CTATCCCAGT
101 TNATGATAAA AGTAATGCTA ATGTNCCTGT NAATATATAT TTNTAAAATT
151 NNATTATAAG CNCTCCTTAA AATTNATACT TACTGAGTAT ATAGTCAATT
201 TNNGGACAAT TACATTAACC TGTCATTAAA TNGATTACTT TTTNNATTAA
251 CAAAAATTAA CATAACATTT AATTAATTNT TTCCNGATAN CAGCAACG
```

Mutant: NT86
Phenotype: temperature sensitivity
Sequence map: Mutant NT86 is complemented by pMP121, which contains a 3.4 kb insert of S. aureus genomic DNA. A partial restriction map is depicted FIG. 53, along with open boxes to indicate the percentage of the clone for which DNA sequence has been obtained. Database searches at both the nucleic acid and peptide levels reveal identity at the nucleic and peptide levels to the dnaK/dnaJ genes, encoding Hsp70 and Hsp40, from S. aureus (Genbank Accession No. D30690; published in Ohta, T. et al. J. Bacteriol. 176 (1994) 4779-4783). Cross complementation studies (plasmid pMP120; data not shown) reveal that the ORF responsible for restoring a wild-type phenotype to mutant NT86 codes for Hsp40. The relative sizes and orientations of the identified genes are depicted in the restriction map by arrows.

DNA sequence data: The following DNA sequence data represent the sequences at the left-most and right-most edges of clone pM121, using standard M13 forward and M13 reverse sequencing primers. The sequences below can be used to design PCR primers for the purpose of amplification from genomic DNA with subsequent DNA sequencing:

```
clone pMP121, a 3400 bp genomic fragment
SEQ ID NO. 55
pMP121.m13f Length: 535 nt
    1 TCCAAATATT CACCAAGCTG TAGTTCAAGA TGATAACCCT NATTTTAANT
  51 CTGGCGAAAT CACTCAAGAN CTACAAAAAG GATACAAGCT TAAAGATAGA
101 GTATTAAGAC CATCANTGGT CAAAGTAAAC CAATAACTTA AATTTGGCGA
151 AAAGACATTG TTTAAAATTA ANTTAATTTA ATGATTAATT GGAGGNATTT
201 TNTTATGAGT AAAATTNTTG GTATAGACTT AGGTACAACA NATTCATGTG
251 TAACAGTATT AGANGGCGAT GAGCCAAAAG TAATTCAAAA CCCTGANGGT
301 TCACGTACAA CACCATCTGT WGTAGCTTTC AAAAATGGAG AAACTCAAGT
351 TGGTGAAGTA GCAAAACGTC AAGCTATTAC AAACCCAAAC ACTGTTCANT
401 CTATTAGNCG TCATATGGGT ACTGNTTATA ANGTAGATAT TGAGGGTAAA
451 TCATACACAC CACAAGNNNT CTCAGCTNTG NTTTTNCAAA ACTTANNANT
501 TNCAGCTGNA GTNATTTAGG TGNGNNNGTT GNCAA
SEQ ID NO. 56
pMP121.m13r Length: 540 nt
    1 ATGACTGCAG GTCGATCCAT GATTTACAAG TATATTGGTA GCCAATTCTA
  51 CTGCTTCATG ATTAATAATA ATTGAAAGCT CTGTCCAGTT CATACTTTAT
101 TCTCCCTTAA AGAATCTTTT TGNTCTATCT TTAAAATTCG AAGGTTGTTC
151 ATTAATTTCT TCACCATTTA ATTGGGCAAA TTCTTTCATT AGTTCTTTNT
201 GTCTATCTGT TAATTTAGTA GGCGTTACTA CTTTAATATC AACATATAAA
251 TCTCCGTATC CATAGCCATG AACATTTTTT ATACCCTTTT CTTTTAAGCG
301 GAATTGCTTA CCTGTTTGTG TACCAGCAGG GGATTGTTAA CATAACTTCA
351 TTATTTAATG TTGGTATTTT TATTTCATCG CCTAAAGCTG CTTGTGGGAA
401 GCTAACATTT AATTTGNAAT AAATATCATC ACCATCACGT TTAAATGTTT
451 CAGATGGTTT AACTCTAAAT ACTACGTATT AATCANCAGG AGGTCCTCCA
501 TTCACGGCTG GAGAGGCTTC AACAGCTAAT CTTATTTGGT
```

The following DNA sequence data were acquired using standard sequencing methods and the commercially-available T7 and SP6 primers and can be used to demonstrate identity to the Hsp40 protein from S. aureus.

```
subclone 1116, a 1400 bp EcoR I/Hind III fragment
SEQ ID NO. 57
1116.sp6 Length: 536 nt
    1 TTTATAATTT CATCTNTTGA AGCATCCTTA CTAATGCCTA AAACTTCATA
  51 ATAATCTCTT TTGGCCACAG CTATCTCTCC TTTNCTNAAT TAACTCATAT
101 AGTTTAACGT AATATGTCAT ACTATCCAAA TAAAAAGCCA AAGCCAATGT
151 NCTATTGACT TTNACTTTTC ANATCATGAC AACATTCTAA TTGTATTGTT
201 TAATTATTTT NTGTCGTCGT CTTTNACTTC TTTAAATTCA GCATCTTCTA
251 CAGTACTATC ATTGTTTTNA CCAGCATTAG CACCTTGTNT TGTTGTTGCT
301 GTTGAGCCGC TTGCTCATAT ACTTTTNCTG NTAATTCTTG ANTCACTTTT
351 TCAAGTTCTT CTTTTTTAGA TTTANTATCT TCTATATNCT TGACCTTTCT
401 AANGCAGTTT TAAGAGCGTC TTTTTTCCTC TTTCTGCAGT TTTNTTATAC
451 TTCCTTTCAC CGThATTTTT CGGCTTATTT CAGTTAAANG TTTTTCCANC
501 TTGGGTNTAN CTATGGCTAG NAAAGNTTCG NTTCCT
SEQ ID NO. 58
1116.t7 LENGTH: 537 nt
    1 AAGATAAAAT GGCATTACAA CGTTTNAAAG ATGCTGCTGA AAAANCTAAA
  51 AAAGACTTAT CAGGTGTATC ACAAACTCAA ATCTCATTAC CATTTATCTC
101 AGCTGGTGAA AACGGTCCAT TACACTTAGA AGTAAACTTA ACTCGTNCTA
151 AATTTGAAGA ATTATCAGAT TCATTAATTA GAAGANCAAT GGAACCTACA
201 CGCCAAGCAA TGAAAGACGC TGGCTTAACA AACTCAGATA TCGATGAAGT
251 TATCTTAGTT GGTGGNTCAA CTCGTATTCC AGCAGTACAA GANGCTGTCA
301 AAAAAGAAAT CGGTAAAGAG CCTAACAAAG GAGTAAACCC GGNCGAAGTA
351 GGTGGCAATG GGNGCTGCAA TCCAAGGTGG CGTTATTCAC AGGTGACGTT
401 TAAAGACGTG TATTATTAGG NCGTAACACC ACTATCTTTA GGTATTGAAA
451 TTTTAGGTGG NCGTATGNAT TACGGTAATT GAACGTAACA CTACGGTTCC
501 TNCATTCTAA NTCTCAAAAT CTNTTCAACA GCAGTT
```

Mutant: NT89
Phenotype: temperature sensitivity
Sequence map: Mutant NT89 is complemented by pMP122, which contains a 0.9 kb insert of S. aureus genomic DNA. A partial restriction map is depicted FIG. 54, along with open boxes to indicate the percentage of the clone for which DNA sequence has been obtained. Database searches at both the nucleic acid and peptide levels reveal a high level of similarity at the peptide level to the trmD gene, encoding (guanine-N1-) methyltransferase (EC 2.1.1.31), from various prokaryotes, including S. marcescens (Genbank Accession No. L23334; published in Jin, S. et al. Gene 1 (1994) 147-148), H. influenzae, E. coli, and S. typhimurium. The predicted size and relative orientation of the TrmD ORF is depicted by an arrow.

DNA sequence data: The following DNA sequence data represent the sequences at the left-most and right-most edges of clone pM122, using standard M13 forward and M13 reverse sequencing primers. The sequence below can be used to design PCR primers for the purpose of amplification from genomic DNA with subsequent DNA sequencing; it can also be used to demonstrate similarity to the trmD gene of S. marcescens:

```
clone pMP122, a 925 bp genomic fragment
SEQ ID NO. 59
pMP122.con Length: 925 nt
    1 CTAGAGTCGA TCTAAAGAAT ATNTAANTCC TNATATKSCT GATGTTGTAA
  51 AAGAAGTGGA TGTTGAAAAT AAAAAAATTA TCATCACGCC AATGGAAGGA
101 TTGTTGGATT AATGAAAATT GATTATTTAA CTTTATTTCC TGAAATGTTT
151 GATGGTGTTT TAAATCATTC AATTATGAAA CGTGCCCANG AAAACAATAA
201 ATTACAAATC AATACGGTTA ATTTTAGAGA TTATGCAATT AACAAGCACA
251 ACCAAGTAGA TGATTATCCG TATGGTGGCG GWCAAGGTAT GGTGTTAAAG
301 CCTGACCCTG TTTTTAATGC GATGGAAGAC TTAGATGTCA CAGAMCAAAC
351 ACGCGTTATT TTAATGTGTC CACAAGGCGA GCCATTTTCA CATCAGAAAG
401 CTGTTGATTT AAGCAAGGCC GACCACATCG TTTTCATATG CGGACATTAT
451 GAAGGTTACG ATGAACGTAT CCGAACACAT CTTGTCACAG RTGAAATATC
501 AATGGGTGAC TATGTTTTAA CTGGTGGAGA ATTGCCAGCG ATGACCATGA
551 CTGATGCTAT TGTTAGACTG ATTCCAGGTG TTTTAGGTAA TGNACAGTCA
601 CATCAAGACG ATTCATTTTC AGATGGGTTA TTAGAGTTTC CGCAATATAC
651 ACGTCCGCGT GAATTTAAGG GTCTAACAGT TCCAGATGTT TTATTGTCTG
701 GAAATCATGC CAATATTGAT GCATGGAGAC ATGAGCAAAA GTTGAACCGC
751 ACATATAATN AAAGACCTGA CTTAATTNNA AAATACCCAT TAANCCAATG
801 GCAGCATAAG GCAAATCATT CAGNAAANAT CATTAAAATC AGGTATTNGT
851 AAAAAGGTTN AGTGATTGTG NNNAACNNAN TNGNATGTGG CAAACATNCN
901 AANTACATCC TGGAAGGACC TCACG
```

Mutant: NT94
Phenotype: temperature sensitivity
Sequence map: Mutant NT94 is complemented by pMP170, which contains a 2.5 kb insert of S. aureus genomic DNA. A partial restriction map is depicted FIG. 55. Database searches at both the nucleic acid and peptide levels reveal strong peptide-level similarities to yabM, a hypothetical ORF of uncharacterized function from B. subtilis, noted as being similar to the spoVB gene from B. subtilis; further similarities are noted to hypothetical ORFs from E. coli and H. influenzae.

DNA sequence data: The following DNA sequence data represents the sequence generated by primer walking through clone pMP170, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP170
SEQ ID NO. 60
pMP170 Length: 2531 nt
      1 TGGYTTRTTT CAACATAATA TAGACATTTY CAATGTTATT CTATTAATTC
    51 TCCACGAAAC TGTTATCTTA TCGTTTTCTG GTTCTAATAT GTGTTTTTTG
  101 GGTGATTTAA TTACTTGTTC CGTTGAACAT TTACAAGGCC TTTTTTAAGT
  151 TAACTGTTTG ACCTCATTAC GTGTACCGAC GCCCATATTT GCTAAAAATT
  201 TATCTATTCT CATCGTAAAA ACCTAACTCT ACGTCTTAAT TTTTCAGGAA
  251 TTTCACCTAA GAATTCGTCC GCAAGACGCG TTTTAATTGT GAWTGTACCG
  301 TAAATTAGAA TACCTACTGT AACACCTAAA ATAATAATGA TTAAGTWACC
  351 AAGTTTTAGT AGGTYCTAAR AATARATTTG CAAGGNAAAA TACTAATTCT
  401 ACACCTAGCA TCATAATNNT GNATACAAGG ATATWTWTGC AAAATGGATC
  451 CCAACTATAG CTGAATTTAA ACTTCGCATA TWTTTTAAGR ATWTAGRAAT
  501 TACATCCMAT TGCAAATAAT TAATGCGATA CTAGTACGTA AAATTGCACC
  551 AGGTGTATGG AATAACATAA TTAATGGATA GTTTAACGCT AACTTGATAA
  601 CTACAGAAGC TAAAATAACA TAAACTGTTA ATTTCTGTTT ATCTATACCT
  651 TGTAANATNG ATGCCGTTAC ACTTAATAGT GAAATYAGTA TTGCTACAGG
  701 CGCATAATAK AATAATAAGC GACTACCATC ATGGTTAGGG TCATGACCTA
  751 WAACAATTGG ATCGTAACCA TAGATAAACT GTGAAATTAA TGGTTGTGCC
  801 AAGGCCATAA TCYCCAATAC TAGCTGGGAA CAGTTATAAA CATTWAGTTA
  851 CACCAATTAG ATGTTCCTAA TTTGATGATG CATTTCATGT AAGCGACCTT
  901 CTGCAAATGT TTTTGTAATA TAAGGAATTA AACTCACTGC AAAACCAGCA
  951 CTTAATGATG TCGGAATCAT TACAATTTTA TTAGTTGACA TATTTAGCAT
1001 ATTAAAGAAT ATATCTTGTA ACTGTGAAGG TATACCAACT AAAGATAAAG
1051 CACCGTTATG TGTAAATTGA TCTACTAAGT TAAATAATGG ATAATTCAAA
1101 CTTACAATAA CGAACGGTGA TACTATAAGC AATAATTTCT TTATACATCT
1151 TGCCATATGA CACATCTATA TCTGTGTAAT CAGATTCGAC CATACGATCA
1201 ATATTATGCT TACGCTTTCT CCAGTAATAC CAGAGTGTGR ATATRCCAAT
1251 AATCGCACCA ACTGCTGCTG CAAAAGTAGC AATACCATTG GCTAATAAAA
1301 TAGAGCCATC AAAGACATTT AGTACTAAAT AACTTCCGAT TAATATGAAA
1351 ATCACGCGTG CAATTTGCTC AGTTACTTCT GACACTGCTG TTGGCCCCAT
1401 AGATTTATAA CCTTGGAATA TCCCTCTCCA TGTCGCTAAT ACAGGAATAA
1451 AGATAACAAC CATACTAATG ATTCTTATAA TCCAAGTTAA TATCATCCGA
1501 CTGACCAACC GTTTTTATCA TGAATGTTTC TAGCTAATGT TAATTCAGAA
1551 ATATAAGGTG YTAAGAAATA CAGTACCAAG AAACCTAAAA CACCGGTAAT
1601 ACTCATTACA ATAAAAYTCG ATTTATAAAA WTTCTGACTT WACTTTAWAT
1651 GCCCCAATAG CATTATATTT CGCAACATAT TTCGAAGCTG CTAATGGTAC
1701 ACCTGCTGTC GCCAACTGCA ATTGCAATAT TATATGGTGC ATAAGCGTWT
1751 GTTGAACGGS GCCATATTTT CTTGTCCCNC CAATTAAATA GTTGAATGGA
1801 ATGATAAAAA GTACGCCCAA TACCTTGGTA ATTAATATAC TAATGGTAAT
1851 TAAAAAGGTT CCACGCACCA TTTCTTTACT TTCACTCATT ACGAATCTCC
1901 CTATCTCATG TTTATTAAAG TTTTGTAAAC TAAAAGCTGT TTCTCTGTAA
1951 AATCATTTTT CATTATTATG AATATATCAC AAAACTTTAT TTCATYGTCG
2001 TATATTTCAA TGGAATTATC CATAACAAAA TTATCAACAC ATTGTCATTG
2051 AATACTAGAT TTTGATTAGA ATATTACGAA ATTTCATATA AACATTATAC
2101 TACTATTTGA GATGAACATC GCATAACAGT AGAAAAATCA TTCTTATCAT
2151 ACACATACAT CTTCATTTTT TATGAAGTTC ACATTATAAA TATATTCAAC
2201 ATAATTGTCA TCTCATAACA CAAGAGATAT AGCAAAGTTT AAAAAAGTAC
2251 TATAAAATAG CAATTGAATG TCCAGTAACA AATTTGGAGG AAGCGTATAT
2301 GTATCAAACA ATTATTATCG GAGGCGGACC TAGCGGCTTA ATGGCGGCAG
2351 TAGCWGCAAG CGAACAAAGT AGCAGTGTGT TACTCATTGA AAAAAAGAAA
2401 GGTCTAGGTC GTAAACTCAA AATATCTGGT GGCGGTAGAT GTAACGTAAC
2451 TAATCGAYTA CCATATGCTG AAATTATTCA AGGAACATTC CCTGGAAATG
2501 GGAAATTTTY ATCATAGTTC CCTTTTCAAT T
```

Mutant: NT96
Phenotype: temperature sensitivity.
Sequence map: Mutant NT96 is complemented by pMP125, which contains a 2.6 kb insert of S. aureus genomic DNA. A partial restriction map is depicted FIG. 56, along with open boxes to indicate the percentage of the clone for which DNA sequence has been obtained. Database searches at both the nucleic acid and peptide levels reveal strong similarities at the peptide level to the murC gene product, encoding UDP-N-Acetyl muramoyl-L-alanine synthase (EC 6.3.2.8), from B. subtilis (Genbank Accession No. L31845).

DNA sequence data: The following DNA sequence data represent the sequences at the left-most and right-most edges of clone pM125, using standard M13 forward and M13 reverse sequencing primers. The sequences below can be used to design PCR primers for the purpose of amplification from genomic DNA with subsequent DNA sequencing:

```
clone pMP125
SEQ ID NO. 61
pMP125.forward Length: 889 nt
    1 TCGAGCTCGG TACCCGGGGA TCCTCTAGAG TCGATCTACA GAGCTGTTTA
  51 ACGTTTGTAC TGAGTCACCG ATACCTTTAA CAGCATCTAC AACTGAGTTT
101 AAACGATCTA CTTTACCTTG GATATCCTCA GTTAAACGGT TTACTTTATG
151 AAGTAAATCT GTTGTTTCAC GAGTAATACC TTGAACTTGA CCTTCTACAC
201 CGTCAAGTGT TTTTGCAACA TAATCTAAGT TTTTCTTAAC AGAATTTAAT
251 ACAGCTACGA TACCGATACA TAAAATTAAG AATGCAATCG CAGCGATAAT
301 TCCAGCAATT GGTAAAATCC AATCCATTAA AAACGCCTCC TAATTAACAT
351 GTAATAATGT CATTAATAAT AAATACCCAT ACTACTCTAT TATAAACATA
401 TTAAAACGCA TTTTTCATGC CTAATTTATC TAAATATGCA TTTTGTAATT
451 TTTGAATATC ACCTGCACCC ATAAATGAAA ATAACAGCAT TATCAAATTG
501 TTCTAATACA TTAATAGAAT CTTCATTAAT TAACGATGCA CCTTCAATTT
551 TATCAATTAA ATCTTGTWTC GTTAATGCGC CAGTATTTTC TCTAATTGAT
601 CCAAAAATTT CACAATAAGA AATACACGAT CTGCTTTACT TAAACTTTCT
651 GCAAATTCAT TTAAAAATGC CTGTGTTCTA GAGAAAGTGT GTGGTTTGAN
701 ATACTGCAAC AACTTCTTTA TGTGGATATT TCTTTCGTGC GGTTTCAATT
751 GNNGCACTAA NTTCTCTTGG ATGGTGTNCA TAATCAGCTA CATTAACTTG
801 ATTTGCGATT GTAGTNTCAT NGANNGACGT TTAACNCCAC CAACGTTTCT
851 AATGCTTCTT TAANATTGGG ACATCTAACT TCTCTAAA
SEQ ID NO. 62
pMP125.reverse Length: 902 nt
    1 GCATGCCTGC AGGTCGATCC AAAAATGGTT GAATTAGCTC CTTATAATGG
  51 TTTGCCMMMT TTRGTTGCCA CCGKTAATTA CAGATGTCMA AGCCAGCTAC
101 ACAGAGTTTG AAAAKGGSCC STWGAAAGGA AATGGAACGA ACGTKATAAG
151 TTATTTGCCA CATTACCATG TACGTAATAT AACAGCCATT TAACAAAAAA
201 GCCACCATAT GATGAAAGAW TGCCAAAAAT TGTCATTGTA ATTGATGAGT
251 TGGCTGATTT AATGATGATG GCTCCGCAAG AAGTTGAACA GTCTATTGCT
301 AGAATTGCTC AAAAAGCGAG AGCATGTGGT ATTCATATGT TAGTAGCTAC
351 GCAAAGACCA TCTGTCAATG TAATTACAGG TTTAATTAAA GCCAACATAC
401 CAACAAGAAT TGCATTTATG GTATCATCAA GTGTAGATTC GAGAACGATA
451 TTAGACAGTG GTGGAGCAGA ACGCTTGTTA GGATATGGCG ATATGTTATA
501 TCTTGGTAGC GGTATGAATA AACCGATTAG AGTTCAAGGT ACATTTGTTT
551 CTGATGACGA AATTGATGAT GTTGTTGATT TTATCAAACA ACAAAGAGAA
601 CCGGACTATC TATTTGAAGA AAAAAGAAAT TGTTGAAAAA AACACAAACA
651 CMATCMCMAG ATGAATTATT TGATGATGTT TGTGCATTTA TGGTTAATGA
701 AGGACATATT TCAACATCAT TAATCCAAAG ACATTTCCAA ATTGGCTATA
751 ATAGAGCAGC AAGAATTATC GATCAATTAG AAGCAACTCG GTTATGTTTC
801 GAGTGCTAAT NGGTTCAAAA ACCNAGGGAT GTTTATGTTA CGGAAGCCGA
851 TTTTAAATAA AGAATAATTT ATGATTAAGG ATTTTTATAT AATGGACACC
901 CC
```

Mutant: NT99
Phenotype: temperature sensitivity
Sequence map: Mutant NT99 is complemented by pMP176, which contains a 3.6 kb insert of S. aureus genomic DNA. A partial restriction map is depicted FIG. 57. Database searches at both the nucleic acid and peptide levels reveal strong similarity at the peptide level to the murG gene, encoding UDP-GlcNAc:undecaprenyl-pyrophosphoryl-pentapeptide transferase, from B. subtilis (Genbank Accession No. D10602; published in Miyao, A. et al. Gene 118 (1992) 147-148.) Cross complementation studies (data not shown) have demonstrated that the minimal amount of clone pMP176 required for restoring a wild-type phenotype to mutant NT99 is contained in the right-half of the clone and contains the entire (predicted) murG ORF; the predicted size and orientation of this ORF is depicted in the restriction map by an arrow.

DNA sequence data: The following DNA sequence data represents the sequence generated by primer walking through clone pMP176, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP176
SEQ ID NO. 63
pMP176 Length: 3592 nt
      1 GATCCTTATT CTGAATATTT AACAAAWGCA ACAAACGAAA TCCCTTTGAA
    51 TGAAAGGTGT TTCAGGTGCA TTTTKTAGGT ATTGGTGCAG AAAATGCAAA
  101 AGAAAAATGA ATCAAATTAT GGTTACTAGT CCTATGAAGG GWTCTCCAGC
  151 AGAACGTGCT GGCATTCGTC CTAAAGATGT CATTACTAAA GTAAATGGAA
  201 AATCAATTAA AGGTAAAGCA TTAGATGAAG TTGTCAAAGA TGTTCGTGGT
  251 AAAGAAAACA CTGAAGTCAC TTTAACTGTT CAACGAGGTA GTGAAGAAAA
  301 AGACGTTAAG ATTAAACGTG RAAAAATTCA TGTTAAAAGT GTTGAGTATW
  351 AGRAAAAAGG TAAAGTTGGA GTTATTACTA TTAATAAATT CCAGANTGAT
  401 ACATCCAGGT GRATTGAAAG ATGCAGTTCT AAAAGCTCAC CAAAGATGGT
  451 TTGWAAAAGA TTGTTTTAGA TTTAAGAAAT AATCCAGGTG GACTACTAGA
  501 TGAAGCTGTT AAAATGGCAA ATATTTTTAT CGATAAAGGA AAAACTGTTG
  551 TTAAACTARA AAAAGGTAAA GATACTGAAG CAATTCNNAC TTCTAATGAT
  601 GCGTTAAAAG AAGCGAAAGA CATGGATATA TCCATCTTAG TGAATGAAGG
  651 TTCNGCTNGC GCTTCTGAAG TGTTTACTGG TGCGCTAAAA GACTNTAATA
  701 AAGCTAAAGT TTATGGGTCA AAAACATTCG GCAAAGGTGT CGTACAAACT
  751 ACAAGAGAGT TTAAGGGATG GTTCATTGTT AAAATATACT GAAATGGAAA
  801 TGGTTAACGC CAGATGGTCA TTATATTCAC NGTACAAGGC ATNAAACCAG
  851 ACGTTACTNT TTGACACACC TGAAATANCA ATCTTTTAAA TGTCATTCCT
  901 AATACGANAA CATTTAAAGT TNGGAGACGA TGAATCTAAA ATATTAAAAC
  951 TATTAAAAWT GGTTTATCAG CTTTAGGTTA TAAAGTTGAT AAATGGAATC
1001 AACGCCAATT TGGATAAAGC TTTAGAAAAT CAAGTTAAAG CTTYCCAMCA
1051 AGCGAATAAA CTTGAGGTAM YKGGKGAWTT TAATAAAGAA ACGAATAATA
1101 AATTTACTGA GTTATTAGTT GAAAAAGCTA ATAAACATGA TGATGTTCTC
1151 GATAAGTTGA TTAATATTTT AAAATAAGCG ATACACACTA CTAAAATTGT
1201 ATTATTATTA TGTTAATGAC ACGCCTCCTA AATTTGCAAA GATAGCAATT
1251 TAGGAGGCGT GTTTATTTTT ATTGACGTCT AACTCTAAAA GATATAAATT
1301 AGACATTTAC AAATGATGTA AATAACGCAA TTTCTATCAT CGCTGATAAC
1351 AATTCATGGT TTAATATGCA ATGAGCATAT ACTTTTTAAA TAGTATTATT
1401 CACTAGTTTT AACAATCAAT TAATTGGTAT ATGATACTTT TATTGGTTAT
1451 TTTTATCCCA TAGTGTGATA AWTACTATTT TTCATTCAYA ATAAAGGTTT
1501 AAAGCATGTT AATAGTGTGT TAAGATTAAC ATGTACTGAA AAACATGTTT
1551 WACAATAATG AATATAAGGA KTGACGTTAC ATGAWCCGTC CTAGGTAAAA
1601 TGTCMGAWTT AGATCAAATC TTAAATCTAG TAGAAGAAGC AAAAGAATTA
1651 ATGAAAGAAC ACGACAACGA GCAATGGGAC GATCAGTACC CACTTTTAGA
1701 ACATTTTGAA GAAGATATTG CTAAAGATTA TTTGTACGTA TTAGAGGAAA
1751 ATGACAAAAT TTATGGCTTT ATTGTTGTCG ACCAAGACCA AGCAGAATGG
1801 TATGATGACA TTGACTGGCC AGTAAATAGA GAAGGCGCCT TTGTTATTCA
1851 TCGATTAACT GGTTCGAAAG AATATAAAGG AGCTGCTACA GAATTATTCA
1901 ATTATGTTAT TGATGTAGTT AAAGCACGTG GTGCAGAAGT TATTTTAACG
1951 GACACCTTTG CGTTAAACAA ACCTGCACAA GGTTTATTTG CCAAATTTGG
2001 ATTTCATAAG GTCGGTGAAC AATTAATGGA ATATCCGCCM TATGATAAAG
2051 GTGAACCATT TTATGCATAT TATAAAAATT TAAAAGAATA GAGGTAATAT
2101 TAATGACGAA AATCGCATTT ACCGGAGGGG GAACAGTTGG ACACGTATCA
2151 GTAAATTTWA RTTTAATTCC AACTGCATTA TCACAAGGTT ATGGARGCGC
2201 TTTATATTGG TTCTAAAAAT GGTATTGAAA GAGAGAATGA TTGAWTCACC
2251 AACTACCCRG AAATTAAGTA TTATCCTATT TCGGAGTGKT AAATTAAGAA
2301 GATATATTTC TTTAGAAAAT GCCAAAGACG TATTTAAAGT ATTGAAAGGT
2351 ATTCTTGATG CTCGTAAAGT TTTGAAAAAA GAAAAACCTG ATCTATTATT
2401 TTCAAAAGGT GGATTTGTAT CTGTGCCTGT TGTTATTGCA GCCAAATCAT
2451 TAAATATACC AACTATTATT CATGAATCTG ACTTAACACC AGGATTAGCG
2501 AATAAGATAG CACTTAAATT TGCCAAGAAA ATATATACAA CATTTGAAGA
2551 AACGCTAAAC TACTTACCTA AAGAGAAAGC TGATTTTATT GGAGCAACAA
2601 TTCGAGAAGA TTTAAAAAAT GGTAATGCAC ATAATGGTTA TCAATTAACA
2651 GGCTTTWATG RAAATAAAAA AGTTTTACTC GTYATGGGTG GAAGCTTWGG
2701 AAGTAAAAAA TTAAATAGCA TTATTCGCGA AAACTTAGAT GCATTTATTA
2751 CAACAATATC AAGTGATACA TTTAACTGGT AAAGGAITAA AAGATGCTCA
2801 AGTTAAAAAA TCAGGATATA TACAATATGA ATTTGTTAAA GNGGATTTAA
2851 CAGATTTATT AGCAATTACG GATACAGTAA TAAGTAGAGC TGGATCAAAT
2901 GCGATTTATG GAGTTCTTAA CATTACGTNT ACCAATGTTA TTAGTACCAT
2951 TAGGTTTAGA TCAATCCCGA GGCGACCAAA TTGACANTGC AAATCATTTT
3001 GCTGATAAAG GATATGCTAA AGCGATTGAT GAAGAACAAT TAACAGCACA
3051 AATTTTATTA CAAGAACTAA ATGAAATGGA ACAGGAAAGA ACTCGAATTA
3101 TCAATAATAT GAAATCGTAT GAACAAAGTT ATACGAAAGA AGCTTTATTT
3151 GATAAGATGA TTAAAGACGC ATTGAATTAA TGGGGGGTAA TGCTTTATGA
3201 GTCAATGGAA ACGTATCTCT TTGCTCATCG TTTTTACATT GGTTTTTGGA
3251 ATTATCGCGT TTTTCCACGA ATCAAGACTT GGGAAATGGA TTGATAATGA
3301 AGTTTATGAG TTTGTATATT CATCAGAGAG CTTTATTACG ACATCTATCA
3351 TGCTTGGGGC TACTAAAGTA GGTGAAGTCT GGGCAATGTT ATGTATTTCA
3401 TTACTTCTTG TGGCATATCT CATGTTAAAG CGCCACAAAA TTGAAGCATT
3451 ATTTTTTGCA TTAACAATGG CATTATCTGG AATTTTGAAT CCAGCATTAA
3501 AAAATATATT CGATAGAGAA AGGACCTGAC ATTGCTGGCG TTTGAATTGG
3551 ATGATTAACA GGRTTTAGTT TTCCTGAGCG GTCATGCTAT GG
```

Mutant: NT102
Phenotype: temperature sensitivity
Sequence map: Mutant NT102 is complemented by pMP129, which contains a 2.5 kb insert of S. aureus genomic DNA. A partial restriction map is depicted FIG. 58 (there are no apparent restriction sites for EcoR I, Hind III, Bam HI or Pst I). Database searches at both the nucleic acid and peptide levels reveal strong similarity to one hypothetical ORF of unknown function from Synechocystis spp.; another ORF with no apparent homolog on the current databases is also predicted to be contained in this clone. The predicted sizes and orientations of these two hypothetical ORFs is depicted in the map.

DNA sequence data: The following DNA sequence data represents the sequence generated by primer walking through clone pMP129, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP129
SEQ ID NO. 64
pMP129 Length: 2573 nt
      1 ATTCGAGCTC GGTACCCGKG GATCCTSYAG AGTCGATCCG CTTGAAACGC
    51 CAGGCACTGG TACTAGAGTT TTGGGTGGTC TTAGTTATAG AGAAAGCCAT
  101 TTTGCATTGG AATTACTGCA TCAATCACAT TTAATTTCCT CAATGGATTT
  151 AGTTGAAGTA AATCCATTGA TTGACAGTAA TAATCATACT GCTGAACAAG
  201 CGGTTTCATT AGTTGGAACA TTTTTTGGTG AAACTTTATT ATAAATAAAT
  251 GATTTGTAGT GTATAAAGTA TATTTTGCTT TTTGCACTAC TTTTTTTAAT
  301 TCACTAAAAT GATTAAGAGT AGTTATAATC TTTAAAATAA TTTTTTTCTA
  351 TTTAAATATA TGTTCGTATG ACAGTGATGT AAATGATTGG TATAATGGGT
  401 ATTATGGAAA AATATTACCC GGAGGAGATG TTATGGATTT TTCCAACTTT
  451 TTTCAAAACC TCAGTACGTT AAAAATTGTA ACGAGTATCC TTGATTTACT
  501 GATAGTTTGG TATGTACTTT ATCTTCTCAT CACGGTCTTT AAGGGAACTA
  551 AAGCGATACA ATTACTTAAA GGGATATTAG TAATTGTTAT TGGTCAGCAG
  601 ATAATTWTGA TATTGAACTT GACTGCMACA TCTAAATTAT YCRAWWYCGT
  651 TATTCMATGG GGGGTATTAG CTTTAANAGT AATATTCCAA CCAGAAATTA
  701 GACGTGCGTT AGAACAACTT GGTANAGGTA GCTTTTTAAA ACGCNATACT
  751 TCTAATACGT ATAGTAAAGA TGAAGAGAAA TTGATTCAAT CGGTTTCAAA
  801 GGCTGTGCAA TATATGGCTA AAAGACGTAT AGGTGCATTA ATTGTCTTTG
  851 AAAAAGAAAC AGGTCTTCAA GATTATATTG AAACAGGTAT TGCCAATGGA
  901 TTCAAATATT TCGCAAGAAC TTTTAATTAA TGTCTTTATA CCTAACACAC
  951 CTTTACATGA TGGTGCAAKG ATTATTCAAG GCACGAAPAT TGCAGCAGCA
1001 GCAAGTTATT TGCCATTGTC TGRWAGTCCT AAGATATCTA AAAGTTGGGT
1051 ACAAGACATA GAGCTGCGGT TGGTATTTCA GAAGTTATCT GATGCATTTA
1101 CCGTTATTGT ATCTGAAGAA ACTGGTGATA TTTCGGTAAC ATTTGATGGA
1151 AAATTACGAC GAGACATTTC AAACCGAAAT TTTTGAAGAA TTGCTTGCTG
1201 AACATTGGTT TGGCACACGC TTTCAAAAGA AAGKKKTGAA ATAATATGCT
1251 AGAAAKTAAA TGGGGCTTGA GATTTATTGC CTTTCTTTTT GGCATTGTTT
1301 TTCTTTTTAT CTGTTAACAA TGTTTTTGGA AATATTCTTT AAACACTGGT
1351 AATTCTTGGT CAAAAGTCTA GTAAAACGGA TTCAAGATGT ACCCGTTGAA
1401 ATTCTTTATA ACAACTAAAG ATTTGCATTT AACAAAAGCG CCTGAAACAG
1451 TTAATGTGAC TATTTCAGGA CCACAATCAA AGATAATAAA AATTGAAAAT
1501 CCAGAAGATT TAAGAGTAGT GATTGATTTA TCAAATGCTA AAGCTGGAAA
1551 ATATCAAGAA GAAGTATCAA GTTAAAGGGT TAGCTGATGA CATTCATTAT
1601 TCTGTAAAAC CTAAATTAGC AAATATTACG CTTGAAAACA AAGTAACTAA
1651 AAAGATGACA GTTCAACCTG ATGTAAGTCA GAGTGATATT GATCCACTTT
1701 ATAAAATTAC AAAGCAAGAA GTTTCACCAC AAACAGTTAA AGTAACAGGT
1751 GGAGAAGAAC AATTGAATGA TATCGCTTAT TTAAAAGCCA CTTTTAAAAC
1801 TAATAAAAAG ATTAATGGTG ACACAAAAGA TGTCGCAGAA GTAACGGCTT
1851 TTGATAAAAA ACTGAATAAA TTAAATGTAT CGATTCAACC TAATGAAGTG
1901 AATTTACAAG TTAAAGTAGA GCCTTTTAGC AAAAAGGTTA AAGTAAATGT
1951 TAAACAGAAA GGTAGTTTRS CAGATGATAA AGAGTTAAGT TCGATTGATT
2001 TAGAAGATAA AGAAATTGAA TCTTCGGTAG TCGAGATGAC TTMCAAAATA
2051 TAAGCGAAGT TGATGCAGAA GTAGATTTAG ATGGTATTTC AGAATCAACT
2101 GAAAAGACTG TAAAAATCAA TTTACCAGAA CATGTCACTA AAGCACAACC
2151 AAGTGAAACG AAGGCTTATA TAAATGTAAA ATAAATAGCT AAATTAAAGG
2201 AGAGTAAACA ATGGGAAAAT ATTTTGGTAC AGACGGAGTA AGAGGTGTCG
2251 CAAACCAAGA ACTAACACCT GAATTGGCAT TTAAATTAGG AAGATACGGT
2301 GGCTATGTTC TAGCACATAA TAAAGGTGAA AAACACCCAC GTGTACTTGT
2351 AGGTCGCGAT ACTAGAGTTT CAGGTGAAAT GTTAGAATCA GCATTAATAG
2401 CTGGTTTGAT TTCAATTGGT GCAGAAGTGA TGCGATTAGG TATTATTTCA
2451 ACACCAGGTG TTGCATATTT AACACGCGAT ATGGGTGCAG AGTTAGGTGT
2501 AATGATTTCA GCCTCTCATA ATCCAGTTGC AGATAATGGT ATTAAATTCT
2551 TTGSCTCGAC CNCCNNGCTN GCA
```

Mutant: NT114
Phenotype: temperature sensitivity
Sequence map: Mutant NT114 is complemented by pMP151, which contains a 3.0 kb insert of S. aureus genomic DNA. A partial restriction map is depicted FIG. 59. Database searches at both the nucleic acid and peptide levels reveal strong similarity at the peptide level to the dfp gene, encoding a flavoprotein affecting pantothenate metabolism and DNA synthesis, from E. coli (Genbank Accession No. L10328; published in Lundberg, L. G. et al. EMBO J. 2 (1983) 967-971). The predicted size and orientation of the Dfp ORF is represented by an arrow in the restriction map.

DNA sequence data: The following DNA sequence data represents the sequence generated by primer walking through clone pMP151, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP151
SEQ ID NO. 65
pMP151 Length: 2976 nt
      1 GRTCGACTCT AGAGTCGATC TTTAAATGGG TCTCTTTCAA CAACCGCGTC
    51 ATATTTTTMA ACATAACCTT TTTTRATAAG TCCATCTAAA CTGGATTTTR
  101 AAAAGCCCAT ATCCTCAATA TCAGTTAAAA ATATTGTTTT ATGTTGTTCT
  151 TCAGACAAGT AAGCATACAA ATCGTATTGT TTAATAACTT TCTCCAACTT
  201 AGCTAATACT TCATCAGGAT GATACCCTTC AATGACACGA ACAGCACGCT
  251 TGGTTTTTTT AGTTATATTT TGTGTGAGAA TCGTTTTTTC TTCAACGATA
  301 TCATCTTTTA ACAACTTCAT AAGCAATTGA ATATCATTAT TTTTTTGCGC
  351 ATCTTTATAA TAATAGTAAC CATGCTTATC AAATTTTTGT AATAAAGCTG
  401 AAGGTAGCTC TATGTCATCT TTCATCTTAA ATGCTTTTTT ATACTTCGCT
  451 TTAATAGCAC TCGGAAGCAT CACTTCTAGC ATAGAAATAC GTTTAATGAC
  501 ATGAGTTGAA CCCATCCACT CACTTAAAGC TATTAATTCT GATGTTAATT
  551 CTGGTTGTAT ATCTTTCACT TCTATGATTT TTTTTAACTT CGAAACGTCA
  601 AGTTGTGCAT CAGGTTCTGC TGTTACTTCC ATTACATAAC CTTGAATCGT
  651 TCTTGGTCCA AAAGGTACAA TTACACGCAC ACCAGGTTGG ATGACAGATT
  701 CGAGTTGTTC GGGAATTATA TAATCAAATT TATAGTCAAC GCTCTTCGAC
  751 GCGACATCGA CTATGACTTT CGCTATCATT ATKGCCACCT AGTTTCTAGT
  801 TCATCTAAAA TTTGTGCAGC WAATACTACK TTTTKNCCTT YCTTGATATT
  851 TACKTTTTCA TTAKTTTTAA AATGCATTGT CAATTCATTA TCATCAGAAC
  901 TAAATCCGAT AGACATATCC CCAACATTAT TTGAAATAAT CACATCTGCA
  951 TTTTTCTTGC GTAATTTTTG TTGTGCATAA TTTTCAATAT CTTCAGTCTC
1001 TGCTGCAAAG CCTATTAAAT ACTGTGATGT TTTATGTTCA CCTAAATATT
1051 TAAGAATGTC TTTAGTACGT TTAAAAGATA CTGACAAATC ACCATCCTGC
1101 TTTTTCATCT TATGTTCCTA ATACATCAAC CGGTGTATAG TCAGATACGG
1151 CTGCTGCTTT TACAACAATA TYTTGTTCCG TYAAATCGGC TTGTCACTTG
1201 GTTCAAACAT TTCTTCAGGC ACTTTGRACA TGAATAACTT CAATATCTTT
1251 TGGATCCTCT AGTGTTGTAG GACCAGCAAC TAACGTCACG ATAGCTCCTC
1301 GATTTCGCAA TGCTTCAGCT ATTGCATAGC CCATTTTTCC AGAAGAACGA
1351 TTGGATACAA ATCTGACTGG ATCGATAACT TCAATAGTTG GTCCTGCTGT
1401 AACCAATGCG CGTTTATCTT GAAATGAACT ATTAGCTAAA CGATTACTAT
1451 TTTGAAAATG AGCATCAATT ACAGAAACGA TTTGAAGCGG TTCTTCCATA
1501 CGTCCTTTAG CAACATAACC ACATGCTAGA AATCCGCTTC CTGGTTCGAT
1551 AAAATGATAC CCATCTTCTT TTAAAATATT AATATTTTGC TGCGTTACGT
1601 TTATTTTCAT ACATATGCAC ATTCATAGCA GGCGCAATAA ATTTCGGTGT
1651 CTCTGTTGCT AGCAACGTTG ATGTCACCAA ATCATCAGCA ATACCTACAC
1701 TCAATTTTGC AATTGTATTT GCCGTTGCAG GTGCAACAAT GATTGCATCK
1751 GCCCAATCCA CCTAATGCAA TATGCTGTAT TTCTGGAAGG ATTTTYTTCT
1801 ATAAAAGTAT CTGTATAAAC AGCATTTCGA MTTATTGCTT GAAATGCTAA
1851 TGGTGTCACA AATTTTTGTG CGTGATTCGT TAAACATAAC GCGAACTTCA
1901 TAACCCAGAT TGTGTTAACT TACTTGTCAA ATCAATTGCT TTATATGCCG
1951 CAATGCCACC TGTAACGGCT AATAATATTT TCTTCATATT CAATCTCCCT
2001 TAAATATCAC TATGACATTT ACGCTTTACA TCATCATATG CGCACAAATG
2051 CTCATTACTT TTTTATAGAT ACAAATTTAG TATTATTATA ACATCAATCA
2101 TTGGATAAAC TAAAAAAACA CACCTACATA GGTGCGTTTG ATTTGGATAT
2151 GCCTTGACGT ATTTGATGTA ACGTCTAGCT TCACATATTT TTAATGGTCG
2201 AAACTATTCT TTACCATAAT AATCACTTGA AATAACAGGG CGAATTTTAC
2251 CGTCAGCAAT TTCTTCTAAC GCTCTACCAA CTGGTTTAAA TGAATGATAT
2301 TCACTTAATA ATTCAGTTTC AGGTTGTTCA TCAATTTCAC GCGCTCTTTT
2351 CGCTGCAGTT GTTGCAATTA AATACTTTGA TTTAATTTGT GACGTTAATT
2401 GGTTTAAAGG TGGATTTAAC ATTATTTTTT AGCCTCCAAA ATCATTTTTC
2451 TATACTTAGC TTCTACGCGC TCTCTTTTTA AGTGCTCAGC TTCTACAATA
2501 CATTGAATTC TATTCTTCGC AAGTTCTACT TCATCATTAA CTACAACGTA
2551 ATCGTATAAA TTCATCATTT CAACTTCTTT ACGCGCTTCG TTAATACGAC
2601 TTTGTATTTT CTCATCAGAT TCTGTTCCTC TACCTACTAA TCGCTCTCTC
2651 AAGTGTTCTA AACTTGGAGG TGCTAAGAAA ATAAATAGCG CATCTGGAAA
2701 TTTCTTTCTA ACTTGCTTTG CACCTTCTAC TTCAATTTCT AAAAATACAT
2751 CATGACCTTC GTCCATTGTA TCTTTAACAT ATTGAACTGG TGTACCATAA
2801 TAGTTGCCTA CATATTCAGC ATATTCTATA AATTGGTCAT CTTTGATTAA
2851 AGCTTCAAAC GCATCCCTAG TTTTAAAAAA GTAATCTACG CCATTCAACW
2901 TCACCTTCAC GCATTTGACG TGTTGTCATT GGAATAGRAG AGCTTRANNG
2951 ATGTATNGNG ATCGACCTGC AGTCAT
```

Mutant: NT124
phenotype: temperature sensitivity
Sequence map: Mutant NT124 is complemented by plasmid pMP677, which carries a 3.0 kb insert of wild-type S. aureus genomic DNA. A partial restriction map is depicted in FIG. 60 with open boxes to depict the current status of the contig project; no apparent restriction sites for EcoR I, HinD III, BamH I or Pst I are present. Database searches at the nucleic acid and (putative) polypeptide levels against currently available databases reveal no significant similarities to known genes at this time.

DNA sequence data: The following DNA sequence data represents the sequence generated from clone pMP677, starting with standard M13 forward and M13 reverse sequencing primers; the sequence contig will be completed later via primer walking strategies. The sequence below can be used to design PCR primers for the purpose of amplification from genomic DNA with subsequent DNA sequencing.

```
clone pMP677
SEQ ID NO. 66
pMP677.forward Length: 540 nt
    1 TACCCGGGGA CCTTGAAAAA TACCTGGTGT ATCATACATA AATGANGTGT
  51 CATCTANAGG AATATCTATC ATATCTNAAG TTGTTCCAGG GANTCTTGAA
101 GTTGTTACTA CATCTTTTTC ACCAACACTA GCTTCAATCA GTTTATTAAT
151 CAATGTAGAT TTCCCAACAT TCGTTGTCCC TACAATATAC ACATCTTCAT
201 TTTCTCGAAT ATTCGCAATT GATGATAATA AGTCNTNTNT GCCCCAGCCT
251 TTTTCAGCTG AAATTAATAC GACATCGTCA GCTTCCAAAC CATATTTTCT
301 TGCTGTTCGT TTTAACCATT CTTTAACTCG ACGTTTATTA ATTTGTTTCG
351 GCAATAAATC CAATTTATTT GCTGCTAAAA TGATTTTTTT GTTTCCGACA
401 ATACGTTTAA CTGCATTAAT AAATGATCCT TCAAAGTCAA ATACATCCAC
451 GACATTGACG ACAATACCCT TTTTATCCGC AAGTCCTGAT AATAATTTTA
501 AAAAGTCTTC ACTTTCTAAT CCTACATCTT GAACTTCGTT
SEQ ID NO. 67
pMP677.reverse Length: 519 nt
    1 GACGCGTAAT TGCTTCATTG AAAAAATATA TTTGTNGAAA GTGGTGCATG
  51 ACAAATGTAC TGCTCTTTTT GTAGTGTATC AGTATTGTGA TGTTTTAATG
101 AGAATATTAT ATGAATCATT ATGAAATTTA ATAAAAATAA AAGAAATGAT
151 TATCATTTTT TCTTATATAC TGTTAAACGG TTTGGAATTT TTAGGTATAC
201 ACTGTATTGG TTGATATAAC TCAACTAATA ATTGCGAACA GAGTATTTCA
251 AATTGAAAAG TATTATGAGC GTGATACATA ATCAAAATTG TAGGCTCAAG
301 AACCACTACA TAATAAACCA TAAGCGGTTC TTTATCATTT ATGTCTCGCT
351 CTCAAATGTA AATTAATAAT TGTTTTGGGG GAGTTTGAAG TTAAATATTT
401 AACAGGATTT ATTTTAATAT TATTGTTAGA AGGAATTTTT ACAAATTCAG
451 CGAGTGCAAT CGAATATTCA GACTTACATC ATAAAAGTAA GTTTGATTCA
501 AAGCGTCCTA AGTTAATGC
```

Mutant: NT125
Phenotype: temperature sensitivity
Sequence map: Mutant NT125 is complemented by plasmid pMP407, which carries a 3.3 kb insert of wild-type S. aureus genomic DNA. A partial restriction map is depicted in FIG. 61. Database searches at the nucleic acid and (putative) polypeptide levels against currently available databases reveal strong peptide level similarities to rnpA (Genbank Accession No. X62539), encoding the protein component of RNAseP (EC 3.1.26.5), and thdF (Genbank Accession No. X62539), a hypothetical ORF with similarities to the thiophene/furan oxidase from E. coli.

DNA sequence data: The following DNA sequence data represents the sequence generated by primer walking through clone pMP407, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP407
SEQ ID NO. 68
pMP407 Length: 3308 nt
      1 ACCAATATAT GCATCTGAAC GACTTAATAT CTTTTCGCCT GTGTTTAACA
    51 CTTTACCTGC AGCGTTAATA CCTGCCATCA ATCCTTGTCC TGCTGCTTCT
  101 TCATAACCAG ATGTACCATT AATTTGACCT GCAGTATATA AGTTTTTAAT
  151 CATTTTCGTT TCAAGTGTAG GCCATAACTG CGTTGGCACA ATCGCATCAT
  201 ATTCAATTGC GTAGCCGGCA CGCATCATAT CTGCTTTTTC AAGACCTGGT
  251 ATCGTCTCTA ACATTTGACG TTGCACATGT TCAGGAAGAC TTGTNGACAA
  301 TCCTTGCACA TATACTTCAT TTGTATTAAC GACCTTCAGG CTCTAAGAAA
  351 AAGTTGATGT CGCGGCTTAT CATTAAATCG AACAAATTTA TCTTCAATTG
  401 AAGGGCAATA ACGTGGCCCG GTTCCTTTAA TCATCCCTGA ATACATTGCA
  451 GATAGATGTA AATTATCATC GATAACTTTG TGTGTTTCAN CATTAGTATA
  501 CGTTAGCCAA CATGGCAATT GATCKAMYAT ATATTCTGTT GTTTCAAAGC
  551 TGAATGCACG ACCTACATCG TCACCTGGTT GTATTTCAGT CTTCGAATAR
  601 TCAATTGTTT TTGAATTGTA CACGGCGGWG GTGTACCTGT TTTAAAACGA
  651 ACAATATCAA AACCAAGTTC TCTTARATGK GKSTGATAAT GTGATTGATG
  701 GTAATTGGTG GATTTGGTCC ACTTGAATAC TTCATATTAC CTAAAATGAT
  751 TTCACCACGT ATRAAATGTT GCCCGTWGTA ATAATTACTG CTTTAGATAA
  801 ATACTCTGTA CCAATATTTG TACGTACACC TTKAACTGTC ATTAWCTTCT
  851 ATAAKAAGTT CGTCTACCAT ACCTTGCATT AATATGCAAA TTTTCTTCAT
  901 CTTCAATCAM GCGTTTCATT TCTTGTTGAT AAAGTACTWT AKCTGCTTGC
  951 GCCKCTWAGT GCTCTTACAR CAGGTCCTTT AACTGTATTT AACATTCTCA
1001 TTTGAATGTG TGTTTTATCG ATTGTTTTTG CCATTTGTCC ACCTAAAGCA
1051 TCAATTTCAC GAACAACGAT ACCTTTAGCT GGTCCACCTA CAGATGGGTT
1101 ACATGGCATA AATGCAATAT TATCTAAATT TATTGTTAGC ATTAATGTTT
1151 TAGCACCACG TCTTGCAGAT GCTAAACCTG CTTCTACACC TGCATGTCCC
1201 GCACCTATAA CGATTACATC ATATTCTTGA ACCACAATAT AAACCTCCTT
1251 ATTTGATATC TTACTAGCCK TCTTAAGACG GTATTCCGTC TATTTCAATT
1301 ACTATTTACC TAAGCAGAAT TGACTGAATA ACTGATCGAT GAGTTCATCA
1351 CTTGCAGTCT CACCAATAAT TTCTCCTAAT ATTTCCCAAG TTCTAGTTAA
1401 ATCAATTTGT ACCATATCCA TAGGCACACC AGATTCTGCT GCATCAATCG
1451 CMTCTWGTAT CGTTTGTCTT GCTTGTTTTA ATAATGAAAT ATGTCTTGAA
1501 TTAGAAACAT AAGTCATATC TTGATTTTTG TACTTCTCCA CCAAAGAACA
1551 AATCTCGAAT TTGTATTTCT AATTCATCAA TACCTCCTTG TTTTAACATT
1601 GAAGTTTGAA TTAATGGCGT ATCACCTATC ATATCTTTAA CTTCATTAAT
1651 ATCTATGTTT TGCTCTAAAT CCATTTTATT AACAATTACG ATTACATCTT
1701 CATTTTTAAC CACTTCATAT AATGTGTAAT CTTCTTGAGT CAATGCTTCG
1751 TTATTGTTTA ATACAAATAA AATTAAGTCT GCTTGGCTAA GAGCCTTTCT
1801 AGAGCGTTCA ACACCAATCT TCTCTACTAT ATCTTCTGTC TCACGTATAC
1851 CAGCAGTATC AACTAATCTT AATGGCACGC CACGAACATT GACGTAMTCT
1901 TCTAAGACAT CTCTAGTAGT ACCTGCTACY TCAGTTACAA TCGCTTTATT
1951 ATCTTGTATT AAATTATTTA ACATCGATGA TTTACCTACG TTTGGTTTAC
2001 CAACAATAAC TGTAGATAAA CCTTCACGCC ATAATTTTAC CCTGCGCACC
2051 GGTATCTAAT AAACGATTAA TTTCCTGTTT GATTTCTTTA GACTGCTCTA
2101 AAAGAAATTC AGTAGTCGCA TCTTCAACAT CATCGTATTC AGGATAATCA
2151 ATATTCACTT CCACTTGAGC GAGTATCTCT AATATAGATT GACGTTGTTT
2201 TTTGATTAAG TCACTTAGAC GACCTTCAAT TTGATTCATC GCAACTTTAG
2251 AAGCTCTATC TGTCTTCGAG CGAWWAAAGT CCATAACTGY TTCAGCTTGA
2301 GATAAATCAA TACGACCATT TAAAAAGGCA MGTTTTGTAA ATTCAACCTG
2351 GCTCAGCCAT TCTAGCGCCA TATGTCATAG TAAGTTCCAG CACTCTATTA
2401 ATCGTTAAAA TACCACCATG ACAATTAATT TCTATAATAT CTTCGCGTGT
2451 AAATGTTTTT GGCGCTCTTA ACACAGACAC CATAACTTNT TCAACCATTC
2501 TTTAGACTCT GGATCAATAA TATGACCGTA ATTAATCGTA TGTGATGGAA
2551 CATCATTTAA AAGATGTTTT CCTTTATATA ATTTGTCAGC AATTTCAACG
2601 GCTTGCGGTC CAGACAATCG AACAATTCCA ATTGCCCCTT CACCCATTGG
2651 TGTTGAAATA CTCGTAATTG TATCTAAATC CATATTGCTA CTCGCCTCCT
2701 TCAACGATGT GAATACATTT TAAAGTAAGT TATTATAACC CTAAGGTCAG
2751 TCTTAACGTT TGTCTGAGGT AAGACTTCGG GATGTGTTGA GTGGTTAATG
2801 TTTTCCTTCC CCTACCCTAT CCTTACTTAA TCTTTTTATT AAAAACTTTG
2851 GCAATTTTAA GTACGTGCTC AAGACTATTC TGTATTTGTA AAGTCGTCAT
2901 ATCTTTAGCT GGCTGTCTTG CTATTACAAT AATATCTTTG GCCAATATAT
2951 GCGACTTATG TACTTTGAAA TTTTCACGTA TTGCTCTTTT AATCTTGTTT
3001 CTTAACACTG CATTACCTAG TTTTTTAGAA ACACTAATAC CTAAGCGAAA
3051 ATGGTCTATT TCTTTATTAT TACAAGTGTA TACAACAAAT TGTCTGTTGG
3101 CTACAGAATG ACCTTTTTTA TATATTCTCT GAAAATCTGC ATTCTTTTTA
3151 ATTCGGTAAG CTTTTTCCAA TAACATCACT CGCTTATTTA TCGTTTTTAT
3201 TTGAAGCTAT ATTTAAACTT CTATTGAGCT TATAACATAA ATTTCTATTT
3251 ATTCTTAATT TAAACGAAAA AAAAGATCGA CTCTAGAGGA TCCCCGGGTA
3301 CCGAGCTC
```

Mutant: NT144
Phenotype: temperature sensitivity
Sequence map: Mutant NT144 is complemented by plasmid pMP414, which carries a 4.5 kb insert of wild-type S. aureus genomic DNA. A partial restriction map is depicted in FIG. 62. Database searches at the nucleic acid and (putative) polypeptide levels against currently available databases reveal identity to the Hsp70 locus from S. aureus (Genbank Accession No. D30690), including an additional 600 bp of unpublished sequence upstream of the Genbank entry. Experiments are underway to determine which ORF in this contig is the essential gene.

DNA sequence data: The following DNA sequence data represents the sequence generated from clone pMP414, starting with standard M13 forward and M13 reverse sequencing primers; the sequence contig will be completed later via primer walking strategies. The sequence below can be used to design PCR primers for the purpose of amplification from genomic DNA with subsequent DNA sequencing.

```
clone pMP414
SEQ ID NO. 69
pMP414.forward Length: 1004 nt
      1 AGTTACGGCT TAATACTTGA ACCNAAAACC CAATTTTATA ATATGTATAG
    51 AAAAGGCTTG CTCAAACTTG CTAATGAGGA TTTAGGTGCT GACATGTATC
  101 AGTTGCTGAT GTCTAANATA GAACAATCTC CTTTCCATCA ATACGAAATA
  151 TCTAATTTTG CATTAGATGG CCATGANTCN NAACATAATA AGGTTTACTG
  201 GTTTAATGAG GAATATTATG GATTTGGAGC AGGTGCAAGT GGTTATGTAN
  251 ATGGTGTGCG TTATACGAAT ATCAATCCAG TGAATCATTA TATCAAAGCT
  301 ATNAATAAAG AAAGTAAAGC AATTTTAGTA TCAAATAAAC CTTCTTTGAC
  351 TGAGAGAATG GAAGAAGAAA TGTTTCTTGG GTTGCGTTTA AATGAAAGTG
  401 TGAGTAGTAG TAGGTTCAAA AAGAAGTTTG ACCAATCTAT TGAAAGTGTC
  451 TTTGGTCAAA CAATAAATAA TTTAAAAGAG AAGGAATTAA TTGTAGAAAA
  501 AGAACGATGT GATTGCACTT ACAAATAGAG GGAAAGTCAT ANGTAATGAG
  551 GTTTTTGAAG CTTTCCTAAT CAATGATTAA GAAAAATTGA AATTTCGAGT
  601 CTTTAACATT GACTTANTTT GACCAATTTG ATAAATTATA ATTAGCACTT
  651 GAGATAAGTG AGTGCTAATG AGGTGAAAAC ATGANTACAG ATAGGCAATT
  701 GAGTATATTA AACGCAATTG TTGAGGATTA TGTTGATTTT GGACAACCCG
  751 TTGGTTCTAA AACACTAATT GAGCGACATA ACTTGAATGT TAGTCCTGCT
  801 ACAATTAGAA ATGAGATGAA ACAGCTTGAA GATTTAAACT ATATCGAGAA
  851 GACACATAGT TCTTCAGGGC GTTCGCCATC ACAATTAGGT TTTAGGTATT
  901 ATGTCAATCG TTTACTTGAA CAAACATCTC ATCAAAAAAC AAATAAATTA
  951 AGACGATTAA ATCAATTGTT AGTTGAGAAC AATATGATGT TTCATCAGCA
1001 TTGA
SEQ ID NO. 70
pMP414.reverse Length: 1021 nt
      1 CCTGCAGGTC GATCCTGACA ACATTCTAAT TGTATTGTTT AATTATTTTT
    51 TGTCGTCGTC TTTTACTTCT TTAAATTCAG CATCTTCTAC AGTACTATCA
  101 TTGTTTTGAC CAGCATTAGC ACCTTGTGCT TGTTGTTGCT GTTGAGCCGC
  151 TTGCTCATAT ACTTTTGCTG ATAATTCTTG AATCACTTTT TCAAGTTCTT
  201 CTTTTTTAGA TTTAATATCT TCTATATCTT GACCTTCTAA AGCAGTTTTA
  251 AGAGCGTCTT TTTTCTCTTC AGCAGATTTT TTATCTTCTT CACCGATATT
  301 TTCGCCTAAA TCAGTTAAAG TTTTTTCAAC TTGGAATACT AGACTGTCAG
  351 CTTCGTTTCT TAAGTCTACT TCTTCACGAC GTTTTTTATC TGCTTCAGCG
  401 TTAACTTCAG CATCTTTTAC CATACGGTCR ATTTCTTCGT CTGATAATGA
  451 AGAACTTGAT TGAATTGTAA TTCTTTGTTC TTTATTTGTA CCTAAGTCTT
  501 TTGGCAGTTA CATTTACAAT ACCGTTTTTA TCGATATCAA ACGTTACTTC
  551 AATTTGGAGG TTTACCACCG TTTCARMWGG TGGAATATCA GTCAATTGGA
  601 ATCTACCAAG TGTTTTATTA TCCGCAGCCA TTGGACGTTC ACCTTGTAAT
  651 ACGTGTACAT CTACTGATGG TTGATTATCT ACTGCTGTTG AATAGATTTG
  701 AGATTTAGAT GTAGGAATCG TAGTGTTACG TTCAATTAAC GTATTCATAC
  751 GTCCACCTAA AATTTCAATA CCTAAAGATA GTGGTGTTAC GTCTAATAAT
  801 ACTACGTCTT TAACGTCACC TGTGATAACG CCACCTTGGA TTGCAGCTCC
  851 CATTGCCACT ACTTCGTCCG GGTTTACTCC TTTGTTAGGC TCTTTACCGA
  901 TTTCTTTTTT GACAGCTTCT TGTACTGCTG GAATACGAAT TGATCCACCA
  951 ACTAAGATAA CTTCATCGAT ATCTGANITT GTTAAGCCAG CGTCTTTCAT
1001 TGCTTGGCGT GTAGGTCCAT C
```

Mutant: NT152
Phenotype: temperature sensitivity
Sequence map: Mutant NT152 is complemented by plasmid pMP418, which carries a 3.0 kb insert of wild-type S. aureus genomic DNA. A partial restriction map is depicted in FIG. 63. Database searches at the nucleic acid and (putative) polypeptide levels against currently available databases reveal limited peptide-level similarity to yacF, a hypothetical ORF, from B. subtilis (Genbank Accession No. D26185).

DNA sequence data: The following DNA sequence data represents the sequence generated by primer walking through clone pMP418, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP4l8
SEQ ID NO. 71
pMP418 Length: 3010 nt
      1 ATGCCTGCAG GTCGATCACG ATGNAAGTCA TTCAATAAGA ATGATTATGA
    51 AAATAGAAAC AGCAGTAAGA TATTTTCTAA TTGAAAATCA TCTCACTGCT
  101 GTTTTTTAAA GGTTTATACC TCATCCTCTA AATTATTTAA AAATAATTAA
  151 TGGTATTTGA GCACGTTTAG CGACTTTATG ACTGACATTA CCAATTTCCA
  201 TTTCTTGCCA GATATTCAAA CCACGTGTAC TCAAAATGAT AGCTTGGTAT
  251 GTACCTCCAA TAGTAATTTC AATAACTTTG TCTGTTGAAC ACTAAGAGCA
  301 ATTTTAATTT CATAATGTGT TGTAAACATT TTTTTTGATT GGAGTTTTTT
  351 TCTGAGTTAA ACGATATCCT GATGTATTTT TAATTTTGCA CCATTTCCAA
  401 AAGGATAAGT GACATAAGTA AAAAGGCATC ATCGGGAGTT ATCCTATCAG
  451 GAAAACCAAG ATAATACCTA AGTAGAAAAG TGTTCAATCC GTGTTAAATT
  501 GGGAAATATC ATCCATAAAC TTTATTACTC ATACTATAAT TCAATTTTAA
  551 CGTCTTCGTC CATTTGGGCT TCAAATTCAT CGAGTARTGC TCGTGCTTCT
  601 GCAATTGATT GTGTGTTCAT CAATTGATGT CGAAGTTCGC TAGCGCCTCT
  651 TATGCCACGC ACATAGATTT TAAAGAATCT ACGCAAGCTC TTGAATTGTC
  701 GTATTTCATC TTTTTCATAT TTGTTAAACA ATGATAAATG CAATCTCAAT
  751 AGATCTAATA GTTCCTTGCT TGTGTGTTCG CGTGGTTCTT TTTCAAAAGC
  801 GAATGGATTG TGGAAAATGC CTCTACCAAT CATGACGCCA TCAATGCCAT
  851 ATTTTTCTGC CAGTTCAAGT CCTGTTTTTC TATCGGGAAT ATCACCGTTA
  901 ATTGTTAACA ATGTATTTGG TGCAATTTCG TCACGTAAAT TTTTAATAGC
  951 TTCGATTAAT TCCCAATGTG CATCTACTTT ACTCATTTCT TTACGTTGTA
1001 CGAAGATGAA TAGATAAATT GGCAATGTCT TGTTCGAAGA CAKTGCTTCA
1051 ACCAATCTTT CCATTCATCG ATTTCATAKT AGCCAAGGCG TGTTTTTAAC
1101 ACTTTACCGG AASCCCACCT GCTTTAGTCG CTTGAATAAT TTCGGCAGCA
1151 ACGTCAGGTC TTAAGATTAA GCCGGANCCC TTACCCTTTT TAGCAACATT
1201 TGCTACAGGA CATCCCATAT TTAAGTCTAT GCCTTTAAAG CCCATTTTAG
1251 CTAATTGAAT ACTCGTTTCA CGGAACTGTT CTGGCTTATC TCCCCATATA
1301 TGAGCGACCA TCGGCTGTTC ATCTTCACTA AAAGTTAAGC GTCCGCGCAC
1351 ACTATGTATG CCTTCAGGGT GGCAAAAGCT TTCAGTATTT GTAAATTCAG
1401 TGAAAAACAC ATCCRGTCTA GNTGCTTCAN TTACAACGTG TCGAAAGACG
1451 ATATCTGTAA CGTCTTCCAT TGGCGCCAAA ATAAAAAATG GACGTGGTAA
1501 TTCACTCCAA AAATTTTCTT TCATAATATA TTTATACCCT CTTTATAATT
1551 AGTATCTCGA TTTTTTATGC ATGATGATAT TACCACAAAA GCNTAACTTA
1601 TACAAAAGGA ATTTCAATAG ATGCAACCAT TKGAAAAGGG AAGTCTAAGA
1651 GTAGTCTAAA ATAAATGTTG TGGTAAGTTG ATCAATACAA AGATCAAGGA
1701 TTATAGTATT AAATTGTTCA TTATTAATGA TACACTACTT ATGAATATGA
1751 TTCAGAATTT TCTTTGGCTA CTNCTTACAG TAAAGCGACC TTTTAGTTAT
1801 CTTATAACAA AGACAAATTT CTAAAGGTGA TATTATGGAA GGTTTAAAGC
1851 ATTCTTTAAA AAGTTTAGGT TGGTGGGATT NATTTTTTGC GATACCTATT
1901 TTTCTGCTAT TCGCATACCT TCCAAACTNT AATTTTATAA NCATATTTCT
1951 TAACATTGTT ATCATTATTT TCTTTTCCNT AGGTTTGATT TTAACTACGC
2001 ATATAATTAT AGATAAAAYT AAGAGCAACA CGAAATGAAT CATTAATACG
2051 GAATGTGATT AAAACATAAA ACTGAAGGAG CGATTACAAT GGCGACTAAG
2101 AAAGATGTAC ATGATTTATT TTTAAATCAT GTGAATTCAA ACGCGGTTAA
2151 GACAAGAAAG ATGATGGGAG AATATATTAT TTATTATGAT GGCGTGGTTA
2201 TAGGTGGTTT GTATGATAAT AGATTATTGG TCAAGGCGAC TAAAAGTGCC
2251 CAGCAGAAAT TGCAAGATAA TACATTAGTT TCGCCATATC CAGGTTTCTA
2301 AAGAAATGAT ATTAATTTTA GACTTTACCG AAGCAACAAA TCTCACTGAT
2351 TTATTTAAGA CCATAAAAAA TGATTTGAAA AAGTGAAGTA GTGAAGTGTG
2401 GGTGCAGAGA GAACTAAGCC CATCGWTAAA TGGTCGCTTG TTAAAGAAGA
2451 GTGACGGTCA CTCTTCTTTA TGTGCATATT TTATTTTGTC TGTTTBGTTA
2501 ACAAGCAGCA GTGTAACAAA TATGAGTAAG GATAAAATGA GTATAATATA
2551 GAAACCGAAT TTATCATTAA TTTCATTAAT CCATCTTCCT AAAAATGGAG
2601 CAATTAAACT TTGCAGTAAC AATGAAATTG ACGTCCATAT CGTAAATGAG
2651 CGACCGACAT ATTTATCTGA AACAGTGTTC ATTATAGCWG TATTCATATA
2701 AATTCTGATT GATGAAATTG AGTAGCCTAG TATAAAKGAT CCTATGAATA
2751 AGTAAAATGC TGAGTTTATC CAAATAAATA GTGCKGAATT TATGACTRRC
2801 TATGAAATAT AACAAAAATA TCACATACTT TAGKTGAGAT TTTCTTSGAA
2851 AGAATAGCTG AAATTAAACC TGCACATAAT CCTCCAATGC CATATAACAT
2901 ATCTGAAMAA CCAAAKTGTA CAGACCGAAA GTTTTAAAAC ATTATAAACA
2951 TATCCTGGTA ATGATATGTT AAAGATCGAC TCTAGAGGAT CCCCGGNTAC
3001 CGAGCTCGAA
```

Mutant: NT156
phenotype: temperature sensitivity
Sequence map: Mutant NT156 is complemented by plasmids pMP672 and pMP679, which carry 4.5 kb inserts of wild-type S. aureus genomic DNA. A partial restriction map is depicted in FIG. 64. Database searches at the nucleic acid and (putative) polypeptide levels against currently available databases reveal identity to the grlBA locus, a known essential gene encoding DNA topoisomerase (EC 5.99.1.3), from S. aureus (Genbank Accession No. L25288; published in Ferrero, L. et al. Mol. Microbiol. 13 (1994) 641-653).

DNA sequence data: The following DNA sequence data represents the sequence generated from clone pMP679, starting with standard M13 forward and M13 reverse sequencing primers; the sequence contig will be completed later via primer walking strategies. The sequence below can be used to design PCR primers for the purpose of amplification from genomic DNA with subsequent DNA sequencing.

```
clones pMP679 and pMP672
SEQ ID NO. 72
pMP679.forward Length: 548 nt
    1 ATCGGTACCC GGGGACCAAT ANACAGAAAG TATATTAAGT TTNGTAAATA
  51 ATGTACGTAC TNAAGATGGT GGTACACATG AAGTTGGTTT TAAAACAGCA
101 ATGACACGTG TATTTAATGA TTATGCACGT CGTATTAATG AACTTAAAAC
151 AAAAGATAAA AACTTAGATG GTAATGATAT TCGTGAAGGT TTAACAGCTG
201 TTGTGTCTGT TCGTATTCCA GAAGAATTAT TGCAATTTGA ANGACAAACG
251 AAATCTAAAT TGGGTACTTC TGAAGCTAGA AGTGCTGTTG ATTCAGTTGT
301 TGCAGACAAA TTGCCATTCT ATTTAGAAGA AAAAGGACAA TTGTCTAAAT
351 CACTTGTGGA AAAAAGCGAT TAAAGCACAA CAAGCAAGGG AAGCTGCACG
401 TAAAGCTCGT GAAGATGCTC GTTCAGGTAA GAAAAACAAG CGTAAAGACA
451 CTTTGCTATC TGGTAAATTA ACACCTGCAC AAAGTTAAAA ACACTGGAAA
501 AAAATGAATT GTATTTAGTC GAAGGTGATT CTGCGGGAAG TTCAGCAA
SEQ ID NO. 73
pMP679.reverse Length: 541 nt
    1 ACTGCAGGTC GAGTCCAGAG GWCTAAATTA AATAGCAATA TTACTAAAAC
  51 CATACCAATG TAAATGATAG CCATAATCGG TACAATTAAC GAAGATGACG
101 TAGCAATACT ACGTACACCA CCAAATATAA TAATAGCTGT TACGATTGCT
151 AAAATAATAC CTGTGATTAC TGGACTAATA TTATATTGCG TATTTAACGA
201 CTCCGCAATT GTATTAGATT GCACTGTGTT AAATACAAAT GCAAATGTAA
251 TTGTAATTAA AATCGCAAAT ACGATACCTA GCCATTTTTG ATTTAAACCT
301 TTAGTAATAT AGTAAGCTGG ACCACCACGG GAATCCACCA TCTTTATCAT
351 GTACTTTATA AACCTGAGCC AAAGTCGCTT CTATAAATGC ACTCGCTGCA
401 CCTATAAATG CAATAACCCA CATCCAAAAT ACTGCACCTG GACCGCCTAA
451 AACAATCGCA GTCGCAACAC CAGCAATATT ACCAGTACCA ACTCTCGAAC
501 CAGCACTAAT CGCAAATGCT TGGAATGGCG AAATACCCTT C
SEQ ID NO. 74
pMP671 forward Length: 558 nt
    1 AGGGTCTNNC ACGGTACCCG GGGNCCAATT WGATGAGGAG GAAATCTAGT
  51 GAGTGAAATA ATKCAAGATT TATCACTTGA AGATGTTTTA GGTGATCGCT
101 TTGGAAGATA TAGTAAATAT ATTATTCAAG AGCGTGCATT GCCAGATGTT
151 CGTGATGGTT TAAAACCAGT ACAACGTCGT ATTTTATATG CAATGTATTC
201 AAGTGGTAAT ACACACGATA AAAATTTCCG TAAAAGTGCG AAAACAGTCG
251 GTGATGTTAT TGGTCAATAT CATCCACATG GGAGACTCCT CAGTGTACGA
301 AGCAATGGTC CGTTTAAGTC AAGACTGGAA GTTACGACAT GTCTTAATAG
351 AAATGCATGG TAATAATGGT AGTATCGATA ATGATCCGCC AGCGGCAATG
401 CGTTACACTG AAGCTAAGTT AAGCTTACTA GCTGAAGAGT TATTACGTGA
451 TATTAATAAA GAGACAGTTT CTTTCATTCC AAACTATGAT GATACGACAC
501 TCCGAACCAA TGGTATTGCC ATCAAGAATT TCCTAACTTA CTAAKTGAAT
551 GGTTCTAC
```

Mutant: NT160
Phenotype: temperature sensitivity
Sequence map: Mutant NT160 is complemented by plasmid pMP423, which carries a 2.2 kb insert of wild-type S. aureus genomic DNA. A partial restriction map is depicted in FIG. 65. Database searches at the nucleic acid and (putative) polypeptide levels against currently available databases reveal identity to the Dlt locus of S. aureus (Genbank Accession No. D86240; unpublished). The pMP423 clone completely contains the genes dltC, encoding a putative D-Alanine carrier protein, and dltD, encoding a putative “extramembranal protein”. Further subcloning and recomplementation experiments already in progress will demonstrate whether one or both of the ORFs encode essential genes.

DNA sequence data: The following DNA sequence data represents the sequence generated by primer walking through clone pMP423, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP423
SEQ ID NO. 75
pMP423 Length: 2234 nt
      1 AGTCGATCTT TATTCTACAT GTCTCGTAAA AAATTATTGA AGAGTCAATT
    51 TGCAATGTCT AACGTGGCAT TCTTAATCAA CTTCTTCATA ATGGGAATTT
  101 GGCATGGTAT CGAAGTGTAT TACATTGTTT ATGGTTTATA CCATGCAGCA
  151 TTGTTTATAG GTTATGGCTA TTATGAACGT TGGCGTAAGA AACATCCGCC
  201 ACGTTGGCAA AATGGTTTCA CAACAGCACT TAGCATTGTG ATTACATTCC
  251 ACTTTGTAAC ATTTGGCTTT TTAATCTTCT CAGGTAAACT TATATAATAA
  301 AGGAGAATTT AATTATGGAA TTTAGAGAAC AAGTATTAAA TTTATTAGCA
  351 GAAGTAGCAG AAAAATGATA TTGTAAAAGA AAATCCAGAC GTAGAAATTT
  401 TTGAAGAAGG TATTATTGAT TCTTTCCAAA CAGTTGGATT ATTATTAGAG
  451 ATTCAAAATA AACTTGATAT CGAAGTATCT ATTATGGACT TTGATAGAAG
  501 ATGAGTGGGC MACACCAAAT AAAATCGTTG AAGCATTAGA AGAGTTACGA
  551 TGAAATTAAA ACCTTTTTTA CCCATTTTAA TTAGTGGAGC GGTATTCATT
  601 GTCTTTCTAT TATTACCTGC TAGTTGGTTT ACAGGATTAG TAAATGAAAA
  651 GACTGTAGAA GATAATAGAA CTTCATTGAC AGATCAAGTA CTAAAAGGCA
  701 CACTCAWTCA AGATAAGTTA TACGAATCAA ACAAGTATTA TCCTATATAC
  751 GGCTCTAGTG AATTAGGTAA AGATGACCCA TTTAATCCTG CAATTGCATT
  801 AAATAAGCAT AACGCCAACA AAAAAGCATT CTTATTAGGT GCTGGTGGTT
  851 CTACAGACTT AATTAACGCA GTTGAACTTG CATCACAGTT ATGATAAATT
  901 AAAAGGTTAA GAAATTAACA TTTATTATTT CACCACAATG GTTTACAAAC
  951 CCATGGTTTA ACGAATCCAA AACTTTGATG CTCSTATGTC TCAAACTCMA
1001 ATTAATCAAA TGTTCCCASC AGAAAAACAT GTCTACTGAA TTAAAACGTC
1051 GTTATGCACA ACGTTTATTA CAGTTTCCAC ATGTACACAA TAAAGAATAC
1101 TTGAAATCTT ATGCTAAAAA CCCTAAAGAA ACTAAAGRTA GTTATATTTC
1151 TGGKTTTWAA RAGAGATCAA TTGATTAAAA TAGAAGCGAT TAAATCATTG
1201 TTTGCAATGG ATAAATCTCC ATTAGAACAT GTTAAACCCT GCTACAAAAC
1251 CAGACGCTTC TTGGGATGAG ATGAAACAAA AAGCAGTTGA AATTGGTAAA
1301 GCTGATACTA CATCGAATAA ATTTGGTATT AGAGATCAAT ACTGGAAATT
1351 AATTCCAAGA AAGTAAGCCG TTAAAGTTAG ACGTTGACTA CGAATTCMAT
1401 GTTWATTCTC CCAGAATTCC MAGATTTAGA ATTACTTGTW AAAAMMATGC
1451 KTGCTGCTGG TGCAGATGTT CAATATGTAA GTATTCCATC AAACGGTGTA
1501 TGGTATGACC ACATTGGTAT CGATAAAGAA CGTCGTCAAG CAGTTTATAA
1551 AAAAATCCAT TCTACTGTTG TAGATAATGG TGGTAAAATT TACGATATGA
1601 CTGATAAAGA TTATGAAAAA TATGTTATCA GTGATGCCGT ACACATCGGT
1651 TGGAAAGGTT GGGTTTATAT GGATGAGCAA ATTGCGAAAC ATATGAAAGG
1701 TGAACCACAA CCTGAAGTAG ATAAACCTAA AAATTAAAAT ACAAATAGCA
1751 CATAACTCAA CGATTTTGAT TGAGCGTATG TGCTATTTTT ATATTTTAAA
1801 TTTCATAGAA TAGAATAGTA ATATGTGCTT GGATATGTGG CAATAATAAA
1851 ATAATTAATC AGATAAATAG TATAAAATAA CTTTCCCATC AGTCCAATTT
1901 GACAGCGAAA AAAGACAGGT AATAACTGAT TATAAATAAT TCAGTATTCC
1951 TGTCTTTGTT GTTATTCATA ATATGTTCTG TTAACTTAAT ATCTTTATAT
2001 TAGAATACTT GTTCTACTTC TATTACACCA GGCACTTCTT CGTGTAATGC
2051 ACGCTCAATA CCAGCTTTAA GAGTGATTGT AGAACTTGGG CATGTACCAC
2101 ATGCACCATG TAATTGTAAT TTAACAATAC CGTCTTCCAC GTCAATCAAT
2151 GAGCAGTCGC CACCATCACG TAATAAAAAT GGACGAAGAC GTTCAATAAC
2201 TTCTGCTACT TGATCGACCT GCAGGCATGC AAGC
```

Mutant: NT166
Phenotype: temperature sensitivity
Sequence map: Mutant NT166 is complemented by plasmid pMP425, which carries a 3.3 kb insert of wild-type S. aureus genomic DNA. A partial restriction map is depicted in FIG. 66. Database searches at the nucleic acid and (putative) polypeptide levels against currently available databases reveal strong peptide-level similarities to nrdE, encoding ribonucleotide diphosphate reductase II (EC 1.17.4.1), from B. subtilis(Genbank Accession No. Z68500), and ymaA, a hypothetical ORF, from B. subtilis (same Genbank entry).

DNA sequence data: The following DNA sequence data represents the sequence generated by primer walking through clone pMP425, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP425
SEQ ID NO. 76
pMP425 Length: 3305 nt
      1 GAGCTCGGTA CCCGGGGATC CTCTAGAGTC GATCCAATGA AAATAATATA
    51 TTTTTCATTT ACTGGAAATG TCCGTCGTTT TATTAAGAGA ACAGAACTTG
  101 AAAATACGCT TGAGATTACA GCAGAAAATT GTATGGAACC AGTTCATGAA
  151 CCGTTTATTA TCGTTACTGG CACTATTGGA TTTGGAGAAG TACCAGAACC
  201 CGTTCAATCT TTTTTAGAAG TTAATCATCA ATACATCAGA GGTGTGGCAG
  251 CTAGCGGTAA TCGAAATTGG GGACTAAATT TCGCAAAAGC GGGTCGCACG
  301 ATATCAGAAG AGTATAATGT CCCTTTATTA ATGAAGTTTG AGTTACATGG
  351 GAAAAAACAA AGACGTTATT GAATTTAAGA ACAAGGTGGG TAATTTTAAT
  401 GAAAACCATG GAAGAGAAAA AGTACAATCA TATTGAATTA AATAATGAGG
  451 TCACTAAACG AAGAGAAGAT GGATTCTTTA GTTTAGAAAA AGACCAAGAA
  501 GCTTTAGTAG CTTATTTAGA AGAAGTAAAA GACAAAACAA TCTTCTTCGA
  551 CACTGAAATC GAGCGTWTAC GTTMTTTAGT AGACMACGAT TTTTATTTCA
  601 ATGTGTTTGA TATWTATAGT GAAGCGGATC TAATTGAAAT CACTGATTAT
  651 GCAAAATCAA TCCCGTTTAA TTTTGCAAGT TATATGTCAG CTAGTAAATT
  701 TTTCAAAGAT TACGCTTTGA AAACAAATGA TAAAAGTCAA TACTTAGAAG
  751 ACTATAATCA ACACGTTGCC ATTGTTGCTT TATACCTAGC AAATGGTAAT
  801 AAAGCACAAG CTAAACAATT TATTTCTGCT ATGGTTGAAC AAAGATATCA
  851 ACCAGCGACA CCAACATTTT TAAACGCAGG CCGTGCGCGT TCGTGGTGGA
  901 GCTAGTGTTC ATTGTTTCCT TATTAGAAGT TGGATGGACA GCTTAAATTC
  951 AATTTAACTT TATTGGATTC AACTGCAAAA CAATTAAGTW AAATTGGGGG
1001 CGGSGTTTGC MATTAACTTA TCTAAATTGC GTGCACGTGG TGAAGCAATT
1051 AAAGGAATTA AAGGCGTAGC GAAAGGCGTT TTACCTATTG CTAAGTCACT
1101 TGAAGGTGGC TTTAGCTATG CAGATCAACT TGGTCAACGC CCTGGTGCTG
1151 GTGCTGTGTA CTTAAATATC TTCCATTATG ATGTAGAAGA ATTTTTAGAT
1201 ACTAAAAAAG TAAATGCGGA TGAAGATTTA CGTTTATCTA CAATATCAAC
1251 TGGTTTAATT GTTCCATCTA AATTCTTCGA TTTAGCTAAA GAAGGTAAGG
1301 ACTTTTATAT GTTTGCACCT CATACAGTTA AAGAAGAATA TGGTGTGACA
1351 TTAGACGATA TCGATTTAGA AAAATATTAT GATGACATGG TTGCAAACCC
1401 AAATGTTGAG AAAAAGAAAA AGAATGCGCG TGAAATGTTG AATTTAATTG
1451 CGCMAACACA ATTACAATCA GGTTATCCAT ATTTAATGTT TAAAGATAAT
1501 GCTAACAGAG TGCATCCGAA TTCAAACATT GGACAAATTA AAATGAGTAA
1551 CTTATGTACG GAAATTTTCC AACTACAAGA AACTTCAATT ATTAATGACT
1601 ATGGTATTGA AGACGAAATT AAACGTGATA TTTCTTGTAA CTTGGGCTCA
1651 TTAAATATTG TTAATGTAAT GGAAAGCGGA AAATTCAGAG ATTCAGTTCA
1701 CTCTGGTATG GACGCATTAA CTGTTGTGAG TGATGTAGCA AATATTCAAA
1751 ATGCACCAGG AGTTAGAAAA GCTAACAGTG AATTACATTC AGTTGKTCTT
1801 GGGTGTGATG AATTWACACG GTTACCTAGC AAAAAATAAA ATTGGTTATG
1851 AGTCAGAAGA AGCAAAAGAT TTTGCAAATA TCTTCTTTAT GATGATGAAT
1901 TTCTACTCAA TCGAACGTTC AATGGAAATC GCTAAAGAGC GTGGTATCAA
1951 ATATCAAGAC TTTGAAAAGT CTGATTATGC TAATGGCAAA TATTTCGAGT
2001 TCTATACAAC TCAAGAATTT GAACCTCAAT TCGAAAAAGT ACGTGAATTA
2051 TTCGATGGTA TGGCTATTCC TACTTCTGAG GATTGGAAGA AACTACAACA
2101 AGATGTTGAA CAATATGGTT TATATCATGC ATATAGATTA GCAATTGCTC
2151 CAACACAAAG TATTTCTTAT GTTCAAAATG CAACAAGTTC TGTAATGCCA
2201 ATCGTTGACC AAATTGAACG TCGTACTTAT GGTAAATGCG GAAACATTTT
2251 ACCCTATGCC ATTCTTATCA CCACAAACAA TGTGGTACTA CAAATCAGCA
2301 TTCAATACTG ATCAGATGAA ATTAATCGAT TTAATTGCGA CAATTCAAAC
2351 GCATATTGAC CAAGGTATCT CAACGATCCT TTATGTTAAT TCTGAAATTT
2401 CTACACGTGA GTTAGCAAGA TTATATGTAT ATGCGCACTA TAAAGGATTA
2451 AAATCACTTT ACTATACTAG AAATAAATTA TTAAGTGTAG AAGAATGTAC
2501 AAGTTGTTCT ATCTAACAAT TAAATGTTGA AAATGACAAA CAGCTAATCA
2551 TCTGGTCTGA ATTAGCAGAT GATTAGACTG CTATGTCTGT ATTTGTCAAT
2601 TATTGAGTAA CATTACAGGA GGAAATTATA TTCATGATAG CTGTTAATTG
2651 GAACACACAA GAAGATATGA CGAATATGTT TTGGAGACAA AATATATCTC
2701 AAATGTGGGT TGAAACAGAA TTTAAAGTAT CAAAAGACAT TGCAAGTTGG
2751 AAGACTTTAT CTGAAGCTGA ACAAGACACA TTTAAAAAAG CATTAGCTGG
2801 TTTAACAGGC TTAGATACAC ATCAAGCAGA TGATGGCATG CCTTTAGTTA
2851 TGCTACATAC GACTGACTTA AGGAAAAAAG CAGTTTATTC ATTTATGGCG
2901 ATGATGGAGC AAATACACGC GAAAAGCTAT TCACATATTT TCACAACACT
2951 ATTACCATCT AGTGAAACAA ACTACCTATT AGATGAATGG GTTTTAGAGG
3001 AACCCCATTT AAAATATAAA TCTGATAAAA TTGTTGCTAA TTATCACAAA
3051 CTTTGGGGTA AAGAAGCTTC GATATACGAC CAATATATGG CCAGAGTTAC
3101 GAGTGTATTT TTAGAAACAT TCTTATTCTT CTCAGGTTTC TATTATCCAC
3151 TATATCTTGC TGGTCAAGGG AAAATGACGA CATCAGGTGA AATCATTCGT
3201 AAAATTCTTT TAGATGAATC TATTCATGGT GTATTTACCG GTTTAGATGC
3251 ACAGCATTTA CGAAATGAAC TATCTGAAAG TGAGAAACAA AAAGCAGATC
3301 GACCT
```

Mutant: NT 199
Phenotype: temperature sensitivity
Sequence map: Mutant NT199 is complemented by plasmid pMP642, which carries a 3.6 kb insert of wild-type S. aureus genomic DNA. A partial restriction map is depicted in FIG. 67. Database searches at the nucleic acid and (putative) polypeptide levels against currently available databases reveal strong peptide-level similarities to yybQ, an uncharacterized ORFs identified in B. subtilis from genomic sequencing efforts.

DNA sequence data: The following DNA sequence data represents the sequence generated by primer walking through clone pMP642, starting with standard M13 forward and M13 reverse sequencing primers and, completing the sequence contig via primer walking strategies. The sequence below can be used to design PCR primers for the purpose of amplification from genomic DNA with subsequent DNA sequencing.

```
clone pMP642
SEQ ID NO. 77
pMP642 Length: 1945 nt
      1 TTGATAGTTT ATTGGAGAGA AAGAAGTATT AATCAAGTCG AAATCGTTGG
    51 TGTATGTACC GATATTTGCG TGTTACATAC AGCAATTTCT GCATACAACT
  101 TAGGTTATAA AATTTCAGTA CCTGCTGAGG GAGTGGCTTC ATTTAATCAA
  151 AAAGGGCATG AATGGGCACT TGCACATTTC AAAAACTCAT TAGGTGCAGA
  201 GGTAGAACAA CACGTTTAAA TCGTGCTAAA ATAATTATAA AGAATACAAT
  251 TTACAAGGGA GATATTTGAC AATGGCTAAA ACATATATTT TCGGACATAA
  301 GAATCCAGAC ACTGATGCAA TTTCATCTGC GATTATTATG GCAGAATTTG
  351 AACAACTTCG AGGTAATTCA GGAGCCAAAG CATACCGTTT AGGTGATGTG
  401 AGTGCARAAA CTCAATTCGC GTTAGATACA TTTAATGTAC CTGCTCCGGA
  451 ATTATTAACA GATGATTTAG ATGGTCAAGA TGTTATCTTA GTTGATCATA
  501 ACGAATTCCA ACAAAGTTCT GATACGATTG CCTCTGCTAC AATTAAGCAT
  551 GTAATTGATC ATCACAGAAT TGCAAATTTC GAAACTGCTG GTCCTTTATG
  601 TTATCGTGCT GAACCAGTTG GTTGTACAGC TACAATTTTA TACAAAATGT
  651 TTAGAGAACG TGGCTTTGAA ATTAAACCTG AAATTGCCGG TTTAATGTTA
  701 TCAGCAATTA TCTCAGATAG CTTACTTTTC AAATCACAAC ATGTACACAA
  751 CAAGATGTTA AAGCAGCTGA AGAATTAAAA GATATTGCTA AAGTTGATAT
  801 TCAAAAGTAC GGCTTAGATA TGTTAAAAGC AGGTGCTTCA ACAACTGATA
  851 AATCAGTTGA ATTCTTATTA AACATGGATG CTAAATCATT TACTATGGGT
  901 GACTATGKGA YTCGTATTGC AACAAGTTAA TGCTGTTGAC CTTGACGAAG
  951 TGTTAAWTCG TAAAGAAGAT TTAGAAAAAG AAATGTTAGC TGTAAGTGCA
1001 CAAGAAAAAT ATGACTTATT TGTACTTGTT GTTACKGACA TCATTAATAG
1051 TGATTCTAAA ATTTTAGTTG TAGGTGCTGA AAAAGATAAA GTTGGCGAAG
1101 CATTCAATGT TCAATTAGAA GATGACATGG CCYTCTTATC TGGTGTCGTW
1151 TCTCGAAAAA AACAAATCGT ACCTCAAATC ACTGAAGCAT TAACAAAATA
1201 ATACTATATT ACTGTCTAAT TATAGACATG TTGTATTTAA CTAACAGTTC
1251 ATTAAAGTAG AATTTATTTC ACTTTCCAAT GAACTGTTTT TTATTTACGT
1301 TTGACTAATT TACAACCCTT TTTCAATAGT AGTTTTTATT CCTTTAGCTA
1351 CCCTAACCCA CAGATTAGTG ATTTCTATAC AATTCCCCTT TTGTCTTAAC
1401 ATTTTCTTAA AATATTTGCG ATGTTGAGTA TAAATTTTTG TTTTCTTCCT
1451 ACCTTTTTCG TTATGATTAA AGTTATAAAT ATTATTATGT ACACGATTCA
1501 TCGCTCTATT TTCAACTTTC AACATATATA ATTCGAAAGA CCATTTAAAA
1551 TTAACGGCCA CAACATTCAA ATCAATTAAT CGCTTTTTCC AAAATAATCA
1601 TATAAGGAGG TTCTTTTCAT TATGAATATC ATTGAGCAAA AATTTTATGA
1651 CAGTAAAGCT TTTTTCAATA CACAACAAAC TAAAGATATT AGTTTTAGAA
1701 AAGAGCAATT AAAGAAGTTA AGCAAAGCTA TTAAATCATA CGAGAGCGAT
1751 ATTTTAGAAG CACTATATAC AGATTTAGGA AAAAATAAAG TCGAAGCTTA
1801 TGCTACTGAA ATTGGCATAA CTTTGAAAAG TATCAAAATT GCCCGTAAGG
1851 AACTTAAAAA CTGGACTAAA ACAAAAAATG TAGACACACC TTTATATTTA
1901 TTTCCAACAA AAAGCTATAT CAAAAAAGAA CCTTATGGAA CAGTT
```

Mutant: NT 201
Phenotype: temperature sensitivity
Sequence map: Mutant NT201 is complemented by plasmid pMP269, which carries a 2.6 kb insert of wild-type S. aureus genomic DNA. A partial restriction map is depicted in FIG. 68. Database searches at the nucleic acid and (putative) polypeptide levels against currently available databases reveal strong peptide-level similarity to ylxC, encoding a putative murB homolog (UDP-N-acetylenolpyruvoylglucosamine reductase), in B. subtilis (Genbank Accession No. M31827). The predicted relative size and orientation of the ylxC gene is depicted by an arrow in the map.

DNA sequence data: The following DNA sequence data represents the sequence generated by primer walking through clone pMP269, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP269
SEQ ID NO. 78
pMP269 Length: 2590 nt
      1 TCGAACTCGG TACCCGGGGA TCCTCTAGAG TCGATCAACT ACAACTACAA
    51 TTAAACAAAT TGAGGAACTT GATAAAGTTG TAAAATAATT TTAAAAGAGG
  101 GGAACAATGG TTAAAGGTCT TAATCATTGC TCCCCTCTTT TCTTTAAAAA
  151 AGGAAATCTG GGACGTCAAT CAATGTCCTA GACTCTAAAA TGTTCTGTTG
  201 TCAGTCGTTG GTTGAATGAA CATGTACTTG TAACAAGTTC ATTTCAATAC
  251 TAGTGGGCTC CAAACATAGA GAAATTTGAT TTTCAATTTC TACTGACAAT
  301 GCAAGTTGGC GGGGCCCAAA CATAGAGAAT TTCAAAAAGG AATTCTACAG
  351 AAGTGGTGCT TTATCATGTC TGACCCACTC CCTATAATGT TTTGACTATG
  401 TTGTTTAAAT TTCAAAATAA ATATGATAGT GATATTTACA GCGATTGTTA
  451 AACCGAGATT GGCAATTTGG ACAACGCTCT ACCATCATAT ATTCATTGAT
  501 TGTTAATTCG TGTTTGCATA CACCGCATAA GATTGCTTTT TCGTTAAATG
  551 AAGGCTCAGA CCAACGCTTA ATGGCGTGCT TTTCAAACTC ATTATGGCAC
  601 TTATAGCATG GATAGTATTT ATTACAACAT TTAAATTTAA TAGCAATAAT
  651 ATCTTCTTCG GTAAAATAAT GGCGACAGCG TGTTTCAGTA TCGATTAATG
  701 AACCATAAAC TTTAGGCATA GACAAAGCTC CTTAACTTAC GATTCCTTTG
  751 GATGTTCACC AATAATGCGA ACTTCACGAT TTAATTCAAT GCCAAWTTTT
  801 TCTTTGACGG TCTTTTGTAC ATAATGAATA AGGTTTTCAT AATCTGTAGC
  851 AGTTCCATTG TCTACATTTA CCATAAAACC AGCGTGTTTG GTTGAAACTT
  901 CAACGCCGCC AATACGGTGA CCTTGCAAAT TAGAATCTTG TATCAATTTA
  951 CCTGCAAAAT GACCAGGCGG TCTTTGGAAT ACACTACCAC ATGAAGGATA
1001 CTCTAAAGGT TGTTTAAATT CTCTACGTTC TGTTAAATCA TCCATTTTAG
1051 CTTGTATTTC AGTCATTTTA CCAGGAGCTA AAGTAAATGC AGCTTCTAAT
1101 ACAACTAANT GTTCTTTTTG AATAATGCTA TTACNATAAT CTAACTCTAA
1151 TTCTTTTGTT GTAAGTTTAA TTAACGAGCC TTGTTCGTTT ACGCAAAGCG
1201 CATRGTCTAT ACAATCTTTA ACTTCGCCAC CATAAGCGCC AGCATTCATA
1251 TACACTGCAC CACCAATTGA ACCTGGAATA CCACATGCAA ATTCAAGGCC
1301 AGTAAGTGCG TAATCACGAG CAACACGTGA GACATCAATA ATTGCAGCGC
1351 CGCTACCGGC TATTATCGCA TCATCAGATA CTTCCGATAT GATCTAGTGA
1401 TAATAAACTA ATTACAATAC CGCGAATACC ACCTTCACGG ATAATAATAT
1451 TTGAGCCATT TCCTAAATAT GTAACAGGAA TCTCATTTTG ATAGGCATAT
1501 TTAACAACTG CTTGTACTTC TTCATTTTTA GTAGGGGTAA TGTAAAAGTC
1551 GGCATTACCA CCTGTTTTAG TATAAGTGTA TCGTTTTAAA GGTTCATCAA
1601 CTTTAATTTT TTCAKTYGRS MTRARKKSWT GYAAAGCTTG ATAGATGTCT
1651 TTATTTATCA CTTCTCAGTA CATCCTTTCT CATGTCTTTA ATATCATATA
1701 GTATTATACC AATTTTAAAA TTCATTTGCG AAAATTGAAA AGRAAGTATT
1751 AGAATTAGTA TAATTATAAA ATACGGCATT ATTGTCGTTA TAAGTATTTT
1801 TTACATAGTT TTTCAAAGTA TTGTTGCTTT TGCATCTCAT ATTGTCTAAT
1851 TGTTAAGCTA TGTTGCAATA TTTGGTGTTT TTTTGTATTG AATTGCAAAG
1901 CAATATCATC ATTAGTTGAT AAGAGGTAAT CAAGTGCAAG ATAAGATTCA
1951 AATGTTTGGG TATTCATTTG AATGATATGT AGACGCACCT GTTGTTTTAG
2001 TTCATGAAAA TTGTTAAACT TCGCCATCAT AACTTTCTTA GTATATTTAT
2051 GATGCAAACG ATAAAACCCT ACATAATTTA AGCGTTTTTC ATCTAAGGAT
2101 GTAATATCAT GCAAATTTTC TACACCTACT AAAATATCTA AAATTGGCTC
2251 TGTTGAATAT TTAAAATGAT GCGTACCGCC AATATGTTTT GTATATTTTA
2201 CTGGGCTGTC TAAGAGGTTG AATAATAATG ATTCAATTTC AGTGTATTGT
2251 GATTGAAAAC AATTAGTTAA ATCACTATTA ATGAATGGTT GAACATTTGA
2301 ATACATGATA AACTCCTTTG ATATTGAAAA TTAATTTAAT CACGATAAAG
2351 TCTGGAATAC TATAACATAA TTCATTTTCA TAATAAACAT GTTTTTGTAT
2401 AATGAATCTG TTAAGGAGTG CAATCATGAA AAAAATTGTT ATTATCGCTG
2451 TTTTAGCGAT TTTATTTGTA GTAATAAGTG CTTGTGGTAA TAAAGAAAAA
2501 GAGGCACAAC ATCMATTTAC TAAGCAATTT AAAGATGTTG AGCAAACACA
2551 WAAAGAATTA CAACATGTCA TGGATAATAT ACATTTGAAA
```

Mutant: NT304
Phenotype: temperature sensitivity
Sequence map: Mutant NT304 is complemented by plasmid pMP450, which carries a 3.3 kb insert of wild-type S. aureus genomic DNA. A partial restriction map is depicted in FIG. 69. Database searches at the nucleic acid and (putative) polypeptide levels against currently available databases reveal strong peptide-level similarities from the left-most contig below and the dod gene product, encoding pentose-5-phosphate epimerase (EC 5.1.3.1), from S. oleraceae (Genbank Accession No. L42328).

DNA sequence data: The following DNA sequence data represents the sequence generated from clone pMP450, starting with standard M13 forward and M13 reverse sequencing primers; the sequence contig will be completed via primer walking strategies. The sequence below can be used to design PCR primers for the purpose of amplification from genomic DNA with subsequent DNA sequencing.

```
clone pMP450
SEQ ID NO. 79
pMP450.forward Length: 1019 nt
      1 ATTCGAGCTC GGTACCCGGG GATCCTCTAG AGTCGCTCGA TAACTTCTAT
    51 ATGAACATCA TGTTTATAAT ATGCTTTTTT CAATAATAAC TGAATTGCCC
  101 CAAAAAAGTG ATCTAATCGT CCGCCTGTTG CACCATAATT TGTAATACTA
  151 TCAAATCCAA GTGCAACAGC TTTATCAACC GCTAAAGCTA AATCCGTATC
  201 AGCTTTTTCA GCTTGAACTG GTTTGATTTG TAACTGTTCT GTTAGAAGTT
  251 GGCGTTCTTC TTTACTGACT GAATCAAAGT CTCCCACTGA GAAAAAAGGG
  301 ATAATTTGAT GCTTCAATAA AATCAAAGCA CCTCTATCAA CGCCGCCCCA
  351 TTTACCTTCA TTACTTTTGG CCCAAATATC TTGCGGCAAG TGTCGATCAG
  401 AACATAATAA ATTTATATGC ATATACACTC AACCTTTCAA TGCTTGTGTT
  451 GACTTTTTTA TAATCCTCTT GTTTAAAGAA AAATGAACCT GTTACTAGCA
  501 TTGTTAGCAC CATTTTCAAC ACAAACTTTC GCTGTTATCG GTATTTACGC
  551 CTCCATCAAC TTCAATATCA AAGTTTAATT GACGTTCCAT TTTAATAGCA
  601 TTAAGACCCG CTATTTTTTC TACGCATTGA TCAATAAATG ATTGACCACC
  651 AAACCCTGGG TTAACTGTCA TCACTAGTAC ATAATCAACA ATGTCTAAAA
  701 TAGGTTCAAT TTGTGATATT GGTGTACCAG GATTAATTAC TACACCAGCT
  751 TTTTTATCTA AATGTTTAAT CATTTGAATA GCACGATGAA ATATGAGGCG
  801 TTGATTCGAC ATGAATTGNA AATCATATCG GCACCATGTT CTGCAAATGA
  851 TGCAATATAC TTTTCTGGAA TTTTCAATCA TCAAATGTAC GTCTATANGT
  901 AATGTTGTGC CTTTTCTTAC TGCATCTAAT ATTGGTAAAC CAATAGATAT
  951 ATTAGGGACA AATTGACCAT CCATAACATC AAAATGAACT CCGTCGAANC
1001 CCGGCTTCTC CAGTCGTTT
SEQ ID NO. 80
pMP450.reverse Length: 1105 nt
      1 CNTGCATGCC TGCAGGTCGA TCTANCAAAG CATATTAGTG AACATAAGTC
    51 GAATCAACCT AAACGTGAAA CGACGCAAGT ACCTATTGTA AATGGGCCTG
  101 CTCATCATCA GCAATTCCAA AAGCCAGAAG GTACGGTGTA CGAACCAAAA
  151 CCTAAAAAGA AATCAACACG AAAGATTGTG CTCTTATCAC TAATCTTTTC
  201 GTTGTTAATG ATTGCACTTG TTTCTTTTGT GGCAATGGCA ATGTTTGGTA
  251 ATAAATACGA AGAGACACCT GATGTAATCG GGAAATCTGT AAAAGAAGCA
  301 GAGCAAATAT TCAATAAAAA CAACCTGAAA TTGGGTAAAA TTTCTAGAAG
  351 TTATAGTGAT AAATATCCTG AAAATGAAAT TATTAAGACA ACTCCTAATA
  401 CTGGTGAACG TGTTGAACGT GGTGACAGTG TTGATGTTGT TATATCAAAG
  451 GGSCCTGAAA AGGTTAAAAT GCCAAATGTC ATTGGTTTAC CTAAGGAGGA
  501 AGCCTTGCAG AAATTAAAAT CCGTTAGGTC TTAAAGATGT TACGATTGAA
  551 AAAGTWTATA ATAATCCAAG CGCCMAAAGG ATACATTGCA AATCAAAKTG
  601 TTAMCCGCAA ATACTGAAAT CGCTATTCAT GATTCTAATA TTAAACTATA
  651 TGAATCTTTA GGCATTAAGC AAGTTTATGT AGAAGACTTT GAACATAAAT
  701 CCTTTAGCAA AGCTAAAAAA GCCTTAGAAG AAAAAGGGTT TAAAGTTGAA
  751 AGTAAGGAAG AGTATAGTGA CGATATTGAT GAGGGTGATG TGATTTCTCA
  801 ATCTCCTAAA GGAAAATCAG TAGATGAGGG GTCAACGATT TCATTTGTTG
  851 TTTCTAAAGG TAAAAAAAGT GACTCATCAG ATGTCNAAAC GACAACTGAA
  901 TCGGTAGATG TTCCATACAC TGGTNAAAAT GATAAGTCAC AAAAAGTTCT
  951 GGTTTATCTT NAAGATAANG ATAATGACGG TTCCACTGAA AAAGGTAGTT
1001 TCGATATTAC TAATGATCAC GTTATAGACA TCCTTTAAGA ATTGAAAAAG
1051 GGAAAACGCA GTTTTATTGT TAAATTGACG GTAAACTGTA CTGAAAAAAA
1101 NTCGC
```

Mutant: NT 310
Phenotype: temperature sensitivity
Sequence map: Mutant NT310 is complemented by plasmid pMP364, which carries a 2.4 kb insert of wild-type S. aureus genomic DNA. A partial restriction map is depicted in FIG. 70; there are no apparent restriction sites for EcoR I, BamH I, HinD III or Pst I. Database searches at the nucleic acid and (putative) polypeptide levels against currently available databases reveal strong similarities to the ddlA gene product from E. hirae, which encodes D-Ala-D-Ala ligase (EC 6.3.2.4); similarities are also noted to the functionally-similar proteins VanA and VanB from E. faecium and the VanC protein from E. gallinarum. The predicted relative size and orientation of the ddlA gene is depicted by an arrow in the restriction map.

DNA sequence data: The following DNA sequence data represents the sequence generated by primer walking through clone pMP364, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP364
SEQ ID NO. 81
pMP364 Length: 2375 nt
      1 AATATGACAG AACCGATAAA GCCAAGTTCC TCTCCAATCA CTGAAAAGAT
    51 AAAGTCAGTA TGATTTTCAG GTATATAAAC TTCACCGTGA TTGTATCCTT
  101 TACCTAGTAA CTGTCCAGAA CCGATAGCTT TAAGTGATTC AGTTAAATGA
  151 TAGCCATCAC CACTACTATA TGTATAGGGG TCAAGCCATG AATTGATTCG
  201 TCCCATTTGA TACAGTTGGA CACCTAATAA ATTTTCAATT AATGCGGGTG
  251 CATATAGAAT ACCTAAAATG ACTGTCATTG CACCAACAAT ACCTGTAATA
  301 AAGATAGGTG CTAAGATACG CCATGTTATA CCACTTACTA ACATCACACC
  351 TGCAATAATA GCAGCTAATA CTAATGTAGT TCCTAGGTCA TTTTGCAGTA
  401 ATATTAAAAT ACTTGGTACT AACGAGACAC CAATAATTTT GAAAAATAAT
  451 AACAAATCAC TTTGGAATGA TTTATTGAAT GTGAATTGAT TATGTCTAGA
  501 AACGACACGC GCTAATGCTA AAATTAAAAT AATTTTCATG AATTCAGATG
  551 GCTGAATACT GATAGGGCCA AACGTGTTYC AACTTTTGGC ACCATTGATA
  601 ATAGGTGTTA TAGGTGACTC AGGAATAACG AACCAGCCTA TTWATAWTAG
  651 ACAGATTAAG AAATACAATA AATATGTATA ATGTTTAATC TTTTTAGGTG
  701 AAATAAACAT GATGATACCT GCAAAAATTG CACCTAAAAT GTAATAAAAA
  751 ATTTGTCTGA TACCGAAATT AGCACTGTAT TGACCACCGC CCATTGCCGA
  801 GTTAATAAGC AGAACACTGA AAATTGCTAA AACAGCTATA GTGGCTACTA
  851 ATACCCAGTC TACTTTGCGA AGCCAATGCT TATCCGGCTG TTGACGAGAT
  901 GAATAATTCA TTGCAAACTC CTTTTATACT CACTAATGTT TATATCAATT
  951 TTACATGACT TTTTAAAAAT TAGCTAGAAT ATCACAGTGA TATCAGCYAT
1001 AGATTTCAAT TTGAATTAGG AATAAAATAG AAGGGAATAT TGTTCTGATT
1051 ATAAATGAAT CAACATAGAT ACAGACACAT AAGTCCTCGT TTTTAAAATG
1101 CAAAATAGCA TTAAAATGTG ATACTATTAA GATTCAAAGA TGCGAATAAA
1151 TCAATTAACA ATAGGACTAA ATCAATATTA ATTTATATTA AGGTAGCAAA
1201 CCCTGATATA TCATTGGAGG GAAAACGAAA TGACAAAAGA AAATATTTGT
1251 ATCGTTTTTG GAGGGAAAAG TGCAGAACAC GAAGTATCGA TTCTGACAGC
1301 AYWAAATGTA TTAAATGCAR TAGATAAAGA CAAATATCAT GTTGATATCA
1351 TTTATATTAC CAATGATGGT GATTGGAGAA AGCAAAATAA TATTACAGCT
1401 GAAATTAAAT CTACTGATGA GCTTCATTTA GAAAAATGGA GAGGCGCTTG
1451 AGATTTCACA GCTATTGAAA GAAAGTAGTT CAGGACAACC ATACGATGCA
1501 GTATTCCCAT TATTACATGG TCCTAATGGT GAAGATGGCA CGATTCAAGG
1551 GCTTTTTGAA GTTTTGGATG TACCATATGT AGGAAATGGT GTATTGTCAG
1601 CTGCAAGTTT CTATGGACAA ACTTGTAATG AAACAATTAT TTGAACATCG
1651 AGGGTTACCA CAGTTACCTT ATATTAGTTT CTTACGTTCT GAATATGAAA
1701 AATATGAACA TAACATTTTA AAATTAGTAA ATGATAAATT AAATTACCCA
1751 GTCTTTGTTA AACCTGCTAA CTTAGGGTCA AGTGTAGGTA TCAGTAAATG
1801 TAATAATGAA GCGGAACTTA AAGGAGGTAT TAAAGAAGCA TTCCAATTTG
1851 ACCGTAAGCT TGTTATAGAA CAAGGCGTTA ACGCAACGTG AAATTGAAGT
1901 AGCAGTTTTA GGAAATGACT ATCCTGAAGC GACATGGCCA GGTGAAGTCG
1951 TAAAAGATGT CGCGTTTTAC GATTACAAAT CAAAATATAA AGGATGGTAA
2001 GGTTCAATTA CAAATTCCAG CTGACTTAGA CGGAAGATGT TCAATTAACG
2051 GCTTAGAAAT ATGGCATTAG AGGCATTCAA AGCGACAGAT TGTTCTGGTT
2101 TAGTCCGTGC TGATTTCTTT GTAACAGAAG ACAACCAAAT ATATATTAAT
2151 GAAACAAATG CAATGCCTGG ATTTACGGCT TTCAGTATGT ATCCAAAGTT
2201 ATGGGAAAAT ATGGGCTTAT CTTATCCAGA ATTGATTACA AAACTTATCG
2251 AGCTTGCTAA AGAACGTCAC CAGGATAAAC AGAAAAATAA ATACAAAATT
2301 SMCTWAMTGA GGTTGTTATK RTGATTAAYG TKACMYTAWA GYAAAWTCAA
2351 TCATGGATTN CCTTGTGAAA TTGAA
```

Mutant: NT 312
Phenotype: temperature sensitivity
Sequence map: Mutant NT312 is complemented by plasmid pMP266, which carries a 1.5 kb insert of wild-type S. aureus genomic DNA. A partial restriction map is depicted in FIG. 71; there are no apparent restriction sites for EcoR I, BamH I, HinD III or Pst I. Database searches at the nucleic acid and (putative) polypeptide levels against currently available databases reveal strong peptide-level similarities to mg442, a hypothetical ORF from M. genetalium, and limited similarities to G-proteins from human and rat clones; this probably indicates a functional domain of a new Staph. protein involved in GTP-binding. The ORF contained within clone pMP266 is novel and likely to be a good candidate for screen development.

DNA sequence data: The following DNA sequence data represents the sequence generated by primer walking through clone pMP266, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP266
SEQ ID NO. 82
pMP266 Length: 1543 nt
      1 AATCATTTTC AGTTTATCAT TAAACAAATA TATTGAACYM MYMAAAATGT
    51 CATACTGATA AAGATGAATG TCACTTAATA AGTAACTTAG ATTTAACAAA
  101 TGATGATTTT TAATTGTAGA AAACTTGAAA TAATCACTTA TACCTAAATC
  151 TAAAGCATTG TTAAGAAGTG TGACAATGTT AAAATAAATA TAGTTGAATT
  201 AATGAATTTG TTCTAYAATT AACAKGTTWT WGAWTTTAAT AATGAGAAAA
  251 GAATTGACGA AAGTAAGGTG AATTGAATGG TTATTCMATG GTATCCAGGA
  301 CMTATGGCGA AAAGCCAAAA GAGAAGTAAG TGAACAATTA AMAAAAGTAG
  351 ATGTAGTGTT TGAACTAGTA GATGCAAGAA TTCCATATAG TTCAAGAAAC
  401 CCTATGATAG ATGAAGTTAT TAACCAAAAA CCACGTGTTG TTATATTAAA
  451 TAAAAAAGAT ATGTCTAATT TAAATGAGAT GTCAAAATGG GAACAATTTT
  501 TTATTGATAA AGGATACTAT CCTGTATCAG TGGATGCTAA GCACGGTAAA
  551 AATTTAAAGA AAGTGGAAGC TGCAGCAATT AAGGCGACTG CTGAAAAATT
  601 TGAACGCGAA AAAGCGAAAG GACTTAAACC TAGAGCGATA AGAGCAATGA
  651 TCGTTGGAAT TCCAAATGTT GGTAAATCCA CATTAATAAA TAAACTGGCA
  701 AAGCGTAGTA TTGCGCAGAC TGGTAATAAA CCAGGTGTGA CCAAACAACA
  751 ACAATGGATT AAAGTTGGTA ATGCATTACA ACTATTAGAC ACACCAGGGA
  801 TACTTTGGCC TAAATTTGAA GATGAAGAAG TCGGTAAGAA GTTGAGTTTA
  851 ACTGGTGCGA TAAAAGATAG TATTGTGCAC TTAGATGAAG TTGCCATCTA
  901 TGGATTAAAC TTTTTAATTC AAAATGATTT AGCGCGATTA AAGTCACATT
  951 ATAATATTGA AGTTCCTGAA GATGCMGAAA TCATAGCGTG GTTTGATGCG
1001 ATAGGGAAAA AACGTGGCTT AATTCGACGT GGTAATGAAA TTGATTACGA
1051 AGCAGTCATT GAACTGATTA TTTATGATAT TCGAAATGCT AAAATAGGAA
1101 ATTATTGTTT TGATATTTTT AAAGATATGA CTGAGGAATT AGCAAATGAC
1151 GCTAACAATT AAAGAAGTTA CGCAGTTGAT TAATGCGGTT AATACAATAG
1201 AAGAATTAGA AAATCATGAA TGCTTTTTAG ATGAGCGAAA AGGTGTTCAA
1251 AATGCCATAG CTAGGCGCAG AAAAGCGTTA GAAAAAGAAC AAGCTTTAAA
1301 AGAAAAGTAT GTTGAAATGA CTTACTTTGA AAATGAAATA TTAAAAGAGC
1351 ATCCTAATGC TATTATTTGT GGGATTGATG AAGTTGGAAG AGGACCTTTA
1401 GCAGGTCCAG TCGTTGCATG CGCAACAATT TTAAATTCAA ATCACAATTA
1451 TTTGGGCCTT GATGACTCGA AAAAAGTACC TGTTACGAAA CGTCTAGAAT
1501 TAAATGAAGC ACTAAAAAAT GAAGTTACTG YTTTTGCATA TGG
```

Mutant: NT 318
Phenotype: temperature sensitivity
Sequence map: Mutant NT318 is complemented by plasmid pMP270, which carries a 2.2 kb insert of wild-type S. aureus genomic DNA. A partial restriction map is depicted in FIG. 72; there are no apparent restriction sites for EcoR I, BamH I, HinD III, or Pst I. Database searches at the nucleic acid and (putative) polypeptide levels against currently available databases reveal strong similarities to the spoVC gene from B. subtilis, a gene identified as being important in sporulation, and the pth gene from E. coli, which encodes aminoacyl-tRNA hydrolase (EC 3.1.1.29). It is highly likely that the spoVC and pth gene products are homologues and that the essential gene identified here is the Staph. equivalent. The predicted relative size and orientation of the spoVC gene is depicted by an arrow in the restriction map.

DNA sequence data: The following DNA sequence data represents the sequence generated by primer walking through clone pMP270, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP270
SEQ ID NO. 83
pMP270 Length: 2185 nt
      1 TTAAACAATT AAGAAAATCT GGTAAAGTAC CAGCASYAGT ATACGGTTAC
    51 GGTACTAAAA ACGTGTCAGT TAAAGTTGAT GAAGTAGAAT TCATCAAAGT
  101 TATCCGTGAA GTAGGTCGTA ACGGTGTTAT CGAATTAGGC GTTGGTTCTA
  151 AAACTATCAA AGTTATGGTT GCAGACTACC AATTCGATCC ACTTAAAAAC
  201 CAAATTACTC ACATTGACTT CTTWKCAATC AATATGAGTG AAGAACGTAC
  251 TGTTGAAGTA CCAGTTCAAT TAGTTGGTGA AGCAGTAGGC GCTAAAGAAA
  301 GGCGGCGTTA GTTGAACAAC CATTATTCAA CTTAGAAAGT AACTGCTACT
  351 CCAGACAATA TTCCAGAAGC AATCGAAGTA GACATTACTG AATTAAACAT
  401 TAACGACAGC TTAACTGTTG CTGATGTTAA AGTAACTGGC GACTTCAAAA
  451 TCGAAAACGA TTCAGCTGAA TCAGTAGTAA CAGTAGTTGC TCCAACTGAA
  501 GAACCAACTG AAGAAGAAAT CGAAGCCTAT GGAAGGCGAA CAMCAAACTG
  551 AAGAACCAGA AGTTGTTGGC GAAAGCAAAG AAGACGAAGA AAAAACTGAA
  601 GAGTAATTTT AATCTGTTAC ATTAAAGTTT TTATACTTTG TTTAACAAGC
  651 ACTGTGCTTA TTTTAATATA AGCATGGTGC TTTTKGTGTT ATTATAAAGC
  701 TTAATTAAAC TTTATWACTT TGTACTAAAG TTTAATTAAT TTTAGTGAGT
  751 AAAAGACATT AAACTCAACA ATGATACATC ATAAAAATTT TAATGTACTC
  801 GATTTTAAAA TACATACTTA CTAAGCTAAA GAATAATGAT AATTGATGGC
  851 AATGGCGGAA AATGGATGTT GTCATTATAA TAATAAATGA AACAATTATG
  901 TTGGAGGTAA ACACGCATGA AATGTATTGT AGGTCTAGGT AATATAGGTA
  951 AACGTTTTGA ACTTACAAGA CATAATATCG GCTTTGAAGT CGTTGATTAT
1001 ATTTTAGAGA AAAATAATTT TTCATTAGAT AAACAAAAGT TTAAAGGTGC
1051 ATATACAATT GAACGAATGA ACGGCGATAA AGTGTTATTT ATCGAACCAA
1101 TGACAATGAT GAATTTGTCA GGTGAAGCAG TTGCACCGAT TATGGATTAT
1151 TACAATGTTA ATCCAGAAGA TTTAATTGTC TTATATGATG ATTTAGATTT
1201 AGAACAAGGA CAAGTTCGCT TAAGACAAAA AGGAAGTGCG GGCGGTCACA
1251 ATGGTATGAA ATCAATTATT AAAATGCTTG GTACAGACCA ATTTAAACGT
1301 ATTCGTATTG GTGTGGGAAG ACCAACGAAT GGTATGACGG TACCTGATTA
1351 TGTTTTACAA CGCTTTTCAA ATGATGAAAT GGTAACGATG GGAAAAAGTT
1401 ATCGAACACG CAGCACGCGC AATTGAAAAG TTTGTTGAAA CATCACRATT
1451 TGACCATGTT ATGAATGAAT TTAATGGTGA AKTGAAATAA TGACAATATT
1501 GACAMCSCTT ATAAAAGAAG ATAATCATTT TCAAGACCTT AATCAGGTAT
1551 TTGGACAAGC AAACACACTA GTAACTGGTC TTTCCCCGTC AGCTAAAGTG
1601 ACGATGATTG CTGAAAAATA TGCACAAAGT AATCAACAGT TATTATTAAT
1651 TACCAATAAT TTATACCAAG CAGATAAATT AGAAACAGAT TTACTTCAAT
1701 TTATAGATGC TGAAGAATTG TATAAGTATC CTGTGCAAGA TATTATGACC
1751 GAAGAGTTTT CAACACAAAG CCCTCAACTG ATGAGTGAAC GTATTAGAAC
1801 TTTAACTGCG TTAGCTCCAA GGTAAGAAAG GGTTATTTAT CGTTCCTTTA
1851 AATGGTTTGA AAAAGTGGTT AACTCCTGTT GAAATGTGGC AAAATCACCA
1901 AATGACATTG CGTGTTGGTG AGGATATCGA TGTGGACCAA TTTMWWAACA
1951 AATTAGTTAA TATGGGGTAC AAACGGGAAT CCGTGGTATC GCATATTGGT
2001 GAATTCTCAT TGCGAGGAGG TATTATCGAT ATCTTTCCGC TAATTGGGGA
2051 ACCAATCAGA ATTGAGCTAT TTGATACCGA AATTGATTCT ATTCGGGATT
2101 TTGATGTTGA AACGCAGCGT TCCAAAGATA ATGTTGAAGA AGTCGATATC
2151 ACAACTGCAA GTGATTATAT CATTACTGAA GAAGT
```

Mutant: NT 321
Phenotype: temperature sensitivity
Sequence map: Mutant NT321 is complemented by plasmid pMP276, which carries a 2.5 kb insert of wild-type S. aureus genomic DNA. A partial restriction map is depicted in FIG. 73; no apparent sites for HinD III, EcoR I, BamH I or Pst I are present. Database searches at the nucleic acid and (putative) polypeptide levels against currently available databases reveal strong peptide-level similarities to a hypothetical ORF of unknown function from M. tuberculosis (Genbank Accession No. Z73902).

DNA sequence data: The following DNA sequence data represents the sequence generated by primer walking through clone pMP276, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP276
SEQ ID NO. 84
pMP276 Length: 2525 nt
      1 AATCTGTTCC TACTACAATA CCTTGTCGGT TTGAAGCACC NGAAAATNGT
    51 ACTTTCATAC GTTCACGCGC TTTTTCATTT CCTTTTTGGA AATCTGTAAG
  101 AACAATACCG GCTTCTTTTA ATGATTGCAC ACTTTGATCA ACTGCAGGCT
  151 TAATATTGAC TGTTACTATT TCATCTGGTT CAATGAATCG CAAAGCTTGC
  201 TCAACTTCAT CAGCATCTTT TTGAACTCCA TAAGGTAATT TAACTGCAAT
  251 AAACGTACAA TCAATGCCTT CTTCACGTAA TTCGTTAACA GACATTTGTA
  301 CTAGTTTTCC AACTAATGTA GAATCCTGTC CTCCTGAAAT ACCTAACACT
  351 AAAGATTTTA TAAATGAATG TGATTGTACA TAATTTTTTA TAAATTGCTT
  401 TAATTCCATA ATTTCTTCAG CACTATCGAT ACGCTTTTTC ACTTTCATTT
  451 CTTGTACAAT AACGTCTTGT AATTTACTCA TTATCTTCTT CCATCTCCTT
  501 AACGTGTTCC GCAACTTCAA AAATACGTTT ATGTTTATTA TCCCAACATG
  551 CCTTGCTTAA ATCGACTGGA TATTCTTGTG GATTCAGGAA ACGCTTATTT
  601 TCATCCCAAA TAGATTGTAA TCCTAGTGCT AAATATTCAC GTGATTCATC
  651 TTCTGTTGGC ATTTGATATA CTAATTTACC ATTTTCATAA ATATTATGAT
  701 GCAAATCAAT GGCTTCGAAA GATTTTATAA ATTTCATTTT ATAAGTATGC
  751 ACTGGATGGA ATAATTTTAA AGGTTGTTCA TCGTATGGAT TTTCATTTTC
  801 CAAAGTAATA TAATCGCCTT CTGCCTTACC TGTTTTCTTG TTTATAATGC
  851 GATATACATT TTTCTTACCT GGCGTCGTAA CCTTTTCAGC GTTATTTGAT
  901 AATTTAATAC GATCACTATA TGAACCATCT TCATTTTCAA TAGCTACAAG
  951 TTTATATACT GCACCTAATG CTGGTTGATC GTATCCTGTA ATCAGCTTTG
1001 TACCAACGCC CCAAGAATCT ACTTTTGCAC CTTGTGCTTT CAAACTCGTA
1051 TTCGTTTCTT CATCCAAATC ATTAGAYGCG ATAATTTTAG TTTCAGTAAA
1101 TCCTGYTTCA TCAAGCATAC GTCTTGCYTC TTTAGATAAA TAAGCGATAT
1151 CTCCAGAATC TAATCGAATA CCTAACAAAG TTAATTTTGT CACCTAATTC
1201 TTTTGCAACT TTTATTGCAT TTGGCACGCC AGATTTTAAA GTATGGAATG
1251 TATCTACTAG GAACACACAA TTTTTATGTC TTTCAGCATA TTTTTTGAAG
1301 GCAACATATT CGTCTCCATA AGTTTGGACA AATGCATGTG CATGTGTACC
1351 AGACACAGGT ATACCAAATA ATTTTCCCCG CCCTAACATT ACTTGTAGAA
1401 TCAAAGCCCC CGATGTAAGC AGCTCTAGCG CCCCACAATG CTGCATCAAT
1451 TTCTTGCGCA CGACGTGTTA CCAAACTCCA TTAATTTATC ATTTGATGCA
1501 ATTTGACGAA ATTCTGCTAG CCTTTGTTGT AATTAATGTA TGGAAATTTA
1551 CAATGTTTAA TAAAATTGTT CTATTAATTG CGCTTGAATC AATGGTGCTT
1601 CTACGCGTAA CAATGGTTCG TTACCAAAGC ATAATTCGCC TTCTTGCATC
1651 GAACGGATGC TGCCTGTGAA TTTTAAATCT TTTAAATATG ATAAGAAATC
1701 ATCCTTGTAG CCAATAGACT TTAAATATTC CAAATCAGAT TCTGAAAATC
1751 CAAAATGTTC TATAAAATCA ATGACGCGTT TTAAACCATT AAAAACAGCA
1801 TAGCCACTAT TAAATGGCAT TTTTCTAAAA TACAAATCAA ATACAGCCAT
1851 TTTTTCATGA ATATTATCAT TCCAATAACT TTCAGCCATA TTTATTTGAT
1901 ATAAGTCATT ATGTAACATT AAACTGTCGT CTTCTAATTG GTACACTTGT
1951 ATCTCTCCAA TCGACCTAAA TATTTTCTTA CATTTTATCA TAATTCATTT
2001 TTTTATATAC ATAAGAGCCC CTTAATTTCC ATACTTTTAA TTAAAATCAA
2051 CCAACAATTT AATGACATAT ACATAATTTT TAAGAGTATT TTAATAATGT
2101 AGACTATAAT ATAAAGCGAG GTGTTGTTAA TGTTATTTAA AGAGGCTCAA
2151 GCTTTCATAG AAAACATGTA TAAAGAGTGT CATTATGAAA CGCAAATTAT
2201 CAATAAACGT TTACATGACA TTGAACTAGA AATAAAAGAA ACTGGGACAT
2251 ATACACATAC AGAAGAAGAA CTTATTTATG GTGCTAAAAT GGCTTGGCGT
2301 AATTCAAATC GTTGCATTGG TCGTTTATTT TGGGATTCGT TAAATGTCAT
2351 TGATGCAAGA GATGTTACTG ACGAAGCATC GTTCTTATCA TCAATTACTT
2401 ATCATATTAC ACAGGCTACA AATGAAGGTA AATTAAAGCC GTATATTACT
2451 ATATATGCTC CAAAGGATGG ACCTAAAATT TTCAACAATC AATTAATTCG
2501 CTATGCTGGC TATGACAATT GTGGT
```

Mutant: NT 325
Phenotype: temperature sensitivity
Sequence map: Mutant NT325 is complemented by plasmid pMP644, which carries a 2.1 kb insert of wild-type S. aureus genomic DNA. A partial restriction map is depicted in FIG. 74; no apparent sites for HinD III, EcoR I, BamH I or Pst I are present. Database searches at the nucleic acid and (putative) polypeptide levels against currently available databases reveal significant peptide-level similarities to the ribC gene product, a protein exhibiting regulatory functions, from B. subtilis (Genbank Accession. No. x95312; unpublished).

DNA sequence data: The following DNA sequence data represents the sequence generated by primer walking through clone pMP644, starting with standard M13 forward and M13 reverse sequencing primers and completing the sequence contig via primer walking strategies. The sequence below can be used to design PCR primers for the purpose of amplification from genomic DNA with subsequent. DNA sequencing.

```
clone pMP644
SEQ ID NO. 85
pMP644 Length: 2181 nt
      1 ATCGATAGGA AGAAGTACAA CGACTGAAGA TCAAACGGGT GATACATTGG
    51 AAACAAAAGG TGTACACTCA GCAGATTTTA ATAAGGACGA TATTGACCGA
  101 TTGTTAGAAA GTTTTAAAGG TATCATTGAA CAAATTCCGC CGATGTACTC
  151 ATCCGTCAAA GTAAATGGTA AAAAATTATA TGAATATGCG CGTAATAATG
  201 AAACAGTTGA AAGACCAAAG CGTAAAGTTA ATATTAAAGA CATTGGGCGT
  251 ATATCTGAAT TAGATTTTAA AGAAAATGAG TGTCATTTTA AAATACGCGT
  301 CATCTGTGGT AAAGGTACAT ATATTAGAAC GCTAGCAACT GATATTGGTG
  351 TGAAATTAGG CTTTCCGGCA CATATGTCGA AATTAACACG AATCGAGTCT
  401 GGTGGATTTG TGTTGAAAGA TAGCCTTACA TTAGAACAAA TAAAAGAACT
  451 TCATGAGCAG GATTCATTGC AAAATAAATT GTTTCCTTTA GAATATGGAT
  501 TAAAGGGTTT GCCAAGCATT AAAATTAAAG ATTCGCACAT AAAAAAACGT
  551 ATTTTAAATG GGCAGAAATT TAATAAAAAT GAATTTGATA ACATAATTAA
  601 AGACCAAATT GTATTTATTG ATGATGATTC AGAAAAAGTA TTAGCAATTT
  651 ATATGGTACA CCCTACGAAA AGAATCAGAA ATTAAACCTA AAAAAGTCTT
  701 TAATTAAAGG AGATAGAATT TATGAAAGTT CATAGAAAGT GACACATCCT
  751 ATACAATCCT AAACAGTTAT ATTACAGGAG GATGTTGCAA TGGGCATTCC
  801 GGATTTTTCG ATGGCATGCA TAAAGGTCAT GACAAAGTCT TTGATATATT
  851 AAACGAAATA GCTGAGGCAC GCAGTTTAAA AAAAGCGGTG ATGACATTTG
  901 ATCCGCATCC GTCTGTCGTG TTTGAATCCT AAAAGAAAAC GAACACGTTT
  951 TTACGCCCCT TTCAGATAAA ATCCGAAAAA TTACCCACAT GATATTGATT
1001 ATTGTATAGT GGTTAATTTT TCATCTAGGT TTGCTAAAGT GAGCGTAGAA
1051 GATTTTGTTG AAAATTATAT AATTAAAAAT AATGTAAAAG AAGTCATTGC
1101 TGGTTTTGAT TTTAACTTTT GGTAAATTTG GAAAAGGTAA TATGACTGTA
1151 ACTTCAAGAA TATGATGCGT TTAATACGAC AATTGTGAGT AAACAAGAAA
1201 TTGAAAATGA AAAAATTTCT ACAACTTCTA TTCGTCAAGG ATTTAATCAA
1251 TGGTGAGTTG CCAAAAAGGC GAATGGATGG CTTTTAGGCT ATATATATTT
1301 CTTATTAAAA GGCACTGTAG TGCAAGGTGA AAAAAGGGGA AGAACTATTG
1351 GCTTCCCCAA CAGCTAACAT TCAACCTAGT GATGATTATT TGTTACCTCG
1401 TAAAGGTGTT TATGCTGTTA GTATTGAAAT CGGCACTGAA AATAAATTAT
1451 ATCGAGGGGT AGCTAACATA GGTGTAAAGC CAACATTTCA TGATCCTAAC
1501 AAAGCAGAAG TTGTCATCGA AGTGAATATC TTTGACTTTG AGGATAATAT
1551 TTATGGTGAA CGAGTGACCG TGAATTGGCA TCATTTCTTA CGTCCTGAGA
1601 TTAAATTTGA TGGTATCGAC CCATTAGTTA AACAAATGAA CGATGATAAA
1651 TCGCGTGCTA AATATTTATT AGCAGTTGAT TTTGGTGATG AAGTAGCTTA
1701 TAATATCTAG AGTTGCGTAT AGTTATATAA ACAATCTATA CCACACCTTT
1751 TTTCTTAGTA GGTCGAATCT CCAACGCCTA ACTCGGATTA AGGAGTATTC
1801 AAACATTTTA AGGAGGAAAT TGATTATGGC AATTTCACAA GAACGTAAAA
1851 ACGAAATCAT TAAAGAATAC CGTGTACACG AAACTGATAC TGGTTCACCA
1901 GAAGTACAAA TCGCTGTACT TACTGCAGAA ATCAACGCAG TAAACGAACA
1951 CTTACGTACA CACAAAAAAG ACCACCATTC ACGTCGTGGA TTATTAAAAA
2001 TGGTAGGTCG TCGTAGACAT TTATTAAACT ACTTACGTAG TAAAGATATT
2051 CAACGTTACC GTGAATTAAT TAAATCACTT GGTATCCGTC GTTAATCTTA
2101 ATATAACGTC TTTGAGGTTG GGGCATATTT ATGTTCCAAC CCTTAATTTA
2151 TATTAAAAAA GCTTTTTRCA WRYMTKMASR T
```

Mutant: NT 333
Phenotype: temperature sensitivity
Sequence map: Mutant NT333 is complemented by plasmid pMP344, which carries a 2.3 kb insert of wild-type S. aureus genomic DNA. A partial restriction map is depicted in FIG. 75; no apparent restriction sites for EcoR I, HinD III, BamH I or Pst I are present. Database searches at the nucleic acid and (putative) polypeptide levels against currently available databases reveal significant similarities to the murD gene product from B. subtilis, which encodes udp-MurNAc-dipeptide::D-Glu ligase (EC 6.3.2.9); similarities are also noted to the equivalent gene products from E. coli and H. influenzae. The predicted relative size and orientation of the murD gene is depicted by an arrow in the map.

DNA sequence data: The following DNA sequence data represents the sequence-generated by primer walking through clone pMP344, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP344
SEQ ID NO. 86
pMP344 Length: 2424 nt
      1 ACATTAAAAA GGATGAAATT TGGTCAAAGT ATTCGAGAAG AAGGTCCACA
    51 AAGCCATATG AAGAAGACTG GTACACCAAC GATGGGTGGA CTAACATTTC
  101 TATTAAGTAT TGTGATAACG TCTTTGGTGG CTATTATATT TGTAGATCAA
  151 GCWAATCCAA TCATACTGTT ATTATTTGTG ACGATTGGTT TTGGGTTAAT
  201 TGGTTCTTAT ACGATGATTA TATTATTGTT GTTAAAAAGA ATAACCAAGG
  251 TTTAACAAGT AAACAGAAGT TTTTGGCGCA AATTGGTATT GCGATTATAT
  301 TCTTTGTTTT AAGTAATGTG TTTCATTTGG TGAATTTTTC TACGAGCATA
  351 CATATTCCAT TTACGAATGT AGCAATCCCA CTATCATTTG CATATGTTAT
  401 TTTCATTGTT TTTTGGCAAG TAGGTTTTTC TAATGCAGTA AATTTAACAG
  451 ATGGTTTAGA TGGATTAGCA ACTGGACTGT CAATTATCGG ATTTACAATG
  501 TATGCCATCA TGAGCTTTGT GTTAGGAGAA ACGGCAATTG GTATTTTCTG
  551 TATCATTATG TTGTTTGCAC TTTTAGGATT TTTACCATAT AACATTAACC
  601 CTGCTAAAGT GTTTATGGGA GATACAGGTA GCTTAGCTTT AGGTGGTATA
  651 TTTGCTACCA TTTCAATCAT GCTTAATCAG GAATTATCAT TAATTTTTAT
  701 AGGTTTAGTA TTCGTAATTG AAACATTATC TGTTATGTTA CAAGTCGCTA
  751 GCTTTAAATT GACTGGAAAG CGTATATTTA AAATGAGTCC GATTCATCAT
  801 CATTTTGAAT TGATAGGATG GAGCGAATGG AAAGTAGTTA CAGTATTTTG
  851 GGCTGTTGGT CTGATTTCAG GTTTAATCGG TTTATGGATT GGAGTTGCAT
  901 TAAGATGCTT AATTATACAG GGTTAGAAAA TAAAAATGTW TTAGTTGTCG
  951 GTTTGGCAAA AAGTGGTTAT GAAGCAGCTA AATTATTAAG TAAATTAGGT
1001 GCGAATGTAA CTGTCAATGA TGGAAAAGAC TTATCACAAG ATGCTCATGC
1051 AAAAGATTTA GAWTCTATGG GCATTTCTGT TGTAAGTGGA AGTCATCCAT
1101 TAACGTTGCT TGATAATAAT CCAATAATTG TTAAAAATCC TGGAATACCC
1151 TTATACAGTA TCTATTATTG ATGAAGCAGT GAAACGAGGT TTGAAAATTT
1201 TAACAGAAGT TGAGTTAAGT TATCTAATCT CTGAAGCACC AATCATAGCT
1251 GTAACGGGTA CAAATGGTAA AACGACAGTT ACTTCTCTAA TTGGAGATAT
1301 GTTTAAAAAA AGTCGCTTAA CTGGAAGATT ATCCGGCAAT ATTGGTTATG
1351 TTTGCATCTA AAGTWGCACA AGAAGTWAAG CCTACAGATT ATTTAGTTAC
1401 AGAGTTGTCG TCATTCCAGT TACTTGGAAT CGAAAAGTAT AAACCACACA
1451 TTGCTATAAT TACTAACATT TATTCGGCGC ATCTAGATTA CCATGRAAAT
1501 TTAGAAAACT ATCAAAATGC TAAAAAGCAA ATATATAAAA ATCAAACGGA
1551 AGAGGATTAT TTGATTTGTA ATTATCATCA AAGACAAGTG ATAGAGTCGG
1601 AAGAATTAAA AGCTAAGACA TTGTATTTCT CAAACTCAAC AAGAAGTTGA
1651 TGGTATTTAT ATTAAAGATG RTTTTATCGT TTATAAAGGT GTTCGTATTA
1701 TTAACACTGA AGATCTAGTA TTGCCTGGTG AACATAATTT AGAAAATATA
1751 TTAGCCAGCT GKGCTKGCTT GTATTTWAGY TGGTGTACCT ATTAAAGCAA
1801 TTATTGATAG TTWAAYWACA TTTTCAGGAA TAGAGCATAG ATTGCAATAT
1851 GTTGGTACTA ATAGAACTTA ATAAATATTA TAATGATTCC AAAGCAACAA
1901 ACACGCTAGC AACACAGTTT GCCTTAAATT CATTTAATCA ACCAATCATT
1951 TGGTTATGTG GTGGTTTGGA TCGGAGGGAA TGAATTTGAC GAACTCATTC
2001 CTTATATGGA AAATGTTCGC GCGATGGTTG TATTCGGACA AACGAAAGCT
2051 AAGTTTGCTA AACTAGGTAA TAGTCAAGGG AAATCGGTCA TTGAAGCGAA
2101 CAATGTCGAA GACGCTGTTG ATAAAGTACA AGATATTATA GAACCAAATG
2151 ATGTTGTATT ATTGTCACCT GCTTGTGCGA GTTGGGATCA ATATAGTACT
2201 TTTGAAGAGC GTGGAGAGAA ATTTATTGAA AGATTCCGTG CCCATTTACC
2251 ATCTTATTAA AGGGTGTGAG TATTGATGGA TGATAAAACG AAGAACGATC
2301 AACAAGAATC AAATGAAGAT AAAGATGAAT TAGAATTATT TACGAGGAAT
2351 ACATCTAAGA AAAGACGGCA AAGAAAAAGW TCCTCTAGAG TCGACCCTGC
2401 AGGCATGCAA GCTTGGCGTA NCC
```

Mutant: NT 346
Phenotype: temperature sensitivity
Sequence map: Mutant NT346 is complemented by plasmid pMP347, which carries a 2.1 kb insert of wild-type S. aureus genomic DNA. A partial restriction map is depicted in FIG. 76; no apparent restriction sites for EcoR I, HinD III, BamH I or Pst I are present. Database searches at the nucleic acid and (putative) polypeptide levels against currently available databases reveal strong similarities to the tpiS gene from B. subtilis, which encodes triose phosphate isomerase (EC 5.3.1.1); similarities are also noted to the equivalent gene products from B. megaterium and B. stearothermophilus. The predicted relative size and orientation of the tpiS gene is depicted by an arrow in the restriction map.

DNA sequence data: The following DNA sequence data represents the sequence generated by primer walking through clone pMP347, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP347
SEQ ID NO. 87
pMP347 Length: 2094 nt
      1 CACATAAACC AGTTGTTGCT ATTTTAGGTG GAGCAAAAGT ATCTGACAAA
    51 ATTAATGTCA TCAAAAACTT AGTTAACATA GCTGATAAAA TTATCATCGG
  101 CGGAGGTATG GCTTATACTT TCTTAAAAGC GCAAGGTAAA GAAATTGGTA
  151 TTTCATTATT AGAAGAAGAT AAAATCGACT TCGCAAAAGA TTTATTAGAA
  201 AAACATGGTG ATAAAATTGT ATTACCAGTA GACACTAAAG TTGCTAAAGA
  251 ATTTTCTAAT GATGCCAAAA TCACTGTAGT ACCATCTGAT TCAATTCCAG
  301 CAGACCAAGA AGGTATGGAT ATTGGACCAA ACACTGTAAA ATTATTTGCA
  351 GATGAATTAG AAGGTGCGCA CACTGTTGTT ATGGAATGGA CCTATGGGTT
  401 GTTATTCGAG TTCAGTAACT TTGCACAAGG TACAATTGGT GTTTGTTAAA
  451 GCAATTGCCA ACCTTAAAGA TGCCATTACG ATTATCGGTG GCGGTGATTC
  501 AGCCTGCAGC AGCCATCTCT TTAGGTTTTT GAAAATGACT TCACTCMTAT
  551 TTCCACTGGT GGCGGCSCKC CATTAGAKTA CCTAGAAGGT WAAGAATGCC
  601 TGGTWTCMAA GCAAYCAWTA WTAAWTAATA AAGTGATAGT TTAAAGTGAT
  651 GTGGCATGTT TGTTTAACAT TGTTACGGGA AAACAGTCAA CAAGATGAAC
  701 ATCGTGTTTC ATCAACTTTT CAAAAATATT TACAAAAACA AGGAGTTGTC
  751 TTTAATGAGA ACACCAATTA TAGCTGGTAA CTGGAAAATG AACAAAACAG
  801 TACAAGAAGC AAAAGACTTC GTCAATACAT TACCAACACT ACCAGATTCA
  851 AAAGAAKTWR AATCAGTWAT TTGTTGCMCC AGCMATTCAA TTAGATGCAT
  901 TAACTACTGC AGTTWAAGAA GGAAAAGCAC AAGGTTTAGA AATCGGTGCT
  951 CAAAATNCGT ATTTCGAAGA AATGGGGCTT MACAGTGAAA KTTTCCAGTT
1001 GCATAGCAGA TTAGGCTTAA AAAGTTGTAT TCGGTCATTC TGAACTTCGT
1051 GAATATTCCA CGGAACCAGA TGAAGAAATT AACAAAAAAG CGCACGTATT
1101 TTCAAACATG GAATGAMTCC AATTATATGT GTTGGTGAAA CAGACGAAGA
1151 GCGTGAAAGT GGTAAAGCTA ACGATGTTGT AGGTGAGCAA GTTAAAGAAA
1201 GCTGTTGCAG GTTTATCTGA AGATCAAACT TAAATCAGTT GTAATTGCTT
1251 ATGAACCAAT CTGGGCAATC GGAACTGGTA AATCATCAAC ATCTGAAGAT
1301 GCAAATGAAA TGTGTGCATT TGTACGTCAA ACTATTGCTG ACTTATCAAG
1351 CAAAGAAGTA TCAGAAGCAA CTCGTATTCA ATATGGTGGT AGTGTTAAAC
1401 CTAACAACAT TAAAGAATAC ATGGCACAAA CTGATATTGA TGGGGCATTA
1451 GTAGGTGGCG CATCACTTAA AGTTGAAGAT TTCGTACAAT TGTTAGAAGG
1501 TGCAAAATAA TCATGGCTAA GAAACCAACT GCGTTAATTA TTTTAGATGG
1551 TTTTGCGAAC CGCGAAAGCG AACATGGTAA TGCGGTAAAA TTAGCAAACA
1601 AGCCTAATTT TTNGATCGGT TNATTACCAA CCAAATATCC CAACCGAACT
1651 TCAAAATTCG AAGGCGAGTG GCTTAAGATG TTGGACTACC CTGAAGGACA
1701 AATGGGTAAC TCAGAAGTTG GTCATATGAA TATCGGTGCA GGACGTATCG
1751 TTTATCAAAG TTTAACTCGA ATCAATAAAT CAATTGAAGA CGGTGATTTC
1801 TTTGAAAATG ATGTTTTAAA TAATGCAATT GCACACGTGA ATTCACATGA
1851 TTCAGCGTTA CACATCTTTG GTTTATTGTC TGACGGTGGT GTACACAGTC
1901 ATTACAAACA TTTATTTGCT TTGTTAGAAC TTGCTAAAAA ACAAGGTGTT
1951 GAAAAAGTTT ACGTACACGC ATTTTTAGAT GGCCGTGACG TAGATCAAAA
2001 ATCCGCTTTG AAATACATCG AAGAGACTGA AGCTAAATTC AATGAATTAG
2051 GCATTGGTCA ATTTGCATCT GTGTCTGGTC GTTATTATGC ANTG
```

Mutant: NT348
phenotype: temperature sensitivity
Sequence map: Mutant NT348 is complemented by plasmid pMP649, which carries a 3.3 kb insert of wild-type S. aureus genomic DNA. A partial restriction map is depicted in FIG. 77; no apparent restriction sites for EcoR I, HinD III, BamH I or Pst I are present. Database searches at the nucleic acid and (putative) polypeptide levels against currently available databases reveal DNA sequence identities to two different Genbank entries for S. aureus DNA. The left-most contig below matches Genbank Accession No. U31979, which includes the complete aroC gene, encoding 5-enolpyruvylshikimate 3-phosphate phospholyase (EC 4.6.1.4), and the N-terminal portion of the aroB gene, encoding 5-dehydroquinate hydrolyase (EC 4.2.1.10); the right-most contig matches Genbank Accession No. L05004, which includes the C-terminal portion of the aroB gene. Neither Genbank entry described contains the complete DNA sequence of pMP649. Further experiments are underway to determine whether one or both of the genes identified in clone pMP649 are essential.

DNA sequence data: The following DNA sequence data represents the sequence generated from clone pMP649, starting with standard M13 forward and M13 reverse sequencing primers; the sequence contig will be completed via primer walking strategies. The sequence below can be used to design PCR primers for the purpose of amplification from genomic DNA with subsequent DNA sequencing.

```
clone pMP649
SEQ ID NO. 88
pMP649.forward Length: 954 nt
    1 GGGGWYYCTC TAGAGYCGAC CTRCAGGCAT SCAAGCTTBA CCAGGWTCAA
  51 TTAGAGGTRA TTWAGGTTTA RCTKTTSGTV GAADTATCAT BMTCGGTTCA
101 GATTCCTGAG AGTCTGCTGA ACGTGAAATT AATCTATGGT TTAATGAAAA
151 TGAAATTACT AGCTATGCTT CACCACGTGA TGCATGGTTA TATGAATAAA
201 ATATAAACTG TAAACCTTTA CGATTTATTT ATAAAGGTAG AAAGGGTTTT
251 GTTATGTGGT TAGTCATTAT GATTATACAT AACAAGGCCC GTTTTTTATG
301 TTGTAGTAAA TTACTTGAAA AATTTTATAG TTTTTTGGTA ACACGTATTA
351 AAAAGAGAGG AATATTCTTT ATCAAATGAA ACTAAACAGA GAGAAGGGGT
401 TGTTAAAATG AAGAATATTA TTTCGATTAT TTTGGGGATT TTAATGTTCT
451 TAAAATTAAT GGAATTACTA TATGGTGCTA TATTTTTAGA TAAACCACTT
501 AATCCTATAA CAAAAATTAT TTTTATACTG ACTCTCATTT ATATTTTTTA
551 TGTATTAGTA AAAGAATTGA TTATATTTTT GAAGTCAAAG TATAACAAAA
601 GCGCTTAACA TATGTTTATT TTAATATCAT AATTTTTTTA AACGGGACTG
651 ATTAACYTTT ATTAATAATT AACAGTTCGT TCTTTTGTAT TAAGAAATGT
701 AGTCAGTATA TTATTTGCTA AAGTTGCGAT ACGATTATAT TAAAACGGCT
751 AATCATTTTT AATTAATGAT TATATGATGC AACTGTTTAG AAATTCATGA
801 TACTTTTCTA CAGACGAATA TATTATAATT AATTTTAGTT CGTTTAATAT
851 TAAGATAATT CTGACATTTA AAATGAGATG TCATCCATTT TCTTAATTGA
901 GCTTGAAAAC AAACATTTAT GAATGCACAA TGAATATGAT AAGATTAACA
951 ACAT
SEQ ID NO. 89
pMP649.reverse Length: 841 nt
    1 CTTTMAWKRC CTRAACCACT TAACAAACCT GCCAATAATC GTGTTGTCGT
  51 ACCAGAATTA CCTGTATACA ATACTTGATG TGGCGTGTTA AAAGATTGAT
101 ATCCTGGGGA AGTCACAACT AATTTTTCAT CATCTTCTTT GATTTCTACA
151 CCTAACAGTC GGAAAATGTC CATCGTACGA CGACAATCTT CGCCAAGTAG
201 TGGCTTATAT ATAGTAGATA CACCTTCAGC TAGCGACGCC AACATGATTG
251 CACGGTGTGT CATTGACTTA TCGCCCGGCA CTTCTATTTC GCCCTTTAAC
301 GGACCTGAAA TATCAATGAT TTGTTCATTT ACCATTTCAT TCACCTACTT
351 AAAATATGTT TTTAATTGTT CACATGCATG TTGTAATGTT AGTTGATCAA
401 CATGTTGTAC AACGATATCT CCAAATTGTC TAATCAAGAC CATTTGTACA
451 CCTTGCTTAT CATTCTTTTT ATCACTTAGC ATATATTGGT ATAACGTTTC
501 AAAATCCAAG TCAGTTATCA TGTCTAAAGG ATAGCCGAGT TGTATTAAAT
551 ATTGAATATA ATGATTAATA TCATGCTTAG RATCAAACAA AGCATTCGCA
601 ACTATAAATT GATAGATAAT GCCAACCATC ACTGACATGA CCATGAGGTA
651 TTTTATGATA GTATTCAACA GCATGACCAA ATGTATGACC TAAATTTAAR
701 AATTTACGTA CACCTTGTTC TTTTTSATCT GGCGAATAAC AATATCCAGC
751 TTSGTTTCAA TACCTTTRGS AATWTATTTR TCCATACCAT TTAATGACTG
801 TAATATCTCT CTATCTTTAA AGTGCTGTTC GATATCTTGC G
```

Mutant: NT359
phenotype: temperature sensitivity
Sequence map: Mutant NT359 is complemented by plasmid pMP456, which carries a 3.2 kb insert of wild-type S. aureus genomic DNA. A partial restriction map is depicted in FIG. 78; no apparent restriction sites for EcoR I, HinD III, BamH I or Pst I are present. Database searches at the nucleic acid and (putative) polypeptide levels against currently available databases reveal identity to the glnRA locus of S. aureus (Genbank Accession No. X76490), also referred to as the femC locus; mutations localized to femC have been reported in the scientific literature to display an increased sensitivity to the bacterial cell-wall synthesis inhibitor methicillin.

DNA sequence data: The following DNA sequence data represents the sequence generated from clone pMP456, starting with standard M13 forward and M13 reverse sequencing primers; the sequence contig will be completed via primer walking strategies. The sequence below can be used to design PCR primers for the purpose of amplification from genomic DNA with subsequent DNA sequencing.

```
clone pMP456
SEQ ID NO. 90
pMP456.forward Length: 568 nt
    1 CCGGGGATCC TCTAGAGTCG ATCTTTGCAT TCTTTAAGCT TAAATTTTCT
  51 ATTCTTCTTT CTCTACGGCG CATAGCATTA ATATTACCGT AACTTATCCC
101 AGTATCTTTA TTAATTTGAT AACTCGATAT CTCTTTGTTT TCTATCAATT
151 CTTTGATTGT ATTGAATATT TCATCATAGC AATTCATAAA TTAGATGAGG
201 CGAAATTTTT AATTTTTTAG AATATCAATA GTANTATAAC TAAAATGAAA
251 ATACCGATCG ATAAACAAAA AGATATTTTT TGTTTTGTTT CTCTTTTCAT
301 ATAGTATTAC CCCCTTAATA ATGCGTAGTA AGGTCCCTCT TTTCGGGGTC
351 TTACCTTANA AACGTTCTGC AAATGAATTC GATGAGAAGT AATATGAATA
401 TGGCTATTTT CAAGTAATAC TCAACGTTTT CGCGACGTTC TTTTATCGCC
451 TCATCTCATC ACCTCCAAAT ATATTAAAAT TCATGTGAAC TAAAATATAA
501 AATGGTCTTC CCCAGCTTTA AAAAAATAAA TACATAAAAC ATTTTACTTG
551 GACCAAAACT TGGACCCC
SEQ ID NO. 91
pMP456.reverse Length: 581 nt
    1 ATGCCTGCAG GTCGATCATT AATTAAAAAC CCTGGCGGTG GTTTAGCTAA
  51 GATTGGTGGA TACATTGCTG GTAGAAAAGA TTTAATTGAA CGATGTGGTT
101 ATAGATTGAC AGCACCTGGT ATTGGTAAAG AAGCGGGTGC ATCATTAAAT
151 GCATTGCTTG AAATGTATCA AGGTTTCTTT TTAGCACCAC ACGTTGTCAG
201 TCAGAGTCTT AAAGGTGCAT TGTTTACTAG TTTATTTTTA GAAAAAATGA
251 ATATGAACAC AACGCCGAAG TACTACGAAA AACGAACTGA TTTAATTCAA
301 ACAGTTAAAT TTGAAACGAA AGAACAAATG ATTTCATTTT GTCAAAGTAT
351 TCAACACGCA TCCCCAATTA ATGCACATTT TAGTCCANAA CCTAGTTATA
401 TGCCTGGTTA CGAAGATGAT GTTATTATGG CAGCTGGTAC GTTTATTCAA
451 GGTTCATCCG ATTGAATTAT CTGCAGATGG ACCTATTCGT CCTCCTTATG
501 AAGCATATGT TCAAGGANGA TTAACATATG AACACGTTAA AATTGCTGTT
551 GACAAGANCT GTTTAATCAG TTTGAAAAAA C
```

Mutant: NT371
phenotype: temperature sensitivity
Sequence map: Mutant NT371 is complemented by plasmid pMP461, which carries a 2.0 kb insert of wild-type S. aureus genomic DNA. A partial restriction map is depicted in FIG. 79. Database searches at the nucleic acid and (putative) polypeptide levels against currently available databases reveal strong peptide-level similarities to yluD, a hypothetical ABC transporter (Genbank Accession No. M90761), and yidA, a hypothetical ORF of unknown function (Genbank Accession No. L10328).

DNA sequence data: The following DNA sequence data represents the sequence generated by primer walking through clone pMP461, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP461
SEQ ID NO. 92
pMP461 Length: 2001 nt
      1 CGGGGATCCT CTAAAGTCGA TCAAATTGGG CGAATGAAGC AAGGAAAAAC
    51 AATTTTAAAA AAGATTTCTT GGCAAATTGC TAAAGGTGAT AAATGGATAT
  101 TATATGGGTT GAATGGTGCT GGCAAGACAA CACTTCTAAA TATTTTAAAT
  151 GCGTATGAGC CTGCAACATC TGGAACTGTT AACCTTTTCG GTAAAATGCC
  201 AGGCAAGGTA GGGTATTCTG CAGAGACTGT ACGACAACAT ATAGGTTTTG
  251 TATCTCATAG TTTACTGGAA AAGTTTCAAG AGGGTGAAAG AGTAATCGAT
  301 GTGGTGATAA GCGGTGCCTT TAAATCAATT GGTGTTTATC AAGATATTGA
  351 TGATGAGATA CGTAATGAAG CACATCAATT ACTTAAATTA GTTGGAATGT
  401 CTGCTAAAGC GCAACAATAT ATTGGTTATT TATCTACCGG TGAAAAACAA
  451 CGAGTGATGA TTGCACGAGC TTTAATGGGG CAACCCCAGG TTTTAATTTT
  501 AGATGAGCCA GCAGCTGGTT TAGACTTTAT TGCACGAGAA TCGTTGTTAA
  551 GTATACTTGA CTCATTGTCA GATTCATATC CAACGCTTGC GATGATTTAT
  601 GTGACGCACT TTATTGAAGA AATAACTGCT AACTTTTCCA AAATTTTACT
  651 GCTAAAAGAT GGCCAAAGTA TTCAACAAGG CGCTGTAGAA GACATATTAA
  701 CTTCTGAAAA CATGTCACGA TTTTTCCAGA AAAATGTAGC AGTTCAAAGA
  751 TGGAATAATC GATTTTCTAT GGCAATGTTA GAGTAAATAT TTTGCAAATA
  801 ATAAGTAATA ATGACAAAAT TTAATTAAGA TAAAATGGAC AGTGGAGGGC
  851 AATATGGATA ACGTTAAAAG CAATATTTTT GGACATGGAT GGAACAATTT
  901 TACATTGAAA ATAATCCAAG CATCCAACGT WTACGAAAGA TGTTCATTAA
  951 TCAATTGGAG AGAGAAAGGA TATWAAGTAT TTTTGGSCAA CAGGACGTTC
1001 GCATTCTGAA ATACATCMAA YTTGTACCTC AAGATTTTGC GGTTAATGGC
1051 ATCATTAGTT CAAATGGAAC AATTGGAGAA GTAGATGGAG AAATTATCTT
1101 CAAGCATGGT TTATCATTGG CTCAAGTGCA ACAAATTACT AATTTAGCTA
1151 AGCGCCAACA AATTTATTAT GAGGTATTTC CTTTTGAAGG TAATAGAGTT
1201 TCTTTAAAAG AAGATGAAAC ATGGATGCGA GATATGATTC GTAGTCAAGA
1251 TCCTATTAAT GGCGTAAGTC ATAGTGAATG GTCTTCAAGA CAAGATGCGC
1301 TTGCTGGTAA GATAGATTGG GTAACTAAGT TTCCTGAAGG TGAATATTCA
1351 AAAATTTATC TATTCAGTTC TAATTTAGAA AAAATAACAG CATTTAGAGA
1401 TGAATTAAAG CAAAATCATG TGCAACTACA GATTAGTGTT TCAAATTCAT
1451 CAAGATTTAA TGCGGAAACA ATGGCTTATC AAACTGATAA AGGTACAGGC
1501 ATTAAAGAAA TGATTGCACA TTTTGGTATT CATCAAGAAG AAACGTTAGT
1551 TATTGGAGAT AGCGACAATG ATAGAGCAAT GTTTGAATTT GGTCATTATA
1601 CAGTTGCTAT GAAAAATGCA CGCCCTGAAA TCCAAGCATT AACTTCAGAT
1651 GTAACGGCAT ACACGAATGA AGAGGATGGC GCAGCAAAAT ATTTAGCAGA
1701 GCATTTTTTA GCTGAATAAT AAAATAGGTA GTTATTTATT ATTTAATTTA
1751 CAATAGTTGA TGAGTAATGT ACAAAGAGCA GTAAAGTTAT TTTCTATTAG
1801 AAAATGTCTT ACTGCTCTTT TGTATGCTTA TAAATATTTG AATCATCTAT
1851 ATTTAATTGG ACAAACTCTA TGAGAATAAA TATTGTTAAA ACTAATAAGA
1901 TAGGAAATTC ATTGATTTTG AATAATATTT CTTGTTTTAA GGTTTAACTA
1951 TTGAATTGTA TACTTCTTTT TTTAGTAGCA ACAGATCGAC CTGCAGGCAT
2001 A
```

Mutant: NT 379
Phenotype: temperature sensitivity
Sequence map: Mutant NT379 is complemented by plasmid pMP389, which carries a 2.5 kb insert of wild-type S. aureus genomic DNA. A partial restriction map is depicted in FIG. 80; no apparent restriction sites for EcoR I, HinD III, BamH I or Pst I are present. Database searches at the nucleic acid and (putative) polypeptide levels against currently available databases reveal strong similarities to the tagF gene from B. subtilis, which encodes a protein involved in the biosynthesis of teichoic acid polymers (Genbank Accession No. X15200). The Tag genes of B. subtilis have been identified as essential and are expected to make good candidates for screen development. The predicted relative size and orientation of the tagF gene is depicted by an arrow in the restriction map.

DNA sequence data: The following DNA sequence data represents the sequence generated by primer walking through clone pMP389, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP389
SEQ ID NO. 93
pMP389 Length: 2522 nt
      1 GANCTCGGTA CCCGGGGATG CCTSYAGAGT CGATCGCTAC CACCTTGAAT
    51 GACTTCAATT CTTTCATCAG AAATTTTGAA TTTTCTAAGT GTATCTTTCG
  101 TATGCGTCAT CCATTGTTGT GGCGTCGCGA TAATAATTTT TTCAAAATCA
  151 TTAATTAAAA TAAATTTTTC TAATGTATGG ATTAAAATCG GTTTGTTGTC
  201 TAAATCTAAA AATTGTTTAG GTAAAGGTAC GTTACCCATT CTTGAGCCTA
  251 TACCTCCAGC TAGAATACCA GCGTATTTCA TAAAATACTT CCTCCATTCA
  301 ACTATATCTA TATTTAATTA TTTAAATTTC GTTGCATTTT CCAATTGAAA
  351 ACTCATTTTA AAATCAAAAC TCTAAATGTC TGTGTATTAC TTAAAATTAT
  401 ACATATTTTG CTTATATTTT AGCATATTTT GTTTAAACCT ATATTACATT
  451 ATATCAGACG TTTTCATACA CAAATAATAA CATACAAGCA AACATTTCGT
  501 TTATTATTTA TATCACTTAA CTAATTAATT TATAATTTTT TATTGTTTTT
  551 AAGTTATCAC TTAAAAATCG TTTGGCAAAT TCGTTGTGAC GCTTGTCCAT
  601 CTTCTAATGA ACAGAATTTT TGATAAAATA CCGTTCGTGC TTCAATATAC
  651 TCATTTGCAG TCTCATCGAT TTGTTTTAAT GCATCAATGA GTGCTGTTTG
  701 ATTTTCAACA ATTGGAMCTG GCAACTCTTT TTTATAATCC ATGTAAAAAC
  751 CTCTAAGCTC ATCGCCATAT TTATCTAAGT CATATGCATA GAAAATTTGC
  801 GGACGCTTTA ATACACCGAA GTCGAACATG ACAGATGAGT AGTCGGTAAC
  851 TAACGCATCG CTGATTAAGT TATAAATCCG AAATGCCTTC ATAATCTGGA
  901 AAMGTCTTTC AACAAAATCA TCAATGTTCA TCAATAACGY GTCAACAACT
  951 AAATAATGCA KGCGTAATAA AATAACATAA TCATCATCCA GCGCTTGACG
1001 CAAAGCTTCT ATATCAAAGT TAACATTAAA TTGATATGAA CCCTTCTCGG
1051 AATCGCTTCA TCGTCAACGC CAAGTTGGCG CGTACATAAT CAACTTTTTT
1101 ATCTAATGGA ATATTTAATC TTGTCTTAAT ACCATTAATA TATTCAGTAT
1151 CATTGCGTTT ATGTGATAAT TTATCATTTC TTGGATAACC TGTTTCCAAA
1201 ATCTTATCTC GACTAACATG AAATGCATTT TGAAATATCG ATGTCGAATA
1251 TGGATTAGGT GACACTAGAT AATCCCACCG TTGGCTTTCT TTTTTAAAGC
1301 CATCTTGGTA ATTTTGAGTA TTTGTTCCTA GCATTTTAAC GTTACTAATA
1351 TCCAAACCAA TCTTTTTTAA TGGCGTGCCA TGCCATGTTT GTAAGTACGT
1401 CGTTCGCGGT GATTTATATA ACCAATCTGG TGTACGTGTG TTAATCATCC
1451 ACGCTTTCGC TCTTGGCATC GCTAAAAACC ATTTCATTGA AAACTTTGTA
1501 ACATATGGTA CATTGTGCTG TTGGAATATG TGTTCATATC CTTTTTTCAC
1551 ACCCCATATT AATTGGGCAT CGCTATGTTC AGTTAAGTAT TCATATAATG
1601 CTTTGGGGTT GTCGCTGTAT TGTTTACCAT GAAAGCTTTC AAAATAAATT
1651 AGATTCTTGT TTGGCAATTT TGGATAGTAA TTTAAAAGTC GTATATATAC
1701 TATGTTCTAT CAATTTTTTA ATTGTATTTT TAATCATGTC GTACCTCCGA
1751 CGTGTTTTTG TAATTATATT AATATGTATG AGCAAGCTCA TTGTAACCAT
1801 GCCTATTATA GCATTTCATC ATAAAATACA TTTAACCATT ACACTTGTCG
1851 TTAATTATCA TACGAAATAC ATGATTAATG TACCACTTTA ACATAACAAA
1901 AAATCGTTAT CCATTCATAA CGTATGTGTT TACACATTTA TGAATTAGAT
1951 AACGATTGGA TCGATTATTT TATTTWACAA AATGACAATT CAGTTGGAAG
2001 GTGATTGCTT TTGATTGAAT CGCCTTATGC ATGAAAAATC AAAAGGTTAT
2051 TCTCATTGTA TAGTCCTGCT TCTCATCATG ACATGTTGCT CACTTCATTG
2101 TCAGAACCCT TCTTGAAAAC TATGCCTTAT GACTCATTTG CATGGCAAGT
2151 AATATATGCC AACATTAGCG TCTAAACAAA TCTTTGACTA AACGTTCACT
2201 TGAGCGACCA TCTTGATATT TAAAATGTTT ATCTAAGAAT GGCACAACTT
2251 TTTCAACCTC ATAATCTTCA TTGTCCAAAG CATCCATTAA TGCATCAAAG
2301 GACTGTACAA TTTTACCTGG AACAAATGAT TCAAATGGTT CATAGAAATC
2351 ACGCGTCGTA ATGTAATCTT CTAAGTCAAA TGCATAGAAA ATCATCGGCT
2401 TTTTAAATAC TGCATATTCA TATATTAAAG ATGAATAATC ACTAATCAAC
2451 AAGTCTGTAA CAAAGAGAAT ATCGTTWACT TCASGRTCGA TCGACTCTAG
2501 AGGATCCCCG GGTACCGAGC TC
```

Mutant: NT 380
Phenotype: temperature sensitivity
Sequence map: Mutant NT380 is complemented by plasmid pMP394, which carries a 1.3 kb insert of wild-type S. aureus genomic DNA. A partial restriction map is depicted in FIG. 81. Database searches at the nucleic acid and (putative) polypeptide levels against currently available databases reveal strong similarities to the cdsA gene product from E. coli (Genbank Accession No. M11330), which encodes phosphatidate cytidylyltransferase (EC 2.7.7.41); the cdsA gene product is involved in membrane biogenesis and is likely to be a good candidate for screen development. The predicted relative size and orientation of the cdsA gene is depicted by an arrow in the restriction map.

DNA sequence data: The following DNA sequence data represents the sequence generated by primer walking through clone pMP394, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP394
SEQ ID NO. 94
pMP394 Length: 1335 nt
      1 CAGAGTTGTT AATTCGTACT TCAGGAGAAC AAAGAATAAG TAATTTCTTG
    51 ATTTGGCAAG TTTCGTATAG TGAATTTATC TTTAATCAAA AATTATGGCC
  101 TGACTTTGAC GAAGATGAAT TAATTAAATG TATAAAAATT TATCAGTCAC
  151 GTCAAAGACG CTTTGGCGGA TTGARTGAKG AGKATRTATA GTATGAAAGT
  201 TAGAACGCTG ACAGCTATTA TTGCCTTAAT CGTATTCTTG CCTATCTTGT
  251 TAAAAGGCGG CCTTGTGTTA ATGATATTTG CTAATATATT AGCATTGATT
  301 GCATTAAAAG AAATTGTTGA ATATGAATAT GATTAAATTT GTTTCAGTTC
  351 CTGGTTTAAT TAGTGCAGTT GGTCTTATCA TCATTATGTT GCCACAACAT
  401 GCAGGGCCAT GGGTACAAGT AATTCAATTA AAAAGTTTAA TTGCAATGAG
  451 CTTTATTGTA TTAAGTTATA CTGTCTTATC TAAAAACAGA TTTAGTTTTA
  501 TGGATGCTGC ATTTTGCTTA ATGTCTGTGG CTTATGTAGG CATTGGTTTT
  551 ATGTTCTTTT ATGAAACGAG ATCAGAAGGA TTACATTACA TATTATATGC
  601 CTTTTTAATT GTTTGGCTTA CAGATACAGG GGCTTACTTG TTTGGTAAAA
  651 TGATGGGTTA AACATAAGCT TTGGCCAGTA ATAAKTCCGA ATAAAACAAT
  701 CCGAAGGATY CATAGGTGGC TTGTTCTGTA GTTTGATAGT ACCACTTGCA
  751 ATGTTATATT TTGTAGATTT CAATATGAAT GTATGGATAT TACTTGGAGT
  801 GACATTGATT TTAAGTTTAT TTGGTCAATT AGGTGATTTA GTGGAATCAG
  851 GATTTAAGCG TCATTTNGGC GTTAAAGACT CAGGTCGAAT ACTACCTGGA
  901 CACGGTGGTA TTTTAGACCG ATTTGACAGC TTTATGTTTG TGTTACCATT
  951 ATTAAATATT TTATTAATAC AATCTTAATG CTGAGAACAA ATCAATAAAC
1001 GTAAAGAGGA GTTGCTGAGA TAATTTAATG AATCCTCAGA ACTCCCTTTT
1051 GAAAATTATA CGCAATATTA ACTTTGAAAA TTATACGCAA TATTAACTTT
1101 GAAAATTAGA CGTTATATTT TGTGATTTGT CAGTATCATA TTATAATGAC
1151 TTATGTTACG TATACAGCAA TCATTTTTAA AATAAAAGAA ATTTATAAAC
1201 AATCGAGGTG TAGCGAGTGA GCTATTTAGT TACAATAATT GCATTTATTA
1251 TTGTTTTTGG TGTACTAGTA ACTGTTCATG AATATGGCCA TATGTTTTTT
1301 GCGAAAAGAG CAGGCATTAT GTGTCCAGAA TTTGC
```

Mutant: NT401
phenotype: temperature sensitivity
Sequence map: Mutant NT401 is complemented by plasmid pMP476, which carries a 2.9 kb insert of wild-type S. aureus genomic DNA. A partial restriction map is depicted in FIG. 82. Database searches at the nucleic acid and (putative) polypeptide levels against currently available databases reveal sequence identity in the middle of the clone to pMP64, the complementing clone to NT31 (described previously). Since pMP64 does not cross complement NT401, and pMP476 contains additional DNA both upstream and downstream, the essential gene is likely to reside in the flanking DNA. The remaining DNA that completely contains an ORF is that coding for yqeJ, a hypothetical ORF from B. subtilis (Genbank Accession No. D84432)

DNA sequence data: The following DNA sequence data represents the sequence generated by primer walking through clone pMP476, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP476
SEQ ID NO. 95
pMP476 Length: 2902 nt
      1 GAGCTCGGTA CCCGGGGATC CTCTAGAGTC GATCATTACC TAATTCGTAT
    51 TGTCGAACAA TTTGATACAT TTTACCTAAA TCATCATATT TACAGAAATC
  101 ATGTAATACA CCTGCTAATT CTACTTTACT AGTGTCTCCA TCATAAATTT
  151 CTGCCRATTT AATCGCTGTT TCTGCAACTC TTAAAGAATG ATTGATRACG
  201 TTTCTCTGGA CAGTTTCTCT TTTGCAAGCC GTTTTGCTTT TTCAATGTWC
  251 ATATAATCCT TCCCCCTTAA TATAGTTTTC AACGGATTTA GGAACAAGAA
  301 CTTGGATAGA TTTCCCTTCA CTAACTCTTT GTCGAATCAT TGTCGAACTT
  351 ATATCTACCC TAGGTATCTG AATTGCAATC ATAGCATTTT CAACATTTTG
  401 ACTATTTTTG TCTCGATTTA CAACTACAAA AGTAACCATT TCTTTTAAGT
  451 ATTCAATTTG ATACCATTTC TCTAGTTGGT TATACTGATC CGTCCCAATA
  501 ACAAAGTACA ACTCACTGTC TTTGTGTTGC TCCTTGAATG CCTTGATCGT
  551 GTCATAGGTA TAACTTTGAC CACCACGTTT AATTTCATCG TCACAAATAT
  601 CTCCAAAACC AAGCTCGTCG ATAATCATCT GTATCATTGT TAATCTGTGC
  651 TGAACGTCTA TAAAATCATG GTGCTTTTTC AATGGAGAMA WAAAAMWARR
  701 WAAAAAATAA AATTCATCTG GCTGTAATTC ATGAAATACT TCGCTAGCTA
  751 CTATCATATG TTGCAGTATG GATAGGGTTA AACTGACCGC CGTAAAGTAC
  801 TATCTTTTTC ATTATTATGG CAATTCAATT TCTTTATTAT CTTTAGATTC
  851 TCTATAAATC ACTATCATAG ATCCAATCAC TTGCACTAAT TCACTATGAA
  901 KTAGCTTCCG CTTAATGTTT CCAGCTAATY CTTTTTTATC ATCAAAGTTT
  951 ATTTTGTTAK TACATGTTAC TTTAATCAAT YCTCTGTTTT CYAACGTTAT
1001 CATCTATTTG TTTAATCATA TTTTCGTTGA TACCGCCTTT TCCAATTTGA
1051 AAAATCGGAT CAATATTGTG TGCTAAACTT CTTAAGTATC TTTTTTGTTT
1101 GCCAGTAAGC ATATGTTATT CTCCTTTTAA TTGTTGTAAA ACTGCTGTTT
1151 TCATAGAATT AATATCAGCA TCTTTATTAG TCCAAATTTT AAAGCTTTCC
1201 GCACCCTGGT AAACAAACAT ATCTAAGCCA TTATAAATAT GGTTTCCCTT
1251 GCGCTCTGCT TCCTCTAAAA TAGGTGTTTT ATACGGTATA TAAACAATAT
1301 CACTCATTAA AGTATTGGGA GAAAGAGCTT TAAATTAATA ATACTTTCGT
1351 TATTTCCAGC CATACCCGCT GGTGTTGTAT TAATAACGAT ATCGAATTCA
1401 GCTAAATACT TTTCAGCATC TGCTAATGAA ATTTGGTTTA TATTTAAATT
1451 CCAAGATTCA AAACGAGCCA TCGTTCTATT CGCAACAGTT AATTTGGGCT
1501 TTACAAATTT TGCTAATTCA TAAGCAATAC CTTTACTTGC ACCACCTGCG
1551 CCCAAAATTA AAATGTATGC ATTTTCTAAA TCTGGATAAA CGCTGTGCAA
1601 TCCTTTAACA TAACCAATAC CATCTGTATT ATACCCTATC CACTTGCCAT
1651 CTTTTATCAA AACAGTGTTA ACTGCACCTG CATTAATCGC TTGTTCATCA
1701 ACATAATCTA AATACGGTAT GATACGTTCT TTATGAGGAA TTGTGATATT
1751 AAASCCTTCT AATTYTTTTT TSGAAATAAT TTCTTTAATT AAATGAAAAA
1801 TTYTTCAATT GGGAATATTT AAAGCTTCAT AAGTATCATC TTAATCCTAA
1851 AGAATTAAAA TTTGCTCTAT GCATAACGGG CGACAAGGAA TGTGAAATAG
1901 GATTTCCTAT AACTGCAAAT TTCATTTTTT TAATCACCTT ATAAAATAGA
1951 ATTYTTTAAT ACAACATCAA CATTTTTAGG AACACGAACG ATTACTTTAG
2001 CCCCTGGTCC TATAGTTATA AAGCCTAGAC CAGAGATCAT AACATCGCGT
2051 TTCTCTTTGC CTGTTTCAAG TCTAACAGCC TTTACCTCAT TAAGATCAAA
2101 ATTTTGTGGA TTTCCAGGTG GCGTTAATAA ATCGCCAAGT TGATTACGCC
2151 ATAAATCATT AGCCTTCTCC GTTTTAGTAC GATGTATATT CAAGTCATTA
2201 GAAAAGAAAC AAACTAACGG ACGTTTACCA CCTGAWACAT AATCTATGCG
2251 CGCTAGACCG CCGAAGAATA ATGTCKGCGC CTCATTTAAT TGATATACGC
2301 GTTGTTTTAT TTCTTTCTTA GGCATAATAA TTTTCAATYC TTTTTCACTA
2351 ACTAAATGCG TCATTTGGTG ATCTTGAATA ATACCTGGTG TATCATACAT
2401 AAATGATGTT TCATCTAAAG GAATATCTAT CATATCTAAA GTTGYTTCCA
2451 GGGAATCTTG AAGTTGTTAC TACATCTTTT TCACCAACAC TAGCTTCAAT
2501 CAGTTTATTA ATCAATGTAG ATTTCCCAAC ATTCGTTGTC CCTACAATAT
2551 ACACATCTTC ATTTTCTCGA ATATTCGCAA TTGATGATAA TAAGTCGTCT
2601 ATGCCCCAGC CTTTTTCAGC TGAAATTAAT ACGACATCGT CAGCTTCCAA
2651 ACCATATTTT CTTGCTGTTC GTTTTAACCA TTCTTTAACT CGACGTTTAT
2701 TAATTTGTTT CGGCAATAAA TCCAATTTAT TTGCTGCTAA AATGATTTTT
2751 TTGTTTCCGA CAATACGTTT AACTGCATTA ATAAATGATC CTTCAAAGTC
2801 AAATACATCC ACGACATTGA CGACAATACC CTTTTTATCC GCAAGTCCTG
2851 ATAATAATTT TAAAAAGTCT TCACTTTCTA ATCCTACATC TTGAACTTCG
2901 TT
```

Mutant: NT423
phenotype: temperature sensitivity
Sequence map: Mutant NT423 is complemented by plasmid pMP499, which carries a 2.0 kb insert of wild-type S. aureus genomic DNA. A partial restriction map is depicted in FIG. 83. Database searches at the nucleic acid and (putative) polypeptide levels against currently available databases reveal strong peptide-level similarities to yqhY, a hyptothetical ORF identified from a genomic sequencing effort in B. subtilis (Genbank Accession No. D84432), and yqhZ, hypothetical ORF from B. subtilis bearing similarity to the nusB gene product from E. coli (Genbank Accession No. M26839; published in Imamoto, F. et al. Adv. Biophys. 21 (1986) 175-192). Since the nusB gene product has been demonstrated to be involved in the regulation of transcription termination in E. coli, it is likely that either one or both of the putative genes identified in this sequence contig encode essential functions.

DNA sequence data: The following DNA sequence data represents the sequence generated by primer walking through clone pMP499, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP499
SEQ ID NO. 96
pMP499 Length: 1916 nt
      1 AGTCGATCAA AGCCAATGTT CCAGTTGTTC CTGGTAGTGA CGGTTTAATG
    51 AAAGACGTCT CAGAAGCTAA GAAAATCGCC AAAAAAATTG GCTATCCGGT
  101 CATCATTAAA GCTACTGCTG GCGGTGGCGG AAAAGGTATC CGTGTTGCTC
  151 GTGATGAAAA AGAACTTGAA ACTGGCTTCC GAATGACAGA ACAAGAAGCT
  201 CAAACTGCAT TTGGTAATGG TGGACTTTAT ATGGAGAAAT TCATCGAAAA
  251 CTTCCGCCAT ATTGAAATCC AAATTGTTGG GGACAGCTAT GGTAATGTAA
  301 TTCATTTAGG AGAACGTGAT TGTACAATTC AAAGACGTNT GCAGAAATTA
  351 GTGGAAGAAG CACCTTCCCC NATTTTAGAT GATGAAACAC GTCGTGAAAT
  401 GGGAAATGCC GCAGTTCGTG CAGCGAAAGC TGTAAATTAT GAAAATGCGG
  451 GAACAATTGA GTTTATATAT GATTTAAATG ATAATAAATT TTATTTTATG
  501 GAAATGAATA CACGTATTCA AGTAGAACAT CCTGTAACTG AAATGGTAAC
  551 AGGAATTGAT TTAGTTAAAT TACAATTACA AGTTGCTATG GGTGACGTGT
  601 TACCGTATAA ACAAGAAGAT ATTAAATTAA CAGGACACGC AATTGAATTT
  651 AGAATTAATG CTGAAAATCC TTACAAGAAC TTTATGCCAT CACCAGGTAA
  701 AATTGAGCAA TATCTTGCAC CAGGTGGATA TGGTGTTCGA ATAGAGTCAG
  751 CATGTTATAC TAATTATACG ATACCGCCAT ATTATGATTC GATGGTAGCG
  801 AAATTAATCA TACATGAACC GACACGAGAT GARGCGATTA TGGSTGGCAT
  851 TCGTGCACTA ARKGPAWTTG TGGTTYTTGG GTATTGATAC AACTATTCCA
  901 TTTCCATATT AAATTATTGA ATAACGGATA TATTTAGGAA GCGGTAAATT
  951 TAATACAAAC TTTTTAGAAG CAAAATAGCA TTATTGAATG ATGAAAGGTT
1001 AATAGGAGGT CMATCCCMTG GTCAAAGTAA CTGATTATTC MAATTCMAAA
1051 TTAGGTAAAG TAGAAATAGC GCCAGAAGTG CTATCTGTTA TTGCAAGTAT
1101 AGCTACTTCG GAAGTCGAAG GCATCACTGG CCATTTTGCT GAATTAAAAG
1151 AAACAAATTT AGAAAAAGTT AGTCGTAAAA ATTTAAGCCG TGATTTAAAA
1201 ATCGAGAGTA AAGAAGATGG CATATATATA GATGTATATT GTGCATTAAA
1251 ACATGGTGTT AATATTTCAA AAACTGCAAA CAAAATTCAA ACGTCAATTT
1301 TTAATTCAAT TTCTAATATG ACAGCGATAG AACCTAAGCA AATTAATATT
1351 CACATTACAC AAATCGTTAT TGAAAAGTAA TGTCATACCT AATTCAGTAA
1401 TTAAATAAAG AAAAATACAA ACGTTTGAAG GAGTTAAAAA TGAGTCGTAA
1451 AGAATCCCGA GTGCAAGCTT TTCAAACTTT ATTTCAATTA GAAATGAAGG
1501 ACAGTGATTT AACGATAAAT GAAGCGATAA GCTTTATTAA AGACGATAAT
1551 CCAGATTTAG ACTTCGAATT TATTCATTGG CTAGTTTCTG GCGTTAAAGA
1601 TCACGAACCT GTATTAGACG AGACAATTAG TCCTTATTTA AAAGATTGGA
1651 CTATTGCACG TTTATTAAAA ACGGATCGTA TTATTTTAAG AATGGCAACA
1701 TATGAAATAT TACACAGTGA TACACCTGCT AAAGTCGTAA TGAATGAAGC
1751 AGTTGAATTA ACAAAACAAT TCAGTGATGA TGATCATTAT AAATTTATAA
1801 ATGGTGTATT GAGTAATATA AAAAAATAAA ATTGAGTGAT GTTATATGTC
1851 AGATTATTTA AGTGTTTCAG CTTTAACGAA ATATATTAAA TATAAATTTG
1901 ATCGACCTGC AGGCAT
```

Mutant: NT432
phenotype: temperature sensitivity
Sequence map: Mutant NT432 is complemented by plasmid pMP500, which carries a 1.9 kb insert of wild-type S. aureus genomic DNA. A partial restriction map is depicted in FIG. 84. Database searches at the nucleic acid and (putative) polypeptide levels against currently available databases reveal strong peptide-level similarities to the pgsA gene product, encoding CDP-diacylglycerol:glycerol-3-phosphate 3-phosphatidyltransferase (PGP synthase; EC 2.7.8.5) from B. subtilis(Genbank Accession No. D50064; published in Kontinen, V. P. et al. FEBS lett. 364 (1995) 157-160).

DNA sequence data: The following DNA sequence data represents the sequence generated by primer walking through clone pMP500, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP500
SEQ ID NO. 97
pMP500 Length: 1932 nt
      1 CGGGGATCCT CTAGAGTCGA TCCGTTTGGT GGTGGTTTTG GTTTCTTCGA
    51 GTAAGTGTAA GGAGGCTATG AATTGARRAC GGTCGGTGAA GCGCTAAAAG
  101 GTANACGTGA AAGGTTAGGA ATGACTTYAA CAGAATTAGA GCAACGTACT
  151 GGAATTAANC GTGAAATGCT AGTGCATATT GAAAATAATG AATTCGATCA
  201 ACTACCGAAT AAAAATTACA GCGAAGGATT TATTAGAAAA TATGCAAGCG
  251 TAGTAAATAT TGAACCTAAC CAATTAATTC AAGCTCATCA AGATGAAATT
  301 CCATCGAACC AGAGCCGAAT GGGACGAAGT AATTACAGTT TTCAATAGAT
  351 AATAAAGACT TACGATTATA AGAGTAAATC AAAGANAGCC AATACAATTA
  401 TTAGTAATCA TGGGTTATTA CAGTTTTAAT AACTTTATTG TTATGGATCA
  451 TGTTAGTTTT AATATTTTAA CAGAAATAAA TTAGTGAGAA ATGAGGATGT
  501 TATAATGAAT ATTCCGAACC AGATTACGGT TTTTAGAGTT AGTGTTAATA
  551 CCAGTTTTTA TATTGTTTGC GTTAGTTGAT TTTGGATTTG GCAATGTGTC
  601 ATTTCTAGGA GGATATGAAA TAAGAATTGA GTTATTAATC AGTGGTTTTA
  651 TTTTTATATT GGCTTCCCTT AGCGATTTTG TTGATGGTTA TTTAGCTAGA
  701 AAATGGAATT TAGTTACAAA TATGGGGAAA TTTTTGGATC CATTAGCGGA
  751 TAAATTATTA GTTGCAAGTG CTTTAATTGT ACTTGTGCAA CTAGGACTAA
  801 CAAATTCTGT AGTAGCAATC ATTATTATTG CCAGAGAATT TGCCGTAACT
  851 GGTTTACGTT TACTACAAAT TGAACAAGGA TTCCGTAAGT TGCAGCTGGT
  901 CCAATTTAGG TWAAAWTWAA AACAGCCAGT TACTATGGTT AGCMAWTWAC
  951 TTGGTTGTTW ATTAAGKTGA TCCCATTGGG CAACATTGAT TGGTTTGTCC
1001 ATTARGACAA ATTTTAATTA TAACATTGGC GTTATWTTTW ACTATCYTAT
1051 CTGGTATTGA ATAACTTTTA TAAAGGTAGA GATGTTTTTA AACAAAAATA
1101 AATATTTGTT TATACTAGAT TTCATTTTCA TATGGAATCT AGTTTTTTTA
1151 ATCCCAATTT TAGAAATTAG CCACGCAATT GTTTATAATG ATATATTGTA
1201 AAACAATATT TGTTCATTTT TTTAGGGAAA ATCTGTAGTA GCATCTGATA
1251 CATTGAATCT AAAATTGATG TGAATTTTTA AATGAAATAC ATGAAAAAAT
1301 GAATTAAACG ATACAAGGGG GATATAAATG TCAATTGCCA TTATTGCTGT
1351 AGGCTCAGAA CTATTGCTAG GTCAAATCGC TAATACCAAC GGACAATTTC
1401 TATCTAAAGT ATTTAATGAA ATTGGACAAA ATGTATTAGA ACATAAAGTT
1451 ATTGGAGATA ATAAAAAACG TTTAGAATCA AGTGTAACGT CATGCGCTAG
1501 AAAAATATGA TACTGTTATT TTAACAGGTG GCTTAGGTCC TACGAAAGAT
1551 GACTTAACGA AGCATACAGT GGCCCAGATT GTTGGTAAAG ATTTAGTTAT
1601 TGATGAGCCT TCTTTAAAAT ATATTGAAAG CTATTTTGAG GAACAAGGAC
1651 AAGAAATGAC ACCTAATAAT AAACAACAGG CTTTAGTAAT TGAAGGTTCA
1701 ACTGTATTAA CAAATCATCA TGGCATGGCT CCAGGAATGA TGGTGAATTT
1751 TGAAAACAAA CAAATTATTT TATTACCAGG TCCACCGAAA GAAATGCAAC
1801 CAATGGTGAA AAATGAATTG TTGTCACATT TTATAAACCA TAATCGAATT
1851 ATACATTCTG AACTATTAAG ATTTGCGGGA ATAGGTGAAT CTAAAGTAGA
1901 AACAATATTA ATAGATCGAC CTGCAGGCAT GC
```

Mutant: NT435
phenotype: temperature sensitivity
Sequence map: Mutant NT435 is complemented by plasmid pMP506, which carries a 3.2 kb insert of wild-type S. aureus genomic DNA. A partial restriction map is depicted in FIG. 85. Database searches at the nucleic acid and (putative) polypeptide levels against currently available databases reveal strong peptide-level similarity from the left-most contig (shown below) to the pdhA gene product, encoding the E1-alpha subunit of pyruvate dehydrogenase, from B. subtilis. The right-most contig below demonstrates DNA sequence identity to the pdhC gene, encoding the E2 chain of dihydrolipoamide acetyltransferase (EC 2.3.1.12), from S. aureus (Genbank Accession No. X58434). This Genbank entry also contains the pdhB gene upstream, encoding the E1-beta subunit of pyruvate dehydrogenase (EC 1.2.4.1); since the pMP506 clone contains the region upstream of pdhC, it is predicted that the essential gene identified by mutant NT435 is pdhB. Further sequencing is currently underway to prove this assertion.

DNA sequence data: The following DNA sequence data represents the sequence generated from clone pMP506, starting with standard M13 forward and M13 reverse sequencing primers; the sequence contig will be completed via primer walking strategies. The sequence below can be used to design PCR primers for the purpose of amplification from genomic DNA with subsequent DNA sequencing.

```
clone pMP506
SEQ ID NO. 98
pMP506.forward Length: 619 nt
    1 ATTCGAGCTC GGTACCCGGG GATCCTCTAN AGTCGATCTT ACGGATGAAC
  51 AATTAGTGGA ATTAATGGAA AGAATGGTAT GGACTCGTAT CCTTGATCAA
101 CGTTCTATCT CATTAAACAG ACAAGGACGT TTAGGTTTCT ATGCACCAAC
151 TGCTGGTCAA GAAGCATCAC AATTAGCGTC ACAATACGCT TTAGAAAAAG
201 AAGATTACAT TTTACCGGGA TACAGAGATG NTCCTCAAAT TATTTGGCAT
251 GGTTTACCAT TAACTGAAGC TTTCTTATTC TCAAGAGGTC ACTTCAAAGG
301 AAATCAATTC CCTGAAGGCG TTAATGCATT AAGCCCACAA ATTATTATCG
351 GTGCACAATA CATTCAAGCT GCTGGTGTTT GCATTTGCAC TTAAAAAACG
401 TTGGTAAAAA TGCAGTTGCA ATCACTTACA CTGGTTGACG GTGGTTCTTC
451 ACAAGGTTGA TTTCTACGAA GGTATTAACT TTGCAGCCAG CTTTATAAAG
501 CACCTGGCAA TTTTCCGTTA TTCAAAACAA TAACTATGCA ATTTCAACAC
551 CCAAGAANCA AGCNAACTGC TGCTGAAACA TTACTCAAAA ACCATTGCTG
601 TAGTTTTCCT GGTATCCAT
SEQ ID NO. 99
pMP506.reverse Length: 616 nt
    1 CTTGCATGCC TGCAGGTCGA TCANCATGTT TAACAACAGG TACTAATAAT
  51 CCTCTATCAG TGTCTGCTGC AATACCGATA TTCCAGTAAT GTTTATGAAC
101 GATTTCACCA GCTTCTTCAT TGAATGAAGT GTTAAGTGCT GGGTATTTTT
151 TCAATGCAGA AACAAGTGCT TTAACAACAT AAGGTAAGAA TGTTAACTTA
201 GTACCTTGTT CAGCTGCGAT TTCTTTAAAT TTCTTACGGT GATCCCATAA
251 TGCTTGAACA TCAATTTCAT CCATTAATGT TACATGAGGT GCAGTATGCT
301 TAGAGTTAAC CATTGCTTTC GCAATTGCTC TACGCATAGC AGGGATTTTT
351 TCAGTTGTTT CTGGGAAGTC GCCTTCTAAT GTTACTGCTG CAGGTGCTGC
401 AGGAGTTTCA GCAACTTCTT CACTTGTAGC TGAAGCAGCT GATTCATTTG
451 AAGCTGTTGG TGCACCACCA TTTAAGTATG CATCTACATC TTCTTTTGTA
501 ATACGACCAT TTTTTACCAG ATCCAGAAAC TGCTTTAATG TTTAACACCT
551 TTTTCACGTG CGTTATTTAC TTACTGAAGG CATTGCTTTA AACAGTCTGT
601 TTTCATCTAC TTCCTC
```

Mutant: NT437
phenotype: temperature sensitivity
Sequence map: Mutant NT437 is complemented by plasmid pMP652, which carries a 3.1 kb insert of wild-type S. aureus genomic DNA. A partial restriction map is depicted in FIG. 86; no apparent restriction sites for EcoR I, HinD III, BamH I or Pst I are present. Database searches at the nucleic acid and (putative) polypeptide levels against currently available databases reveal no significant similarities at this time. Current efforts are underway to complete the sequence contig and identify the essential gene contained in clone pMP652.

DNA sequence data: The following DNA sequence data represents the sequence generated from clone pMP652, starting with standard M13 forward and M13 reverse sequencing primers; the sequence contig will be completed via primer walking strategies. The sequence below can be used to design PCR primers for the purpose of amplification from genomic DNA with subsequent DNA sequencing.

```
clone pMP652
SEQ ID NO. 100
pMP652.forward Length: 655 nt
    1 GTACCGGGGA TCGTCACTTA NCCTCTCTAT TTCAATTTCA ACTTATTTCG
  51 TCATCAAGTA TATGTGTTAT GCTTTTATAA CTTTGATTTC AATTCTATCA
101 ATATCTGTGA CATTGATAAC ATCGGACATA CGGTCTTCTT GTAACTTTTT
151 ATCCAATTCA AATGTATACT TTCCATAGTA TTTCTTTTTG ACTGTAATTT
201 TTCCTGTACT CATTTCACCG TAAAGACCAT AATTATCAAT AAGGTATTTT
251 CTTAATTTAA AATCAATCTC TTTCAATGAC ATCGCTTCTT TATCTATTTT
301 AAATGGGAAA AAGTCATAAT CATATTCACC AGTATGATCT TCTTTAATAA
351 CTCTTGCTTC TGCTATTAGG TCGACAGCTT TATCGTTTGC ACTCGTGATA
401 CCCCCAATAG AGTACTTTGC ACCTTCAAAT CTCTTATCCT CATTAACGTA
451 AAATATATTA AGAWTACGAW KKTACACCCG TATGATAATG TTTGCTTATC
501 TTTGCCAATT AAAGCAATAT TATTAACAGA ATTACCATCT ATGATATTCA
551 TAAATTTAAT ACTTGGTTGA ATGAAACTGG ATATAACCTG TCMCATTTTT
601 AATATTCMAT ACTAGGTTGA ATWATAATAA GCTTTTAATT TTTKGCTATT
651 TTCCC
SEQ ID NO. 101
pMP652.reverse Length: 650 nt
    1 GTCGACTCTA GAGGACTGCG TAATAACCTA TGAAAAATGA TATGAGCAAC
  51 GCCGCTCTGC TTTGCCGCAT ATACTAAATT TTCCACTTCA GGAATACGTT
101 TGAATGATGG ATGGATAATA CTTGGAATAA ACACAACGGT ATCCATTCCT
151 TTAAATGCTT CTACCATGCT TTCTTGATTA AAATAATCTA ATTGTCGAAC
201 AGGAACTTTT CCGCGCCAAT CTTCTGGAAC TTTCTCAACA TTTCTAACAC
251 CAATGTGAAA ATGATCTATG TGATTTGCAA TGGCTTGATT TGTAATATGT
301 GTGCCTAAAT GACCTGTAGC ACCTGTTAAC ATAATATTCA TTCACTTCAT
351 CTCCTAATCT TTATATACAT AACATAATAC TTATTTGATG GTTTTCAAAA
401 CATTTGATTT TATAAAAAAT TCTAATCTGT ATTTATTGTC GACGTGTATA
451 GTAAATACGT AAATATTANT AATGTTGAAA ATGCCGTAAT GACGCGTTTT
501 AGTTGATGTG TTTCACTAAT ATCATTGAAA ATTTTAATCA GGTACTACGA
551 CAATATGAAG TCTGTTTTGT GTCTGAAAAT TTTACAGTTT TTAAAATAAA
601 AATGGTATAA GTTGTGATTT GGTTTAAAAA ANAATCTCGA CGGATAANAA
```

Mutant: NT438
phenotype: temperature sensitivity
Sequence map: Mutant NT438 is complemented by plasmid pMP511, which carries a 2.3 kb insert of wild-type S. aureus genomic DNA. A partial restriction map is depicted in FIG. 87; no apparent restriction sites for EcoR I, HinD III, BamH I or Pst I are present. Database searches at the nucleic acid and (putative) polypeptide levels against currently available databases reveal strong peptide-level similarities to the nifS gene product, encoding a protein involved in the response pathway for nitrogen assimilation, from A. azollae (Genbank Accession No L34879; published in Jackman, D. M. et al. Microbiology 141, pt. 9 (1995) 2235-2244).

DNA sequence data: The following DNA sequence data represents the sequence generated by primer walking through clone pMP511, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP511
SEQ ID NO. 102
pMP511 Length: 2341 nt
      1 CTTGCATGCC TGCAGGTCGA TCTTTATTAT NATCTACACC ACGTANCATT
    51 TCAACATGAC CACGNTCATG ACGATGTATG CGTGCGTAAW GTCCTGTKGY
  101 WACATAATCK GCACCTAAAT TCATCGCATG ATCTAAAAAG GCTTTAAACT
  151 TAATTTCTTT ATWAMACATA ACGTCTGGAT TTGGAGTACG ACCTTTTTTG
  201 TATTCATCTA AGAAATACGT AAAGACTTTA TCCCAATATT CTTTTTCAAA
  251 ATTAACAGCG TAATACGGAA TGCCAATTTG ATTACACACT TCAATAACAT
  301 CGTTGTAATC TTCAGTTGCA GTACATACGC CATTTTCGTC AGTGTCATCC
  351 CAGTTTTTCA TAAATATGCC AATGACATCA TAACCTTGTT CTTTTAAGAC
  401 GTGGGCTGTT ACAGAACTAT CTACACCGCC TGACATACCA ACGACAACAC
  451 GTTATATCTT TATTTGACAA TTATGACTCC TCCTTAAATT TAAAATATAT
  501 TTTATGAATT TCAGCTACAA TTGCATTAAT TTCATTTTCA GTAGTCAATT
  551 CGTTAAAACT AAATCGAATC GAATGATTTG ATCGCTCCTC ATCTTCGAAC
  601 ATTGCATCTA AAACATGCGA CGGTTGTGTA GAGCCTGCTG TACATGCAGA
  651 TCCAGACGAC ACATAGATTT GTGCCATATC CAACAATGTT AACATCGTTT
  701 CAACTTCAAC AAACGGAAAA TATAGATTTA CAATATGGCC TGTAGCATCC
  751 GTCATTGAAC CATTTAATTC AAATGGAATC GCTCTTTCTT GTAATTTAAC
  801 TAAAAATTGT TCTTTTAAAT TCATTAAATG AATATTGTTA TCGTCTCGAT
  851 TCTTTTCTGC TAATTGTAAT GCTTTAGCCA TCCCAACAAT TTGCGCAAGA
  901 TTTTCAKTGC CTAGCACGGC GTTTCAATTC TTGTTCACCG CCAAGTTGAG
  951 GATAATCTAG TGTAACATGG TCTTTAACTA GTAATGCACC GACACCTTTT
1001 GGTCCGCCAA ACTTATGAGC AGTAATACTC ATTGCGTCGA TCTCAAATTC
1051 GTCAAWCTTA ACATCAAGAT GTCCAATTGC TTGAACCGCA TCAACATGGA
1101 AATATGCATT TGTCTCAGCA ATAATATCTT GAATATCATA AATTTGTTGC
1151 ACTGTGCCAA CTTCATTATT TACAAACATA ATAGATACTA AAATCGTCTT
1201 ATCTGTAATT GTTTCTTCAA GTTTGATCTA AATCAATAGC ACCTGTATCA
1251 TCARCATCTA GATATGTTTA CATCAAAACC TYCTCGCTCT AATTGTTCAA
1301 AAACATGTAA CACAGAATGA TGTTCAATCT TCGATGTGAT AATGTGATTA
1351 CCCAATTGTT CATTTGCTTT TACTATGCCT TTAATTGCCG TATTATTCGA
1401 TTCTGTTGCG CCACTCGTAA ATATAATTTC ATGTGTATCT GCACCAAGTA
1451 ATTGTGCAAT TTGACGTCTT GACTCATCTA AATATTTACG CGCATCTCTT
1501 CCCTTAGCAT GTATTGATGA TGGATTACCA TAATGCGAAT TGTAAATCGT
1551 CATCATCGCA TCTACTAACT TCAGGTTTTA CTGGTGTGGT CGCAGCATAA
1601 TCTGCATAAA TTTCCCATGT TTGGACAACT CCTCACAATT TTATCAATGT
1651 TCCAATAATA GCACCTTAAC ATACTATTTT TCTAACTTTT CTGTTTAACT
1701 TTATTTATAA TGTTTTTAAT TATATTTTAC CATTTTCTAC ACATGCTTTT
1751 CGATAGGCTT TTTTAAGTTT ATCGCTTTAT TCTTGTCTTT TTTATAAATT
1801 TTAGTATTTG CAGATATTTT TTTATTTGTA AAATGTAACG TACTATTATT
1851 TTGGTTATGA GCAATTTAAT ATTTATCTGG TTATTCGGAT TGGTATACTT
1901 CTTATATCAT AAAAAAGGAA GGACGATATA AAAATGGCGG ATTAAATATT
1951 CAGCAKKRAA CCTTGTCCCT ATTCGAGAAG GTGAAGATGA ACAAACAGCA
2001 ATTAATAATA TGGTTAATCT CGCACAACAT TTAGACGAAT TATCATATGA
2051 AAGATATTGG ATTGCTGAAC ACCATAACGC TCCCAACCTA GTAAGTTCAG
2101 CAACTGCTTT ATTAATTCAA CATACGTTAG AACATACGAA ACACATACGT
2151 GTAGGTTCTG GAGGCATCAT GTTACCTAAT CATGCTCCAT TAATCGTTGC
2201 GGAACAATTT GGCACGATGG CAACATTATT TCCAAATCGT GTCGATTTAG
2251 GATTAGGACG TGCACCTGGA ACAGATATGA TGACCGCAAG TGCATTAAGA
2301 CGAGATCGAC TNTAGAGGAT CCCCGGGTAC CGAGCTCGAA T
```

Mutant: NT462
phenotype: temperature sensitivity
Sequence map: Mutant NT462 is complemented by plasmid pMP540, which carries a 2.0 kb insert of wild-type S. aureus genomic DNA. A partial restriction map is depicted in FIG. 88; no apparent restriction sites for EcoR I, HinD III, BamH I or Pst I are present. Database searches at the nucleic acid and (putative) polypeptide levels against currently available databases reveal limited peptide-level similarity to a transposase-like protein from S. aureus; the putative function of the ORF contained in clone pMP540 is unclear and will require further characterization.

DNA sequence data: The following DNA sequence data represents the sequence generated by primer walking through clone pMP540, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP540
SEQ ID NO. 103
pMP540 Length: 2026 nt
      1 AAGGAAACCA CCAACACCTG CGCCAACTAA ACCKCCTGTT AGTGCAGAAA
    51 TAACGCTAAT AGCCCCCGCA CCTAAAGCAG CTRKNGTTTT TGTATATGCA
  101 GAAGAAAGAT ATAATGTTGC AGTATCTTTA CCTGTTTCTA CATATTGAGT
  151 TTTACCCGCT CTCAATTGGT CTTCAGCTTT ATATTTNTWT ATTTCTTCTW
  201 TAGTAAATAT ATCTTCCRGT TTATAACCTT TTTTCTCAAG TTCATCAAAT
  251 AAATTTWGGT TACTCAAATA TATTACCTTT GCTTGAGAAT GGTCTAACTT
  301 ATCTTCAGCA TGAGCTACAT CTGAATTATA GAGATAATGA AATTGGACTA
  351 ACAAATAATA CACCAGCAGC TRRTAATAAG AGATTTTTAA TTCGTTTTTC
  401 ATTAGTTTCT TTTAGATGAT TTTTGTATTT AGATTTCGTA TAAACAGAAA
  451 CTAGATTTTT TCATGATCGA CCTATCTTTT GTCCAGATAC AGTGAGACCT
  501 TGTCATTTAA ATGATTTTTA ATTCGTCTTG TACCAGAGAC TTTTCTATTA
  551 GAATTAAAAA TATTTATGAC GGCTGTTCTA TGTTTGAATC ATCTTTAGTG
  601 ATTTTATTAT CTTTTCTTTT TATAGAATCA TAATAGGTAC TTCTTAGTAT
  651 TATCAGGACT TTACACATTG NTGATACTGA ATANTGATGT GCATTCTTTT
  701 GAATGACTTC TATTTTTGCC CCATAATCAG CGCTACTTGC TTTAAAATAT
  751 CGTGCTCCAT TTTAAAATGT TGAACTTCTT TGCGTAATTT AATCAGGTCT
  801 TTTTCTTCAT CCGATAAGTT ATCTTGGTGA TTGAATGTAC CCGTGTTTTG
  851 ATGTTGCTTT ATCCATTTTC CTACATTTTA TAACCGCCAT TTACAAACGT
  901 CGAAKGTGTG AAATCATACT CGCGTWTAAT TTCATTCCTA GGCTTACCAT
  951 TTTTATATAA TCTAACCATT TGTAACTTAA ACTCTGAACT AAATGATCTT
1001 CTTTCTCTTG TCATAATAPA ATCGCCTACT TTCTTAAATT AACAATATCT
1051 ATTCTCATAG AATTTGTCCA ATTAAGTGTA GACGATTCAA TCTATCAGCT
1101 AGAATCATAT AACTTATCAG AAGCAAGTGA CTGTGCWTGT ATATTTGCCG
1151 MTGATATAAT AGTAGAGTCG CCTATCTCTC AGGCGTCAAT TTAGACGCAG
1201 AGAGGAGGTG TATAAGGTGA TGCTYMTTTT CGTTCAACAT CATAGCACCA
1251 GTCATCAGTG GCTGTGCCAT TGCGTTTTTY TCCTTATTGG CTAAGTTAGA
1301 CGCAATACAA AATAGGTGAC ATATAGCCGC ACCAATAAAA ATCCCCTCAC
1351 TACCGCAAAT AGTGAGGGGA TTGGTGTATA AGTAAATACT TATTTTCGTT
1401 GTCTTAATTA TACTGCTAAT TTTTCTTTTT GTAAAATATG CAAGGTTTTA
1451 AAGAGAAACA TCAAGAACTA AAAAAGGCTY TATGTCAAAT TGGACTGATG
1501 CGTTCAATAT CCGAAGTTAA GCAACTAAAC ATTGCTTAAC TTCCTTTTTA
1551 CTTTTTGGAG CGTAAAGTTT TGAACATAAT AATATTCGAT TGCGCAAATG
1601 ATTGTAACTT CCATAACCAA AAGATGTACG TTTAATTAAT TTTATTTTGT
1651 TATTTATACC TTCTAAAGGA CCATTTGATA AATTGTAATA ATCAATGGTT
1701 ACACTATTAA AAGTGTCACA AATTCTTATG AATCTGGCAT AAACTTTGAA
1751 TTAACTAAAT AAGTAAGAAA ACCTCGGCAC TTTATCATTT TAATAGTGTC
1801 GAGATTTTTA TAGATACTAC AAATATTTAT AACATAGTTA AACTCATCTA
1851 ATGACTTATA TTTTTGTTTC ATCACAATAT GAACAATTAT TTATTGGACG
1901 TATTTTGCTC TTTTTTTATT TCAGAAACTG ACTTAGGATT TTTATTAAAT
1951 TTTCTACCCA ATTCATCTGT ATAAGAAATA TCGGTATCAA ATTGAAAATC
2001 ATCAACAGAT CGACCTGCAG GCATGC
```

Mutant: NT482
phenotype: temperature sensitivity
Sequence map: Mutant NT482 is complemented by plasmid pMP560, which carries a 2.7 kb insert of wild-type S. aureus genomic DNA. A partial restriction map is depicted in FIG. 89. Database searches at the nucleic acid and (putative) polypeptide levels against currently available databases reveal strong similarity at the peptide-level to the folC gene product, encoding folyl polyglutamate synthase (FGPS), from B. subtilis (Genbank Accession No. L04520; published in Mohan, S. et al., J. Bacteriol. 171 (1989) 6043-6051.)

DNA sequence data: The following DNA sequence data represents the sequence generated by primer walking through clone pMP560, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP560
SEQ ID NO. 104
pMP560 Length: 2736 nt
      1 TGCCTGCAGG TCGATCTTCT ATGTAAATAA TCAAATGACG TTTCTTCTAT
    51 AGATATAAAT TGATATASAA AACTAAAAAT ACAACTGCAA CTATAAGATA
  101 ACAATACTAC CAAATGACAA CCTCCTTATG TAAATTATAG TTAGTTATTA
  151 CCAAAATGTA AATATACACT ATTTTTCAAG AATTGAACCG CTTTTTCATT
  201 TAAATTTTTC AATATTGCTA AGCATAATTG ATGGATACTT TAACAACCCA
  251 TTACTGCTCG GCAAAATTAA TAATGGCAAG AAATTGAACC TTATAAACAC
  301 ATACGATTTA GAGCATAAAA AATAACCATG AAGCTCTACC TATTGATTAA
  351 ATARATTCTT CATGGCTATT TTAGTTTTAG TTTTATAATG CTTCAAAGTC
  401 TAATTTTGAT TTAACTTCAC TTATGAAATA CAGACTACCG GTAATTACTA
  451 ATGTATCACC TTGATAATTT TTTATAAATT CAACGTAGTC ATCTACTAAT
  501 TGTATTTCAT CATTTTCAAT ACTACCTACA ATTTCTTCTT TGCGTAACGC
  551 TTTCGGAAAA TCAAATTCAG TTGCATAAAA CGTATGCGCA ATTAAACTTA
  601 AATGTTTGAC CATCTCGTTA ATCGGTTTTC CGTTTATTGC TGASAACAAA
  651 ATATCTACTT TTTCTTTATC ATGGTACTGT TTAATTGTAT CAATTAGAGC
  701 ATCTATACTC TCTGAATTAT GYGCGCCATC CAAAATGATT AAAGGYTTGT
  751 CATGCACCTG CTCAATACGT CCAGTCCAAC GAACTGATTC AATACCGTCT
  801 ATCATCTTAT TGAAATCTAA TTCAATTAAT CCTTGTTCAT TTAATTCAAT
  851 AAGAGCTGTT ATGGCTAATG CAGCAAWTTT GTTTCTGATG TTTCACCTAA
  901 CATGCTTAAA ATGATTGTTT CTAATTCATA ATCTTTATAA CGGTAAGTTA
  951 AATTCATCAT TTTGCGATAC AACAACAATT TCTCTATCTA ATTCAATGGC
1001 TTTGCATGTT GTTCAATTGC GCGTTCACGA ACATATTTTA ATGCATCTTC
1051 ATTTTTTACA GCATATATCA CTGGAACKTT AGGSTTTATA ATCGCGCCYT
1101 TATCCCTAGC AATATCTAGA TAAGTACCAC CTAAAATATC TGTATGGTCT
1151 AGACCGATAC TAGTTAAGAT TGATAAAACC GGTGTAAAGA CATTTGTCGA
1201 ATCGTTCTTT ATACCCAATC CAGCCTCAAC AATGACAAAA TCAACAGGAT
1251 GTATTTCACC AAAATATAAA AACATCATCG CTGTGATTAT TTCGAATTCA
1301 GTTGCAAMMM CTAAATCTGT TTCAMSTTCC ATCATTTCAA TTAACTGGTT
1351 TAATACGTGA TACTAATTCT AACAATAGCG TCATTTGATA TTGGCAACAC
1401 CATTTAGRAT AATTCGTTCA TTAAATGTTT CAATAAACGG CGACGTAAAT
1451 GTACCTACTT CATAACCATT TTCAACTAAA GCTGTTCTAA GGTAAGCAAC
1501 TGTAGAGCCT TTACCATTTG TGCCACSKAC ATGAATACCC TTAATGWTAT
1551 TTTGAGGATT ATTAAATTGT GCTAGCATCC ATTCCATACG TTTAACACCT
1601 GGTTTGATGC CAAATTTAGT TCTTTCGTGT ATCCAATACA AGCTCTCTAG
1651 GTAATTCATT GTTACTAACT CCTATGCTTT TAATTGTTCA ATTCTTGCCT
1701 TCACACCATC ATATTTTTCT TGATAATCTT GTTTTTTACG TTTTTCTTCA
1751 TTTATAACCT TTTCAGGTGC TTTACTTACA AAGTTTTCAT TAGAGAGCTT
1801 TTTATCTACT CTATCTAATT CGCTTTGAAG TTTAGCTAAT TCTTTTTCCA
1851 AACGGCTGAT TTCCTTATCC ATATCAATTA GCCCTTCTTA ATGGTAATAC
1901 CCACTTTACC TGCAATTACA ACTGATGTCA TTGCTTTCTC AGGAATTTCC
1951 AACGTCAGTG CTAATATTTA AGGTACTAGG ATTACAGAAT TTGATTAAAT
2001 AATCTTTGTT TTGTGATAAA GTTGTTTCAA TTTCTTTATC TTTAGCTTGA
2051 ATTAAAATAG GTATTTCTTT AGACAATGGC GTATTTACTT CTACACGTGA
2101 TTGTCTTACA GATTTAATGA TTTCAACAAG TGGTKGCATT GTTTGTTAAC
2151 TTTCTTCAAA AATCAATGAT TCACGCACTT CTGGCCATGA AGCTTTAACA
2201 ATTGTGTCAC CTTCATGTGG TAAACTTTGC CATATTTTCT CTGTTACAAA
2251 TGGCATGAAT GGATGTAGCA TTCTCATAAT ATTGTCTAAA GTATAACTCA
2301 ATACTGAACG TGTAACTTGT TTTTGTTCTT CATCATTACT ATTCATTGGA
2351 ATTTTACTCA TTTCAATGTA CCAATCACAG AAATCATCCC AAATGAAATT
2401 ATATAATGCA CGTCCAACTT CGCCGAATTC ATATTTGTCA CTTAAATCAG
2451 TAACTGTTGC AATCGTTTCA TTTAAACGTG TTAGAATCCA TTTATCTGCT
2501 AATGATAAGT TACCACTTAA ATCGATATCT TCAACTTTAA AGTCTTCACC
2551 GATATTCATT AAACTGAAAC GTGCCCCATT CCAGATTTTA TTGATAAAGT
2601 TCCACACTGA CTCAACTTTT TCAGTTGAGT ATCTTAAATC ATGTCCTGGA
2651 GATGAACCTG TTGCTAAGAA GTAACGCAAG CTATCAGCAC CGTATTCGTC
2701 AATAACATCC ATTGGATCGA CCTGCAGGCA TGCAAG
```

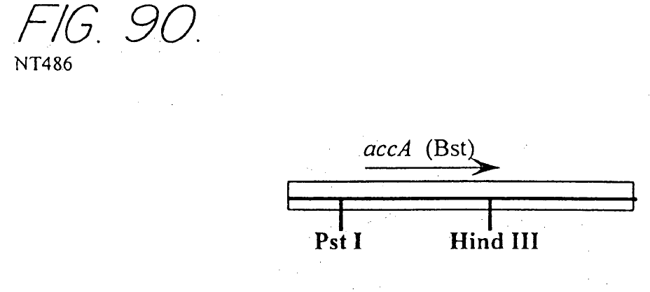

Mutant: NT486
phenotype: temperature sensitivity
Sequence map: Mutant NT486 is complemented by plasmid pMP567, which carries a 2.3 kb insert of wild-type S. aureus genomic DNA. A partial restriction map is depicted in FIG. 90; no apparent restriction sites for EcoR I, HinD III, BamH I or Pst I are present. Database searches at the nucleic acid and (putative) polypeptide levels against currently available databases reveal strong peptide-level similarities to the accA gene product, encoding the alpha subunit of acetyl-CoA-carboxylase carboxyl transferase (EC 6.4.1.2), from B. stearothermophilus (Genbank Accession No. D13095); this gene product forms part of an enzyme complex responsible for fatty acid biosynthesis and is thought to be essential.

DNA sequence data: The following DNA sequence data represents the sequence generated by primer walking through clone pMP567, starting with standard M13 forward and M13 reverse sequencing primers and completing the sequence contig via primer walking strategies. The sequence below can be used to design PCR primers for the purpose of amplification from genomic DNA with subsequent DNA sequencing.

```
clone pMP567
SEQ ID NO. 105
pMP567 Length: 2255 nt
      1 CNCGNNAGCG ANGTNGCCGA GGATCCTCTA GAGTCNATCG GTTATCGGTG
    51 AAAAGATATG TCGCATCATT GATTACTGCA CTGAGAACCG TTTACCATTT
  101 ATTCTTTTCT CTGCAAGTGG TGGTGCACGT ATGCAAGAAG GTATTATTTC
  151 CTTGATGCAA ATGGGTAAAA CCAGTGTATC TTTAAAACGT CATTCTGACG
  201 CTGGACTATT ATATATATCA TATTTAACAC ATCCAACTAC TGGTGGTGTA
  251 TCTGCAAGTT TTGCATCAGT TGGTGATATA AATTTAAGTG AGCCAAAAGC
  301 GTTGATAGGT TTTGCAGGTC GTCGAGTTAT TGAACAGACA ATAAACGAAA
  351 AATTGCCAGA TGATTTCCAA ACTGCAGAAT TTTTATTAGA GCATGGACAA
  401 TTGGATAAAG TTGTACATCG TAATGATATG CGTCAAACAT TGTCTGAAAT
  451 TCTAAAAATC CATCAAGAGG TGACTAAATA ATGTTAGATT TTGAAAAACC
  501 ACTTTTTGAA ATTCGAAATA AAATTGAATC TTTAAAAGAA TCTCAAGATA
  551 AAAATGATGT GGATTTACCA AAGAAGAATT TGACATGCCT TGAARCGTCM
  601 TTGGRACGAG AAACTAAAAA AATATATACA AATCTAAAAC CATGGGATCG
  651 TGTGCAAATT GCGCGTTTGC AAGAAAGACC TACGACCCTA GATTATATTC
  701 CATATATCTT TGATTCGTTT ATGGAACTAC ATGGTGATCG TAATTTTAGA
  751 GATGATCCAG CAATGATTGG TGGTATTGGC TTTTTAAATG GTCGTGCTGT
  801 TACAGTYRTK GGACAACAAC GTGGAAAAGA TACWAAAGAT RATATTTATC
  851 GAAATTTTKG GTATGGCGCA TCCAGAAGGT TATCGAAAAG CATTACGTTT
  901 AATGAAACAA GCTGAAAAAT TCAATCGTCC TATCTTTACA TTTATAGATA
  951 CAAAAGGTGC ATATCCTGGT AAAGCTGCTG AAGAACGTGG ACAAAGTGAA
1001 TCTATCGCAA CAAATTTGAT TGAGATGGCT TCATTAAAAG TACCAGTTAT
1051 TGCGATTGTC ATTGKYGAAG GTGGCAGTGG AGGTGCTCTA GGTATTGGTA
1101 TTGCCAATAA AGYATTGATG TTAGAGAATA GTACTTACTC TGWTATATCT
1151 CCTGAAGGTG CAGCGGCATT ATTATGGAAA GACAGTAATT TGGCTAAAAT
1201 YGCAGCTGAA ACAATGAAWA TTACTGCCCA TGATATTAAG CAATTAGGTA
1251 TTATAGATGA TGYCATTTCT GAACCACTTG GCGGTGCACA TAAAGATATT
1301 GAACAGCAAG CTTTAGCTAT TAAATCAGCG TTTGTTGCAC AGTTAGATTC
1351 ACTTGAGTCA TTATCAACGT GATGAAATTG CTAATGATCG CTTTGAAAAA
1401 TTCAGAAATA TCGGTTCTTA TATAGAATAA TCAACTTGAG CATTTTTATG
1451 TTAAATCGAT ACTGGGTTTT ACCATAAATT GAAGTACATT AAAACAATAA
1501 TTTAATATTT AGATACTGAA TTTTTAACTA AGATTAGTAG TCAAAATTGT
1551 GGCTACTAAT CTTTTTTTAA TTAAGTTAAA ATAAAATTCA ATATTTAAAA
1601 CGTTTACATC AATTCAATAC ATTAGTTTTG ATGGAATGAC ATATCAATTT
1651 GTGGTAATTT AGAGTTAAAG ATAAATCAGT TATAGAAAGG TATGTCGTCA
1701 TGAAGAAAAT TGCAGTTTTA ACTAGTGGTG GAGATTCACC TGGAATGAAT
1751 GCTGCCGTAA GAGCAGTTGT TCGTACAGCA ATTTACAATG AAATTGAAGT
1801 TTATGGTGTG TATCATGGTT ACCAAGGATT GTTAAATGAT GATATTCATA
1851 AACTTGAATT AGGATCRAGT TGGGGATACG ATTCAGCGTG GAGGTACATT
1901 CTTGTATTCA GCAAGATGTC CAGAGTTTAA GGAGCAAGAA GTACGTAAAG
1951 TTGCAATCGA AAACTTACGT AAAAGAGGGA TTGAGGGCCT TGTAGTTATT
2001 GGTGGTGACG GTAGTTATCG CGGTGCACAA CGCATCAGTG AGGAATGTAA
2051 AGAAATTCAA ACTATCGGTA TTCCTGGTAC GATTGACAAT GATATCAATG
2101 GTACTGATTT TACAATTGGA TTTGACACAG CATTAAATAC GATTATTGGC
2151 TTAGTCGACA AAATTAGAGA TACTGCGTCA AGTCACGCAC GAACATTTAT
2201 CATTGAAGCA ATGGGCCGTG ATTGTGGAGT CATCTGGAGT CGACCTGCTA
2251 GTCTT
```

II. Homologous Genes

As described above, the use of genes from other pathogenic bacterial strains and species which are homologous to the identified genes from Staphylococcus aureus is also provided. Such homologous genes not only have a high level of sequence similarity with the particular S. aureus genes, but also are functional equivalents. This means that the gene product has essentially the same biological activity. Therefore, the homologous genes are identifiable, for example, based on a combination of hybridization of all or a portion of one gene to its homologous counterpart, and the ability of the homologous gene to complement the growth conditional mutant of S. aureus under non-permissive conditions. The ability of the homologous gene to hybridize with sequences from the S. aureus gene provides that homologous gene using generally accepted and used cloning techniques. The ability of the homologous gene to complement a defective S. aureus gene demonstrates that the genes are essentially equivalent genes found in different bacteria.

Specific examples of methods for identifying homologous genes are described in Van Dijl et al., U.S. Pat. No. 5,246,838, issued Sep. 21, 1993. In addition to the direct hybridization methods for identifying and isolating homologous genes mentioned above, Van Dijl et al. describe the isolation of homologous genes by isolating clones of a host bacterial strain which contain random DNA fragments from a donor microorganism. In those clones a specific host gene has been inactivated (such as by linkage with a regulatable promoter), and inserted homologous genes are identified by the complementation of the inactivated gene function. Homologous genes identified in this way can then be sequenced.

If the function of the product of a specific host gene is known, homologous gene products can often be isolated (by assaying for the appropriate activity) and at least partially sequenced (e.g., N-terminal sequencing). The amino acid sequence so obtained can then be used to deduce the degenerate DNA base sequence, which can be used to synthesize a probe(s) for the homologous gene. A DNA library from another microorganism is then probed to identify a clone(s) containing a homologous gene, and the clone insert sequenced.

These and other methods for identifying homologous genes are well-known to those skilled in the art. Therefore, other persons can readily obtain such genes which are homologous to the genes corresponding to SEQ ID NO. 1-105.

III. Evaluation of Gene as Therapeutic Target

A. General Considerations

While the identification of a particular bacterial gene as an essential gene for growth in a rich medium characterizes that gene as an antibacterial target, it is useful to characterize the gene further in order to prioritize the targets. This process is useful since it allows further work to be focused on those targets with the greatest therapeutic potential. Thus, target genes are prioritized according to which are more likely to allow identification of antibacterial agents which are:

1. Highly inhibitory to the target in relevant pathogenic species;

2. Cause rapid loss of bacterial viability;

3. Not have frequently arising resistance mechanisms;

4. Have high selectivity for the bacterial target and little, or preferably no, effect on the related mammalian targets;

5. Have low non-specific toxicity to mammals; and

6. Have appropriate pharmacodynamic and physical properties for use as a drug.

Consequently, target genes are prioritized using a variety of methods, such as those described below.

B. Methods for Recognizing Good Targets

Essential genes can be characterized as either bactericidal or bacteriostatic. Earlier work with Salmonella mutants established that the bactericidal/bacteriostatic distinction was a characteristic of inhibition of the specific gene, rather than of a mutant allele, and could be characterized in vitro. (Schmid et al., 1989, Genetics 123:625-633.) Therefore, preferred targets (high priority) are those which are highly bactericidal when inhibited, causing cell death. A subset of the bactericidal essential genes can be identified as strongly bactericidal, resulting in rapid cell death when inhibited.

In S. typhimurium, inhibition of strongly bactericidal genes was shown to result in one of the following effects:

1. Cell lysis (such genes generally involved in cell wall biosynthesis);

2. Inhibition of protein synthesis;

3. DNA degradation; or

4. Entry into non-recoverable state involving cell cycle related genes.

In Vivo Switch

In addition to the prioritization of gene targets based on the observed in vitro phenotypes, further evaluation of a specific gene as a potential therapeutic target is performed based on the effects observed with loss of that gene function in vivo. One approach is the use of null mutants in which the mutant gene product is inactive at 37° C. In the case of essential genes for which temperature sensitive mutants were previously isolated, those mutant strains can be used in this evaluation if the gene product is essentially inactive at 37° C. If such a temperature sensitive mutant has not previously been isolated but a complementing clone of some growth conditional mutant is available, then the required null mutants can generally be isolated through the use of localized mutagenesis techniques (Hong and Ames, 1971, Proc. Natl. Acad. Sci. USA 68:3158-3162). The evaluation then involves the comparison of the in vivo effects of the normal strain and the mutant strain. The comparison involves determinations of the relative growth in vivo, relative bactericidal phenotype in vivo and differences in response in various infection models.

In addition to gene target evaluations using null mutant experiments, related evaluations can be performed using “in vivo switch” methods. Such methods allow control of the expression of a gene in vivo, and so provide information on the effects of inhibiting the specific gene at various time points during the course of an infection in a model infection system. In effect, an in vivo switch provides a mimic of the administration of an inhibitor of a gene, even if such an inhibitor has not yet been identified.

Such in vivo switch methods can be carried out by using recombinant strains of a pathogenic bacterium, which carry a test gene transcriptionally linked with an artificially controllable promoter. One technique for doing this is to use the natural promoter for the test gene, and insert an operator site in a position so that transcription will be blocked if a repressor molecule is bound to the operator. Expression of the repressor molecule is then placed under artificial control by linking the gene for the repressor with a promoter which can be controlled by the addition of a small molecule. For example, a β-lactamase receptor/repressor/promoter system can be used to control expression of a lac repressor, which, in turn, will bind to a lac operator site inserted in the test gene. These DNA constructs are then inserted into bacteria in which the endogenous copy of the test gene has been inactivated, and those bacteria are used in various infection models. Therefore, for this system, the test gene will be expressed prior to administration of a β-lactam. However, when a β-lactam with little or no intrinsic antibacterial activity (e.g., CBAP) is administered to an animal infected with the recombinant bacteria, the β-lactam induces production of lac repressor. The lac repressor molecule then binds to the lac operator, stopping (turning off) expression of the test gene.

The method can be extended by administering the β-lactam (or other appropriate controller molecule) at different times during the course of an infection, and/or according to different schedules of multiple dosing. Also, many different designs of in vivo switch may be used to provide control over the test gene. In general, however, such a method of target evaluation provides information such as:

1. a measure of the “cidalness” of the target gene following inhibition of that gene;

2. a benchmark against which to measure chemical inhibitors as they are identified, since the in vivo switch can mimic complete inhibition of the gene;

3. an estimate of the efficacy of inhibitor use at different time points in an infection process; and

4. an estimate of the efficacy of inhibitor use in various types of infections, in various in vivo environments.

Information of this nature is again useful for focusing on the gene targets which are likely to be the best therapeutic targets.

C. In Vivo Evaluation of Microbial Virulence and Pathogenicity

Using gene target evaluation methods such as the null mutant and in vivo switch methods described above, the identified target genes are evaluated in an infection model system. (References herein to the use of animals or mammals should be understood to refer to particular infection models. Other infection systems may be used, such as cell-based systems as surrogates for whole organism models, or systems to evaluate possible antimicrobial targets of pathogens of organisms other than animals (e.g., plants). The criteria for evaluation include the ability of the microbe to replicate, the ability to produce specific exoproducts involved in virulence of the organism, and the ability to cause symptoms of disease in the animals.

The infection models, e.g., animal infection models, are selected primarily on the basis of the ability of the model to mimic the natural pathogenic state of the pathogen in an organism to be treated and to distinguish the effects produced by activity or by loss of activity of a gene product (e.g., a switch in the expression state of the gene). Secondarily, the models are selected for efficiency, reproducibility, and cost containment. For mammal models, rodents, especially mice, rats, and rabbits, are generally the preferred species. Experimentalists have the greatest experience with these species. Manipulations are more convenient and the amount of materials which are required are relatively small due to the size of the rodents.

Each pathogenic microbe (e.g., bacterium) used in these methods will likely need to be examined using a variety of infection models in order to adequately understand the importance of the function of a particular target gene.

A number of animal models suitable for use with bacteria are described below. However, these models are only examples which are suitable for a variety of bacterial species; even for those bacterial species other models may be found to be superior, at least for some gene targets and possibly for all. In addition, modifications of these models, or perhaps completely different animal models are appropriate with certain bacteria.

Six animal models are currently used with bacteria to appreciate the effects of specific genes, and are briefly described below.

1. Mouse Soft Tissue Model

The mouse soft tissue infection model is a sensitive and effective method for measurement of bacterial proliferation. In these models (Vogelman et al., 1988, J. Infect. Dis. 157: 287-298) anesthetized mice are infected with the bacteria in the muscle of the hind thigh. The mice can be either chemically immune compromised (e.g., cytoxan treated at 125 mg/kg on days −4, −2, and 0) or immunocompetent. The dose of microbe necessary to cause an infection is variable and depends on the individual microbe, but commonly is on the order of 10⁵-10⁶ colony forming units per injection for bacteria. A variety of mouse strains are useful in this model although Swiss Webster and DBA2 lines are most commonly used. Once infected the animals are conscious and show no overt ill effects of the infections for approximately 12 hours. After that time virulent strains cause swelling of the thigh muscle, and the animals can become bacteremic within approximately 24 hours. This model most effectively measures proliferation of the microbe, and this proliferation is measured by sacrifice of the infected animal and counting colonies from homogenized thighs.

2. Diffusion Chamber Model

A second model useful for assessing the virulence of microbes is the diffusion chamber model (Malouin et al., 1990, Infect. Immun. 58: 1247-1253; Doy et al., 1980, J. Infect. Dis. 2: 39-51; Kelly et al., 1989, Infect. Immun. 57: 344-350. In this model rodents have a diffusion chamber surgically placed in the peritoneal cavity. The chamber consists of a polypropylene cylinder with semipermeable membranes covering the chamber ends. Diffusion of peritoneal fluid into and out of the chamber provides nutrients for the microbes. The progression of the “infection” can be followed by examining growth, the exoproduct production or RNA messages. The time experiments are done by sampling multiple chambers.

3. Endocarditis Model

For bacteria, an important animal model effective in assessing pathogenicity and virulence is the endocarditis model (J. Santoro and M. E. Levinson, 1978, Infect. Immun. 19: 915-918). A rat endocarditis model can be used to assess colonization, virulence and proliferation.

4. Osteomyelitis Model

A fourth model useful in the evaluation of pathogenesis is the osteomyelitis model (Spagnolo et al., 1993, Infect. Immun. 61: 5225-5230). Rabbits are used for these experiments. Anesthetized animals have a small segment of the tibia removed and microorganisms are microinjected into the wound. The excised bone segment is replaced and the progression of the disease is monitored. Clinical signs, particularly inflammation and swelling are monitored. Termination of the experiment allows histolic and pathologic examination of the infection site to complement the assessment procedure.

5. Murine Septic Arthritis Model

A fifth model relevant to the study of microbial pathogenesis is a murine septic arthritis model (Abdelnour et al., 1993, Infect. Immun. 61: 3879-3885). In this model mice are infected intravenously and pathogenic organisms are found to cause inflammation in distal limb joints. Monitoring of the inflammation and comparison of inflammation vs. inocula allows assessment of the virulence of related strains.

6. Bacterial Peritonitis Model

Finally, bacterial peritonitis offers rapid and predictive data on the virulence of strains (M. G. Bergeron, 1978, Scand. J. Infect. Dis. Suppl. 14: 189-206; S. D. Davis, 1975, Antimicrob. Agents Chemother. 8: 50-53). Peritonitis in rodents, preferably mice, can provide essential data on the importance of targets. The end point may be lethality or clinical signs can be monitored. Variation in infection dose in comparison to outcome allows evaluation of the virulence of individual strains.

A variety of other in vivo models are available and may be used when appropriate for specific pathogens or specific genes. For example, target organ recovery assays (Gordee et al., 1984, J. Antibiotics 37:1054-1065; Bannatyne et al., 1992, Infect. 20:168-170) may be useful for fungi and for bacterial pathogens which are not acutely virulent to animals. For additional information the book by Zak and Sande (EXPERIMENTAL MODELS IN ANTIMICROBIAL CHEMOTHERAPY, O. Zak and M. A. Sande (eds.), Academic Press, London (1986) is considered a standard.

It is also relevant to note that the species of animal used for an infection model, and the specific genetic make-up of that animal, may contribute to the effective evaluation of the effects of a particular gene. For example, immuno-incompetent animals may, in some instances, be preferable to immuno-competent animals. For example, the action of a competent immune system may, to some degree, mask the effects of altering the level of activity of the test gene product as compared to a similar infection in an immuno-incompetent animal. In addition, many opportunistic infections, in fact, occur in immuno-compromised patients, so modeling an infection in a similar immunological environment is appropriate.

In addition to these in vivo test systems, a variety of ex vivo models for assessing bacterial virulence may be employed (Falkow et al., 1992, Ann. Rev. Cell Biol. 8:333-363). These include, but are not limited to, assays which measure bacterial attachment to, and invasion of, tissue culture cell monolayers. With specific regard to S. aureus, it is well documented that this organism adheres to and invades cultured endothelial cell monolayers (Ogawa et al., 1985, Infect. Immun. 50: 218-224; Hamill et al., 1986, Infect. and Imm. 54:833-836) and that the cytotoxicity of ingested S. aureus is sensitive to the expression of known virulence factors (Vann and Proctor, 1988, Micro. Patho. 4:443-453). Such ex vivo models may afford more rapid and cost effective measurements of the efficacy of the experiments, and may be employed as preliminary analyses prior to testing in one or more of the animal models described above.

IV. Screening Methods for Antibacterial Agents

A. Use of Growth Conditional Mutant Strains

1. Hypersensitivity and TS Mutant Phenoprints

In addition to identifying new targets for drug discovery, the growth conditional mutants are useful for screening for inhibitors of the identified targets, even before the novel genes or biochemical targets are fully characterized. The methodology can be whole-cell based, is more sensitive than traditional screens searching for strict growth inhibitors, can be tuned to provide high target specificity, and can be structured so that more biological information on test compounds is available early for evaluation and relative prioritization of hits.

Certain of the screening methods are based on the hypersensitivity of growth conditional mutants. For example, conditionally lethal ts mutants having temperature sensitive essential gene functions are partially defective at a semi-permissive temperature. As the growth temperature is raised, the mutated gene causes a progressively crippled cellular function. It is the inherent phenotypic properties of such ts mutants that are exploited for inhibitor screening.

Each temperature sensitive mutant has secondary phenotypes arising from the genetic and physiological effects of the defective cellular component. The genetic defect causes a partially functional protein that is more readily inhibited by drugs than the wild type protein. This specific hypersensitivity can be exploited for screening purposes by establishing “genetic potentiation” screens. In such screens, compounds are sought that cause growth inhibition of a mutant strain, but not of wild type, or greater inhibition of the growth of a mutant strain than of a wild type strain. Such compounds are often (or always) inhibitors of the wild type strain at higher concentrations.

Also, the primary genetic defect can cause far-reaching physiological changes in the mutant cells, even in semi-permissive conditions. Necessity for full function of biochemically related proteins upstream and downstream of the primary target may arise. Such effects cause hypersensitivity to agents that inhibit these related proteins, in addition to agents that inhibit the genetically defective cellular component. The effects of the physiological imbalance will occur through metabolic interrelationships that can be referred to as the “metabolic web”. Thus, in some cases, the initial genetic potentiation screen has the ability to identify inhibitors of either the primary target, or biochemically related essential gene targets.

With sufficient phenotypic sensors, a metabolic fingerprint of specific target inhibition can be established. Therefore, the mutant strains are evaluated to identify a diverse repertoire of phenotypes to provide this phenotypic fingerprint, or “phenoprint”. These evaluations include hypersensitivities to known toxic agents and inhibitors, carbon source utilization, and other markers designed to measure specific or general metabolic activities for establishing a mutant phenoprint that will aid in interpretation of inhibitor profiles.

2. Determination of Hypersusceptibility Profiles

As an illustration of the hypersusceptibility profiles for a group of bacterial ts mutant strains, the minimal inhibitory concentrations (MICs) of various drugs and toxic agents were determined for a set of Salmonella typhimurium temperature-sensitive essential gene mutants.

The MICs were measured by using a standard micro broth dilution technique following the recommendations of the National Committee for Clinical Laboratory Standards (1994). Bacteria were first grown in Mueller-Hinton broth at 30° C., diluted to 10⁵ cfu/ml and used to inoculate 96-microwell plates containing two-fold dilutions of antibiotics in Mueller-Hinton broth. Plates were incubated for 20 h at a semi-permissive temperature (35° C.) and the MIC was determined as the lowest dilution of antibiotic preventing visible growth.

A two-fold difference in the susceptibility level of the mutant strain compared to that of the parental strain is within the limits of the experimental variation and thus a ≧4-fold decrease in MIC was considered as a significant hypersusceptibility.

### EXAMPLE 1

### Hypersensitivity of S. aureus secA Mutants

The secA mutant strain NT65 was found to be more sensitive to compound MC-201,250. The MIC of this compound on NT65 is 0.62 μg/ml and that on the wild type strain is 50 μg/ml. The inhibitory effect of MC-201,250 on secA mutants increased as screening temperatures increased. Other secA mutants, which may represent different alleles of the gene, are also hypersensitive to this compound by varying degrees, examples are shown in Table 1 below.

TABLE 1
Hypersensitivity of secA Alleles to MC201,250

| Strain | MIC (μg/ml) |
|---|---|
| NT65 | 0.62 |
| NT328 | 1.25 |
| NT74 | 2.5 |
| NT142 | 5 |
| NT15 | 10 |
| NT67 | 10 |
| NT122 | 10 |
| NT112 | 20 |
| NT368 | 20 |
| NT413 | 20 |
| Wild Type (WT) | 50 |

Furthermore, introduction of the wild type secA allele into NT65 raised the MIC to the wild type level. These data suggest that the hypersensitivity results from the secA mutation in the mutants.

To further demonstrate that the hypersensitivity to MC-201,250 is due to the secA mutation that causes the temperature sensitivity, heat-resistant revertants, both spontaneous and UV-induced, were isolated from NT6S and tested for their responses to the compound. In a parallel experiment, MC-201250-resistant revertants were also isolated from NT65 and tested for their growth at nonpermissive temperatures. The results showed that revertants able to grow at 43° C. were all resistant to MC-201250 at the wild type level (MIC=50 μg/ml) and vice versa. Revertants able to grow at 39° C. but not at 43° C. showed intermediate resistance to MC-201,250 (MIC=1.25-2.5 μg/ml and vice versa The correlation between the heat-sensitivity and MC-201,250-sensitivity strongly suggests that the secA gene product may be the direct target for MC-201,250.

The benefits of using hypersensitive mutants for screening is apparent, as this inhibitor would have not been identified and its specificity on secA would have not been known if wild type cells rather than the mutants were used in whole cell screening at a compound concentration of 10 μg/ml or lower.

### EXAMPLE 2

### Hypersensitivity of S. typhimurium gyr Mutants

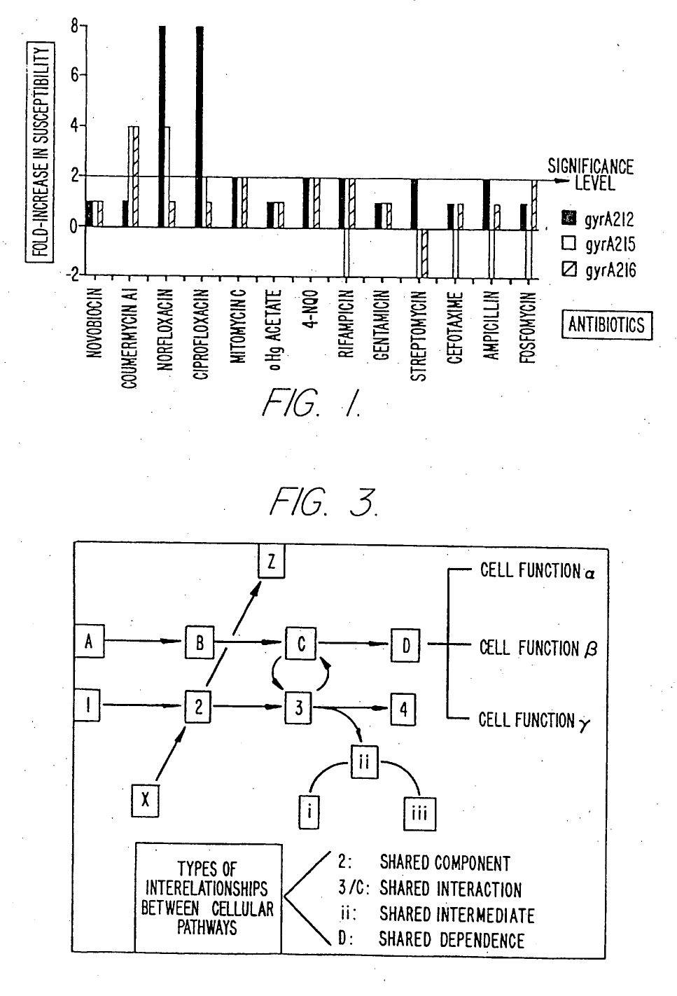

The specific hypersensitivity of temperature sensitive mutations in a known target to inhibitors of that target is shown in FIG. 1 with the susceptibility profile of three ts S. typhimurium mutant alleles of the gyrase subunit A (gyrA212, gyrA215 and gyrA216) grown at a semi-permissive temperature (35° C.). The graph shows the fold-increases in susceptibility to various characterized antibacterial agents compared to that observed with the wild-type parent strain. The data demonstrate the highly specific hypersusceptibility of these mutants to agents acting on DNA gyrase. Susceptibility to other classes of drug or toxic agents is not significantly different from the parent strain (within 2-fold).

In addition, different mutant alleles show unique hypersensitivity profiles to gyrase inhibitors. Coumermycin inhibits the B-subunit of the gyrase, while norfloxacin, ciprofloxacin, and nalidixic acid inhibit the A-subunit. One mutant shows hypersusceptibility to coumermycin (gyrA216), one to coumermycin and norfloxacin (gyrA215), and another to norfloxacin and ciprofloxacin (gyrA212). Note that a mutation in the gyrase subunit A (gyrA215) can cause hypersensitivity to B-subunit inhibitors and could be used to identify such compounds in a screen. In addition, some gyrA mutant strains show no hypersensitivity to known inhibitors; potentially, these strains could be used to identify novel classes of gyrase inhibitors. Overall these results show that a selection of mutated alleles may be useful to identify new classes of compounds that affect gyrase function including structural subunit-to-subunit interactions. Thus, use of the properties of the crippled gyrase mutants in a screen provides a great advantage over biochemical-based screens which assay a single specific function of the target protein in vitro.

### EXAMPLE 3

### Hypersensitivity Profiles of Salmonella ts Mutants

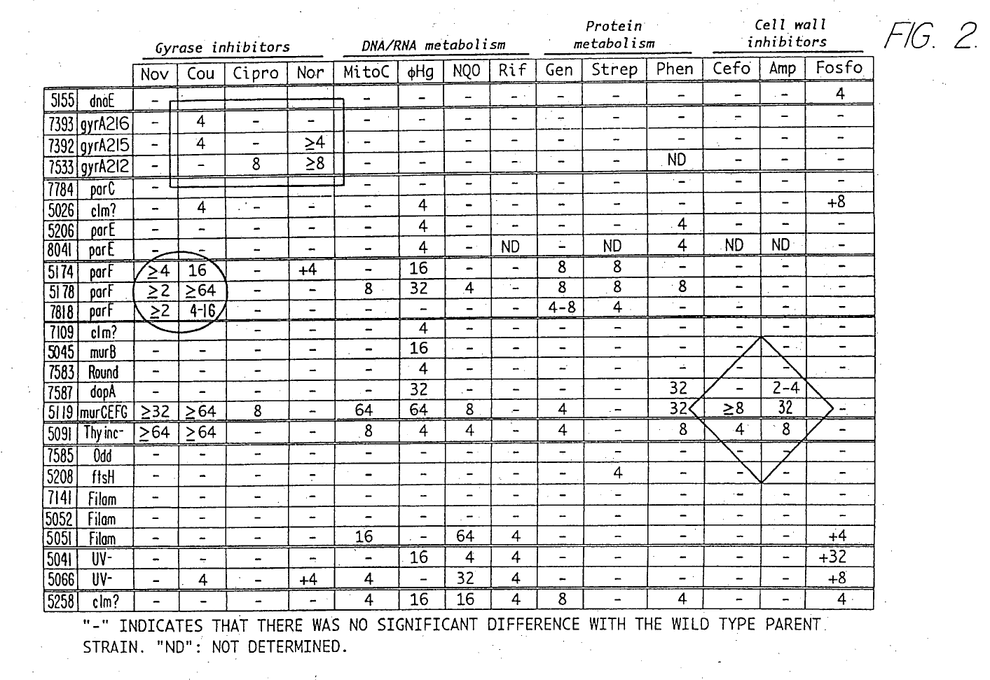

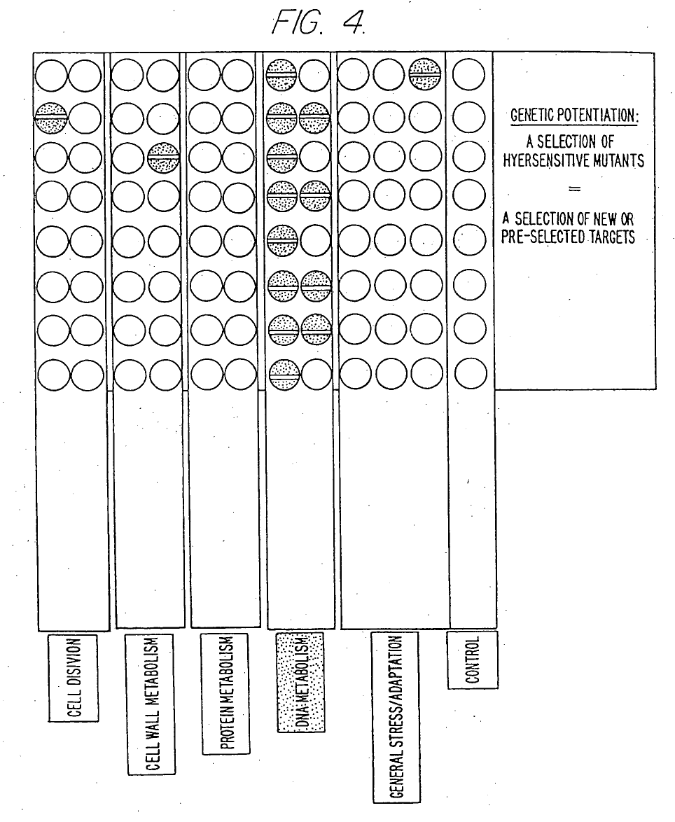

Demonstration of the generalized utility of hypersensitive screening with the conditional lethal mutants has been obtained (FIG. 2) by collecting hypersensitivity profiles from partly characterized Salmonella conditional ts mutants. The table shows the increased susceptibility of the mutant strains to various characterized antibacterial agents compared to the wild-type parent strain. A two-fold difference in the susceptibility level is within the limits of the experimental variation and thus a ≧4-fold difference is significant.

A variety of hypersusceptibility profiles is observed among the ts mutants. These profiles are distinct from one another, yet mutants with related defects share similar profiles. The parF mutants, which have mutations closely linked to the Salmonella topoisomerase IV gene, are hypersusceptible to gyrase subunit B inhibitors (black circle), although these mutants are also susceptible to drugs affecting DNA or protein metabolism. Similarly, specificity within the hypersusceptibility profiles of two out of four ts mutants (SE7583, SE7587, SE5119 and SE5045) having possible defects in the cell wall biosynthesis machinery are also observed (mutants dapA and murCEFG, black diamond). The latter mutants are also susceptible to other agents and share their hypersusceptibility profile with a mutant having a defect in the incorporation of radioactive thymidine (SE5091).

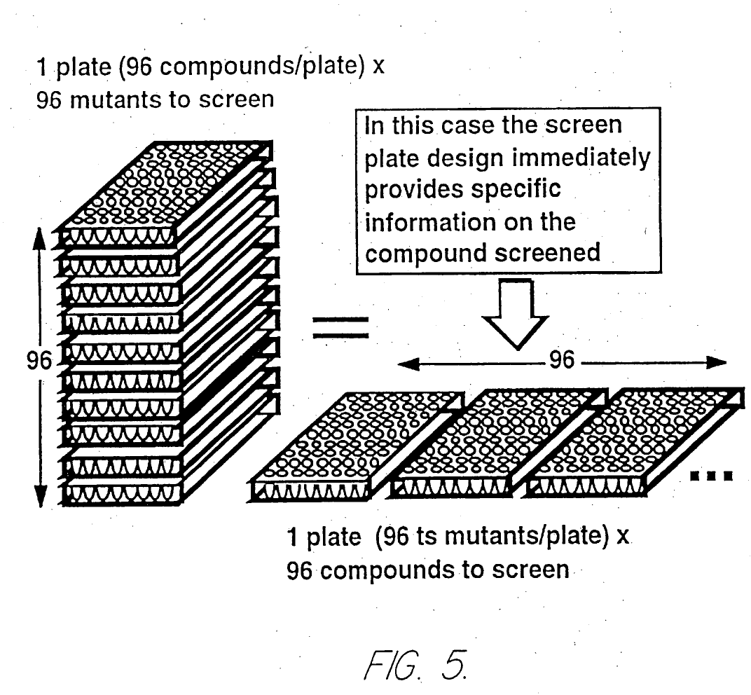

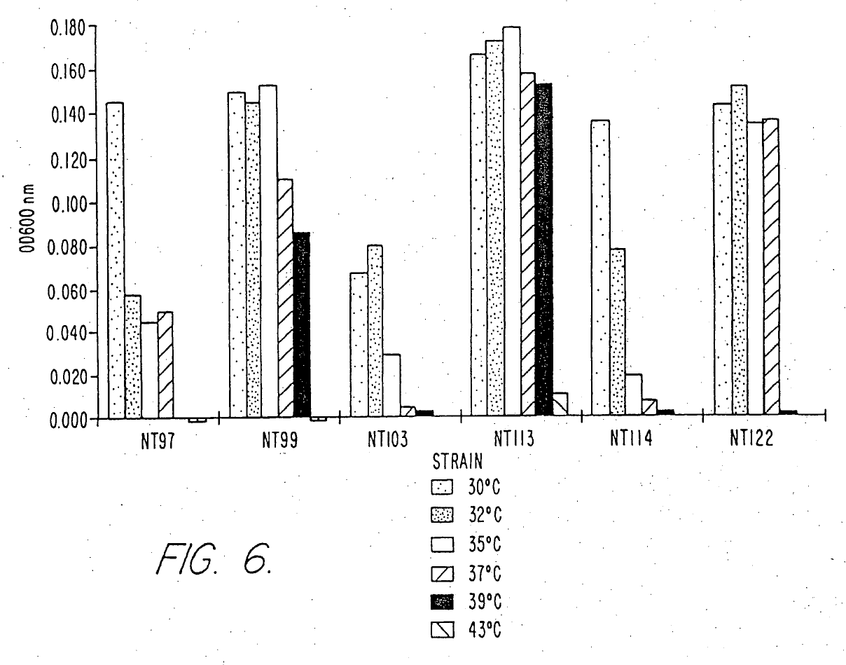

Thus, the hypersensitivity profiles actually represent recognizable interrelationships between cellular pathways, involving several types of interactions as illustrated in FIG. 3. The patterns created by these profiles become signatures for targets within the genetic/metabolic system being sensitized. This provides a powerful tool for characterizing targets, and ultimately for dereplication of screening hits. The hypersusceptibility profiles have been established for 120 Salmonella and 14 Staphylococcus aureus ts mutants with a selection of 37 known drugs or toxic agents

The growth conditional mutants are also used in gene sensor methodology, e.g., using carbon utilization profiles. Ts mutants fail to metabolize different carbon sources in semi-permissive growth conditions. The carbon sources not utilized by a specific mutant or group of mutants provide additional phenotypes associated with the crippled essential function. Moreover, some of these carbon source markers were also not used by the wild type strain exposed to sub-MIC concentrations of known drugs affecting the same specific cellular targets or pathways. For example, a sublethal concentration of cefamandole prevented the Salmonella wild type parent strain from metabolizing the same carbon source that was not used by either the dapA or the murCEFG mutant.

In combination, interrelationships within and between essential cellular pathways are manifested in hypersensitivity and biosensor profiles that together are employed for highly discriminatory recognition of targets and inhibitors. This information provides recognition of the target or pathway of compound action.

B. Screening Strategy and Prototypes

1. Strain Validation and Screening Conditions

Hypersensitive strains (not growth conditional) have been successfully used in the past for discovery of new drugs targeting specific cellular pathways. (Kamogashira and Takegata, 1988, J. Antibiotics 41:803-806; Mumata et al., 1986, J. Antibiotics 39:994-1000.) The specific hypersensitivities displayed by ts-conditional mutants indicates that use of these mutants in whole cell screening provides a rapid method to develop target-specific screens for the identification of novel compounds. However, it is beneficial to eliminate mutants that will not be useful in semi-permissive growth conditions. Such mutant alleles may have nearly wild type function at the screening assay temperature. The simplest method for validating the use of ts mutants is to select those which show a reduced growth rate at the semi-restrictive growth temperature. A reduced growth rate indicates that the essential gene function is partially defective. More specific methods of characterizing the partial defect of a mutant strain are available by biochemical or physiological assays.

2. Multi-Channel Screening Approach

The phenoprint results above, demonstrate that ts mutants show specific hypersusceptibility profiles in semi-permissive growth conditions. As a screening tool, the mutant inhibition profile characterizes the effects of test compounds on specific bacterial pathways. Because the mutants are more sensitive than wild type strains, compounds with weak inhibition activity can be identified.

An example of a multi-channel screen for inhibitors of essential genes is shown in FIG. 4. In this screen design, one plate serves to evaluate one compound. Each well provides a separate whole-mutant cell assay (i.e., there are many targets per screening plate). The assays are genetic potentiation in nature, that is, ts-hypersensitive mutants reveal compounds that are growth inhibitors at concentrations that do not inhibit the growth of the wildtype strain. The profile of mutant inhibition provides insight into the compound's target of inhibition. The ts mutants are grouped by their hypersensitivity profiles to known drugs or by their related defective genes. The figure illustrates the hypothetical growth inhibition results (indicated by “-”) that would be obtained with a new antibacterial agent targeting DNA/RNA metabolism.

Different multi-channel screen designs can fit specific needs or purposes. The choice of a broadly-designed screen (such as in FIG. 4), or one focused on specific cellular pathways, or even specific targets can be made by the appropriate choice of mutants. More specific screen plates would use mutants of a specific gene target like DNA gyrase, or mutants in a specific pathway, such as the cell division pathway.

The use of the 96-well multi-channel screen format allows up to 96 different assays to characterize a single compound. As shown in FIG. 5, this format provides an immediate characterization or profile of a single compound. The more traditional format, using up to 96 different compounds per plate, and a single assay can also be readily accommodated by the genetic potentiation assays.

In comparing the two formats, the multi-channel screen format is generally compound-focused: prioritization of compounds run through the screen will occur, as decisions are made about which compounds to screen first. Each plate provides an immediate profile of a compound. The more traditional format is target-focused: prioritization of targets will occur, as decisions are made about the order of targets or genetic potentiation screens to implement.

In a preferred strategy for screening large compound libraries, a “sub-library” approach is taken. In this approach, the compound library is divided into a number of blocks or “sub-libraries”. All of the selected ts mutants are screened against one block of the compounds. The screen is carried out in 96-well plates and each plate serves to test 80 compounds (one compound per well) on one mutant strain. After a block of compounds are screened, the mutant collection is moved on to test the next compound block.

The advantage of this strategy is that the effect of a compound on all the selected mutant strains can be obtained within a relatively short time. This provides compound-focused information for prioritization of compounds in follow-up studies. Since this strategy has only one mutant instead of many mutants on a plate, cross contamination between different strains and the testing of different mutants at different temperatures (or with other changes in assay conditions) are no longer problems. Moreover, this strategy retains the same compound arrangement in all compound plates, thus saving time, effort and compounds as compared to screening one compound against many mutants on one plate, for compound focused analysis.

### EXAMPLE 4

### Prototype Screening Protocol

S. aureus bacterial cells from pre-prepared frozen stocks are diluted into Mueller-Hinton (MH) broth to an OD600 of about 0.01 and grown at 30° C. till OD600=0.5. Cells are diluted 1,000-fold into MH broth and 50 μl is added to each well of 96-well plates to which 40 μl of MH broth and 10 μl of test compound (varying concentrations) are added. No-compound wells with or without cells are included as controls. The total volume in each well is 100 μl. The plates are incubated at an appropriate screening temperature for 20 hr and OD600 are read. The effect of each compound on a mutant is measured against the growth control and % of inhibition is calculated. Wild type cells are screened at the same conditions. The a of inhibition of a compound on a mutant and that on the wild type cell are compared, and compounds that show higher inhibition on the mutant than on the wild type are identified.

3. Screening Method Refinement

Certain testing parameters for the genetic potentiation screening methods can significantly affect the identification of growth inhibitors, and thus can be manipulated to optimize screening efficiency and/or reliability. Notable among these factors are variable thermosensitivity of different ts mutants, increasing hypersensitivity with increasing temperature, and “apparent” increase in hypersensitivity with increasing compound concentration.

a. Variable Thermosensitivity

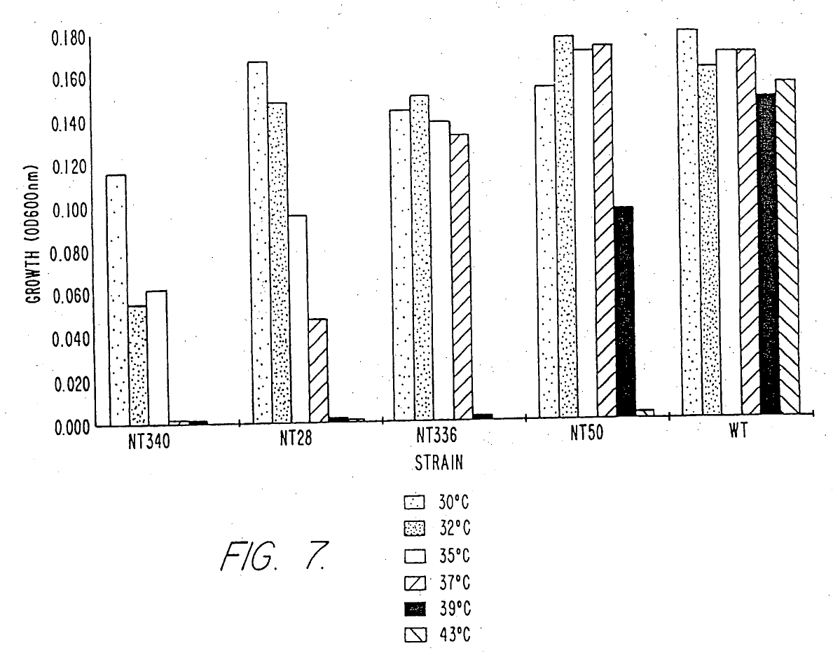

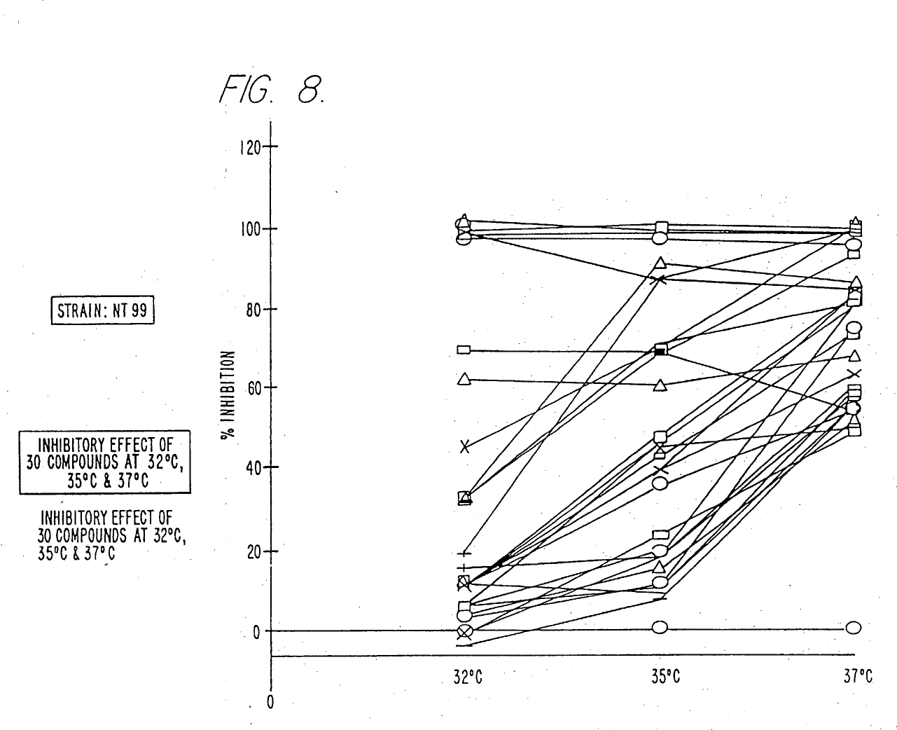

To use S. aureus ts mutants in genetic potentiation screening, the growth of these mutants at different temperatures were measured to determine screening temperatures for each of these mutants. The results showed that different ts mutants have quite different maximum growth temperatures (MGT). The MGTs of some mutants are as high as 39° C. while those of others are 37° C. 35° C. 32° C. or even 30° C. (FIG. 6). Furthermore, different mutants that have mutations in the same gene may have quite different MGTs, as illustrated in FIG. 7 for several polC mutants. Thus, different screening temperatures should be chosen for these mutants in order to accommodate the different growth preferences.

b. Raising Screening Temperature Makes ts Mutants More Sensitive to Certain Compounds

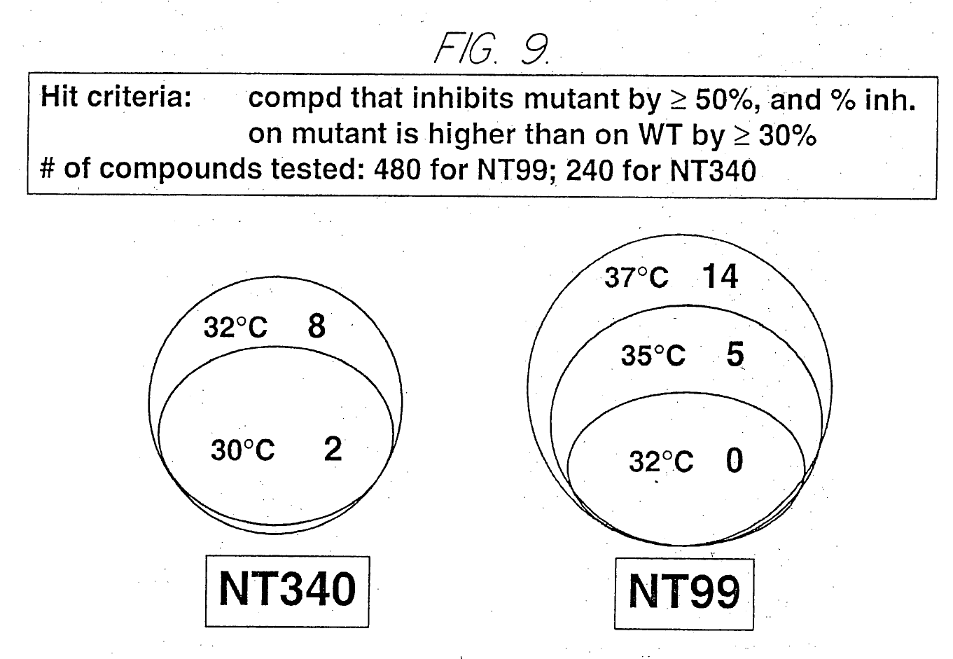

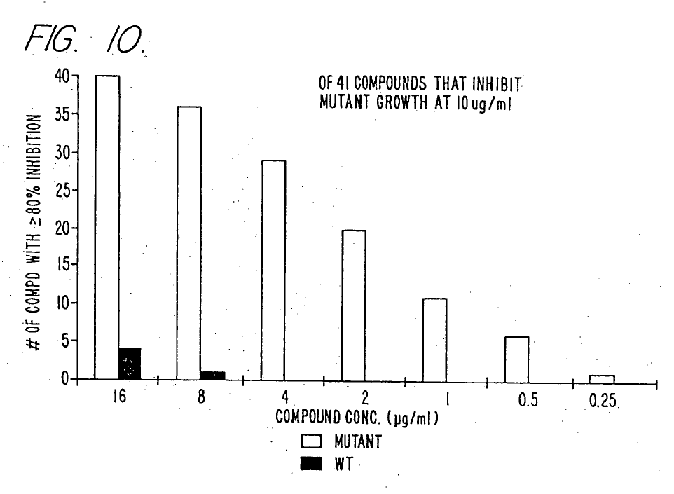

To demonstrate that the ts mutants are more sensitive to potential inhibitors at elevated temperature, the effect of different temperatures on the sensitivity of several ts mutants to a subset of compounds was examined. FIG. 8 shows the inhibitory effect of 30 compounds on mutant NT99 at 3 different temperatures, 32° C. 35° C. and 37° C. Most of these compounds showed increasing inhibitory effect as temperature increased from 320 to 35° C. then to 37° C. Consequently, more hits were identified at 37° C. (FIG. 9). In fact, all the hits identified at 32° C. and 35° C. were included in the 37° C. hits. On the other hand, little difference was observed when the compounds were tested on wild type cells at the same three different temperatures (data not shown).

The temperature effect as mentioned above can be used to control hit rates in the screening. Higher screening temperature can be used to produce more hits for mutants that have low hit rates. Similarly, if a mutant shows a very high hit rate, the number of hits can be reduced by using lower screening temperatures to facilitate hit prioritization.

c. Increasing Compound Concentrations Affect Apparent Hypersensitivity

The concentration of compounds used in the screening is an important parameter in determining the hit rates and the amount of follow-up studies. The concentration of 10 μg/ml has been used in piloting screening studies. To examine whether screening at lower concentrations can identify a similar set of hits, 41 compounds previously scored as hits were screened against their corresponding hypersensitive mutants at lower concentrations. Results in FIG. 10 showed that the number of compounds to which the target mutants were still hypersensitive (≧80% inhibition) decreased as the screening concentrations decreased. At 2 μg/ml, only 20 out of 41 hit compounds were able to be identified as hits that inhibit the mutants by ≧80%, and at 1 μg/ml only 11, or 27%, of the compounds still fell into this category. These data suggest that screening at concentrations <2 μg/ml may miss at least half of the hits that would be identified at 10 μg/ml. On the other hand, screening at concentrations higher than 10 μg/ml may result in large number of low quality hits and create too much work in hit confirmation and follow-up studies. At 10 μg/ml, a hit may appear as a growth inhibitor for both the mutant and wild type strains. This should not be a major problem since lower concentrations of the compound can be tested in the follow-up studies to differentiate its effect on the mutant and the wild type.

4. Evaluation of Uncharacterized Known Growth Inhibitors

In addition to testing known inhibitors of cellular pathways, uncharacterized growth inhibitors identified in other whole-cell screens were also evaluated using temperature sensitive mutants. These growth inhibitors had uncharacterized targets of action. These compounds were previously shown to cause some growth inhibition of the S. aureus strain 8325-4 at 5 mg/ml. The compounds were subsequently tested using a range of concentrations against a collection of S. aureus ts mutants (all derived from S. aureus 8325-4), to determine the MIC values, relative to wild type. FIG. 12 summarizes the data generated using 52 S. aureus ts mutants and 65 growth inhibitor compounds (47 compounds not shown). The table reports the fold-increase in susceptibility of the ts mutants compared with the wild-type parent strain; values within two-fold of wildtype have been left blank in the table for ease of identifying the significant hypersensitive values.

The effects of the 65 test compounds on the ts mutants were mostly selective: for most compounds, a limited number of mutants were hypersensitive. Approximately one-third of all compounds showed identical inhibition of mutant and wild type strains (i.e., no mutants were hypersensitive to these compounds). Two compounds in FIG. 12 showed strong inhibitory effects on about 50% of the mutants tested (compounds 00-2002 and 00-0167). Two additional compounds showed identical inhibition profiles (compounds 30-0014 and 20-0348, FIG. 12). A preliminary analysis of these profiles is provided below.

The genetic basis of the hypersensitivity has been substantiated by two criteria. First, one compound (10-0797) strongly inhibited two mutants (NT52 and NT69) that both affect the same gene. Secondly, complementation of the temperature sensitive phenotype of these mutants resulted in loss of hypersensitivity.

Furthermore, the two compounds that had identical inhibition profiles (30-0014 and 20-0348) have very similar structures (FIG. 11). Thus, the hypersensitivity profile provides a pattern that allows recognition of compounds with similar targets of action, even when the target may be poorly defined. The strong similarity in the structures of these compounds makes their common target of action likely. Based on the mutants that were inhibited (secA, dnaG, and 3 uncharacterized mutants) the target of action of these compounds is not yet defined.

It is preferable to perform a screen of the uncharacterized inhibitors against a larger number of ts mutants. This screen employs preset compound concentrations and obtains the mutant inhibition profile for each compound. Computing the difference in the relative growth of parent and mutant strains in the presence of compounds provides a compound profile similar to that obtained by the MIC determinations of the first screen above.

A wide range of test compounds can be screened. Test compounds that are inhibitory for the wild type parent strain at the pre-selected concentration in the first screening run are retested at a lower concentration to generate an inhibition profile. Data analysis from the screens described above showed that a significant growth reduction of mutant strains compared to the parent strain in the presence of the test compounds is a reasonable indicator of selective compound activity.

Further, compounds for testing can include compounds that show no growth inhibition of the wild type strain. The hypersensitivity of the mutant strains provides the ability to identify compounds that target an essential cellular function, but which lack sufficient potency to inhibit the growth of the wild type strain. Such compounds are modified using medicinal chemistry to produce analogs with increased potency.

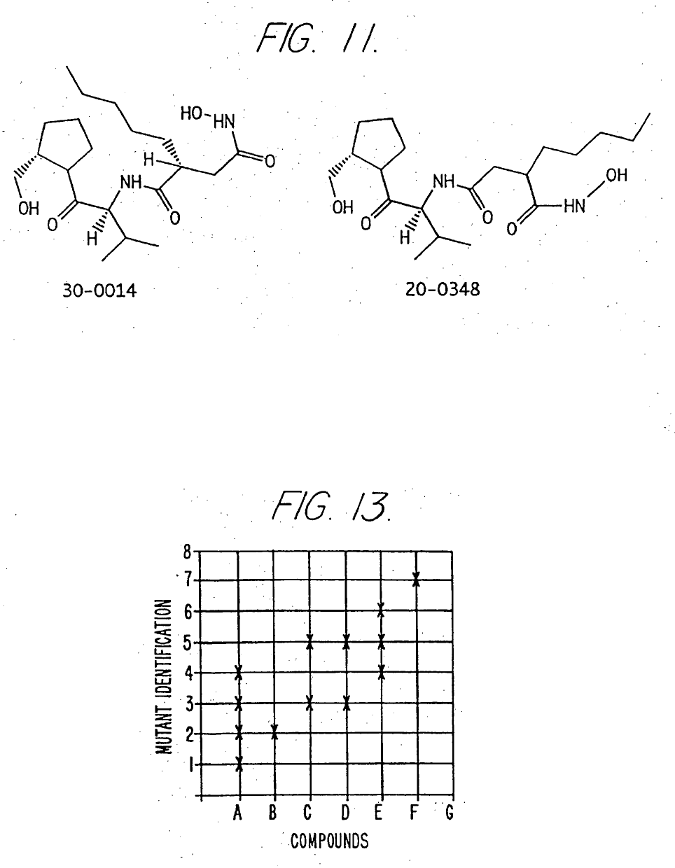

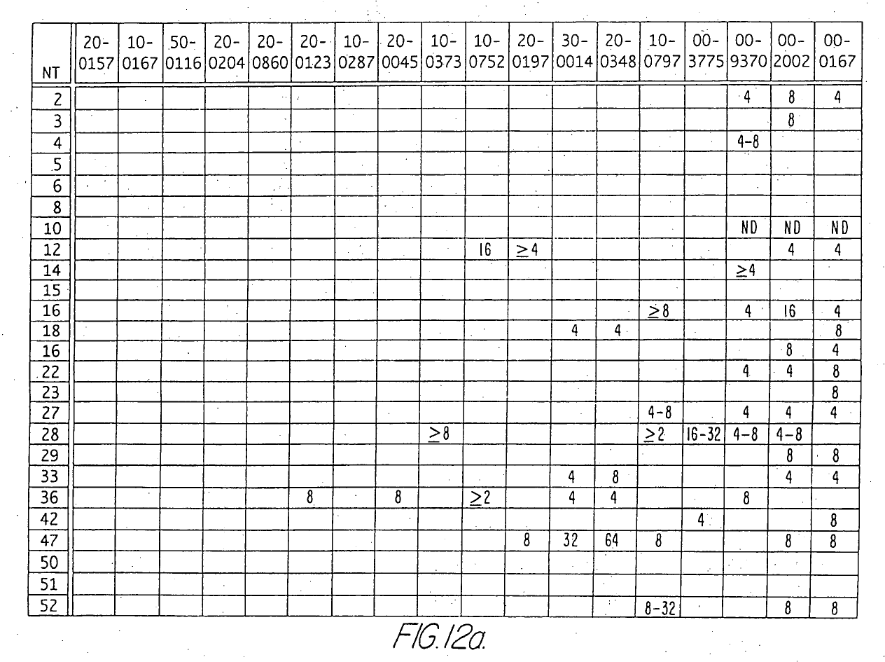

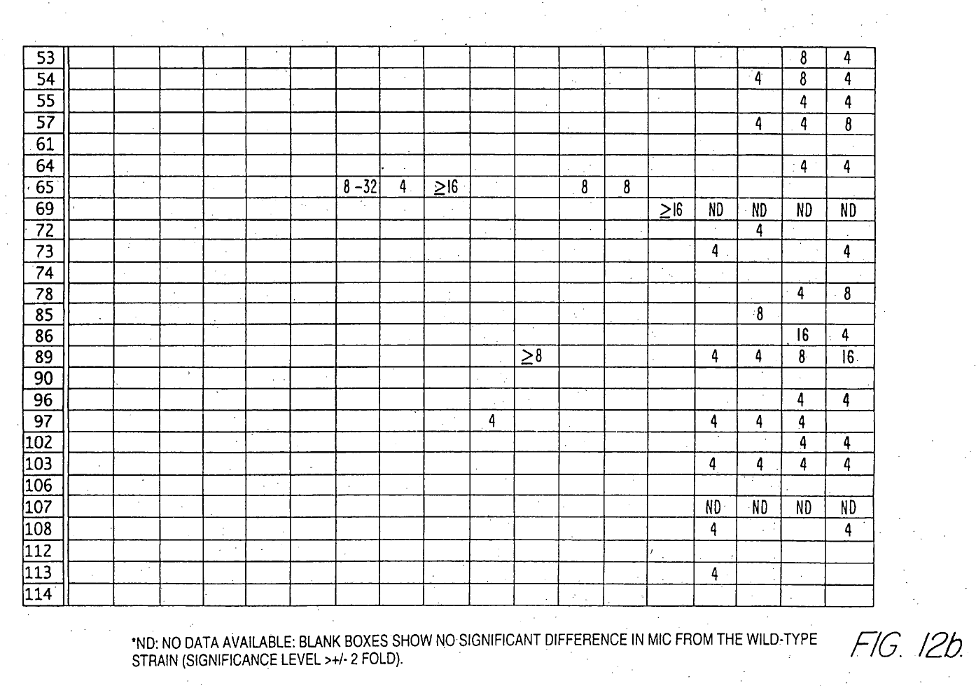

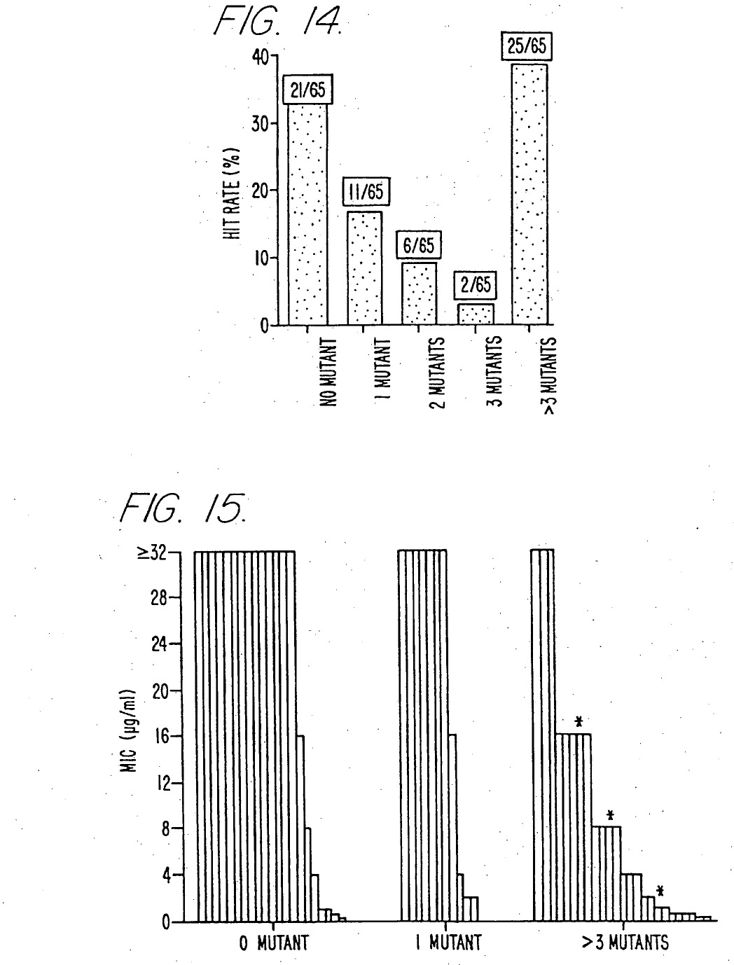

The grid shown in FIG. 13 represents different mutant inhibition profiles anticipated from screening of growth inhibitors, where “x” denotes inhibition of a particular mutant by a particular compound at concentrations much lower than for wildtype.

This grid shows compounds that cause growth inhibition of more than one mutant (compounds A, C, D, E), compounds that inhibit just one mutant (compounds B, F) and one compound that inhibits no mutants (compound G). In addition, this profile identifies mutants inhibited by no compound (mutant 8), a single compound (mutants 1, 6, 7), and several compounds (mutants 2, 3, 4, 5). In the preliminary screens described above, compounds were identified that fit some of these anticipated inhibition profiles (see FIG. 14).

In the preliminary screen, compounds that inhibit the growth of the wild type strain were diluted to a point where growth inhibition of wild type no longer occurred. In this situation, only mutants that are hypersensitive to a particular compound will fail to grow. Thus, even compounds considered “generally toxic” should show some specificity of action, when assayed with the hypersensitive mutant strains.

In the simplest interpretation, compounds that cause growth inhibition inhibit the function of one essential macromolecule. Some compounds may specifically inhibit more than one target macromolecule. However, since one of the targets will be most sensitive to inhibition, one target can be considered the primary target. Thus, a one-to-one correspondence between inhibitors and targets can be established. However, both the data, and less simplistic reasoning provide exceptions to the simple one-to-one relationship between targets and inhibitors. Further analysis and understanding of the complicating effects is necessary to make full use of the data. Some of the complicating effects are discussed below.

a. Compounds that affect many mutants. Certain compounds, such as detergents that target membrane integrity, or DNA intercalators, will have “general”, rather than specific targets. These “general targets” are not the product of a single gene product, but rather are created by the action of many gene products. Thus, in analyzing hypersensitivity profiles, compounds that affect many mutants may indicate action on a “general target”. The profiles of known membrane active agents, and intercalators will provide information to recognize uncharacterized compounds with similar effects.

Compounds that cause growth inhibition of more than one mutant may also arise when the affected mutants are metabolically related. These mutants may affect the same gene, or the same biochemical pathway. For example, mutants defective in one of many cell wall biosynthetic steps may show hypersensitivity to compounds that inhibit any of these steps. Evidence for this type of effect was observed in the hypersensitivity patterns of known inhibitors (see FIG. 2). This concept can be broadened to include effects caused by the “metabolic web”, in which far-reaching consequences may arise through characterized and uncharacterized interrelationships between gene products and their functions.

Overall, the hit rate was high when we considered all compounds that were more active on mutants than on the parent strain. The histogram in FIG. 14 shows the hit rate for compounds that affected one, two, three, or more than three mutants in our prototype screen. The large number of compounds that affected more than three different mutants was at least partly explained by the greater potency of this group of compounds. FIG. 15 illustrates the potency of some of the hits found in the screen as evaluated by the MIC obtained for the parent strain S. aureus 8325-4.

In the prototype screen, compounds affecting more than 3 mutants were generally more potent but some may also be considered broadly toxic. The columns identified by an asterisk in FIG. 15 represent 3 out of 4 compounds that were also shown to be inhibitors of Salmonella typhimurium in another whole cell screen. Consequently, only the most hypersusceptible strain of a group of mutants affected by the same compound should be considered as the primary target. However, the entire mutant inhibition profile of a specific compound is very useful and should be considered as its actual fingerprint in pattern recognition analysis.

b. Compounds that affect few (or no) mutants. Since all compounds assayed in the preliminary screen inhibit the growth of the wild type strain to some degree (initial basis of pre-selection), such compounds indicate that the mutant population is not sufficiently rich to provide a strain with a corresponding hypersensitive target.

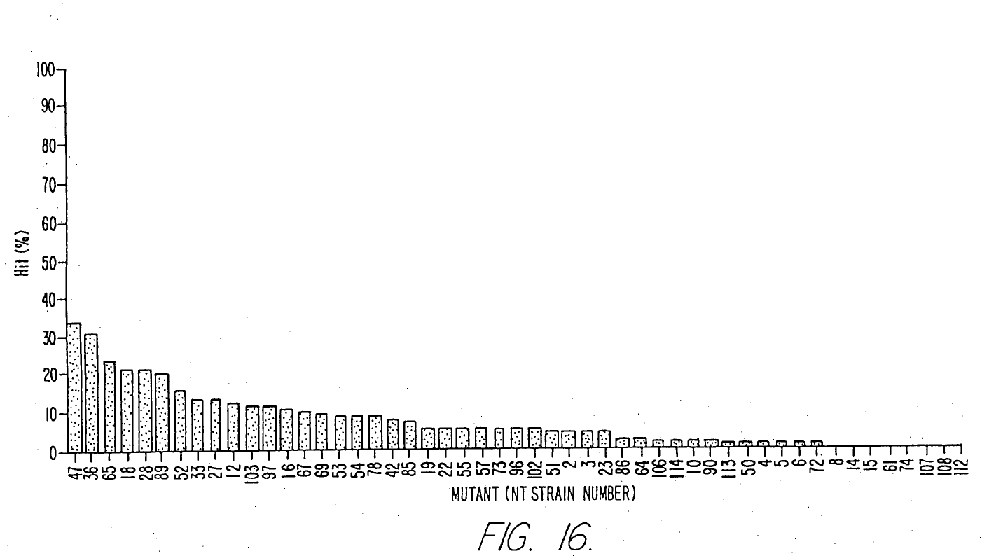

c. Mutants affected by many compounds. Another complication of the simple one-to-one compound/target relationship will arise because of mutants that are inhibited by many different compounds. The relative number of compounds (% hits) that inhibited the growth of each mutant in the S. aureus pilot is shown in FIG. 16. Several mutants were affected by many compounds. Several distinct causes of this are apparent. First, some mutants may have defects in the membrane/barrier that cause hyperpermeability to many different compounds. Such mutants will have higher intracellular concentrations of many compounds, which will inhibit metabolically unrelated targets. Other mutants may have defects that have far-reaching consequences, because their gene products sit at critical points in the metabolic web. Still other mutants may have specific alleles that are highly crippled at the assay temperature. For these mutants, the metabolic web consequences are large because the specific allele has created a highly hypersensitive strain.

d. Mutants affected by few or no compounds. For the mutants that were hypersusceptible to fewer compounds, it is possible that their mutations affect a limited metabolic web, that mutations provide a true specificity that was yet not revealed by any compound, or that these mutants have nearly full activity at the assay temperature. This analysis stresses the importance of strain validation as indicated above.

In interpreting these patterns, the number of mutants screened and the total number of targets are also important variables. These numbers provide a simple probabilistic estimate of the fraction of the compounds that should have a one-to-one correspondence with a mutant target in the sample that was screened.

6. Prioritization of Hits and Downstream Development

The early steps in a multi-channel genetic potentiation screen include the following:

Pre-selection of mutant strains for screening

Pre-selection of desired test compounds based on structural features, biological activity, etc. (optional)

Testing of the chosen compounds at a pre-determined concentration, preferably in the range 1-10 μg/ml.

Analysis of inhibitory profiles of compounds against the mutant population and selection of interesting hits

Confirmation of the selective inhibitory activity of the interesting hits against specific mutants

Secondary evaluation of prioritized hits.

Genetic potentiation assays provide a rapid method to implement a large number of screens for inhibitors of a large number of targets. This screening format will test the capacity of rapid high-throughput screening. The capability to screen large numbers of compounds should generate a large number of “hits” from this screening. Limitations in downstream development through medicinal chemistry, pharmacology and clinical development will necessitate the prioritization of the hits. When large numbers of hits are available, each with reasonable in vitro activity, prioritization of hits can proceed based on different criteria. Some of the criteria for hit characterization include:

chemical novelty

chemical complexity, modifiability

pharmacological profile

toxicity profile

target desirability, ubiquity, selectivity

Secondary tests will be required not only for the initial evaluation of hits, but also to support medicinal chemistry efforts. While the initial genetic potentiation tests will be sufficient to identify and confirm hits, selection of hits for further development will necessitate establishment, of the specific target of action. Equipped with the gene clones, selection of resistant alleles provides early evidence for the specific target. Subsequent efforts to establish a biochemical assay for rapid, specific and sensitive tests of derivative compounds will be aided by the over-expression and purification of the target protein, sequence analysis of the ORF to provide early insight into novel target function, as well as a variety of physiological and biochemical tests comparing the mutant and wild type strain to confirm the novel target function, and aid in the establishment of biochemical assays for the targets.

7. Identification of Specific Inhibitors of Gene Having Unknown Function

In a piloting screening study, a number of compounds were identified as inhibitors for mutants with mutations located in open reading frames whose functions are not known. Some of the open reading frames have been previously identified in other bacteria while others show little homology to the current Genbank sequence collection. An example is mutant NT94, whose complementing clones contain an open reading frame that is homologous to a spoVB-like gene in B. subtilis. While the function of the gene is not clear in either B. subtilis or S. aureus, NT94 is hypersensitive to many compounds tested, as illustrated in Table 2 below.

TABLE 2
Hit Rates in Genetic Potentiation Screen

| | |
|---|---|
| Number of | |
| mutants n, on | Confirmed Hits |

| which cmpds active | | 39 mutants | NT94 |
|---|---|---|---|
| n = 1 or 2 | Average hit | 0.03% | 1.06% |
| | rate | | |
| | Hit rate range | 0-0.31% | |
| | among mutants | | |
| n => 3 | Average hit | 0.17% | 1.39% |
| | rate | | |
| | Hit rate range | 0-0.72% | |
| | among mutants | | |

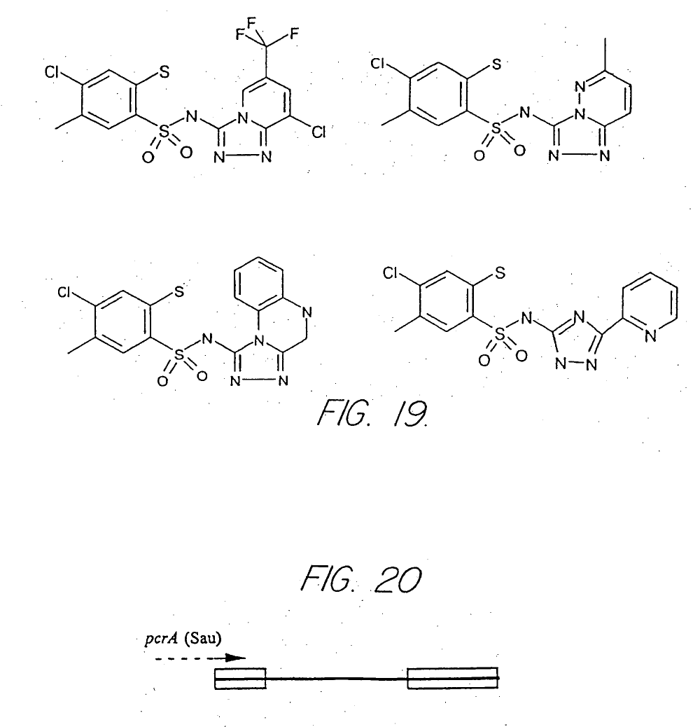

In fact, NT94 had the highest hit rate among the 40 mutant strains tested. Among the NT94 hits, 4 compounds share similar chemical structures (FIGS. 19A-D) The MICs of these compounds on NT94 are 0.25-2 μg/ml, which are 16-256 fold lower than those on the wild type cells (32-64 μg/ml). The similarity in the compound structures suggests a common and specific mechanism of the inhibitory effect on NT94.

Furthermore, the hypersensitivity to these compounds can be abolished by introducing 2 or more copies of the wild type gene into NT94. A correlation between the copy number of the wild type gene and the tolerance to the compounds has been observed. Cells with 2 copies of the wild type gene are slightly more resistant (2-fold increase in MIC) to MC-207,301 and MC-207,330 than the wild type cells which has one gene copy; cells carrying complementing plasmids (about 20-50 copies per cell) are much more resistant (8-16 fold increase in MIC). Such a gene dosage effect further suggests that either the gene product itself or its closely related functions of the open reading frame affected in NT94 is the target of the hit compounds.

8. Multi-Channel Screen Advantages

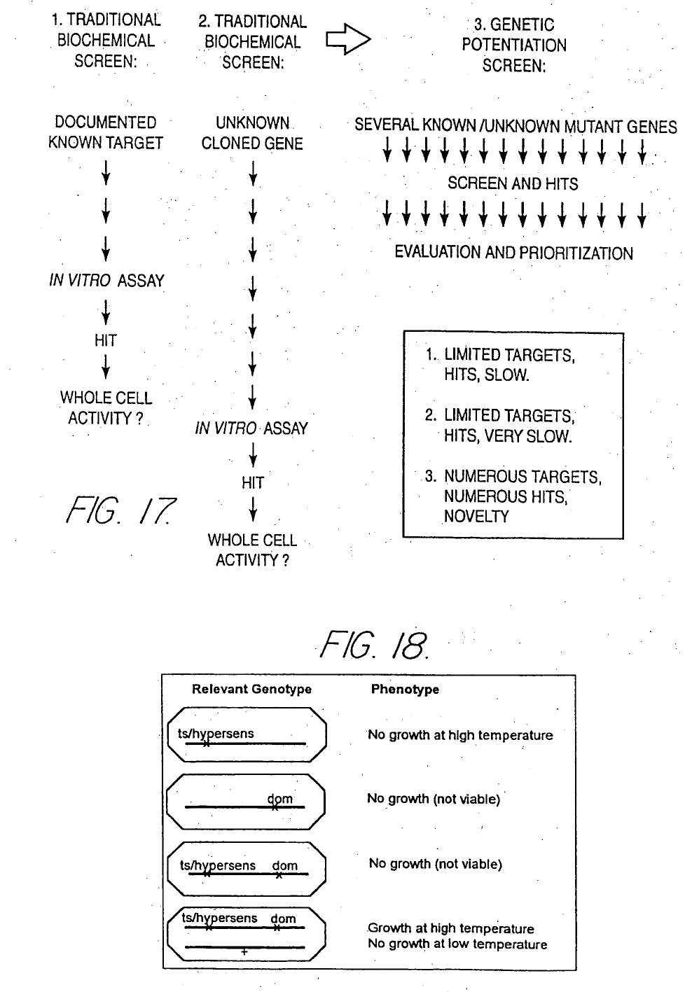

As depicted by the S. aureus example shown above, multi-channel screen design rapidly leads to the identification of hits and provide some of the necessary specificity information to prioritize compounds for further evaluation. FIG. 17 illustrates the advantages of a genetic potentiation approach as the basis of a screen design.

Overall, an approach using whole-cell genetic potentiation of ts mutants includes the selectivity of the biochemical screens (it is target-specific, or at least pathway-specific) and it is more sensitive than traditional screens looking for growth inhibitors due to the hypersensitive nature of the mutants. This genetic potentiation approach also provides a rapid gene-to-screen technology and identifies hits even before the genes or biochemical targets are fully characterized.

9. Alternatives to Ts Hypersensitivity Screening

There are a number of additional strategies that can be undertaken to devise target-based whole cell screens, as well as binding or biochemical type screens. In order to implement these strategies, knowledge of the existence of the gene, the DNA sequence of the gene, the hypersensitivity phenotype profile, and the conditional mutant alleles will provide significant information and reagents. Alternative strategies are based on:

over- and under-expression of the target gene

dominant mutant alleles

hypersensitive mutant alleles

a. Over- and Under-expression of Target Genes. There are numerous examples of over-expression phenotypes that range from those caused by 2-fold increases in gene dosage (Anderson and Roth, 1977, Ann. Rev. Microbiol. 31:473-505; Stark and Wahl, 1984, Ann. Rev. Biochem. 53:447-491) to multi-fold increases in dosage which can be either chromosomal-encoded (Normark et al., 1977, J. Bacteriol. 132:912-922), or plasmid-encoded (Tokunaga et al., 1983, J. Biol. Chem. 258:12102-12105). The phenotypes observed can be analog resistance (positive selection for multiple copies, negative selection for inhibition phenotype) or growth defects (negative selection for multiple copies, but positive selection for inhibition phenotype).

Over-expression can be achieved most readily by artificial promoter control. Such screens can be undertaken in E. coli where the breadth of controllable promoters is high. However, this method loses the advantage gained by whole cell screening, that of assurance that the compound enters the pathogen of interest. Establishing controllable promoters in S. aureus will provide a tool for screening not only in S. aureus but most likely in other Gram-positive organisms. An example of such a controllable promoter is shown by controlled expression of the agr P3 promoter in the in vivo switch construction.

b. Dominant alleles. Dominant alleles can provide a rich source of screening capabilities. Dominant alleles in essential genes will prevent growth unless conditions are established in which the alleles are non-functional or non-expressed. Methods for controlled expression (primarily transcriptional control) will provide the opportunity to identify dominant mutant alleles that prevent cell growth under conditions of gene product expression.

Equally useful will be mutant alleles that are dominant, but conditionally functional. A single mutation may provide both the dominant and conditional-growth phenotype. However, utilizing the existing collection of temperature sensitive alleles, mutagenesis with subsequent selection for a dominant allele may provide more mutational opportunities for obtaining the necessary dominant conditional alleles. There is precedent for such additive effects of mutations on the protein phenotype (T. Alber, 1989, Ann. rev. Biochem. 58:765-798) as well as evidence to suggest that heat-sensitive mutations, which generally affect internal residues (Hecht et al., 1983, Proc. Natl. Acad. Sci. USA 80:2676-2680), will occur at different locations in the protein different than dominant mutations, one type of which will affect protein-protein interactions, which are more likely on the protein surface.

The use of dominant conditional double mutants may have an additional advantage, since the hypersensitivity phenotypes may remain the same in the double mutant as in the single conditional mutant allele. In this case, a merodiploid carrying two copies of the target gene—one wild type, and one carrying the dominant conditional doubly mutant gene—would provide a sophisticated screening strain (see FIG. 18). The screen would rely on the hypersensitivity of the dominant protein to inhibitor compounds. Under conditions of the dominant protein's function, cells will not grow, while inhibition of the dominant protein will allow cell growth. The temperature sensitive allele provides a basis for hypersensitivity of the dominant protein, relative to the wild type protein.

c. Hypersensitive mutant alleles—Additional mutants that display more pronounced hypersensitivities than the original conditional lethal mutants can be sought. Selection or screening procedures are based on the initial secondary phenotype profiles. These new highly hypersensitive alleles need not have a conditional growth defect other than that observed in the presence of the toxic agent or inhibitor. Such highly hypersensitive alleles provide strong target specificity, and high sensitivity to weak inhibitors. Such hypersensitive alleles can readily be adapted for screens with natural products, and with synthetic or combinatorial libraries of compounds in traditional screen formats.

d. Compound Binding and Molecular Based Assays and Screens

As indicated above, knowledge and possession of a sequence encoding an essential gene also provides knowledge and possession of the encoded product. The sequence of the gene product is provided due to the known genetic code. In addition, possession of a nucleic acid sequence encoding a polypeptide provides the polypeptide, since the polypeptide can be readily produced by routine methods by expressing the corresponding coding sequence in any of a variety of expression systems suitable for expressing procaryotic genes, and isolating the resulting product. The identity of the isolated polypeptide can be confirmed by routine amino acid sequencing methods.

Alternatively, once the identity of a polypeptide is known, and an assay for the presence of the polypeptide is determined, the polypeptide can generally be isolated from natural sources, without the necessity for a recombinant coding sequence. Such assays include those based on antibody binding, enzymatic activity, and competitive binding of substrate analogs or other compounds. Consequently, this invention provides purified, enriched, or isolated products of the identified essential genes, which may be produced from recombinant coding sequences or by purification from cells naturally expressing the gene.

For use of binding assays in screening for compounds active on a specific polypeptide, it is generally preferred that the binding be at a substrate binding site, or at a binding site for an allosteric modulator, or at another site which alters the relevant biological activity of the molecule. However, simple detection of binding is often useful as a preliminary indicator of an active compound; the initial indication should then be confirmed by other verification methods.

Binding assays can be provided in a variety of different formats. These can include, for example, formats which involve direct determination of the amount of bound molecule, either while bound or after release; formats involving indirect detection of binding, such as by determination of a change in a relevant activity, and formats which involve competitive binding. In addition, one or more components of the assay may be immobilized to a support, though in other assays, the assays are performed in solution. Further, often binding assays can be performed using only a portion of a polypeptide which includes the relevant binding site. Such fragments can be constructed, for example, by expressing a gene fragment which includes the sequence coding for a particular polypeptide fragment and isolating the polypeptide fragment, though other methods known to those skilled in the art can also be used. Thus, essential genes identified herein provide polypeptides which can be utilized in such binding assays. Those skilled in the art can readily determine the suitable polypeptides, appropriate binding conditions, and appropriate detection methods.

Provision of a purified, enriched, or isolated polypeptide product of an essential gene can also allow use of a molecular based (i.e., biochemical) method for screening or for assays of the amount of the polypeptide or activity present in a sample. Once the biological activities of such a polypeptide are identified, one or more of those activities can form the basis of an assay for the presence of active molecules of that polypeptide. Such assays can be used in a variety of ways, for example, in screens to identify compounds which alter the level of activity of the polypeptide, in assays to evaluate the sensitivity of the polypeptide to a particular compound, and in assays to quantify the concentration of the polypeptide in a sample.

10. Antibacterial Compounds Identified by Hypersensitive Mutant Screening

Using the genetic potentiation screening methods described above, a number of compounds have been identified which inhibit growth of S. aureus cell. These compounds were identified as having activity on the NT94 mutant described above, and so illustrate the effectiveness of the claimed screening methods. These results further illustrate that the genes identified by the temperature sensitive mutants are effective targets for antibacterial agents. The identified compounds have related structures, as shown in FIGS. 19A-D

These compounds can be generally described by the structure shown below:

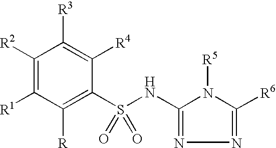

in which

R, R¹, R² and R³ are independently H, alkyl (C₁-C₅), or halogen;

R⁴ is H, alkyl (C₁-C₅), halogen, SH, or S-alkyl (C₁-C₃);

R⁵ is H, alkyl (C¹-C⁵), or aryl (C₆-C₁₀);

R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);

or

R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;

in which

R⁷, R⁸, R⁹, and R¹⁰ are independently H, alkyl (C₁-C₅) halogen, fluoroalkyl (C₁-C₅);

or

R⁷ and R⁸ together are —CH═CH—CH═CH—.

Thus, the invention includes antibacterial compositions containing the described compounds, and the use of such compositions in methods for inhibiting the growth of bacteria and methods for treating a bacterial infection in an animal.

V. Description of Compound Screening Sources and Sub-Structure Search Method

The methods of this invention are suitable and useful for screening a variety of sources for possible activity as inhibitors. For example, compound libraries can be screened, such as natural product libraries, combinatorial libraries, or other small molecule libraries. In addition, compounds from commercial sources can be tested, this testing is particularly appropriate for commercially available analogs of identified inhibitors of particular bacterial genes.

Compounds with identified structures from commercial sources can be efficiently screened for activity against a particular target by first restricting the compounds to be screened to those with preferred structural characteristics. As an example, compounds with structural characteristics causing high gross toxicity can be excluded. Similarly, once a number of inhibitors of a specific target have been found, a sub-library may be generated consisting of compounds which have structural features in common with the identified inhibitors. In order to expedite this effort, the ISIS computer program (MDL Information Systems, Inc.) is suitable to perform a 2D-substructure search of the Available Chemicals Directory database (MDL Information Systems, Inc.). This database contains structural and ordering information on approximately 175,000 commercially available chemical compounds. Other publicly accessible chemical databases may similarly be used.

VI. In Vivo Modeling: Gross Toxicity

Gross acute toxicity of an identified inhibitor of a specific gene target may be assessed in a mouse model. The inhibitor is administered at a range of doses, including high doses, (typically 0-100 mg/kg, but preferably to at least 100 times the expected therapeutic dose) subcutaneously or orally, as appropriate, to healthy mice. The mice are observed for 3-10 days. In the same way, a combination of such an inhibitor with any additional therapeutic components is tested for possible acute toxicity.

VII. Pharmaceutical Compositions and Modes of Administration

The particular compound that is an antibacterial agent can be administered to a patient either by itself, or in combination with another antibacterial agent, or in pharmaceutical compositions where it is mixed with suitable carriers or excipient(s). A combination of an inhibitor of a particular gene with another antibacterial agent can be of at least two different types. In one, a quantity of an inhibitor is combined with a quantity of the other antibacterial agent in a mixture, e.g., in a solution or powder mixture. In such mixtures, the relative quantities of the inhibitor and the other antibacterial agent may be varied as appropriate for the specific combination and expected treatment. In a second type of combination an inhibitor and another antibacterial agent can be covalently linked in such manner that the linked molecule can be cleaved within the cell. However, the term “in combination” can also refer to other possibilities, including serial administration of an inhibitor and another antibacterial agent. In addition, an inhibitor and/or another antibacterial agent may be administered in pro-drug forms, i.e. the compound is administered in a form which is modified within the cell to produce the functional form. In treating a patient exhibiting a disorder of interest, a therapeutically effective amount of an agent or agents such as these is administered. A therapeutically effective dose refers to that amount of the compound(s) that results in amelioration of symptoms or a prolongation of survival in a patient, and may include elimination of a microbial infection.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. It is preferable that the therapeutic serum concentration of an efflux pump inhibitor should be in the range of 0.1-100 μg/ml.

For any compound used in the method of the invention, the therapeutically effective dose can be estimated initially from cell culture assays. For example, a dose can be formulated in animal models to achieve a circulating plasma concentration range that includes the IC₅₀ as determined in cell culture Such information can be used to more accurately determine useful doses in humans. Levels in plasma may be measured, for example, by HPLC.

The exact formulation, route of administration and dosage can be chosen by the individual physician in view of the patient's condition. (See, e.g., Fingl et al., in THE PHARMACOLOGICAL BASIS OF THERAPEUTICS, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific infection being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art, into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

VIII. Use of Gene Sequences as Probes and Primers

In addition to the use of the growth conditional mutant strains as described above, DNA sequences derived from the identified genes are also useful as probes to identify the presence of bacteria having the particular gene or, under suitable conditions, a homologous gene. Similarly, such probes are useful as reagents to identify DNA chains which contain a sequence corresponding to the probe, such as for identifying clones having a recombinant DNA insert (such as in a plasmid). For identifying the presence of a particular DNA sequence or bacterium having that sequence it is preferable that a probe is used which will uniquely hybridize with that sequence. This can be accomplished, for example, by selecting probe sequences from variable regions, using hybridization conditions of suitably high stringency, and using a sufficiently long probe (but still short enough for convenient preparation and manipulation. Preferably, such probes are greater than 10 nucleotides in length, and more preferably greater than 15 nucleotides in length. In some cases, it is preferable that a probe be greater than 25 nucleotides in length. Those skilled in the art understand how to select the length and sequence of such probes to achieve specific hybridization. In addition, probes based on the specific genes and sequences identified herein can be used to identify the presence of homologous sequences (from homologous genes) For such purposes it is preferable to select probe sequences from portions of the gene which are not highly variable between homologous genes. In addition, the stringency of the hybridization conditions can be reduced to allow a low level of base mismatch.

As mentioned above, similar sequences are also useful as primers for PCR. Such primers are useful as reagents to amplify the number of copies of one of the identified genes or of a homologous gene. As with probes, it is preferable that the primers specifically hybridize with the corresponding sequence associated with one of the genes corresponding to SEQ ID NO. 1-105. Those skilled in the art understand how to select and utilize such primers.

The embodiments herein described are not meant to be limiting to the invention. Those of skill in the art will appreciate the invention may be practiced by using any of the specified genes or homologous genes, for uses and by methods other than those specifically discussed, all within the breadth of the claims.

Other embodiments are within the following claims.

What is claimed is:

1. A method of treating a bacterial infection of a mammal, comprising administering to a mammal suffering from a bacterial infection an amount of a compound active against a bacterial gene selected from the group consisting of the genes corresponding to SEQ ID NO. 1-105 sufficient to inhibit the growth of bacteria involved in said infection.

2. The method of claim 1, wherein said bacterial infection involves a bacterial strain expressing a gene selected from the group consisting of the genes corresponding to SEQ ID NO. 1-105 or a homologous gene.

3. The method of claim 2, wherein said gene corresponds to SEQ ID NO. 60 and wherein said compound has the structure: 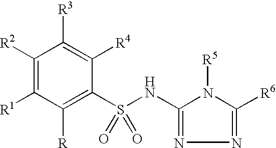
wherein
R, R¹, R², and R³ are independently H, alkyl (C₁-C₅), or halogen;
R⁴ is H, alkyl (C₁-C₅), halogen, SH, or S-alkyl (C₁-C₃);
R⁵ is H, alkyl (C¹-C⁵) or aryl (C₆-C₁₀);
R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);
or
R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;
wherein R⁷, R⁸, R⁹, and R¹⁰ are independently H, alkyl (C₁-C₅), halogen, fluoroalkyl (C₁-C₅);
or
R⁷ and R⁸ together are —CH═CH—CH═CH—.

4. A method of treating a bacterial infection in a mammal comprising administering to said mammal an amount of an antibacterial agent effective to reduce said infection,
wherein said antibacterial agent specifically inhibits a biochemical pathway requiring the expression product of a gene selected from the group consisting of the genes corresponding to SEQ ID NO. 1-105, and
wherein inhibition of said biochemical pathway inhibits the growth of said bacterium in vivo.

5. A method of inhibiting the growth of a pathogenic bacterium comprising contacting said bacterium with an antibacterial agent which specifically inhibits a biochemical pathway requiring the expression product of a gene selected from the group consisting of the genes corresponding to SEQ ID NO. 1-105,
wherein inhibition of said biochemical pathway inhibits the growth of said bacterium.

6. The method of claim 5, wherein said gene corresponds to SEQ ID NO. 60 and wherein said compound has the structure: 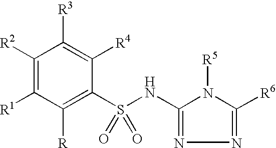
wherein
R, R¹, R², R², and R³ are independently H, alkyl (C₁-C₅), or halogen;
R⁴ is H, alkyl (C₁-C₅), halogen, SH, or S-alkyl (C₁-C₃);
R⁵ is H, alkyl (C¹-C⁵), or aryl (C₆-C₁₀);
R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);
or
R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;
wherein R⁷, R⁸, R⁹, and R¹⁰ are independently H, alkyl (C₁-C₅), halogen, fluoroalkyl (C₁-C₅);
or
R⁷ and R⁸ together are —CH═CH—CH═CH—.

7. The method of claim 4 or 5 wherein said antibacterial agent inhibits the activity of an expression product of a bacterial gene selected from the group consisting of the genes corresponding to SEQ ID NO. 1-105.

8. A method of prophylactic treatment of a mammal, comprising administering to a mammal at risk of a bacterial infection a compound active against a bacterial gene selected from the group consisting of the genes corresponding to SEQ ID NO. 1-105.

9. The method of claim 8, wherein said gene corresponds to SEQ ID NO. 60 and wherein said compound has the structure: 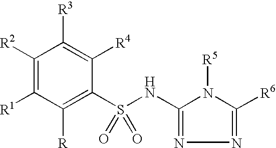
wherein
R, R¹, R², and R³ are independently H, alkyl (C₁-C₅), or halogen;
R⁴ is H, alkyl (C₁-C₅), halogen, SH, or S-alkyl (C₁-C₃);
R⁵ is H, alkyl (C¹-C⁵), or aryl (C₆-C₁₀);
R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);
or
R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;
wherein R⁷, R⁸, R⁹, and R¹⁰ are independently H, alkyl (C₁-C₅), halogen, fluoroalkyl (C₁-C₅);
or
R⁷ and R⁸ together are —CH═CH—CH═CH—.

10. A method of screening for an antibacterial agent,
comprising determining whether a test compound is active against a bacterial gene selected from the group consisting of the genes corresponding to SEQ ID NO. 1-105.

11. A method of claim 10, comprising the steps of:
a. providing a bacterial strain having a mutant form of a gene selected from a group consisting of the genes corresponding to SEQ ID NO. 1-105, or a gene homologous thereto, wherein said mutant form of the gene confers a growth conditional phenotype;
b. providing comparison bacteria of a bacterial strain having a normal form of said gene;
b. contacting bacteria of said bacterial strains with a test compound in semi-permissive growth conditions;
c. determining whether the growth of said bacteria having said mutant form of a gene is reduced in the presence of said test compound compared to the growth of said comparison bacteria.

12. A method of screening for an antibacterial agent, comprising the steps of:
a) contacting a cell expressing a polypeptide encoded by a gene selected from the group consisting of the genes corresponding to SEQ ID NO. 1-105 with a test compound; and
b) determining whether the amount or level of activity of said polypeptide is altered;
wherein an alteration in said amount or level of activity of said polypeptide is indicative of a useful antibacterial agent.

13. A method of screening for an antibacterial agent, comprising the steps of:
a) contacting a polypeptide or a biologically active fragment thereof with a test compound, wherein said polypeptide is encoded by a gene selected from a group consisting of the genes corresponding to SEQ ID NO. 1-105; and
b) determining whether said test compound binds to said polypeptide or said fragment;
wherein binding of said test compound to said polypeptide or said fragment is indicative of a useful antibacterial agent.

14. A method for evaluating an agent active on a gene selected from a group consisting of the genes corresponding to SEQ ID NO. 1-105, comprising the steps of:
a) contacting a sample containing an expression product of said gene with said agent; and
b) determining the amount or level of activity of said expression product in said sample.

15. A method of diagnosing the presence of a bacterial strain having a gene selected from the group consisting of the genes corresponding to SEQ ID NO. 1-105, comprising probing with an oligonucleotide at least 15 nucleotides in length which specifically hybridizes to a nucleotide sequence which is the same as or complementary to a portion of the sequence of a bacterial gene selected from the group consisting of the genes corresponding to SEQ ID NO. 1-105.

16. A method of diagnosing the presence of a bacterial strain, comprising specifically detecting the presence of the transcriptional or translational product of a gene selected from the group consisting of the genes corresponding to SEQ ID NO. 1-105.

11. A pharmaceutical composition comprising a pharmaceutically acceptable carrier and a compound active on a bacterial gene selected from the group consisting of the genes corresponding to SEQ ID NO. 1-105.

18. The pharmaceutical composition of claim 17, wherein said bacterial gene corresponds to SEQ ID NO. 60 and wherein said compound has the structure: 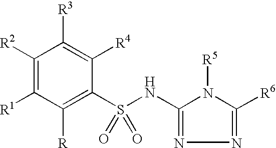
wherein
R, R¹, R², and R³ are independently H, alkyl (C₁-C₅), or halogen;
R⁴ is H, alkyl (C₁-C₅), halogen, SH, or S-alkyl (C₁-C₃);
R⁵ is H, alkyl (C¹-C⁵), or aryl (C₆-C₁₀);
R⁶ is CH2NH2, alkyl (C1-C4), 2-pyridyl, 3-pyridyl, 4-pyridyl, 2-furyl, 3-furyl, 2-thienyl, 3-thienyl, or aryl (C₆-C₁₀);
or
R⁵ and R⁶ together are —C(R⁷)═C(R⁸)—C(R⁹)═C(R¹⁰)—, —N═C(R⁸)—C(R⁹)═C(R¹⁰)—, —C(R⁷)═N—C(R⁹)═C(R¹⁰)—, —C(R⁷)═C(R⁸)—N═C(R¹⁰)—, or —C(R⁷)═C(R⁸)—C(R⁹)═N—;
wherein R⁷, R⁸, R⁹, and R¹⁰ are independently H, alkyl (C₁-C₅), halogen, fluoroalkyl (C₁-C₅);
or
R⁷ and R⁸ together are —CH═CH—CH═CH—.

19. A method for making an antibacterial agent, comprising the steps of:
a. screening for an agent active on one of the genes corresponding to SEQ ID NO. 1-105 by
providing a bacterial strain having a mutant form of a gene selected from a group consisting of the genes corresponding to SEQ ID NO. 1-105, or a gene homologous thereto, wherein said mutant form of the gene confers a growth conditional phenotype,
providing comparison bacteria of a bacterial strain having a normal form of said gene,
contacting bacteria of said bacterial strains with a test compound in semi-permissive growth conditions, and
determining whether the growth of said bacteria having said mutant form of a gene is reduced in the presence of said test compound compared to the growth of said comparison bacteria; and
b. synthesizing said agent in an amount sufficient to provide said agent in a therapeutically effective amount to a patient.

20. A novel compound having antibacterial activity, wherein said antibacterial activity is against a bacterial gene selected from the group consisting of the genes corresponding to SEQ ID NO. 1-105 or a product thereof.

21. A purified bacterial strain expressing a mutated gene selected from the group consisting of the genes corresponding to SEQ ID NO. 1-105,
wherein said mutated gene confers a growth conditional phenotype.

22. A recombinant bacterial cell containing an artificially inserted DNA construct comprising a DNA sequence which is the same as or complementary to a bacterial gene selected from the group consisting of the genes corresponding to SEQ ID NO. 1-3, 8, 11-20, 31-48, 59-68, 71, 76-87, 92-97, and 100-105.

23. A recombinant cell containing an artificially inserted DNA construct comprising a DNA sequence which is the same as or complementary to a portion at least 15 nucleotides in length, of a bacterial gene selected from the group consisting of the genes corresponding to SEQ ID NO. 1-3, 8, 11-20, 31-48, 59-68, 71, 76-87, 92-97, and 100-105.

24. An oligonucleotide probe at least 15 nucleotides in length which specifically hybridizes to a nucleotide sequence which is the same as or complementary to a bacterial gene selected from the group consisting of the genes corresponding to SEQ ID NO. 1-3, 8, 11-20, 31-48, 59-68, 71, 76-87, 92-97, and 100-105.

25. An isolated or purified DNA sequence at least 15 nucleotides in length, comprising a nucleotide base sequence which is the same as or complementary to a portion of the base sequence of a bacterial gene corresponding to SEQ ID NO. 1-3, 8, 11-20, 31-48, 59-68, 71, 76-87, 92-97, and 100-105.

26. A DNA sequence of claim 25, the base sequence of which is the same as or complementary to the base sequence of the coding region of a bacterial gene selected from the group consisting of the genes corresponding to SEQ ID NO. 1-3, 8, 11-20, 31-48, 59-68, 71, 76-87, 92-97, and 100-105.

27. An isolated or purified DNA sequence, the base sequence of which is the same as or complementary to a bacterial gene which is homologous to a bacterial gene selected from the group consisting of the genes corresponding to SEQ ID NO. 1-105,
wherein the function of the expression product of said homologous gene is the same as the function of the product of said gene selected from the group consisting of the genes corresponding to SEQ ID NO. 1-105.

28. An isolated or purified DNA sequence, the base sequence of which is the same as the base sequence of a mutated bacterial gene selected from the group consisting of the genes corresponding to SEQ ID NO. 1-105,
wherein expression of said DNA sequence or of said mutated bacterial gene confers a growth conditional phenotype in the absence of expression of a gene which complements said mutation.

29. A purified, enriched, or isolated polypeptide encoded by a gene selected from the group consisting of the genes corresponding to SEQ ID NO. 1-3, 8, 11-20, 31-48, 59-68, 71, 76-87, 92-97, and 100-105.

30. The polypeptide of claim 29, wherein said polypeptide is expressed from a recombinant gene.
